(19) United States
(12) Patent Application Publication
(10) Pub. No.: US 2024/0000938 A1
(43) Pub. Date: Jan. 4, 2024

(54) ADOPTIVE CELL THERAPY FOR TREATMENT OF CANCER ASSOCIATED WITH LOSS OF HETEROZYGOSITY

(71) Applicant: A2 Biotherapeutics, Inc., Agoura Hills, CA (US)

(72) Inventors: Carl Alexander Kamb, Thousand Oaks, CA (US); Agnes Hamburger, Newbury Park, CA (US); Breanna Diandreth, Agoura Hills, CA (US); Han Xu, Agoura Hills, CA (US); Julyun Oh, Agoura Hills, CA (US)

(21) Appl. No.: 18/253,721

(22) PCT Filed: Nov. 23, 2021
(86) PCT No.: PCT/US2021/060607

§ 371 Date: May 19, 2023

Related U.S. Application Data
(60) Provisional application No. 63/117,893, filed on Nov. 24, 2020.
(60) Provisional application No. 63/131,731, filed on Dec. 29, 2020.
(60) Provisional application No. 63/141,372, filed on Jan. 25, 2021.
(60) Provisional application No. 63/166,765, filed on Mar. 26, 2021.
(60) Provisional application No. 63/175,987, filed on Apr. 16, 2021.

(51) Int. Cl. A61K 39/00; C07K 14/705; C07K 14/725; C07K 16/28; C07K 16/32; C07K 16/30; A61K 45/06; A61P 35/00; C12N 15/86; C12N 15/11; C12N 9/22

(52) U.S. Cl. CPC A61K 39/4632; C07K 14/70503; C07K 14/7051; C07K 16/2863; C07K 16/32; C07K 16/3007; C07K 16/2803; A61K 45/06; A61K 39/4611; A61K 39/4612; A61K 39/4613; A61K 39/464482; A61K 39/464468; A61K 39/464404; A61P 35/00; C12N 15/86; C12N 15/111; C12N 9/22; C07K 2319/03; A61K 2239/21; A61K 2239/22; A61K 2239/13; C12N 2310/20; C12N 2740/15043

(57) ABSTRACT

The disclosure relates to immune cells comprising systems of two engineered receptors each having a ligand binding domain, collectively designed to target cells identified by loss of heterozygosity and used to treat a disease or disorder, for example, cancer. The disclosure provides immune cells expressing two engineered receptors, methods of making same, and polynucleotides and vectors encoding same.

### CROSS-REFERENCE TO RELATED APPLICATION

This application claims the benefit of U.S. Provisional Patent Applications No. 63,117,893, filed Nov. 24, 2020; No. 63/131,731, filed Dec. 29, 2020; No. 63/141,372, filed Jan. 25, 2021; No. 63/175,987, filed Apr. 16, 2021; and U.S. Pat. No. 63,166,765, filed Mar. 26, 2021, each of which is incorporated by reference herein in its entirety.

### INCORPORATION BY REFERENCE OF SEQUENCE LISTING

The present application is being filed with a Sequence Listing in electronic format. The Sequence Listing is provided as a file entitled A2BI-032_01WO_SeqList_ST25.txt, created on Nov. 22, 2021 and is 4.94 MB in size. The information in electronic format of the Sequence Listing is incorporated by reference in its entirety.

### BACKGROUND

Cell therapy is a powerful tool for the treatment of various diseases, particularly cancers. In conventional adoptive cell therapies, immune cells are engineered to express specific receptors, for example chimeric antigen receptors (CARs) or T Cell Receptors (TCRs), which direct the activity of the immune cells to cellular targets via interaction of the receptor with a ligand expressed by the target cell. Transplant of donor-derived T cells may be used to treat solid tumors and hematological malignancies. For example, T cells genetically modified to express a chimeric antigen receptor specific to CD19 effectively treat B-cell lymphoma when transplanted into patients. Specificity of transplanted T cells for target cells, such as cancer cells, can be increased by modifying the cells to express not only a first activator receptor specific to a target cell type, but also a second inhibitory receptor that prevents activation of the immune cells by non-target cells that express the ligand for the inhibitory receptor. The presence of an inhibitory receptor can result in unintentional inactivation or activation of the immune cell through autocrine binding/signaling mechanisms, or failure of the inhibitory mechanism by in cis blocking from the endogenous blocker antigen (e.g., the antigen for the inhibitory receptor). Autocrine signaling-mediated inactivation/activation of an immune cell used in cell therapy reduces the therapy's efficacy or raises the risk of unchecked activation. Thus, there remains a need for immune cells that express inhibitory receptors and are not functionally affected through autocrine signaling mechanisms.

Immune cell products expressing this dual receptor system may be made using cells from a donor other than the patient who will receive the immune cell product. When the transplanted immune cells are not derived from the recipient, the transplant is termed an “allogeneic” transplant. Allogeneic transplant is reviewed in Ruella et al. BioDrugs. 31:473-81 (2017) PMID: 29143249; Kim et al. Biomolecules. 10:263 (2020) PMID: 32050611; and Yang et al. Curr Opin Hematol. 22:509-15 (2015) PMID: 26390167.

Allogeneic transplant of immune cells can cause complications in the recipient, decreasing the effectiveness of adoptive cell therapy and causing potentially life threatening side effects. For example, transplanted allogeneic T cells can view healthy cells of the recipient as foreign, which leads to a cytotoxic T-cell lymphocyte (CTL) response against the cells of the recipient (Graft versus Host Disease, or GvHD). In addition, the recipient's own immune system may recognize the transplanted allogeneic cells as foreign, and eliminate the transplanted cells (Host versus Graft Disease, or HvGD).

The disclosure provides compositions and methods to reduce autocrine signaling in, increase the efficacy and safety of, and reduce potential complications of, allogeneic immune cells that have been engineered to express a combination of activator and inhibitor receptors that specifically direct the transplanted allogeneic immune cells to target particular target cells, such as cancer cells.

### SUMMARY

The disclosure provides an allogeneic immune cell comprising: (a) a first engineered receptor, the first engineered receptor comprising a transmembrane region and an extracellular region, the extracellular region comprising a first ligand binding domain capable of specifically binding a first ligand; and (b) a second engineered receptor, the second engineered receptor comprising a transmembrane region and an extracellular region, the extracellular region comprising a second ligand binding domain capable of specifically binding a second ligand, wherein binding of the first ligand binding domain to the first ligand activates or promotes activation of the immune cell by the first receptor, wherein binding of the second ligand binding domain to the second ligand inhibits activation of the immune cell by the first receptor, and wherein the second ligand is expressed by a host immune cell.

In some embodiments of the allogeneic immune cells of the disclosure, the allogeneic immune cell expresses one or more endogenous T cell receptors (TCRs). In some embodiments, the allogeneic immune cell has not been modified to reduce or eliminate the expression of an endogenous TRCA, TRB, CD3D, CD3E, CD3G and/or CD3Z gene product.

In some embodiments, binding of the second ligand binding domain to the second ligand inhibits activation of the immune cell by the endogenous TCR.

In some embodiments, expression of the second engineered receptor reduces graft versus host disease when a plurality of the allogeneic immune cells are administered to a subject.

In some embodiments, the allogeneic immune cell comprises a first modification that reduces or eliminates expression or function of a component of the major histocompatibility complex class I (MHC I). In some embodiments, the component of MHC I is human leukocyte antigen A (HLA-A), human leukocyte antigen B (HLA-B), human leukocyte antigen C (HLA-C) or beta-2-microglobulin (B2M). In some embodiments, the first modification comprises a genetic modification of a HLA-A, HLA-B, HLA-C or B2M locus of the allogeneic immune cell genome. In some embodiments, the genetic modification comprises a deletion, insertion, substitution or frameshift mutation in the HLA-A, HLA-B, HLA-C or B2M locus. In some embodiments, the first modification reduces expression of a functional protein encoded by the HLA-A, HLA-B, HLA-C or B2M locus.

In some embodiments, the first modification comprises using a nucleic acid guided endonuclease, a zinc finger nuclease or a TALEN. In some embodiments, the nucleic acid guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasY, Csx3, Csx1, Csx15, Csf1, Csf2, Csf3, and Csf4. In some embodiments, the nucleic acid guided endonuclease is Cas9.

In some embodiments, the allogeneic immune cell is modified with a nucleic acid guided endonuclease in a complex with a guide nucleic acid (gNA) that specifically targets a sequence of the HLA-A, HLA-B, HLA-C or B2M locus. In some embodiments, the allogeneic immune cell is modified with a nucleic acid guided endonuclease in a complex with at least one guide gNA that specifically targets a sequence within the B2M locus and/or a promoter of the B2M gene. In some embodiments, the at least one gNA comprises a sequence that shares about 80%, about 85%, about 90%, about 95%, about 96%, about 97%, about 98%, or about 99% identity to a sequence selected from the group consisting of SEQ ID NOs: 8357-8470. In some embodiments, the at least one gNA comprises a sequence selected from the group consisting of SEQ ID NOs: 8357-8470. In some embodiments, the at least one gNA specifically targets a coding sequence (CDS) of the B2M gene. In some embodiments, the at least one gNA comprises a sequence that shares about 80%, about 85%, about 90%, about 95%, about 96%, about 97%, about 98%, or about 99% identity to a sequence selected from the group consisting of SEQ ID NOs: 8357-8397. In some embodiments, the gNA comprises a sequence selected from the group consisting of SEQ ID NOs: 8357-8397. In some embodiments, the gNA comprises a sequence selected from the group consisting of SEQ ID NOs: 8357-8365.

In some embodiments, the allogeneic immune cell is modified with a nucleic acid guided endonuclease in a complex with at least one guide gNA that specifically targets a sequence within the HLA-A locus and/or a promoter of the HLA-A gene. In some embodiments, the at least one gNA is specific to a target sequence that shares about 90%, about 95%, about 96%, about 97%, about 98%, or about 99% identity to a sequence selected from the group consisting of SEQ ID NOs: 390-3276. In some embodiments, the at least one gNA is specific to a target sequence selected from the group consisting of SEQ ID NOs: 390-3276. In some embodiments, the allogeneic immune cell is modified with a nucleic acid guided endonuclease in a complex with at least one gNA that specifically targets a sequence of HLA-A*02 alleles. In some embodiments, the at least one gNA is specific to a target sequence that shares about 90%, about 95%, about 96%, about 97%, about 98%, or about 99% identity to a sequence selected from the group consisting of SEQ ID NOs: 390-1585. In some embodiments, the at least one gNA is specific to a target sequence selected from the group consisting of SEQ ID NOs: 390-1585. In some embodiments, the allogeneic immune cell is modified with a nucleic acid guided endonuclease in a complex with at least one gNA that specifically targets a sequence of HLA-A*02:01 alleles. In some embodiments, the at least one gNA is specific to a sequence that shares about 90%, about 95%, about 96%, about 97%, about 98%, or about 99% identity to target a sequence selected from the group consisting of SEQ ID NOs: 390-1174. In some embodiments, the at least one gNA is specific to a target sequence selected from the group consisting of SEQ ID NOs: 390-1174. In some embodiments, the allogeneic immune cell is modified with a nucleic acid guided endonuclease in a complex with at least one gNA that specifically targets a sequence of HLA-A*02:01:01 alleles. In some embodiments, the at least one gNA is specific to a target sequence that shares about 90%, about 95%, about 96%, about 97%, about 98%, or about 99% identity to a sequence selected from the group consisting of SEQ ID NOs: 390-1166. In some embodiments, the at least one gNA is specific to a target sequence selected from the group consisting of SEQ ID NOs: 390-1166. In some embodiments, the allogeneic immune cell is modified with a nucleic acid guided endonuclease in a complex with at least one gNA that specifically targets a sequence of HLA-A*02:01:01:01 alleles. In some embodiments, the at least one gNA is specific to a target sequence that shares about 90%, about 95%, about 96%, about 97%, about 98%, or about 99% identity to a sequence selected from the group consisting of SEQ ID NOs: 390-1126. In some embodiments, the at least one gNA is specific to a target sequence selected from the group consisting of SEQ ID NOs: 390-1126. In some embodiments, the allogeneic immune cell is modified with a nucleic acid guided endonuclease in a complex with at least one guide nucleic acid (gNA) that specifically targets a coding DNA sequence of HLA-A*02. In some embodiments, the at least gNA is specific to a target sequence that shares about 90%, about 95%, about 96%, about 97%, about 98%, or about 99% identity to a sequence selected from the group consisting of SEQ ID NOs: 390-509. In some embodiments, the at least one gNA is specific to a target sequence selected from the group consisting of SEQ ID NOs: 390-509.

the immune cell is modified with a nucleic acid guided endonuclease in a complex with at least one gNA that specifically targets a coding DNA sequence that is shared by more than 1000 HLA-A*02 alleles. In some embodiments, the at least one gNA is specific to a target sequence that shares about 90%, about 95%, about 96%, about 97%, about 98%, or about 99% identity to a sequence selected from the group consisting of SEQ ID NOs: 390-455. In some embodiments, the at least one gNA is specific to a target sequence selected from the group consisting of SEQ ID NOs: 390-455. In some embodiments, the at least one gNA is specific to a target sequence selected from the group consisting of SEQ ID NOs: 426, 394, 407-408, 414, 423, 421-422, 429, 433, 435,438, 440, 448, 451, and 454.

In some embodiments, the first modification comprises expression of an interfering RNA. In some embodiments, the interfering RNA is a small interfering RNA (siRNA), a short hairpin RNA (shRNA) or a microRNA. In some embodiments, the interfering RNA comprises a sequence complementary to a target sequence of HLA-A, HLA-B, HLA-C or B2M. In some embodiments, the target sequence of HLA-A, HLA-B, HLA-C or B2M is between 18 and 27 bp in length.

In some embodiments, the interfering RNA comprises an shRNA capable of inducing RNAi-mediated degradation of an HLA-A*02:01:01 mRNA.

In some embodiments, the shRNA comprises (a) a first sequence, having from 5′ end to 3′ end a sequence complementary to the HLA-A*02:01:01:01 mRNA; and (b) a second sequence, having from 5′ end to 3′ end a sequence complementary to the first sequence, wherein the first sequence and the second sequence form the shRNA. In some embodiments, the first sequence is 18, 19, 20, 21, or 22 nucleotides. In some embodiments, the first sequence is complementary to a sequence selected from SEQ ID NOs: 8476-16870. In some embodiments, the first sequence and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA. In some embodiments, the loop region comprises a sequence selected from SEQ ID NOs: 16872-16884 and 16895. In some embodiments, the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence. In some embodiments, the 5′ flank sequence is selected from the group consisting of SEQ ID NO: 16885-16887. In some embodiments, the 3′ flank sequence is selected from the group consisting of SEQ ID NO: 16888, 16889, and 16896.

In some embodiments, the interfering RNA comprises an shRNA capable of inducing RNAi-mediated degradation of a B2M mRNA. In some embodiments, the shRNA comprises (a) a first sequence, having from 5′ end to 3′ end a sequence complementary to the B2M mRNA; and (b) a second sequence, having from 5′ end to 3′ end a sequence complementary to the first sequence, wherein the first sequence and the second sequence form the shRNA. In some embodiments, the first sequence is complementary to a sequence selected from SEQ ID NOs: 16897-21508, 847-8474, and 8368-8370. In some embodiments, the first sequence is complementary to a sequence selected from the group consisting of SEQ ID NOs: 16897-20484. In some embodiments, the first sequence is complementary to a sequence selected from the group consisting of SEQ ID NOs: 16897-19888. In some embodiments, the first sequence is complementary to a sequence selected from the group consisting of SEQ ID NOs: 16897-17478. In some embodiments, the first sequence is selected from the group consisting of SEQ ID NOs: 16897-17178 or SEQ ID NOs: 16897-17034. In some embodiments, the first sequence and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA. In some embodiments, the loop region comprises a sequence selected from SEQ ID NOs: 16872-16884, and 16895. In some embodiments, the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence. In some embodiments, the 5′ flank sequence is selected from SEQ ID NO: 16885-16887 and 16894. In some embodiments, the 3′ flank sequence is selected from SEQ ID NO: 16888, 16889, and 16896. In some embodiments, the shRNA comprises SEQ ID NOs: 21899-21901.

In some embodiments, the interfering RNA is operably linked to a promoter.

In some embodiments, the allogeneic immune cell comprises a second modification that reduces or eliminates expression or function of CD52. In some embodiments, the second modification comprises a deletion, insertion, substitution or frameshift mutation in the CD52 locus of the allogeneic immune cell genome. In some embodiments, the second modification comprises using a nucleic acid guided endonuclease, a zinc finger nuclease or a TALEN. In some embodiments, the second modification comprises expression of an interfering RNA. In some embodiments, the interfering RNA is a small interfering RNA (siRNA), a short hairpin RNA (shRNA) or a microRNA. In some embodiments, the interfering RNA comprises a sequence complementary to a target sequence of CD52.

In some embodiments, the allogeneic immune cell comprises a third modification that reduces targeting of the allogeneic immune cell by NK cells of a subject. the allogeneic immune cell comprises the third modification comprises overexpression of HLA-E, HLA-G or NKG2A.

In some embodiments, the second ligand is not expressed in a target cell due to loss of heterozygosity of a gene encoding the second ligand.

In some embodiments, the first ligand and second ligand are not the same.

In some embodiments, the first ligand is expressed by target cells. In some embodiments, the first ligand is expressed by target cells and a plurality of non-target cells. In some embodiments, the plurality of non-target cells express both the first and second ligands. In some embodiments, the second ligand is not expressed by the target cells, and is expressed by the plurality of non-target cells. In some embodiments, the target cells are cancer cells and the non-target cells are non-cancerous cells.

In some embodiments, the first ligand is selected from the group consisting of a cell adhesion molecule, a cell-cell signaling molecule, an extracellular domain, a molecule involved in chemotaxis, a glycoprotein, a G protein-coupled receptor, a transmembrane protein, a receptor for a neurotransmitter and a voltage gated ion channel, or a peptide antigen thereof. In some embodiments, the first ligand is a cancer antigen. In some embodiments, the first ligand is selected from the group of antigens in Table 1. In some embodiments, the first ligand binding domain is isolated or derived from the antigen binding domain of an antibody in Table 1. In some embodiments, the first ligand is selected from the group consisting of transferrin receptor (TFRC), epidermal growth factor receptor (EGFR), CEA cell adhesion molecule 5 (CEA), CD19 molecule (CD19), erb-b2 receptor tyrosine kinase 2 (HER2), and mesothelin (MSLN), or a peptide antigen thereof. In some embodiments, the first ligand is a pan-HLA ligand. In some embodiments, the first ligand comprises HLA-A, HLA-B, HLA-C, HLA-E, HLA-F, of HLA-G.

In some embodiments, the first engineered receptor is a T cell receptor (TCR) or a chimeric antigen receptor (CAR).

In some embodiments, the second engineered receptor is a T cell receptor (TCR) or a chimeric antigen receptor (CAR).

In some embodiments, the first ligand binding domain comprises a single chain Fv antibody fragment (ScFv) or a β chain variable domain (Vβ). In some embodiments, the first ligand binding domain comprises a TCR α chain variable domain and a TCR β chain variable domain. In some embodiments, the first ligand binding domain comprises a variable heavy chain (VH) domain and a variable light chain (VL) domain. In some embodiments, the first ligand is EGFR or a peptide antigen thereof, and the first ligand binding domain comprises a sequence of SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115 or SEQ ID NO: 381, or a sequence having at least 90%, at least 95% or at least 99% identity thereto. In some embodiments, the first ligand is MSLN or a peptide antigen thereof, and the first ligand binding domain comprises a sequence of SEQ ID NO: 83, SEQ ID NO: 85, SEQ ID NO: 87 or SEQ ID NO: 89, or a sequence having at least 90%, at least 95% or at least 99% identity thereto. In some embodiments, the first ligand is CEA or a peptide antigen thereof, and the first ligand binding domain comprises SEQ ID NO: 91, SEQ ID NO: 93, SEQ ID NO: 95, SEQ ID NO: 97, SEQ ID NO: 273, SEQ ID NO: 275, or SEQ ID NO: 277, or a sequence having at least 90%, at least 95% or at least 99% identity thereto. In some embodiments, the first ligand is CD19 or a peptide antigen thereof, and the first ligand binding domain comprises SEQ ID NO: 266 or SEQ ID NO: 268, or a sequence having at least 90%, at least 95% or at least 99% identity thereto. In some embodiments, the first ligand comprises a pan-HLA ligand, and the first ligand binding domain comprises a sequence of SEQ ID NO: 163, SEQ ID NO: 165, SEQ ID NO: 167, SEQ ID NO: 169, SEQ ID NO: 171, or SEQ ID NO: 173, or a sequence having at least 90%, at least 95% or at least 99% identity thereto. In some embodiments, the first ligand comprises EGFR or a peptide antigen thereof, and the first ligand binding domain comprises CDRs selected from SEQ ID NOs: 129-162. In some embodiments, the first ligand comprises a CEA ligand, or a peptide antigen thereof, and the first ligand binding domain comprises CDRs selected from SEQ ID NOs: 285-293.

In some embodiments, the second ligand binding domain comprises an ScFv, a VP domain, or a TCR α chain variable domain and a TCR β chain variable domain. In some embodiments, the second ligand binding domain comprises a variable heavy chain (VH) domain and a variable light chain (VL) domain. In some embodiments, the second ligand comprises an HLA-A*02 allele, and wherein the second ligand binding domain comprises any one of SEQ ID NOs: 50-61 or a sequence having at least 90%, at least 95%, or at least 99% identity thereto. In some embodiments, the second ligand comprises an HLA-A*02 allele, and the second ligand binding domain comprises CDRs selected from SEQ ID NOs: 39-49.

In some embodiments, the second engineered receptor comprises at least one immunoreceptor tyrosine-based inhibitory motif (ITIM). In some embodiments, the second engineered receptor comprises a LILRB1 intracellular domain or a functional variant thereof. In some embodiments, the LILRB1 intracellular domain comprises a sequence at least 95% identical to SEQ ID NO: 73. In some embodiments, the second engineered receptor comprises a LILRB1 transmembrane domain or a functional variant thereof. In some embodiments, the LILRB1 transmembrane domain or a functional variant thereof comprises a sequence at least 95% identical to SEQ ID NO: 82. In some embodiments, the second engineered receptor comprises a LILRB1 hinge domain or functional fragment or variant thereof. In some embodiments, the LILRB1 hinge domain comprises a sequence at least 95% identical to SEQ ID NO: 81, SEQ ID NO: 74 or SEQ ID NO: 75. In some embodiments, the second engineered receptor comprises a LILRB1 intracellular domain and a LILRB1 transmembrane domain, or a functional variant thereof. In some embodiments, the LILRB1 intracellular domain and LILRB1 transmembrane domain comprises SEQ ID NO: 77 or a sequence at least 95% identical to SEQ ID NO: 77. In some embodiments, the second inhibitory receptor comprises a sequence of SEQ ID NO: 21902 or a sequence having at least 90%, at least 95% or at least 99% identity thereto.

In some embodiments, the immune cell is selected form the group consisting of T cells, B cells and Natural Killer (NK) cells. In some embodiments, the immune cell is non-natural. In some embodiments, the immune cell is isolated.

The disclosure provides allogeneic immune cells of the disclosure, for use as a medicament. In some embodiments, the medicament is for the treatment of cancer in a subject.

The disclosure provides pharmaceutical compositions comprising a plurality of the allogeneic immune cells of the disclosure. In some embodiments, the pharmaceutical compositions comprise a pharmaceutically acceptable carrier, diluent or excipient. In some embodiments, the pharmaceutical compositions comprise a therapeutically effective amount of the allogeneic immune cells.

The disclosure provides methods of increasing the specificity of an adoptive cell therapy in a subject, comprising administering to the subject a plurality of the allogeneic immune cells of or pharmaceutical composition of the disclosure.

The disclosure provides methods of treating a subject with cancer with an adoptive cell therapy, comprising administering to the subject a plurality of the allogeneic immune cells of or pharmaceutical composition of the disclosure. In some embodiments, cells of the cancer express the first ligand. In some embodiments, cells of the cancer do not express the second ligand due to loss of heterozygosity. In some embodiments, non-target cells express both the first ligand and the second ligand. In some embodiments, immune cells of the subject express the second ligand. In some embodiments, the methods comprise administering a lymphodepletion agent to the subject. In some embodiments, the lymphodepletion agent specifically targets CD52.

The disclosure provides methods of making the allogeneic immune cell of the disclosure, comprising (a) providing a plurality of allogeneic immune cells; and (b) contacting the immune cells with a vector comprising sequences encoding: (i) a first engineered receptor comprising a transmembrane region and an extracellular region, the extracellular region comprising a first ligand binding domain capable of specifically binding a first ligand, and (ii) a second engineered receptor comprising a transmembrane region and an extracellular region, the extracellular region comprising a second ligand binding domain capable of specifically binding a second ligand;

wherein binding of the first ligand binding domain to the first ligand activates or promotes activation of the immune cell, and wherein binding of the second ligand binding domain to a second ligand inhibits activation of the immune cell by the first ligand. In some embodiments, the sequences of the first and second engineered receptors are operably linked to a first promoter. In some embodiments, the vector further comprises a sequence encoding a self-cleaving peptide between the sequence encoding the first engineered receptor and the sequence encoding the second engineered receptor. In some embodiments, the vector further comprises a sequence encoding a B2M or HLA-A shRNA operably linked to a sequence promoter. In some embodiments, the vector further comprises a sequence encoding a guide nucleic acid (gNA) comprising a targeting sequence specific to a B2M or HLA-A*02 target sequence, wherein the sequence encoding the gNA is operably linked to a second promoter. In some embodiments, the vector is a lentiviral vector, and contacting the immune cells with the vector comprises transducing the immune cells. In some embodiments, the methods further comprise transfecting the immune cells with a Cas9 protein or a nucleic acid comprising a sequence encoding a Cas9 protein.

The disclosure provides kits comprising the allogeneic immune cells or pharmaceutical compositions of the disclosure. In some embodiments, the kits further comprise instructions for use.

The disclosure provides vectors comprising: (a) a sequence encoding a first engineered receptor, the first engineered receptor comprising a transmembrane region and an extracellular region, the extracellular region comprising a first ligand binding domain capable of specifically binding a first ligand; (b) a self-cleaving polypeptide sequence; and (c) a sequence encoding second engineered receptor, the second engineered receptor comprising a transmembrane region and an extracellular region, the extracellular region comprising a second ligand binding domain capable of specifically binding a second ligand, wherein binding of the first ligand binding domain to the first ligand activates or promotes activation of the immune cell by the first receptor, and wherein binding of the second ligand binding domain to the second ligand inhibits activation of the immune cell by the first receptor.

The disclosure provides vectors comprising: (a) a first promoter operably linked to (i) a sequence encoding a first engineered receptor, the first engineered receptor comprising a transmembrane region and an extracellular region, the extracellular region comprising a first ligand binding domain capable of specifically binding a first ligand, (ii) a self-cleaving polypeptide sequence, and (iii) a sequence encoding second engineered receptor, the second engineered receptor comprising a transmembrane region and an extracellular region, the extracellular region comprising a second ligand binding domain capable of specifically binding a second ligand; and (b) a second promoter operably linked to a sequence encoding a guide nucleic acid or an short interfering RNA (shRNA) capable of reducing expression of HLA-A or B2M by an immune cell; wherein binding of the first ligand binding domain to the first ligand activates or promotes activation of the immune cell by the first receptor, wherein binding of the second ligand binding domain to the second ligand inhibits activation of the immune cell by the first receptor.

The disclosure provides an immune cell comprising an inhibitory receptor comprising a ligand binding domain specific to a class I major histocompatibility complex (MHC-I) molecule, or a peptide-MHC complex thereof; wherein the immune cell comprises one or more modifications that reduce autocrine binding/signaling by the receptor. In some embodiments, the immune cell further comprises an activator receptor as described herein. In some embodiments, the one or more modifications comprise an inactivating mutation in an endogenous gene encoding an allele of an endogenous MHC class I polypeptide specifically bound by the inhibitory receptor. In some embodiments, the one or more modifications comprise expression of an interfering RNA, wherein expression and/or function of a human leukocyte antigen (HLA) polypeptide, or an allele thereof, or B2M in said immune cell has been reduced or eliminated by expression of the interfering RNA. In some embodiments, the immune cell is autologous.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a diagram illustrating hemizygous tumor cells forming a tumor against a background of heterozygous cells that compose normal tissue. The hemizygous tumor cells express only Target A and have lost Target B due to loss of heterozygosity (LOH), while the normal cells express both Target A and Target B. This genetic difference can be exploited to create tumor-selective cytotoxic therapeutics that are blocked by Target B and activated by Target A, thereby selectively killing tumors.

FIG. 2A is a diagram showing an exemplary architecture of a dual targeted therapeutic based on LOH in tumors. In this example, there is cell-based integration of activator and blocker signals.

FIG. 2B is a series of diagrams showing various activator and receptor formats and combinations.

FIG. 3A is a pair of diagrams that show exemplary dual receptor constructs of the disclosure in TCR format. In this example, activator and inhibitor (blocker) ligand binding domains (LBDs) are each fused separately to the CD3 gamma subunit of the TCR.

FIG. 3B is diagram and a table that show exemplary dual receptor constructs of the disclosure in CAR format. Exemplary ITIM and inhibitor domains of the inhibitor CAR are shown in the table at right.

FIG. 4A is a plot showing the RNA-Seq expression of the transferrin receptor (TFRC) in human tissues from the GTEx database. Transferrin receptor (TFRC) is a candidate for Target A (the activator). Expression of TFRC at the RNA level is ubiquitous and relatively even. TRFC is an essential gene: Loss-of-function homozygous mutations are embryonic lethal in mice.

FIG. 4B is a plot showing the RNA-Seq expression profiles of HLA-A and HLA-B.

FIGS. 5A-5H show that the LIR-1 blocker receptor is modular and mediates large EC50 shifts. FIG. 5A shows schematic of T2-Jurkat experiments to evaluate blocker constructs. FIG. 5B shows the effect of various NY-ESO-1 scFv LBD blocker modules (PD-1, CTLA-4, LIR-1) on EC50 of MAGE-A3 CAR activator (MP1-CAR) when loaded with NY-ESO-1 blocker peptide. Error bars indicate±SD (n=2). FIG. 5C shows the effect of an LIR-1 blocker module with various scFv LBDs (ESO, MP1 LBD 1, MP1 LBD 2, HPV E6 LBD 1, HPV E6 LBD 2, HPV E7) on EC50 of MAGE-A3 CAR activator (MP1-CAR) when loaded with corresponding peptide. Error bars indicate±SD (n=2). FIG. 5D shows the effect of an LIR-1 blocker module with NY-ESO-1 scFv LBD on EC50 of different MAGE-A3 CAR activators (MP1-CAR or MP2-CAR) when loaded with NY-ESO-1 blocker peptide. Error bars indicate±SD (n=2). FIG. shows the effect of an LIR-1 blocker module with NY-ESO-1 scFv LBD on EC50 of different TCR activators (MP1-TCR, MP2-TCR, HPV E6-TCR) when loaded with NY-ESO-1 blocker peptide. Error bars indicate±SD (n=2). Three different TCR activators are blocked by NY-ESO-LIR-1, which has an ESO scFv, LIR-1 hinge, LIR-1 TM and LIR-1 ICD. FIG. 5F shows the effect of an LIR-1 blocker module with NY-ESO-1 Ftcr LBDs on EC50 of MAGE-A3 CAR and TCR activators (MP1-CAR, MP1-TCR). Error bars indicate±SD (n=2). Both a third generation CAR activator or a regular TCR activator can be blocked by NY-ESO-1 Ftcr-LIR-1, which has TCRa ECD, LIR-1 TM, a LIR-1 ICD and a TCRb ECD, LIR-1 TM and LIR-1 ICD. FIG. 5G is a pair of diagrams (top and left) and a pair of plots that show that Jurkat cells transfected with either HPV E7-CAR or HPV E7-CAR & A2-LIR-1 co-cultured with beads displaying various ratios of activator (HPV E7) and blocker (NY-ESO-1) antigen demonstrates blocking in cis but not trans. FIG. 5H shows that the A2-LIR-1 blocker module blocks CD19-CAR activator at various activator to blocker ratios. E:T ratio: effector:target ratio.

FIGS. 6A-6E show that primary T cells expressing a LIR-1 blocker receptor selectively kill tumor cells with pMHC and non-pMHC proof-of-concept targets. FIG. 6A shows primary T cells transduced with HPV E7-TCR activator and ESO-LIR-1 blocker shifts EC50˜100 fold in primary T cell killing assay. Error bars indicate+/−SD (n=2). FIG. 6B shows that HLA-A*02-LIR-1 blocks NY-ESO-1 CAR activator at various activator:blocker DNA ratios in Jurkat cells. FIG. 6C shows that primary T cells transduced with CD19 CAR activator and HLA-A*02 blocker distinguish “tumor” cells from “normal” cells in in vitro cytotoxicity assay and demonstrate selective killing of “tumor” cells in mixed target cell assay at 3:1 E:T. A2-LIR-1: LIR-1 based receptor with an HLA-A2*02 LBD. FIGS. 6D-6E show that primary T cells transduced with CD19 CAR activator and HLA-A*02 blocker demonstrate reversible blockade (FIG. 6D) and activation (FIG. 6E) after 3 rounds of antigen exposure (AB-A-AB and A-AB-A) in an in vitro cytotoxicity assay at 3:1 E:T. The primary T cell cytotoxicity assay was reproduced with three HLA-A*02-negative donors.

FIGS. 7A-7E show that modified CAR-T cells (i.e., CAR-T cells expressing both an activator and a blocker receptor) selectively kill tumors in xenograft model. FIG. 7A shows primary T cells transduced with CD19 CAR activator and HLA-A*02 blocker demonstrate ˜20-fold expansion with CD3/28 stimulation over 10 days. FIG. 7B shows a schematic of in vivo study design: HLA-A*02 NSG mice were administered either “tumor cells” (A2-negative Raji cells) or “normal cells” (A2-positive Raji cells) subcutaneously and primary T cells (human, HLA-A*02-negative donor) were injected into the tail vein when Raji xenografts averaged ˜70 mm³. FIGS. 7C-7E show readouts of tumor size by caliper measurement (FIG. 7C), human blood T cell count by flow cytometry (FIG. 7D), and survival (FIG. 7E). Error bars are standard error of the mean (s.e.m.). UTD: untransduced.

FIG. 8 shows that the peptide-loading shift of activation EC50 is typically less than ˜10×. The effect of blocker peptide loading (50 uM each of NY-ESO-1, MAGE-A3, HPV E6, and HPV E7) on activating MAGE-A3 CAR (MP2 CAR) is shown.

FIG. 9 shows that the LIR-1 blocker receptor is ligand dependent. The effect of NY-ESO-1-LIR-1 blocker on EC50 of activating MAGE-A3 CAR (MP1-CAR) when loaded with various concentrations of NY-ESO-1 blocker peptide is shown.

FIG. 10 shows that blocker receptors without an intracellular domain (ICD) or with a mutated, non-functional ICD do not block activation. Effect of a modified LIR-1 blocker modules containing no ICD (blue) or a mutated ICD (purple) with NY-ESO-1 scFv LBD on EC50 of MAGE-A3 CAR activator (MP2-CAR) when loaded with 10 uM of NY-ESO-1 blocker peptide is shown.

FIG. 11 shows that CD19 activates & A2-LIR-1 blocks Jurkat activation in HLA-A*02+(A2+) Raji cells. Jurkat cells transfected with either CD19 or CD19 & A2-LIR-1 were co-cultured with either WT (A2−) Raji cells or A2+ Raji cells at various cell ratios.

FIG. 12 is four plots that show the correlation of hCD3+ T cells in mouse blood to tumor growth. Shown are graphs of hCD3+ T cells compared to tumor volume 10 days and 17 days after T cell injection with A2− and A2+ Raji cells. UTD: untransduced.

FIG. 13 shows that Jurkat cells expressing an EGFR CAR activator and an HLA-A*02 LIR-1 blocker are activated by EGFR+/HLA-A*02− HeLa target cells but not EGFR+/HLA-A*02+ HeLa target cells.

FIG. 14A shows the expression of HLA-A*02 on HeLa cells transduced with HLA-A*02, and HCT116 cells. HeLa and HCT1116 cells were labeled with the anti-HLA-A2 antibody BB7.2 and FACs sorted. Green: unlabeled HeLa; orange: unlabeled HCT116; blue: wild type HCT116 labeled with BB7.2; red: HeLa cells transduced with HLA-A*02 and labeled with BB7.2.

FIG. 14B shows expression of EGFR on HeLa cells and HCT116 cells. HeLa and HCT1116 cells were labeled with anti-EGFR antibody and FACs sorted. Green: unlabeled HeLa; orange: unlabeled HCT1116; blue: wild type HCT116 labeled with anti-EGFR; red: HeLa cells transduced with HLA-A*02 and labeled with anti-EGFR.

FIG. 15A shows EGFR CAR activation of Jurkat cells expressing an EGFR CAR, and HCT116 target cells.

FIG. 15B shows that EGFR CAR activation of Jurkat cells can be blocked by an HLA-A*02 LIR-1 inhibitory receptor. Co-expression of the EGFR CAR and HLA-A*02 LIR-1 inhibitory receptor by Jurkat cells leads to a shift in the CAR E_{MAX} of approximately 1.8× when Jurkat cells are presented with HCT116 target cells expressing EGFR and HLA-A*02.

FIG. 16A shows titration of activator antigen in a bead-based assay to determine the optimal ratio of activator to blocker antigen.

FIG. 16B shows titration of blocker (inhibitory) antigen in the presence of a constant amount of activator antigen in a bead based assay to determine the optimal ratio of activator to blocker antigen.

FIG. 17 is a diagram (left) and a plot (right) showing that a NY-ESO-1 ScFv LIR-1 based inhibitory receptor can inhibit activation of Jurkat cell activation by a MP1 MAGE-A3 TCR using the solid tumor cell line A375 as target cells.

FIG. 18 is a diagram (left) and a plot (right) showing that a pMHC HLA-A*02 ScFv LIR-1 based inhibitory receptor can inhibit activation of Jurkat cell activation by a CD19 ScFv CAR using the B cell leukemia line NALM6 as target cells.

FIG. 19 is a diagram (left) and a plot (right) showing that a pMHC HLA-A*02 ScFv LIR-1 based inhibitory receptor can inhibit activation of Jurkat cells by a NY-ESO-1 ScFv CAR activator in a dose dependent manner.

FIG. 20 shows that a pan HLA (pan class I) ScFv CAR is blocked by expression of an HAL-A*02 LIR-1 blocker with tunable strength when assayed in Jurkat cells using T2 target cells and a luciferase assay.

FIG. 21A shows that a pMHC HLA-A*02 ScFv LIR-1 based inhibitory receptor can inhibit activation of Jurkat cells in cis in a cell-free bead based assay.

FIG. 21B that a pMHC HLA-A*02 ScFv LIR-1 based inhibitory receptor can inhibit activation of Jurkat cells by a MSLN ScFv CAR using the leukemia cell line K562 as target cells.

FIG. 22 is a diagram (left) and a chart (right) showing that a pMHC HLA-A*02 ScFv LIR-1 based inhibitory receptor can inhibit activation of Jurkat cells, as measured by fold induction of IFNγ, by a MSLN ScFv CAR using a pMHC HLA-A*02 ScFv LIR-1 based inhibitory receptor and HLA-A*02+ HeLa and SiHa cells as target cells.

FIG. 23 shows that a pMHC HLA-A*02 ScFv LIR-1 based inhibitory receptor inhibits killing by MSLN CAR activators using HLA-A*02+ SiHa cells but not HLA-A*02− SiHa cells.

FIG. 24 shows that activation of Jurkat cells expressing an EGFR ScFv CAR using a bead based assay can be blocked by a pMHC HLA-A*02 ScFv LIR-1 based inhibitory receptor when the activator and inhibitor antigens are present on beads in cis, but not when the activator and inhibitor antigens are present on the beads in trans.

FIG. 25A shows that activation of Jurkat cells by an EGFR ScFv CAR can be blocked by a pMHC HLA-A*02 ScFv LIR-1 based inhibitory receptor using SiHa target cells expressing HLA-A*02 (SiHa A02), but not by SiHa cells that do not express HLA-A*02 (SiHa WT).

FIG. 25B shows that activation of Jurkat cells by an EGFR ScFv CAR can be blocked by a pMHC HLA-A*02 ScFv LIR-1 based inhibitory receptor using HeLa target cells expressing HLA-A*02 (HeLa A02), but not by HeLa cells that do not express HLA-A*02 (HeLa WT).

FIG. 26 shows that additional ScFvs fused to a LIR-1 inhibitory domain inhibit a constitutive CAR activator in a dose dependent manner. Jurkat-NFAT luciferase reporter cells were transfected with an activating CAR construct that exhibits high tonic signaling and an inhibitory construct recognizing various pMHCs. The effect on activation of NFAT-luciferase was measured by co-culturing transfected Jurkat cells with T2 cells loaded with varying amounts of inhibiting peptide.

FIG. 27 is a diagram (left) and a plot (right) showing that an inhibitory receptor comprising a MiHA-b surrogate ScFv ligand binding domain (KRAS G12V ScFv-blocker) inhibits Jurkat effector cell activation by an activator TCR targeting a MiHA-a surrogate (KRAS G12D TCR, C-891), using T2 target cells.

FIG. 28 is a diagram (left) and a plot (right) showing that an inhibitory receptor comprising a MiHA-b surrogate ScFv ligand binding domain fused a LIR-1 hinge, TM and ICD (KRAS G12D ScFv-blocker) inhibits Jurkat effector cell activation by a TCR targeting a MiHA-a surrogate (KRAS G12V TCR, C-913), using T2 target cells.

FIG. 29 is a diagram (left) and a plot (right) showing that an inhibitory receptor comprising a MiHA-b surrogate Ftcr binding domain fused to a LIR1 TM and ICD (KRAS G12V Ftcr-blocker) inhibits Jurkat effector cell activation by a TCR targeting a MiHA-a surrogate (KRAS G12D TCR), using T2 target cells.

FIG. 30 is a diagram (left) and a plot (right) showing that an inhibitory receptor comprising a MiHA-b surrogate Ftcr binding domain fused to a LIR-1 TM and ICD (KRAS G12D Ftcr-blocker) inhibits Jurkat effector cell activation by a TCR targeting a MiHA-a surrogate (KRAS G12V TCR), using T2 target cells.

FIG. 31A is a plot showing inhibition of Jurkat cell activation by a MiHA-a TCR using an inhibitory receptor comprising a MiHA-b ScFv ligand binding domain that binds one mutant KRAS peptide [KRAS G1213] and a LIR-1 hinge, transmembrane domain and intracellular domain (ICD) that binds another mutant KRAS peptide (KRAS G12V). Black: C-891 activator; Blue: C-891 activator, C-1761 inhibitor; Red: C-891 activator, C-2371 and C2369 inhibitor.

FIG. 31B is a plot showing inhibition of Jurkat cell activation by a MiHA-a TCR using an inhibitory receptor comprising a MiHA-b Ftcr ligand binding domain and a LIR-1 transmembrane domain and intracellular domain (ICD).Black: C-913 activator; Blue: C-913 activator, C-1761 inhibitor; Red: C-913 activator, C2365 and C2367 inhibitor.

FIG. 32 is a plot showing that mouse MiHA-Y TCRs can activate Jurkat effector cells.

FIG. 33A is a plot and a table showing that an HA-1 Ftcr can block NY-ESO-1 TCR specifically in the presence of HA-1(H) peptide.

FIG. 33B is a plot and a table showing that there is essentially no blocking of NY-ESO-1 TCR by the HA-1 Ftcr in the presence of the non-specific, allelic variant HA-1(R) peptide.

FIG. 34A is a plot and a table showing that an HA-1 Ftcr can block a KRAS TCR specifically in the presence of HA-1(H) blocker peptide.

FIG. 34B is a plot and at table showing that there is essentially no blocking of a KRAS TCR by the HA-1 Ftcr in the presence of the non-specific, allelic variant HA-1(R) peptide.

FIG. 35 is a plot comparing peptide loading of HA-1(R), HA-1(H) and NY-ESO-1 peptides in T2 cells by flow cytometry.

FIG. 36A is a plot and a table showing an activation dose response using a MAGE-A3 MP1 ScFv CAR and a NY-ESO-1 ScFv LIR1 blocker.

FIG. 36B is a plot and a table showing an inhibition dose response using a MAGE-A3 MP1 ScFv CAR and a NY-ESO-1 ScFv LIR1 blocker.

FIG. 36C is a plot showing the x-value blocker NY-ESO-1 peptide concentrations from FIG. 36B that were normalized to the constant activator MAGE peptide concentrations used for each curve and plotted on the x-axis. B: NY-ESO-1 LIR1 blocker, A: MAGE-A3 peptide 2 ScFv CAR.

FIG. 37 is a series of plots and a table that shows that a different degree of blocking is observed when an HLA-A*02 ScFv LIR1 inhibitor is used with different EGFR ScFv CAR activators.

FIG. 38A is a series of fluorescence activated cell sorting (FACS) plots showing expression of EGFR ScFv CAR activator receptor by T cells following incubation of T cells expressing different EGFR ScFv CAR and an HLA-A*02 ScFv LIR1 inhibitor with HeLa cells expressing EGFR activator alone (Target A), inhibitor target alone (Target B) or activator and inhibitor targets (Target AB).

FIG. 38B is a plot showing quantification activator receptor expression before exposure to target cells, and after 120 hours co-culture with target cells expressing activator ligand alone (Target A), or target cells expressing both activator and blocker ligands (Target AB).

FIG. 39A is a plot showing cell surface expression of the activator receptor on T cells expressing an EGFR ScFv CAR (CT-482) activator and HLA-A*02 ScFv LIR1 inhibitor (C1765) following co-culture with to populations of HeLa cells expressing EGFR (Target A), HLA-A*02 (Target B), a combination of EGFR and HLA-A*02 on the same cell (Target AB), a mixed population of HeLa cells expressing Target A and Target AB on different cells, or a mixed population of HeLa cells expressing Target B and Target AB on different cells.

FIG. 39B is a plot showing cell surface expression of the inhibitor receptor on T cells expressing an EGFR ScFv CAR (CT-482) activator and HLA-A*02 ScFv LIR1 inhibitor (C1765) following co-culture with to populations of HeLa cells expressing EGFR (Target A), HLA-A*02 (Target B), a combination of EGFR and HLA-A*02 on the same cell (Target AB), a mixed population of HeLa cells expressing Target A and Target AB on different cells, or a mixed population of HeLa cells expressing Target B and Target AB on different cells.

FIG. 40 is a diagram of an experiment to determine if loss of expression of activator receptor by T cells was reversible.

FIG. 41A is a series of plots showing that activator surface loss of expression is reversible and corresponds to T cell cytotoxicity. At top: percent killing of target HeLa cells by T cells is shown. At bottom: activator and inhibitor receptor expression as assayed by FACS.

FIG. 41B is a series of plots showing that activator surface loss of expression is reversible and corresponds to T cell cytotoxicity. At top: percent killing of target HeLa cells by T cells is shown. At bottom: activator and inhibitor receptor expression as assayed by FACS.

FIG. 42 shows an embodiment of an effector T cell.

FIG. 43 is bar graphs demonstrating that the cytotoxic T-cell lymphocyte (CTL) response of engineered effector T cells is suppressed by normal cells (rounds 1, 3, 5 and 7). Exposure to tumor cells (rounds 2, 4, and 6) activates the CTL response. UTD: untransduced.

FIG. 44A is a set of plots showing that engineered receptors with HLA-A*02 blocker LBDs lose blocking ability in the presence of HLA-A*02 donors (1) and A*02-blocker is expressed but occupied by HLA-A*02 cis interaction (2). A*02-blocker binds to A*02 in cis and hinders blocker function in Jurkat and in primary T cells.

FIG. 44B is a diagram showing an autocrine signaling mechanism in an immune cell expressing an inhibitory receptor. The diagram shows that knockout of natively expressed MHC class I polypeptides reduces inhibition mediated by autocrine signaling.

FIG. 45 is a diagram showing the process for selecting HLA-A*02 targeting guide sequences.

FIG. 46 is a series of histograms and plots derived from FACS analysis of cells transfected with guide nucleic acids (gRNA) of the disclosure. The gRNA knockout efficiency (% indel) of HLA-A*02 was measured by staining Jurkat cells with fluorescently labeled anti-HLA-A*02 antibody (HLA-A*02 staining) following transfection with gRNA. Knockout efficiency of HLA-A, HLA-B, and HLA-C was also determined by staining the Jurkat cells with a fluorescently labeled anti-HLA-A/B/C antibody (Class I HLA staining). Predicted “on- and off-” target coverage for Class I HLA genes and off-targets is shown in the heatmap.

FIG. 47 is a series of FACS-derived histograms depicting rescue of HLA-A*02 ligand binding capacity in cells expressing an HLA-A*02-specific blocking receptor. gRNA-16 mediated knockout was performed in T cells from three HLA-A*02 positive (A*02+) donors (D A2-16, D 5886, and D 1042) and one HLA-A*02 negative (A*02−) donor. T cells were stained with an HLA-A*02 binding probe.

FIG. 48 is a schematic showing illustrative regions of the HLA-A*02 mRNA targeted by interfering shRNAs of the disclosure.

FIG. 49 is a series of histograms (left) and a plot (right) derived from fluorescence activated cell sorting (FACS) analysis of HLA-A*02 expressing Jurkat cells transfected with shRNA of the disclosure that target coding sequence (CDS) region of the HLA-A*02 mRNA. The shRNA knockdown efficiency of HLA-A*02 expression was measured by staining Jurkat cells with fluorescently labeled anti-HLA-A*02 antibody (HLA-A*02), following transfection with shRNA.

FIG. 50 is a series of histograms derived from fluorescence activated cell sorting (FACS) analysis of Jurkat cells co-transfected with HLA-A*02 and shRNA of the disclosure that target the 5′ and 3′ untranslated regions of HLA-A*02 mRNA. The shRNA-mediated reduction of HLA-A, HLA-B, and HLA-C(HLA Class I) expression was measured by staining Jurkat cells with fluorescently labeled anti-HLA-A/B/C antibody.

FIG. 51 is a pair of plots and inset histograms showing Jurkat cell activation and the binding capacity of the HLA-A*02 blocker module to its ligand, pMHC tetramer, in the presence (right) and absence (left) of HLA-A targeting shRNA.

FIG. 52 is a schematic showing gRNA targeting sequences of the disclosure mapped onto the target sequences of the B2M gene.

FIG. 53 is a pair of histogram series derived from fluorescence activated cell sorting (FACS) analysis of HLA-A*02 expressing Jurkat cells transfected with guide nucleic acids (gRNA) of the disclosure that target the B2M gene. The gRNA knockout efficiency of HLA-A*02 was measured by staining Jurkat cells with fluorescently labeled anti-HLA-A*02 antibody (HLA-A*02) following transfection with single guide RNA (sgRNA):Cas9 complexes. Knockout efficiency of HLA-A, HLA-B, and HLA-C(HLA Class I) was also determined by staining the Jurkat cells with a fluorescently labeled anti-HLA-A/B/C antibody.

FIG. 54 is a pair of histogram series derived from FACS analysis of HLA-A*02 expressing primary T cells transfected with guide nucleic acids (gRNA) of the disclosure that target the B2M gene. The gRNA knockout efficiency of HLA-A*02 was measured by staining the cells with fluorescently labeled anti-HLA-A*02 antibody (HLA-A*02) following transfection with sgRNA:Cas9 complexes. Knockout efficiency of HLA-A, HLA-B, and HLA-C (HLA Class I) was also determined by staining the primary T cells with a fluorescently labeled anti-HLA-A/B/C antibody.

FIG. 55 is a series of plots and inset histograms showing Jurkat cell activation and the binding capacity of the HLA-A*02 blocker module to its ligand, pMHC tetramer, in the presence and absence of B2M targeting Cas9:sgRNA complexes.

FIG. 56 is a series of FACS plots showing the effect on cell surface expression of HLA Class I complex, HLA-A*02 and binding capacity of the HLA-A*02 blocker receptor to its ligand, pMHC tetramer, in primary T cells in the presence and absence of B2M targeting Cas9:sgRNA complexes.

FIG. 57 is a diagram showing the effect of truncating the putative B2M promoter region on cell surface expression of HLA Class I complexes.

FIG. 58 is a diagram showing the regions of the putative B2M promoter region targeted by gRNA targeting sequences of the disclosure.

FIG. 59 is a pair of histogram series derived from fluorescence activated cell sorting (FACS) analysis of HLA-A*02-positive primary T cells transfected with guide nucleic acids (gRNA) of the disclosure that target either the B2M coding sequence (B2M 3, B2M 5, and B2M 6) or the putative B2M gene promoter region. Staining was done at 72 hours (left panel) or 144 hours (right panel) post-transfection.

FIG. 60 is a schematic showing illustrative regions of the B2M mRNA targeted by interfering shRNAs of the disclosure.

FIG. 61 is a pair of series of histograms and plots derived from fluorescence activated cell sorting (FACS) analysis of HLA-A*02 expressing Jurkat cells transfected with shRNA of the disclosure that target B2M mRNA. The shRNA knockout efficiency of HLA-A*02 expression was measured by staining Jurkat cells with fluorescently labeled anti-HLA-A*02 antibody (HLA-A*02), following transfection with shRNA. Knockout efficiency of HLA-A, HLA-B, and HLA-C(HLA Class I) was also determined by staining the Jurkat cells with a fluorescently labeled anti-HLA-A/B/C (HLA Class I) antibody.

FIG. 62 is a series of plots and insert histograms showing Jurkat cell activation and the binding capacity of the HLA-A*02 blocker module (inhibitory receptor) to its ligand, pMHC tetramer, in the presence (right) and absence (left) of B2M targeting shRNA.

FIG. 63 is a cartoon showing a modified T cell of the disclosure. The T cell expresses both an activator and inhibitory (blocker) receptor of the disclosure, in which beta-2− microglobulin (B2M) has been knocked out. Knock-out or knock down of B2M (B2M(−)) controls the host versus graft allogeneic response (host versus graft disease, HvG or HvGD), while the presence of the inhibitory receptor on the T cell controls the graft versus host allogeneic response (graft versus host diseases, GvH or GvHD).

FIG. 64 is a cartoon showing a modified T cell of the disclosure (graft cell, top) and a host T cell (bottom) following transplantation, and the interactions between the two cells. CTL: cytotoxic T lymphocyte. The T cell receptor on the graft TCR is indicated with the alpha, beta, gamma, delta and epsilon units (lower portion of the cell), while the activator and blocker receptors are as indicated. The host T cell expresses the indicated class I MHC complexes which can activate both the TCR and the blocker receptor on the grafted T cell.

FIG. 65A are a pair of cartoons (top) and fluorescence activated cell sorting (FACS) plots showing primary T cell transduced with f an NY-ESO-1 TCR (left), or co-transduced with an NY-ESO-1 TCR and HLA-A*02 LIR1 inhibitory receptor (right).

FIG. 65B is a pair of plots showing that the NY-ESO-1 TCR mediated killing of effector T cells co-cultured with A375 target cells expressing NY-ESO-1 and HLA-A*02 is blocked when the effectors cells also express an HLA-A*02 LIR1 blocker receptor.

FIG. 65C is a plot showing that the KRAS TCR mediated killing of effector T cells co-cultured with HuCTT1 target cells expressing KRAS-G12D/A11 and HLA-A*02 is blocked when the effectors cells also express an HLA-A*02 LIR1 blocker receptor.

FIG. 66 is a series of plots showing that the blocker receptor controls the allogeneic cytolytic response similarly to T cells in which TRAC (T cell receptor alpha constant) has been knocked out (KO). UTD: untransduced, TRAC+ cells; Blocker: blocker receptor; KO: knock out; allo: allogeneic; E:T, effector:target ratio

FIG. 67A is a series of plots showing proliferation (top row) and activation (bottom row) of primary T cells from donor 1 that were co-cultured with T cell depleted PBMCs from a second (donor 2). From left to right: Allo, primary T cells from donor 1; Allo+Blocker, primary T cells from donor 1 expressing the blocker receptor; Allo no TCR, primary T cells from donor 1 in which TRAC has been knocked out; Autologous, T cells from donor 1 cultured with T cell depleted PBMCs from donor 1.

FIG. 67B is a plot showing cytokine (interferon gamma, or IFNG) production by T cells without (T-cell only) and with co-culture with T cell depleted PBMCs (MLR). Allo, primary T cells from donor 1; Allo+Blocker, primary T cells from donor 1 expressing the blocker receptor; Allo no TCR, primary T cells from donor 1 in which TRAC has been knocked out; Autologous, T cells from donor 1 cultured with T cell depleted PBMCs from donor 1; D2 target only, D2 target cells (T cell depleted PBMCs) only.

FIG. 68A is a series of plots showing proliferation (top row) and activation (bottom row) of primary T cells from donor 1 expressing an EGFR chimeric antigen receptor (CAR) that were co-cultured with T cell depleted PBMCs from a second (donor 2). From left to right: UTD, untransduced; EGFR CAR, primary T cells from donor 1 expressing EGFR CAR; EGFR CAR+Blocker, primary T cells from donor 1 expressing the EGFR CAR and the blocker receptor; Non-allo, T cells from donor 1 cultured with T cell depleted PBMCs from donor 1.

FIG. 68B is a plot showing cytokine (interferon gamma, or IFNG) production by T cells expressing the EGFR CAR, without (T-cell only) and with co-culture with T cell depleted PBMCs (MLR). From left to right: UTD, untransduced; EGFR CAR, primary T cells from donor 1 expressing EGFR CAR; EGFR CAR+Blocker, primary T cells from donor 1 expressing the EGFR CAR and the blocker receptor; Autologous, T cells from donor 1 cultured with T cell depleted PBMCs from donor 1; D2 target cells only, D2 target cells (T cell depleted PBMCs) only.

FIG. 69 is a diagram showing an in vivo experiment to assay the allogeneic graft versus host effect of T cells expressing the blocker and activator receptor pair of the disclosure.

FIG. 70A is a diagram showing one method of generating B2M(−) T cells expressing the blocker and activator receptor pair of the disclosure.

FIG. 70B is a plot showing that B2M knockout using CRISPR/Cas9 and the methods shown in FIG. X8A reduced HLA expression on the surface of T cells expressing activator and blocker receptors. T cells from 8 donors are shown.

FIG. 71 is a series of plots showing that T cells in which B2M has been knocked out are protected from T cell killing and show limited susceptibility to NK cell killing.

FIG. 72 is a diagram showing one method of generating B2M(−) T cells expressing the blocker and activator receptor pair of the disclosure.

FIG. 73A is a diagram showing one method of generating B2M(−) T cells expressing the blocker and activator receptor pair of the disclosure using an shRNA (short hairpin RNA) specific to B2M.

FIG. 73B is a plot showing that transducing T cells from four donors with a lentiviral vector comprising an activator receptor, an inhibitory receptor and a B2M shRNA reduced HLA expression on the T cells.

FIG. 74 is a diagram showing an experiment to test the effect of B2M knock-out or knock down in graft T cells on T or NK cell killing from host cells. Allo: allogeneic; E:T, ratio of host to graft cells.

FIG. 75 is a series of plots showing the effect of B2M knock down or knock-out on host NK or T cell mediated killing graft T cells expressing the activator and blocker receptors of the disclosure.

FIG. 76 is a series of plots that show that HLA-A*02 knock out efficiency is similar across multiple HLA-targeting gRNAs, and is similar to that achieved with two B2M-targeting gRNAs.

FIG. 77 is a series of plots that show that the HLA-16 gRNA improves blocking in an A*02(+) donor and does not negatively impact blocking in A*02(−) donors. SCR: cells were transfected with a scrambled gRNA sequence.

FIG. 78 is a series of FACS plots that show that knock out of B2M and HLA-A with B2M and HLA-A targeting gRNAs restores tetramer binding. SCR: cells were transfected with a scrambled gRNA sequence.

### DETAILED DESCRIPTION

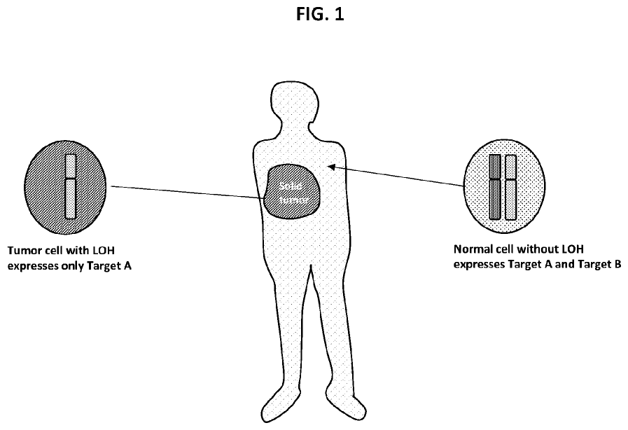

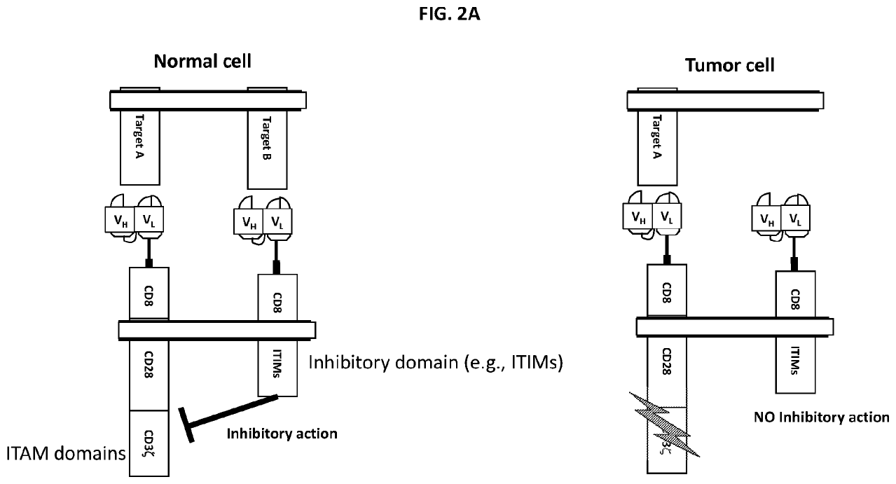

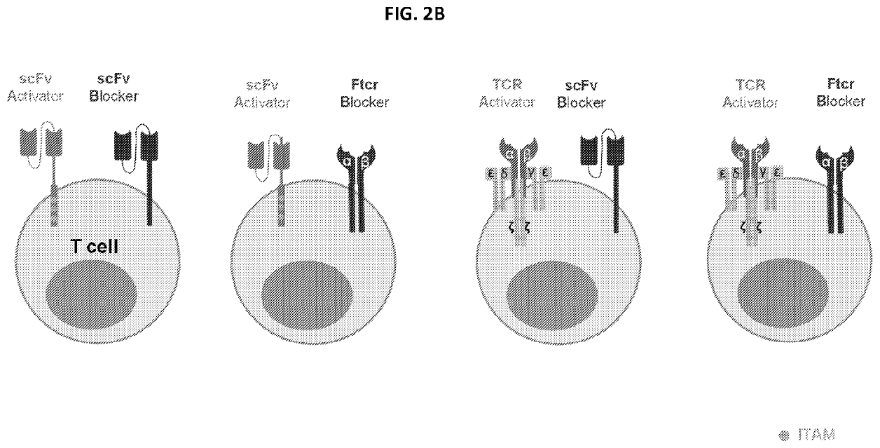

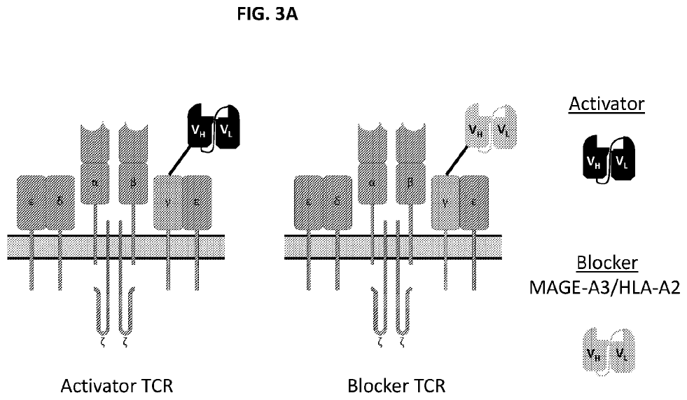

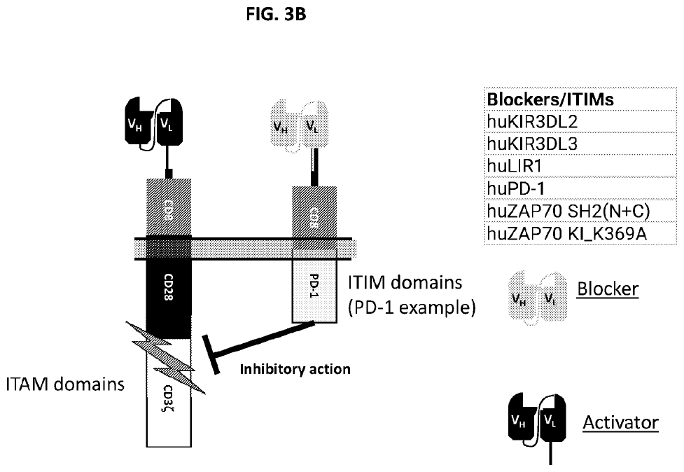

The inventors have developed a solution to the problems of identifying suitable markers and therapeutic targets and achieving cell selectivity in the treatment of diseases, particularly cancers, with adoptive cellular therapy. The primary object of the invention is an immune cell used in adoptive cell therapy that has reduced autocrine signaling. The immune cell can target cells, for example, based on loss of heterozygosity (FIG. 1). The immune cells use a two receptor system, in which activator and inhibitory signals are integrated at the cellular level within the immune cell (FIGS. 2A, 2B, 3A and 3B), by which selective targeting of tumor but not non-tumor cells is achieved. Differences in expression of surface proteins that are absent or lost in target cells but present in normal cells are thereby converted to a targeted anti-tumor cell therapy. These differences improve targeting by cell therapies, and protect normal cells from the cytotoxic effects of effector cells used for adoptive cell therapies.

The two receptor system described herein can be expressed in allogeneic immune cells specifically engineered to decrease complications such as graft versus host disease (GvHD) and host versus graft disease (HvG).

The two receptor system described herein can also be expressed in autologous immune cells. These autologous immune cells can be specifically engineered to decrease complications such as inhibitory signals between the immune cells used in the adoptive therapy.

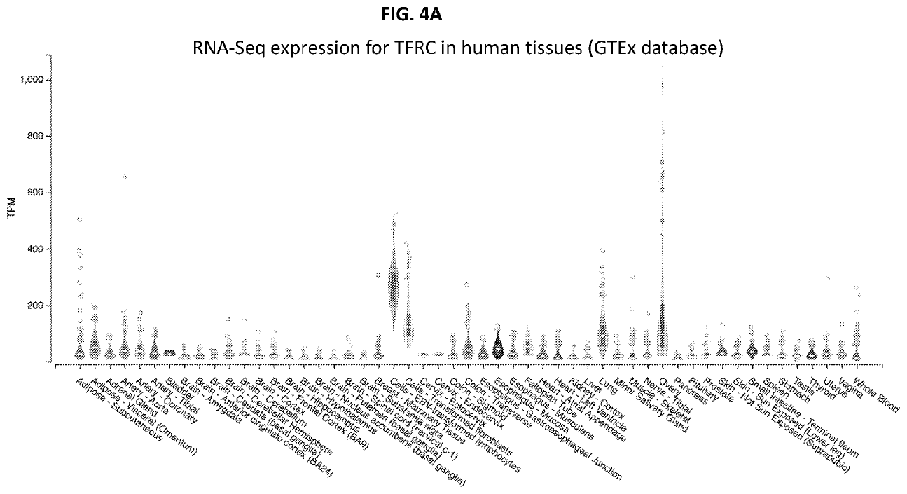

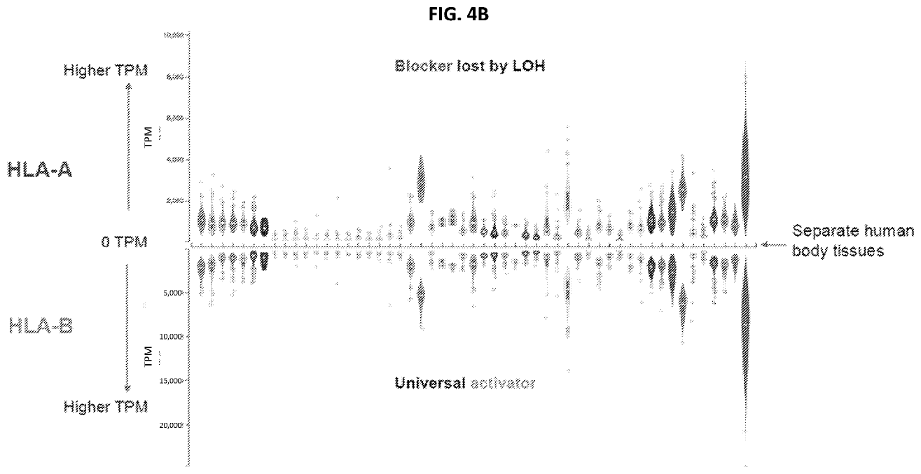

The inhibitory signals described herein, in some embodiments, are mediated by an inhibitory receptor, expressed by immune cells described herein, comprising a ligand binding domain specific to a class I major histocompatibility complex (MHC-I) molecule, or a peptide-MHC complex thereof. Native expression of an MHC-I molecule by the immune cells can potentially bind to and activate or inactivate the inhibitory receptor. Such binding and activation/inactivation can occur through both inter- and intra-cellular interactions. Inter-cellular binding and activation/inactivation occurs when a natively expressed MHC-I molecule on immune cell binds and activates/inactivates an inhibitory receptor on a separate engineered immune cell expressing an inhibitory receptor. Intra-cellar inhibitory receptor binding can occur when, for example, the inhibitory receptor binds to an MHC-I molecule natively expressed on the same immune cell (FIGS. 4A and 4B). As described herein, both the inter- and intra-cellular binding/signaling of the inhibitory receptor on or among engineered immune cells are referred to as autocrine signaling or binding. Both intra- and inter-cellular inactivation can result in undesired inhibition at, for example, the site of a target activator or in a preparation or composition comprising a plurality of the immune cell. The inventors of the present disclosure have recognized that the undesired autocrine signaling and binding can be suppressed if the immune cell comprises one or more modification that reduce autocrine signaling by the receptor (FIG. 4B). In some embodiments, the one or more modifications to the immune cell comprises an inactivating mutation in an endogenous gene encoding an allele of an endogenous MHC class I polypeptide specifically bound by the inhibitory receptor (FIGS. 4A-4B).

The inventors of the present disclosure have recognized that the undesired autocrine binding/signaling can be suppressed if the immune cell has reduced or eliminated human leukocyte antigen (HLA) expression or function. HLA expression or function can be reduced or eliminated by targeting an HLA-A gene or allele thereof, e.g. HLA-A*02, with an interfering RNA molecule. In some embodiments, the immune cell comprises an interfering RNA, e.g. an shRNA complementary to a portion of the HLA-A messenger RNA (mRNA) transcript. HLA-A is a component of MHC class I complex. In the MHC class I complex, B2M binds the a chain to form a complex on the cell surface. Viewed across the cell membrane with the cytoplasm down, the α1 domain is directly above B2M, and α1 and B2M lie adjacent to α2 and α3, the latter of which is linked to a transmembrane domain. Without wishing to be bound by theory, it is thought that reduced or eliminated expression or function of HLA-A interferes with the formation of the MHC class I complex, leading to immune cells with greatly reduced or absent MHC class I on the cell surface.

In some embodiments, the one or more modifications to the immune cell comprises an inactivating mutation in beta-2-microglobulin (B2M, or β2m). B2M is a component of MHC class I molecules. In the MHC class I complex, B2M lies beside the α3 chain on the cell surface. The α1 chain is directly above B2M, and α1 and B2M lie adjacent to α2 and α3, the latter of which includes a transmembrane domain. Without wishing to be bound by theory, it is thought that in the absence of B2M, the MHC class I complex is unable to form, leading to immune cells with greatly reduced or absent MHC class I on the cell surface.

The inventors have developed a solution to the problems of identifying suitable therapeutic targets and achieving cell selectivity in the treatment of diseases, particularly cancers, with adoptive cellular therapy. The immune cell used in the adoptive cell therapies of the disclosure can target cells, for example, based on loss of heterozygosity (FIG. 1). The immune cells use a two receptor system, in which activator and inhibitory signals are integrated at the cellular level within the immune cells (FIGS. 2A, 2B, 3A and 3B), by which selective targeting of tumor but not non-tumor cells is achieved. Differences in expression of surface proteins that are absent or lost in target cells but present in normal cells are thereby converted to a targeted anti-tumor cell therapy. These differences improve targeting by cell therapies, and protect normal cells from the cytotoxic effects of effector cells used for adoptive cell therapies.

The two receptor system described herein can be expressed in allogeneic immune cells specifically engineered to reduce host versus graft disease. Two challenges to allogeneic adoptive cell therapy include graft versus host disease (GvHD), and host versus graft disease (HvG). In GvHD, donor immune cells attack healthy cells of the recipient. In HvG, host immune cells attack the transplanted cells, decreasing their persistence and reducing the effectiveness of the adoptive cell therapy. In conventional approaches to allogeneic adoptive cell therapy, donor immune cells, such as T cells, are engineered such that the cells do not express functional endogenous T cell receptors, whose activation can lead to GvHD. The donor T cells are further engineered to remove expression of the major histocompatibility class I complex (MHC I), which can induce a cytotoxic response in host immune cells leading to HvG. Thus, allogeneic immune cells used in conventional approaches typically undergo multiple genetic modifications in addition to those required to express an activating receptor. For example, these allogeneic immune cells may have one or more TCR subunits, such as TRAC, knocked out or down, and one or more MHC class I subunits, such as B2M, knocked out or down. However, it has unexpectedly been found that some embodiments of the blocker receptor of the dual receptor system described herein can block activation of donor immune cells that via endogenous TCRs. Thus, allogeneic immune cells (donor cells) of the disclosure that express the dual receptor system described herein can be advantageously rendered suitable for allogeneic transplant with fewer modifications compared to conventional allogeneic approaches. For example, allogeneic immune cells of the disclosure comprising the activator and blocker (also referred to as inhibitory) receptors described herein, in which MHC I has been reduced or eliminated, but which still express endogenous TCR receptors, can be suitable for allogeneic transplantation.

The two receptor system described herein can also be expressed in autologous immune cells, and can decrease the toxicity associated with certain adoptive cell therapies and two expand the range of molecular targets available for cancer. The inventors of the present disclosure have recognized that the undesired autocrine signaling and binding can be suppressed if the immune has reduced or eliminated beta-2-microglobulin (B2M) expression or function. B2M expression or function can be reduced or eliminated by targeting B2M with an interfering RNA molecule. (FIG. 4B). In some embodiments, the immune cell comprises an interfering RNA, e.g. an shRNA complementary to a portion of the beta-2-microglobulin (B2M, or β2m) messenger RNA (mRNA) transcript. B2M is a component of MHC class I complex. In the MHC class I complex, B2M lies beside the α3 domain of the a chain on the cell surface. The α1 domain is directly above B2M oriented in a typical structure diagram of the protein complex, and α1 and B2M lie adjacent to α2 and α3, the latter of which is connected to a transmembrane domain. Without wishing to be bound by theory, it is thought that reduced or eliminated expression or function of B2M interferes with the formation of the MHC class I complex-, leading to immune cells with greatly reduced or absent MHC class I on the cell surface.

In some embodiments, the immune cell described herein comprises one or more modifications. In some embodiments, the modifications comprise inactivating mutations in an endogenous gene encoding an allele of an endogenous MHC class I polypeptide. While it is advantageous for the immune cells disclosed herein to be modified with an inactivating mutation targeting a single allele of an endogenous MHC class I polypeptide, inactivating mutations to both alleles of an endogenous MHC class polypeptide is also encompassed by the disclosure. In addition, it may be advantageous to inactivate several loci of class I MHC gene complex and the alleles of those loci in the immune cells. In some embodiments, the modifications comprise inactivating mutations in an endogenous gene encoding both alleles of an endogenous MHC class I polypeptide. In some embodiments, the endogenous MHC class I polypeptide is bound specifically by an inhibitory receptor expressed by the immune cell described herein. In some embodiments, the MHC class I polypeptide is HLA-A, HLA-B, and/or HLA-C. In some embodiments, the MHC class I polypeptide is HLA-A. In some embodiments, the MHC class I polypeptide is HLA-A*02. In some polypeptides the MHC class I polypeptide is HLA-A*02:01.

The immune cell described herein can be modified using any methods known in the art. In a particular embodiment, the immune cells are modified using a CRISPR/Cas gene editing system. The immune cells described herein, according to an embodiment, can be modified to express or be transiently exposed to a CRISPR/Cas gene editing system comprising a nucleic acid guided endonuclease. In some embodiments, the nucleic acid guided endonuclease is a class II endonuclease, such as Cas9 or Cas12. In some embodiments, the nucleic acid guided endonuclease is directed to a target gene locus using a target-specific guide nucleic acid. The gene editing system can be used to modify the immune cell described herein to delete, inactivate, reduce expression, or otherwise inhibit function of a target gene or a target gene product.

In some embodiments, the immune cell described herein comprises an inhibitory receptor comprising a binding domain specific to a class I major histocompatibility complex (MHC-I) molecule, or a peptide-MHC complex thereof, wherein expression and/or function of human leukocyte antigen (HLA) in said immune cell has been reduced or eliminated. In particular embodiments, the immune cells comprise an interfering RNA, comprising a sequence complementary to a sequence of a HLA-A*02 mRNA. In some embodiments, the interfering RNA is capable of inducing RNA interference (RNAi) mediated degradation of the HLA-A*02 mRNA. The immune cells described herein, according to some embodiments, comprise a short hairpin RNA. In some embodiments, the shRNA comprises a first sequence and a second sequence, wherein the first sequence, has from 5′ to 3′ a sequence complementary to the HLA-A*02 mRNA; and wherein the second sequence, has from 5′ to 3′ end a sequence complementary to the first sequence, wherein the first sequence and second sequence form the shRNA. In an immune cell comprising the shRNA, the HLA-A gene product is reduced or eliminated in expression and/or function. In some embodiments, an immune cell comprising the shRNA has reduced or eliminated expression, — or function of an MHC-I molecule.

In one aspect, the disclosure provides guide nucleic acids targeting an endogenous MHC class I polypeptide. In some embodiments, the guide nucleic acids target the B2M gene.

In some embodiments, the immune cell described herein comprises an inhibitory receptor comprising a binding domain specific to a class I major histocompatibility complex (MHC-I) molecule, or a peptide-MHC complex thereof, wherein expression and/or function of beta-2-microglobulin (B2M) in said immune cell has been reduced or eliminated. In particular embodiments, the immune cells comprise an interfering RNA, comprising a sequence complementary to a sequence of a B2M mRNA. In some embodiments, the interfering RNA is capable of inducing RNA interference (RNAi) mediated degradation of the B2M mRNA. The immune cells described herein, according to some embodiments, comprise a short hairpin RNA. In some embodiments, the shRNA comprises a first sequence and a second sequence, wherein the first sequence, has from 5′ to 3′ a sequence complementary to the B2M mRNA; and wherein the second sequence, has from 5′ to 3′ end a sequence complementary to the first sequence, wherein the first sequence and second sequence form the shRNA. In an immune cell comprising the shRNA, the B2M gene product is reduced or eliminated in expression and/or function. In some embodiments, an immune cell comprising the shRNA is reduced or eliminated in expression or function of an MHC-I molecule.

In one aspect, the disclosure provides vectors for expressing the shRNA described herein in an immune cell. In another aspect, the disclosure provides methods of manufacturing the immune cells described herein.

In one aspect, disclosure provides compositions comprising the immune cells described herein.

In one aspect, the disclosure provides methods of treatment comprising administering the immune cells described herein to a subject in need thereof. In another aspect, the disclosure provides compositions comprising the immune cells described herein for use as a medicament in the treatment of a subject in need. In some embodiments, the subject suffers from or is at risk of cancer or a hematological malignancy.

This approach disclosed herein uses, in some embodiments, two engineered receptors, the first comprising a ligand binding domain for an activator ligand and the second comprising a ligand binding domain for an inhibitor ligand, which is selectively activated in target cells using an “AND NOT” Boolean logic (FIGS. 2A, 2B, 3A and 3B). Normal cells express both the activator and the inhibitor ligands, but activation of effector cells through the first receptor is blocked by binding of the second receptor comprising the inhibitor LBD to the inhibitor ligand, which exerts a protective effect and dominates the activity of the first, activator receptor. In contrast, in target cells that express the activator ligand but do not express the inhibitor ligand, binding of the activator ligand by the activator LBD leads to activation of the cell. Advantages of the dual activator/inhibitor receptor strategy of the instant disclosure include the ability to tune the activator and inhibitor combination to create a potent, but specific tumor-targeted adoptive cell therapy. Further, this approach can overcome the challenges of a variable effector to target cell ratio (E:T ratio) in the body, and the potentially massive excess of normal versus tumor cells seen when targeting tumor cells with adoptive cell therapies (e.g., 10¹³ normal cells versus 10⁹ tumor cells). Still further, the inventors have identified activators and inhibitors that cover large potential patient combinations, rendering this a commercially feasible approach.

Specificity of the adoptive cell therapy for a specific cell type can be achieved through the different activities of the first and second receptors, and the differential expression of the first and second ligands for the first and second receptors, respectively. Binding of the first ligand to the first receptor provides an activation signal, while binding of the second ligand to the second receptor prevents or reduces activation of effector cells even in the presence of the first ligand. The first ligand can be expressed more broadly than the second ligand, for example in both cells targeted by an adoptive cell therapy, and in healthy cells that are not target cells for an adoptive cell therapy (non-target cells). In contrast, the second ligand is expressed in the non-target cells, and is not expressed in the target cells. Only the target cells and not the non-target express the first and not the second ligand, thereby activating effector cells comprising the dual receptors of the disclosure in the presence of these cells.

The disclosure provides compositions and methods for targeting cells (e.g. tumor cells) based on loss of heterozygosity through use of two engineered receptors. The two engineered receptors, one an inhibitor and one activator, each comprise a different ligand binding domain that recognizes a different ligand. Differences in expression of the first and second ligands are used to selectively activate effector cells expressing the two receptors when only the first, activator ligand is present. Accordingly, in some embodiments, the first ligand binding domain and the second ligand binding domain are on different receptor molecules; i.e., separate receptors that are not part of a single genetic construct, fusion protein or protein complex. In some embodiments, one of the receptors activates the cell and other receptor inhibits the cell when each binds its cognate ligand. In some embodiments, the receptor comprising the second, inhibitor ligand binding domain dominates signaling so that if a target cell expresses both targets, the result is inhibition of the effector cell. Only when the inhibitory target is absent from the cell, does the first, activator ligand induce activation of the effector cell through the receptor comprising the first, activator ligand binding domain. In some embodiments, the first ligand is an activator ligand and the second ligand is an inhibitory ligand.

Any widely expressed cell surface molecule, for example a cell adhesion molecule, a cell-cell signaling molecule, an extracellular domain, a molecule involved in chemotaxis, a glycoprotein, a G protein-coupled receptor, a transmembrane, a receptor for a neurotransmitter or a voltage gated ion channel, or a peptide antigen of any of these, can be used as a first ligand. As a further example, the first ligand can be the transferrin receptor (TFRC). Any cell surface molecule not expressed on the surface of the target cell can be used as a second ligand. In those embodiments where an engineered receptor is used in the adoptive cell therapy to treat cancer, and the target cells are cancer cells, a second ligand may be chosen based on the loss of heterozygosity of the second ligand in cancer cells. Exemplary genes whose expression is frequently lost in cancer cells, for example due to mutations leading to loss of heterozygosity, include HLA class I alleles, minor histocompatibility antigens (MiHAs), and Y chromosome genes. In some embodiments, the first ligand is an activator ligand and the second ligand is an inhibitory ligand. In some embodiments, the HLA class I allele comprises HLA-A*02.

The disclosure further provides vectors and polynucleotides encoding the engineered receptors described herein.

The disclosure further provides methods of making immune cell populations comprising the engineered receptors described herein, and methods of treating disorders using the same.

### Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below. Additional definitions are set forth throughout this disclosure.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” “some embodiments,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

As used herein, the term “isolated” means material that is substantially or essentially free from components that normally accompany it in its native state. In particular embodiments, the term “obtained” or “derived” is used synonymously with isolated.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

The term “allogeneic” refers to donor tissues or cells that are genetically dissimilar to the recipient receiving the cells or tissues, and which are therefore immunologically incompatible with the recipient.

The term “autologous” refers to cells or tissues obtained from the same subject.

As used herein “treatment” or “treating,” includes any beneficial or desirable effect, and may include even minimal improvement in symptoms. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of a symptom of disease. It also refers to delaying the onset or recurrence of a disease or condition or delaying the occurrence or recurrence of the symptoms of a disease. As used herein, “prevention” and similar words also includes reducing the intensity, effect, symptoms and/or burden of disease prior to onset or recurrence.

As used herein, the term “amount” refers to “an amount effective” or “an effective amount” of a virus to achieve a beneficial or desired prophylactic or therapeutic result, including clinical results.

A “prophylactically effective amount” refers to an amount of a virus effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or cell may vary according to factors such as the disease state, age, sex, and weight of the individual, and the ability of the virus or cell to elicit a desired response in the individual. A therapeutically effective amount is also one in which any toxic or detrimental effects of the virus or cell are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decrease” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

As used herein, a “subsequence” refers to a length of contiguous amino acids or nucleotides that form a part of a sequence described herein. A subsequence may be identical to a part of a full length sequence when aligned to the full length sequence, or less than 100% identical to the part of the full length sequence to which it aligns (e.g., 90% identical to 50% of the full sequence, or the like).

As used herein, a “polynucleotide system” refers to one or more polynucleotides. The one or more polynucleotides may be designed to work in concert for a particular application, or to produce a desired transformed cell.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

The term “MOI” is used herein to refer to multiplicity of infection, which is the ratio of agents (e.g. viral particles) to infection targets (e.g. cells).

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

As used herein, a “target cell” refers to cell that is targeted by an adoptive cell therapy. For example, a target cell can be cancer cell, which can be killed by the transplanted T cells of the adoptive cell therapy. Target cells of the disclosure express an activator ligand as described herein, and do not express an inhibitor ligand.

As used herein, a “non-target cell” refers to cell that is not targeted by an adoptive cell therapy. For example, in an adoptive cell targeting cancer cells, normal, healthy, non-cancerous cells are non-target cells. Some, or all, non-target cells in a subject may express both the target antigen and the non-target antigen. Non-target cells in a subject may express the non-target antigen irrespective of whether or not these cells also express the target antigen.

The present description includes artificial receptors with activating and inhibiting activity. The artificial receptors are sometimes referred to as “activator receptors,” “inhibitor receptors,” or “engineered receptors.” Inhibitor receptors are sometimes referred to as “blockers,” “blocking receptors,” and the like.

“RNAi” or “RNA interference” refers to the process of sequence-specific post-transcriptional gene silencing, mediated by double-stranded RNA (dsRNA). Duplex RNAs such as siRNA (small interfering RNA), miRNA (micro RNA), shRNA (short hairpin RNA), ddRNA (DNA-directed RNA), piRNA (Piwi-interacting RNA), or rasiRNA (repeat associated siRNA) and modified forms thereof are all capable of mediating RNA interference. These dsRNA molecules may be commercially available or may be designed and prepared based on known sequence information. The anti-sense strand of these molecules can include RNA, DNA, PNA, or a combination thereof. DNA/RNA chimeric polynucleotides include, but are not limited to, a double-strand polynucleotide composed of DNA and RNA that inhibits the expression of a target gene. dsRNA molecules can also include one or more modified nucleotides, as described herein, which can be incorporated on either or both strands.

In RNAi gene silencing or knockdown, dsRNA comprising a first (anti-sense) strand that is complementary to a portion of a target gene and a second (sense) strand that is fully or partially complementary to the first anti-sense strand is introduced into an organism. After introduction into the organism, the target gene-specific dsRNA is processed into relatively small fragments (siRNAs) and can subsequently become distributed throughout the organism, decrease messenger RNA of target gene, leading to a phenotype that may come to closely resemble the phenotype arising from a complete or partial deletion of the target gene.

Certain dsRNAs in cells can undergo the action of Dicer enzyme, a ribonuclease III enzyme. Dicer can process the dsRNA into shorter pieces of dsRNA, i.e. siRNAs. RNAi also involves an endonuclease complex known as the RNA induced silencing complex (RISC). Following cleavage by Dicer, siRNAs enter the RISC complex and direct cleavage of a single stranded RNA target having a sequence complementary to the anti-sense strand of the siRNA duplex. The other strand of the siRNA is the passenger strand. Cleavage of the target RNA takes place in the middle of the region complementary to the anti-sense strand of the siRNA duplex. siRNAs can thus down regulate or knock down gene expression by mediating RNA interference in a sequence-specific manner.

As used herein, “target gene” or “target sequence” of an interfering refers to a gene or gene sequence whose corresponding RNA is targeted for degradation through the RNAi pathway using dsRNAs or siRNAs as described herein. To target a gene, for example using an siRNA, the siRNA comprises an anti-sense region complementary to, or substantially complementary to, at least a portion of the target gene or sequence, and sense strand complementary to the anti-sense strand. Once introduced into a cell, the siRNA directs the RISC complex to cleave an RNA comprising a target sequence, thereby degrading the RNA.

The term “chimeric antigen receptors” or “CARs” as used herein, may refer to artificial T-cell receptors, chimeric T-cell receptors, or chimeric immunoreceptors, for example, and encompass engineered receptors that graft an artificial specificity onto a particular immune effector cell, such as a helper T cell (CD4+), cytotoxic T cell (CD8+) or NK cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen such as an HLA-E antigen. In some embodiments, CARs comprise an intracellular signaling domain, a transmembrane domain, and an extracellular domain comprising an antigen-binding region. In some embodiments, CARs comprise fusions of single-chain variable fragments (scFvs) or scFabs derived from monoclonal antibodies, fused to a transmembrane domain and intracellular signaling domain(s). The fusion may also comprise a hinge. Either heavy-light (H-L) and light-heavy (L-H) scFvs may be used. The specificity of CAR designs may be derived from antigens of receptors (e.g., peptides). Depending on the type of intracellular domain, a CAR can be an activator receptor or an inhibitory receptor. In some embodiments, for example when the CAR is an activator receptor, the CAR comprises domains for additional co-stimulatory signaling, such as CD3, FcR, CD27, CD28, CD137, DAP10, and/or OX40. In some embodiments, molecules can be co-expressed with the CAR, including co-stimulatory molecules, reporter genes for imaging (e.g., for positron emission tomography), gene products that conditionally ablate the T cells upon addition of a pro-drug, homing receptors, cytokines, and cytokine receptors. As used herein, characteristics attributed to a chimeric antigen receptor may be understood to refer to the receptor itself or to a host cell comprising the receptor.

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon (CD3E) and CD3 delta (CD3D) form a heterodimer, CD3 epsilon and CD3 gamma (CD3G) for a heterodimer, and two CD3 zeta (CD3Z) form a homodimer.

The term “stimulation” refers to a primary response induced by binding of a stimulatory domain or stimulatory molecule (e.g., a TCR/CD3 complex) with its cognate antigen thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof that, when natively expressed by a T-cell, provides the primary cytoplasmic signaling sequence(s) that regulate activation of the TCR complex in a stimulatory way for at least some aspect of the T-cell signaling pathway. TCR alpha and/or TCR beta chains of wild type TCR complexes do not contain stimulatory domains and require association with CD3 subunits such as CD3 zeta to initiate signaling. In one aspect, the primary stimulatory signal is initiated by, for instance, binding of a TCR/CD3 complex with a major histocompatibility complex (MHC) bound to peptide, and which leads to mediation of a T-cell response, including, but not limited to, proliferation, activation, differentiation, and the like. One or more stimulatory domains, as described herein, can be fused to the intracellular portion of any one or more subunits of the TCR complex, including TCR alpha, TCR beta, CD3 delta, CD3 gamma and CD3 epsilon.

As used herein, a “domain capable of providing a stimulatory signal” refers to any domain that, either directly or indirectly, can provide a stimulatory signal that enhances or increases the effectiveness of signaling mediated by the TCR complex to enhance at least some aspect of T-cell signaling. The domain capable of providing a stimulatory signal can provide this signal directly, for example with the domain capable of providing the stimulatory signal is a primary stimulatory domain or co-stimulatory domain. Alternatively, or in addition, the domain capable of providing the stimulatory signal can act indirectly. For example, the domain can be a scaffold that recruits stimulatory proteins to the TCR, or provide an enzymatic activity, such as kinase activity, that acts through downstream targets to provide a stimulatory signal.

As used herein, “activation” of an immune cell or an immune cell that is “activated” is an immune cell that can carry out one or more functions characteristic of an immune response. These functions include proliferation, release of cytokines, and cytotoxicity, i.e. killing of a target cell. Activated immune cells express markers that will be apparent to persons of skill in the art. For example, activated T cells can express one or more of CD69, CD71, CD25 and HLA-DR. An immune cell expressing an activator receptor (e.g. a HLA-E CAR) can be activated by the activator receptor when it becomes responsive to the binding of the receptor to a target antigen (e.g. HLA-E) expressed by the target cell. A “target antigen” can also be referred to as an “activator antigen” and may be isolated or expressed by a target cell. Activation of an immune cell expressing an inhibitory receptor can be prevented when the inhibitory receptor becomes responsive to the binding of anon-target antigen (e.g. HLA-A*02), even when the activator receptor is bound to the target activator ligand. A “non-target antigen” can also be referred to as an “inhibitory ligand” or a “blocker”, and may be isolated or expressed by a target cell.

As used herein, a “domain capable of providing an inhibitory signal” refers to any domain that, either directly or indirectly, can provide an inhibitory signal that inhibits or decreases the effectiveness signaling mediated by the TCR complex. The domain capable of providing an inhibitory signal can reduce, or block, totally or partially, at least some aspect of T-cell signaling or function. The domain capable of providing an inhibitory signal can provide this signal directly, for example with the domain capable of providing the inhibitory signal provides a primary inhibitory signal. Alternatively, or in addition, the domain capable of providing the stimulatory signal can act indirectly. For example, the domain can recruit additional inhibitory proteins to the TCR, or can provide an enzymatic activity that acts through downstream targets to provide an inhibitory signal.

As used herein, “intracellular domain” refers to the cytoplasmic or intracellular domain of a protein, such as a receptor, that interacts with the interior of the cell, and carries out a cytosolic function. As used herein, “cytosolic function” refers to a function of a protein or protein complex that is carried out in the cytosol of a cell. For example, intracellular signal transduction cascades are cytosolic functions.

A polynucleotide is “operably linked” to another polynucleotide when it is placed into a functional relationship with the other polynucleotide. For example, a promoter or enhancer is operably linked to a coding sequence if it affects the transcription of the sequence. A peptide is “operably linked” to another peptide when the polynucleotides encoding them are operably linked, preferably they are in the same open reading frame.

A “promoter” is a sequence of DNA needed to turn a gene on or off. Promoters are located immediately upstream and/or overlapping the transcription start site, and are usually between about one hundred to several hundred base pairs in length.

Polymorphism refers to the presence of two or more variants of a nucleotide sequence in a population. A polymorphism may comprise one or more base changes, an insertion, a repeat, or a deletion. A polymorphism includes e.g. a simple sequence repeat (SSR) and a single nucleotide polymorphism (SNP), which is a variation, occurring when a single nucleotide of adenine (A), thymine (T), cytosine (C) or guanine (G) is altered.

As used herein, “specific to” or “specifically binds to” when used with respect to a ligand binding domain, such as an antigen binding domain, refers to a ligand binding domain that has a high specificity for a named target. Antibody specificity can viewed as a measure of the goodness of fit between the ligand binding domain and the corresponding ligand, or the ability of the ligand binding domain to discriminate between similar or even dissimilar ligands. In comparison with specificity, affinity is a measure of the strength of the binding between the ligand binding domain and ligand, such that a low-affinity ligand binding domain binds weakly and high-affinity ligand binding domain binds firmly. A ligand binding domain that is specific to a target allele is one that can discriminate between different alleles of a gene. For example, a ligand binding domain that is specific to HLA-A*02 will not bind, or bind more weakly to other HLA-A alleles such as HLA-A*01 or HLA-A*03. The person of skill in the art will appreciate that a ligand binding domain can be said to be specific to a particular target, and yet still have low levels of binding to one or more additional targets that do not affect its function in the receptor systems described herein. In the context of guide nucleic acids (gNA), a gNA with a targeting sequence that is “specific to” a target sequence is capable of binding to the target sequence and localizing the ribonucleoprotein (RNP) comprising the gNA to the target sequence.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

As used herein, a “target antigen,” whether referred to using the term antigen or the name of a specific antigen, refers to an antigen expressed by a target cell, such as a cancer cell. Expression of target antigen is not limited to target cells. Target antigens may be expressed by both cancer cells and normal, non-cancer cells in a subject.

As used herein, a “non-target antigen” (or “blocker antigen”) whether referred to using the term antigen or the name of a specific antigen, refers to an antigen that is expressed by normal, non-cancer cells and is not expressed in cancer cells. This difference in expression allows the inhibitory receptor to inhibit immune cell activation in the presence of non-target cells, but not in the presence of target cells.

As used herein, “affinity” refers to strength of binding of a ligand to a single ligand binding site on a receptor, for example an antigen for the antigen binding domain of any of the receptors described herein. Ligand binding domains can have a weaker interaction (low affinity) with their ligand, or a stronger interaction (high affinity).

Kd, or dissociation constant, is a type of equilibrium constant that measures the propensity of a larger object to separate reversibly into smaller components, such as, for example, when a macromolecular complex comprising receptor and its cognate ligand separates into the ligand and the receptor. When the Kd is high, it means that a high concentration of ligand is need to occupy the receptor, and the affinity of the receptor for the ligand is low. Conversely, a low Kd means that the ligand has a high affinity for the receptor.

As used herein, a receptor that is “responsive” or “responsive to” refers to a receptor comprising an intracellular domain, that when bound by a ligand (i.e. antigen) generates a signal corresponding to the known function of the intracellular domain. An activator receptor bound to a target antigen can generate a signal that causes activation of an immune cell expressing the activator receptor. An inhibitory receptor bound to a non-target antigen can generate an inhibitory signal that prevents or reduces activation of an immune cell expressing the activator receptor. Responsiveness of receptors, and their ability to activate or inhibit immune cells expressing the receptors, can be assayed by any means known in the art and described herein, including, but not limited to, reporter assays and cytotoxicity assays.

Receptor expression on an immune cell can be verified by assays that report the presence of the activator receptors and inhibitory receptors described herein. For example, a population of immune cells can be stained with a labeled molecule (e.g. a fluorophore labeled receptor-specific antibody or a fluorophore-labeled receptor-specific ligand), and quantified using fluorescence activated cell sorting (FACS) flow cytometry. This method allows a percentage of immune cells in a population of immune cells to be characterized as expressing an activator receptor, an inhibitory receptor, or both receptors. The ratio of activator receptor and inhibitory receptors expressed by the immune cells described herein can be determined by, for example, digital droplet PCR. These approaches can be used to characterize the population of cells for the production and manufacturing of the immune cells, pharmaceutical compositions, and kits described herein. For the immune cells, pharmaceutical compositions, and kits described herein, it is understood that a suitable percentage of immune cells expressing both an activator receptor and an inhibitory receptor is determined specifically for the methods described herein. For example, a suitable percentage of immune cells expressing both an activator receptor and in inhibitory receptor can be at least 50%, at least 55%, at least 60%, at least 65%, at least 70%, at least 75%, at least 80%, at least 85%, at least 90%, or at least 95%. For example, a suitable percentage of immune cells expressing both an activator receptor and an inhibitory receptor can be at most 50%, at most 55%, at most 60%, at most 65%, at most 70%, at most 75%, at most 80%, at most 85%, at most 90%, or at most 95%. For example, a suitable ratio of activator receptor and inhibitory receptor in an immune cell can be about 5:1, about 4:1, about 3:1, about 2:1, about 1:1, about 1:2, about 1:3, about 1:4, or about 1:5. It is understood that purification, enrichment, and/or depletion steps can be used on populations of immune cells to meet suitable values for the immune cells, pharmaceutical compositions, and kits described herein.

A responsive receptor expressed by the immune cells described herein can be verified by assays that measure the generation of a signal expected to be generated by the intracellular domain of the receptor. Reporter cell lines, such as Jurkat-Luciferase NFAT cells (Jurkat cells), can be used to characterize a responsive receptor. Jurkat cells are derived from T cells and comprise a stably integrated nuclear factor of activated T-cells (NFAT)-inducible luciferase reporter system. NFAT is a family of transcription factors required for immune cell activation, whose activation can be used as a signaling marker for T cell activation. Jurkat cells can be transduced or transfected with the activator receptors and/or inhibitory receptors described herein. The activator receptor is responsive to the binding of a ligand if the Jurkat cell expresses a luciferase reporter gene, and the level of responsiveness can be determined by the level of reporter gene expression. The presence of luciferase can be determined using any known luciferase detection reagent, such as luciferin. An inhibitory receptor is responsive to the binding of a ligand if, when co-expressed with an activator receptor in Jurkat cells, it prevents a normally responsive immune cell from expressing luciferase in response to the activator receptor. For example, the responsiveness of an inhibitory receptor can be determined and quantified in a Jurkat cell expressing both an activator and an inhibitor by observing the following: 1) the Jurkat cell expresses luciferase in the presence of activator receptor ligand and absence of inhibitory receptor ligand; and 2) luciferase expression in the Jurkat cell is reduced or eliminated in the presence of both an activator receptor ligand and an inhibitory receptor ligand. This approach can be used to determine the sensitivity, potency, and selectivity of activator receptors and specific pairs of activator receptors and inhibitory receptors. The sensitivity, potency, and selectivity can be quantified by EC50 or IC50 values using dose-response experiments, where an activator receptor ligand and/or inhibitory receptor ligand is titrated into a culture of Jurkat cells expressing an activator receptor or a specific pair of activator and inhibitory receptors. Alternatively, the EC50 and IC50 values can be determined in a co-culture of immune cells (e.g. Jurkat cells or primary immune cells) expressing an activator receptor or a specific pair of activator and inhibitory receptors and target cells expressing an increasing amount of an activator ligand or inhibitor ligand. An increasing amount of activator ligand or inhibitor ligand can be accomplished in the target cell by, for example, titration of activator ligand or inhibitor ligand encoding mRNA into target cells, or use of target cells that naturally express different levels of the target ligands.

Activation of the immune cells described herein that express an activator receptor or specific pairs of activator and inhibitory receptors can be further determined by assays that measure the viability of a target cell following co-incubation with said immune cells. The immune cells, sometimes referred to as effector cells, are co-incubated with target cells that express an activator receptor ligand, an inhibitory receptor ligand, or both an activator and inhibitory receptor ligand. Following co-incubation, viability of the target cell is measured using any method to measure viability in a cell culture. For example, viability can be determined using a mitochondrial function assay that uses a tetrazolium salt substrate to measure active mitochondrial enzymes. Viability can also be determined using imaging based methods. Target cells can express a fluorescent protein, such as green fluorescent protein or red fluorescent protein. Reduction in total cell fluorescence indicates a reduction in viability of the target cell. A reduction in viability of the target cell following incubation with immune cells expressing an activator receptor or a specific pair of activator and inhibitory receptors is interpreted as target cell-mediated activation of the immune cell. A measure of the selectivity of the immune cells can also be determined using this approach. The immune cell expressing a pair of activator and inhibitory receptors is selective if the following is observed: 1) viability is reduced in target cells expressing the activator receptor ligand but not the inhibitory receptor ligand; 2) viability is not reduced in target cells expressing both an activator receptor ligand and an inhibitory receptor ligand. From these measurements, a “specific killing” value can be derived that quantifies the percentage of immune cell activation based on the reduction in viability of target cell as a percentage of a negative control (immune cells that do not express an activator receptor). Further, from these measurements a “selectivity ratio” value can be derived that represents the ratio of the specific killing observed in target cells expressing an activator receptor ligand in the absence of inhibitory receptor ligand to the specific killing observed in target cells expressing both an activator receptor ligand and an inhibitory receptor ligand. This approach can be used to characterize the population of cells for the production and manufacturing of the immune cells, pharmaceutical compositions, and kits described herein.

A suitable specific killing value for the immune cells, pharmaceutical compositions, and kits can be, for example, the following criteria: 1) at least 40%, at least 50%, at least 60%, at least 70%, at least 80%, at least 90%, at least 95%, at least 97% or at least 99% specific killing following a 48 hour co-incubation of immune cells and target cells expressing activator receptor ligand in the absence of inhibitory receptor ligand; and 2) less than or equal to 40%, less than or equal to 35%, less than or equal to 30%, less than or equal to 25%, less than or equal to 20%, less than or equal to 15%, less than or equal to 10%, less than or equal to 5%, less than or equal to 3% or less than or equal to 1% specific killing of target cell expressing both an activator receptor ligand and an inhibitory receptor ligand.

As a further example, a suitable specific killing value for the immune cells, pharmaceutical compositions and kits can be the following criteria: 1) between 30% and 99%, between 40% and 99%, between 50% and 99%, between 55% and 95%, between 60% and 95%, between 60% and 90%, between 50% and 80%, between 50% and 70% or between 50% and 60% of target cells expressing the activator ligand but not the inhibitor ligand are killed; and 2), between 1% and 40%, between 3% and 40%, between 5% and 40%, between 5% and 30%, between 10% and 30%, between 15% and 30% or between 5% and 20% of target cells expressing the activator ligand and the inhibitor ligand are killed.

As a still further example, a suitable specific killing value for the immune cells, pharmaceutical compositions, and kits can be, for example, the following criteria: 1) at least 50% specific killing following a 48 hour co-incubation of immune cells and target cells expressing activator receptor ligand in the absence of inhibitory receptor ligand; and 2) less than or equal to 20% specific killing of target cell expressing both an activator receptor ligand and an inhibitory receptor ligand. As a further example, the immune cells are capable of killing at least 30%, at least 40%, at least 50%, at least 60%, at least 70%, at least 80%, at least 90%, at least 95%, at least 97% or at least 99% of target cells expressing the activator ligand and not the inhibitor ligand over a period of 6 hours, 12 hours, 18 hours, 24 hours, 30 hours, 36 hours, 42 hours, 48 hours, 54 hours, or 60 hours, while killing less than 40%, less than 30%, less than 20%, less than 10%, less than 5%, less than 3% or less than 1% of target cells expressing the activator and inhibitor ligands over the same time period.

A suitable specific killing value of the target cell expressing an activator ligand in the absence of an inhibitory ligand value for the immune cells, pharmaceutical compositions, and kits can be, for example, at least about 50% to at least about 95%. A suitable specific killing value of the target cell expressing an activator ligand in the absence of an inhibitory ligand value for the immune cells, pharmaceutical compositions, and kits can be, for example, at least about 50%, at least about 55%, at least about 60%, at least about 65%, at least about 70%, at least about 75%, at least about 80%, at least about 85%, at least about 90%, or at least about 95%. A suitable specific killing value of the target cell expressing an activator ligand in the absence of an inhibitory ligand value for the immune cells, pharmaceutical compositions, and kits can be, for example, at most about 50%, at most about 55%, at most about 60%, at most about 65%, at most about 70%, at most about 75%, at most about 80%, at most about 85%, at most about 90%, or at most about 95%. A suitable specific killing value of target cells expressing both an activator receptor ligand and an inhibitory receptor ligand for the immune cells, pharmaceutical compositions, and kits can be can be less than about 50%, less than about 45%, less than about 40%, less than about 35%, less than about 30%, less than about 25%, less than about 20%, less than about 15%, less than about 10%, or less than about 5%. The suitable specific killing value for the immune cells, pharmaceutical compositions, and kits can be can be determined following about 6 hours, about 12 hours, about 18 hours, about 24, about 30 hours, about 36 hours, about 42 hours, about 48 hours, about 54 hours, about 60 hours, about 66 hours, or about 72 hours of co-incubation of immune cells with target cells.

A suitable specific killing value of the target cell expressing an activator ligand in the absence of an inhibitory ligand value for the immune cells, pharmaceutical compositions, and kits can be, for example, at least about 50% to at least about 95%. A suitable specific killing value of the target cell expressing an activator ligand in the absence of an inhibitory ligand value for the immune cells, pharmaceutical compositions, and kits can be, for example, at least about 50%, at least about 55%, at least about 60%, at least about 65%, at least about 70%, at least about 75%, at least about 80%, at least about 85%, at least about 90%, or at least about 95%. A suitable specific killing value of the target cell expressing an activator ligand in the absence of an inhibitory ligand value for the immune cells, pharmaceutical compositions, and kits can be, for example, at most about 50%, at most about 55%, at most about 60%, at most about 65%, at most about 70%, at most about 75%, at most about 80%, at most about 85%, at most about 90%, or at most about 95%. A suitable specific killing value of target cells expressing both an activator receptor ligand and an inhibitory receptor ligand for the immune cells, pharmaceutical compositions, and kits can be can be less than about 50%, less than about 45%, less than about 40%, less than about 35%, less than about 30%, less than about 25%, less than about 20%, less than about 15%, less than about 10%, or less than about 5%. The suitable specific killing value for the immune cells, pharmaceutical compositions, and kits can be can be determined following about 6 hours, about 12 hours, about 18 hours, about 24, about 30 hours, about 36 hours, about 42 hours, about 48 hours, about 54 hours, about 60 hours, about 66 hours, or about 72 hours of co-incubation of immune cells with target cells.

As used herein, the term “functional variant” refers to a protein that has one or more amino-acid substitutions, insertions, or deletions as compared to a parental protein, and which retains one or more desired activities of the parental protein. A functional variant may be a fragment of the protein (i.e. a variant having N- and/or C-terminal deletions) that retain the one or more desired activities of the parental protein.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

### Allogeneic Immune Cells

The disclosure provides genetically engineered allogeneic immune cells for use in adoptive cell therapies. The allogeneic immune cells described herein can be modified using any strategies known in the art such as, and without limitation, gene editing to generate targeted gene knockdowns, knockouts, disruptions, insertions, deletions, frameshift mutations, mis-sense mutations, nonsense mutations or substitutions in a target gene, expression of RNAs such as for RNA interference, or expression of protein products which can disrupt gene function. Any technique known in the art that can be used to modify cells to impact on the expression of a target gene or the function of a target gene product is envisaged as within the scope of the instant methods. Genetic engineering strategies include use of gene editing technologies known in the art such as, without limitation, RNA interference (RNAi), Clustered Regularly Interspaced Short Palindromic Repeats (CRISPR)/Cas, transcription activator-like effector nuclease (TALEN), zinc finger nucleases (ZFN), or any technology used to achieve the goals of genetic editing allogeneic immune cells described herein. Genes targeted for editing in the allogeneic immune cells of the disclosure include TCRα, TCRβCD52, B2M, CD3, and/or human leukocyte antigen (HLA), such as HLA-A, HLA-B and/or HLA-C. The allogeneic immune cells of the disclosure can be engineered to modify a target locus leading to disruption of a target gene, or to express a construct, such as an RNAi or protein construct, that disrupts that the target gene or its gene product in trans.

Allogeneic therapeutic cells can serve as pre-manufactured cells, characterized in detail and available for immediate administration to patients in adoptive cell therapy. By allogeneic it is meant that the cells are obtained from individuals belonging to the same species but are genetically dissimilar. However, the use of allogeneic cells presently has many drawbacks. In immune-competent hosts allogeneic cells are rapidly rejected, a process termed host versus graft rejection (HvGD), and this substantially limits the efficacy of the transferred cells. In immune-incompetent hosts, allogeneic cells are able to engraft, but their endogenous TCR specificities recognize the host tissue as foreign, resulting in graft versus host disease (GvHD), which can lead to serious tissue damage and death. In order to effectively use allogeneic cells, both of these problems must be overcome. One strategy used to overcome these problems is targeted disruption of one or more genes encoding TCR subunits in allogeneic therapeutic cells for adoptive cell therapy. Additional strategies include targeted disruption of one or more genes encoding components of the major histocompatibility complex class I (MHC class I) such as HLA-A, HLA-B, HLA-C or beta-2-microglobulin (B2M), and knockout of CD52 in the allogeneic cells to facilitate lymphodepletion of host cells. In a further strategy, the presence of the inhibitory receptor described herein reduces the graft versus host effect, such that allogeneic cells can be employed in which the endogenous TCR receptor subunits are intact.

In some embodiments, the allogeneic immune cells are T cells. In some embodiments, the allogeneic T cells are cytotoxic T cells.

In some embodiments, the allogeneic immune cells express an endogenous TCR receptor. For example, the allogeneic immune cells express endogenous TCR gene products capable of reconstituting an endogenous TCR receptor. The present inventors have discovered that expressing an inhibitory receptor, as described herein, can inhibit the function of endogenous TCR receptors expressed by allogeneic immune cells. This approach allows for the expression of a TCR gene or gene product in allogeneic immune cells used for adoptive gene therapy. Avoiding the need to eliminate donor grafted T cell expression of endogenous TCRs is an advantage over allogeneic adoptive cell therapy methods known in the art, which generally require deletion or inhibition of endogenous TCRs.

In some embodiments, the allogeneic immune cells comprise an activator receptor, an inhibitory receptor, and an endogenous TCR (i.e., the allogeneic immune cells have not been modified to reduce or eliminate expression of one or more TCR subunits). In some embodiments, expression of an inhibitory receptor as described herein reduces GvHD. In some embodiments, expression of an inhibitory receptor by the allogeneic immune cells, for example an inhibitory receptor that targets HLA-A*02, reduces or eliminates GvHD.

In some embodiments, the allogeneic immune cells are modified to knock down, or knock out, one or more subunits of the endogenous TCR receptor by any of the methods described herein.

In immunocompetent hosts, allogeneic cells are rapidly rejected by the host immune system. For example, it has been demonstrated that allogeneic leukocytes present in non-irradiated blood products will persist for no more than 5 to 6 days. Thus, to prevent rejection of allogeneic cells, the host's immune system must be effectively suppressed. Glucocorticoidsteroids are widely used therapeutically for immunosuppression. This class of steroid hormones binds to the glucocorticoid receptor (GR) present in the cytosol of T cells resulting in the translocation into the nucleus and the binding of specific DNA motifs that regulate the expression of a number of genes involved in the immunologic process. Treatment of T cells with glucocorticoid steroids results in reduced levels of cytokine production leading to T cell anergy and interfering in T cell activation. Alemtuzumab, also known as CAMPATH1-H, is a humanized monoclonal antibody targeting CD52, a 12 amino acid glycosylphosphatidyl-inositol-(GPI) linked glycoprotein. CD52 is expressed at high levels on T and B lymphocytes and lower levels on monocytes while being absent on granulocytes and bone marrow precursors. Treatment with Alemtuzumab, a humanized monoclonal antibody directed against CD52, has been shown to induce a rapid depletion of circulating lymphocytes and monocytes. It is frequently used in the treatment of T cell lymphomas and in certain cases as part of a conditioning regimen for transplantation. However, in the case of adoptive immunotherapy, the use of immunosuppressive drugs will also have a detrimental effect on the introduced therapeutic T cells. Therefore, to effectively use an adoptive immunotherapy approach in these conditions, the introduced cells would need to be resistant to the immunosuppressive treatment.

Accordingly, the disclosure provides allogeneic immune cells that have been engineered to be resistant to targeted therapies specific to CD52, such as Alemtuzumab. Allogeneic immune cells can be engineered to be resistant to CD52 targeting therapies by knocking down, or knocking out, CD52 in the allogeneic immune cells using any of the methods described herein.

Modified T cells that lack expression of a target gene can be obtained by any suitable means, including a knock out or knock down of one or more subunit of a target gene. For example, the T cell can include a knock down of a target gene using siRNA, shRNA, clustered regularly interspaced short palindromic repeats (CRISPR), transcription-activator like effector nuclease (TALEN), or zinc finger endonuclease (ZFN).

The disclosure provides genetically engineered allogeneic immune cells. In some embodiments, the allogeneic immune cells are allogeneic T cells. In some embodiments, the allogeneic T cells described herein are genetically modified to express an activator receptor and an inhibitory receptor. In some embodiments, the allogeneic T cells described herein comprise additional genetic modifications. In some embodiments, the allogeneic T cells comprise reduced or eliminated expression of the gene encoding TCRα (e.g. T cell receptor alpha constant region, or TRAC). In some embodiments, the allogeneic T cells comprise reduced or eliminated expression or function of the gene encoding TCRβ (e.g. T cell receptor beta locus, or TRB). In some embodiments, the allogeneic T cells comprise reduced or eliminated expression or function of a gene encoding a CD3 subunit of a TCR (e.g., CD3D, CD3E, CD3G or CD3Z). In some embodiments, the allogeneic T cells described herein are genetically modified to contain a natural killer (NK) cell inhibitor component. The activator component can be, for example, an engineered receptor comprising a ligand binding domain that binds to an activator ligand, as described herein. The blocker component can be, for example, an engineered receptor comprising a second ligand binding domain that binds to an inhibitor ligand, as described herein. The NK cell inhibitor component can be, for example, human leukocyte antigen E (HLA-E), human leukocyte antigen G (HLA-G) or killer cell lectin like receptor C1 (KLRC1 or NKG2A) expression.

In some embodiments, the allogeneic immune cells described herein are genetically modified to reduce or eliminate expression of function of CD52.

In immune-incompetent hosts, allogeneic cells are able to engraft, but their endogenous TCR specificities recognize the host tissue as foreign, resulting in graft versus host disease (GvHD), which can lead to serious tissue damage and death. In some embodiments, the genetically engineered allogeneic immune cells comprise genetic edits to reduce or eliminate graft versus host disease (GvHD). In some embodiments, the genetically engineered allogeneic T cells comprise genetic edits to make TRAC gene (i.e., a gene encoding TCR alpha) to reduce or eliminate GvHD. In some embodiments, the genetically engineered allogeneic T cells comprise genetic edits to delete the TRB gene to reduce or eliminate GvHD. In some embodiments, the genetically engineered allogeneic immune cells comprise genetic edits to delete the gene encoding one or more CD3 T cell receptor subunits to reduce to eliminate GvHD.

In immune-competent hosts, allogeneic cells can rapidly be rejected, a process termed host versus graft rejection (HvGD), and this substantially limits the efficacy of the transferred cells. In some embodiments, the genetically engineered allogeneic T cells comprise genetic edits to reduce or eliminate host versus graft disease (HvGD) by reducing or eliminating one or more MHC class one components, and/or expressing an NK cell inhibitor such as HLA-E, HLA-G or NKG2A. In some embodiments, the genetically engineered allogeneic T cells comprise genetic edits to delete the B2M gene to reduce or eliminate HvGD. In some embodiments, expression of a B2M-targeting scFv reduces or eliminates HvGD.

In some embodiments, the genetically engineered allogeneic T cells comprise genetic edits to reduce or eliminate expression of the CD52 gene.

In some embodiments, the allogeneic immune cell comprises one or more modifications that reduce immune cell exhaustion, for example T cell exhaustion. As used herein, “T cell exhaustion” refers to the response of T cells to chronic antigen stimulation. Exhaustion is characterized by a step-wise and progressive loss of T cell function, and can culminate in physical elimination of the T cells. Exhaustion is regulated by a variety of inhibitory molecules such as immune checkpoint regulators, and, without wishing to be bound by theory, it is thought that by modifying expression of these molecules by the allogeneic immune cells, exhaustion of these cells can be ameliorated.

In some embodiments, the allogeneic immune cell can be a cell which does not express, or expresses at low levels, an inhibitory molecule that prevents T cell exhaustion. The allogeneic immune cell can be modified by any method described herein or known in the art. For example, the cell can be a cell that does not express, or expresses at low levels, an inhibitory molecule that can decrease the ability of the allogeneic immune cell to mount an immune effector response. Examples of inhibitory molecules include PD1, PD-L1, CTLA4, TIM3, CEACAM (e.g., CEACAM-1, CEACAM-3 and/or CEACAM-5), LAG3, VISTA, BTLA, TIGIT, LAIR1, CD160, 2B4 and TGFR beta. Modification of the allogeneic immune cell, e.g., by modification at DNA, RNA or protein level by any of the methods described herein, can optimize allogeneic immune cell performance. In some embodiments, an inhibitory nucleic acid, e.g., a dsRNA, siRNA or shRNA can be used. In some embodiments, the allogeneic immune cell can be modified using any of the methods described herein to mutate (full or partial deletion, substitution, insertion, frameshift mutation and the like) the locus encoding the inhibitory molecule.

Allogeneic immune cells, for example those that do not express a functional MHC class I complex, may also be targeted by host NK cells. Killing of allogeneic immune cells by host NK cells can reduce the survival, and therefore the efficacy, of the transplanted cells. One approach to mitigate NK cell mediated killing of transplanted allogeneic cells is through expression of a B2M-HLA fusion protein, for example B2M-HLA-E or B2M-HLA-G fusion proteins, by the transplanted cells. Exemplary fusion proteins that can protect allogeneic T cells from NK-cell mediated lysis are described in Guo et al., European Journal of Immunology (2021) 51: 2513-2521, the contents of which are incorporated by reference herein.

Accordingly, the disclosure provides a fusion protein comprising B2M protein fused to an HLA protein. In some embodiments, the HLA protein comprises HLA-E. In some embodiments, the HLA protein comprises HLA-G. In some embodiments, the B2M protein and HLA protein are separated by a linker, for example a glycine-serine linker as described herein. In some embodiments, the fusion protein further comprises a peptide, for example a peptide of 3-30 amino acids, 5-25, 5-20 or 5-15 amino acids. In some embodiments, the peptide is linked to B2M by a linker, such as a glycine-serine linker as described herein. Exemplary arrangements include, from N to C terminus, peptide-linker-B2M-linker-HLA-E, B2M-linker-HLA-E, peptide-linker-B2M-linker-HLA-G, and B2M-linker-HLA-G.

In some embodiments, the B2M and HLA portions of the fusion protein form a complex on the surface of an allogeneic immune cell. In some embodiments, the peptide, B2M and HLA portions of the fusion protein form a complex on the surface of an allogeneic immune cell.

The disclosure further provides immune cells comprising the B2M-HLA fusion proteins described herein, and vectors encoding the B2M-HLA fusion proteins described herein. In some embodiments, the vector comprises a promoter operably linked to the fusion protein, for example a constitutive promoter.

The skilled artisan will be able to select a suitable fusion protein based on genotype of the donor cells with respect to HLA, and the particular blocker receptor expressed by the allogeneic cells.

### Gene Targets

In some embodiments, the genetically engineered allogeneic immune cells described herein are modified to reduce or eliminate expression or function of a target gene. Exemplary target genes are described below.

### MHC Class I Genes and Polypeptides

In some embodiments, the genetically engineered allogeneic immune cells described herein are modified to reduce or eliminate expression of the B2M gene. The beta-2 microglobulin (B2M) gene encodes a protein that associates with the major histocompatibility complex (MHC) class I, i.e. MHC-I complex. The MHC-I complex is required for presentation of antigens on the cell surface. HLA-A, HLA-B and HLA-C genes that form part of MHC I are highly polymorphic, and when expressed on the surface of cells used in adoptive cell therapy can present as “non-self” and facilitate host rejection (HvGD). The MHC-I complex is disrupted and non-functional when the B2M is deleted (Wang D et al. Stem Cells Transl Med. 4:1234-1245 (2015)). Furthermore, the B2M gene can be disrupted with high efficiency using gene editing techniques known in the art (Ren et al. Clin. Cancer Res. 23:2255-2266 (2017)). Reducing or eliminating B2M can reduce, or eliminate functional MHC I on the surface of the allogeneic immune cell. The major histocompatibility complex (MHC) is a locus on the vertebrate genome that encodes a set of polypeptides required for the adaptive immune system. Among these are MHC class I polypeptides that include HLA-A, HLA-B, and HLA-C and alleles thereof. MHC class I alleles are highly polymorphic and expressed in all nucleated cells. MHC class I polypeptides encoded by HLA-A, HLA-B, and HLA-C and alleles thereof form heterodimers with (32 microglobulin (B2M) and present in complex with antigens on the surface of cells. In some embodiments, the immune cells of the disclosure are inactivated by an inhibitor ligand comprising an MHC class I polypeptide, e.g. HLA-A, HLA-B, and HLA-C and alleles thereof. HLA-A alleles can be, for example and without limitation, HLA-A*02, HLA-A*02:01, HLA-A*02:01:01, HLA-A*02:01:01:01, and/or any gene that encodes protein identical or similar to HLA-A*02 protein. Thus, to prevent autocrine signaling/binding as described herein, it is desirable to eliminate or reduce expression of polypeptides encoded by HLA-A, HLA-B, and HLA-C and alleles thereof in the immune cells.

In some embodiments, the genetically engineered allogeneic immune cells described herein are modified to inactivate or reduce or eliminate expression or function of an endogenous gene encoding an allele of an endogenous MHC class I polypeptide. In some embodiments, the gene encoding the MHC class I polypeptide is HLA-A, HLA-B and/or HLA-C. HLA-A, HLA-B and HLA-C are encoded by the HLA-A, HLA-B and HLA-C loci. Each of HLA-A, HLA-B and HLA-C includes many variant alleles, all of which are envisaged as within the scope of the instant disclosure. In some embodiments, the gene encoding the MHC class I polypeptide is HLA-A. In some embodiments, the gene encoding the MHC class I polypeptide is HLA-A*02. In some embodiments, the gene encoding the MHC class I polypeptide is HLA-A*02:01. In some embodiments, the gene encoding the MHC class I polypeptide is HLA-A*02:01:01. In some embodiments, the gene encoding the MHC class I polypeptide is HLA-A*02:01:01:01.

### CD52 Gene

In some embodiments, the genetically engineered allogeneic immune cells described herein are modified to reduce or eliminate expression or function of the CD52 gene. CD52 is a 12 amino acid glycosylphosphatidyl-inositol-(GPI) linked glycoprotein (Waldmann and Hale 2005). CD52 is expressed at high levels on T and B lymphocytes and lower levels on monocytes while being absent on granulocytes and bone marrow precursors. Alemtuzumab, also known as CAMPATH1-H, is a humanized monoclonal antibody targeting CD52. Treatment with Alemtuzumab, a humanized monoclonal antibody directed against CD52, has been shown to induce a rapid depletion of circulating lymphocytes and monocytes. It is frequently used in the treatment of T cell lymphomas and in certain cases as part of a conditioning regimen for transplantation and adoptive cell therapies. In the case of adoptive immunotherapy, the use of immunosuppressive drugs will also have a detrimental effect on the introduced therapeutic allogeneic immune cells. Therefore, to effectively use an adoptive immunotherapy approach in these conditions, the introduced cells, e.g. allogeneic immune cells, need to be resistant to the immunosuppressive treatment. Resistance to alemtuzumab-based lymphodepletion or conditioning can be achieved by reducing or eliminating CD52 expression in the allogeneic cells administered to the subject.

### TCR Subunits

In some embodiments, the genetically engineered allogeneic immune cells described herein comprise genetic edits to reduce or eliminate expression of the TCRα gene (TRAC). In some embodiments, the genetically engineered allogeneic immune cells described herein comprise genetic edits to reduce or eliminate expression of the TCRβ gene (TRB). T cell receptors (TCR) are cell surface receptors that participate in the activation of T cells in response to the presentation of antigen. The TCR includes two protein chains, alpha (TCRα) and beta (TCRβ), which assemble to form a heterodimer and associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of GvHD. Normal surface expression of the TCR depends on the coordinated synthesis and assembly of all seven components of the complex. The disruption of TCRα and/or beta TCRβ can result in the elimination of the TCR from the surface of T cells, thereby preventing recognition of alloantigen and thus GvHD.

In some embodiments, the genetically engineered allogeneic immune cells described herein are modified to reduce or eliminate expression of a CD3 gene (CD3D, CD3E, CD3G and/or CD3Z). The CD3 genes form part of the TCR complex, and are required for activation of TCR. CD3 genes as referred to herein comprise the genes encoding the CD3γ, CD3δ, CD3ε, and CD3ξ proteins. The CD3 complex makes up the signaling component of an activated TCR complex, resulting in T cell expansion/proliferation, upregulation of activation markers on the T cell surface, and induction of cytotoxicity or cytokine secretion. Native T cell signaling in allogeneic immune cells can recognize normal host tissue as foreign, and thus result in GvHD. Targeted reduction or elimination of native CD3 genes in allogeneic immune cells can eliminate native T cell signaling and thus reduce or eliminate GvHD.

### Target Gene Sequences

The disclosure provides an immune cell comprising an inhibitory receptor comprising a ligand binding domain specific to a class I major histocompatibility complex (MHC-I) molecule, or a peptide-MHC complex thereof; wherein expression and/or function of beta-2− microgloobulin (B2M) in said immune cell has been reduced or eliminated. In some embodiments, the immune cell comprises an interfering RNA, comprising a sequence complementary an RNA sequence transcribed from the B2M gene

In some embodiments, the target gene is the B2M gene. In some embodiments, the interfering RNA is complementary to at least a portion of a B2M mRNA-B2M RNA comprises a coding sequence. In some embodiments, the B2M mRNA sequence comprises an untranslated region.

The disclosure provides an immune cell comprising an inhibitory receptor comprising a ligand binding domain specific to a class I major histocompatibility complex (MHC-I) molecule, or a peptide-MHC complex thereof; wherein the immune cell comprises one or more modifications that reduce autocrine signaling/binding by the receptor. In some embodiments, the modifications comprise an inactivating mutation in an endogenous target gene. In some embodiments, the endogenous target gene is B2M.

Target gene sequences include, but are not limited to, gene elements such as promoters, enhancers, introns, exons, intron/exon junctions, transcription products (pre-mRNA, mRNA, and splice variants), and/or 3′ and 5′ untranslated regions (UTRs). Any gene element or combination of gene elements may be targeted for the purpose of genetic editing in the allogeneic immune cells described herein.

Interfering RNAs of the disclosure target and bind to a target sequence through base pair complementarity. The interfering RNA has a complementary sequence to any region of a target gene sequence that is transcribed into RNA. In some embodiments, the target gene is HLA. In some embodiments, the target gene is B2M. Transcribed RNA can include intronic regions (introns), expressed regions (exons), untranslated regions (UTRs), coding sequences (CDS), or any other region of the target gene that undergoes transcription. Transcribed RNA can include primary transcripts, pre-mRNA, mature mRNA, and/or mRNA splice variants. RNA can include regulatory signals, such as polyA sites or polyA signal sequences. Transcribed RNA can include any non-coding region of the target gene that is not transcribed as part of an mRNA transcript, e.g. long noncoding RNA (lnRNA) or micro RNA (miRNA). Any transcribed region of the target gene may be targeted for the purpose of reducing or eliminating expression of the target gene in the immune cells described herein. Delivery of interfering RNA can be accomplished using any method known in the art to target gene transcripts that results in altered, disrupted, reduced, or eliminated expression or function the target gene or gene product. Examples of target gene sequences are set forth in SEQ ID NOs: 382-389.

The disclosure provides an immune cell comprising an inhibitory receptor comprising a ligand binding domain specific to a class I major histocompatibility complex (MHC-I) molecule, or a peptide-MHC complex thereof; wherein the immune cell comprises one or more modifications that reduce autocrine signaling/binding by the receptor. In some embodiments, the modifications comprise an inactivating mutation in an endogenous target gene.

Modifications to target genes can be accomplished using any method known in the art to edit the target gene that results in altered or disrupted expression or function the target gene or gene product.

In some embodiments, modifying the gene encoding the MHC class I polypeptide comprises deleting all or a portion of the gene. In some embodiments, modifying the gene encoding the MHC class I polypeptide comprises introducing a mutation in the gene. In some embodiments, the mutation comprises a deletion, insertion, substitution, or frameshift mutation. In some embodiments, modifying the gene comprises using a nucleic acid guided endonuclease.

The disclosure provides an immune cell comprising an inhibitory receptor comprising a ligand binding domain specific to a class I major histocompatibility complex (MHC-I) molecule, or a peptide-MHC complex thereof; wherein expression and/or function of human leukocyte antigen (HLA) in said immune cell has been reduced or eliminated. In some embodiments, the—immune cell comprises an interfering RNA, comprising a sequence complementary an RNA sequence transcribed from the HLA gene.

In some embodiments, the target gene is an allele of an endogenous MHC class I polypeptide specifically bound by the inhibitory receptor. In some embodiments, the gene encoding the MHC class I polypeptide is HLA-A, HLA-B, HLA-C, or a combination thereof. In some embodiments, the gene encoding the MHC class I polypeptide is HLA-A. HLA-A is a polymorphic gene whose various alleles may also be target genes for modification. The alleles may also be referred to as genes, and can include, for example, the HLA-A*02, HLA-A*02:01, HLA-A*02:01:01, and/or the HLA-A*02:01:01:01 alleles. In some embodiments, the gene encoding the MHC class I polypeptide is HLA-A*02. In some embodiments, the gene encoding the MHC class I polypeptide is HLA-A*02:01. In some embodiments, the gene encoding the MHC class I polypeptide is HLA-A*02:01:01. In some embodiments, the gene encoding the MHC class I polypeptide is HLA-A*02:01:01:01.

In some embodiments, the target gene is an HLA gene. In some embodiments, the HLA gene is the HLA-A gene. When used herein as a reference to a target gene or an associated transcript (e.g., mRNA), the HLA may refer to HLA-A, the HLA-A*02 allele, the HLA-A*02:01 allele, the HLA-A*02:01:01 allele, and/or the HLA-A*02:01:01:01 allele. In some embodiments, the HLA gene is the HLA-A*02 allele. In some embodiments, the HLA gene is the HLA-A*02:01 allele. In some embodiments, the HLA gene is the HLA-A*02:01:01 allele. In some embodiments, the HLA gene is the HLA-A*02:01:01:01 allele. In some embodiments, the interfering RNA is complementary to at least a portion of an mRNA transcribed from an HLA gene. In some embodiments, the mRNA is transcribed from an HLA-A*02 allele. In some embodiments, the mRNA is transcribed from an HLA-A*02:01 allele. In some embodiments, the mRNA is transcribed from an HLA-A*02:01:01 allele. In some embodiments, the mRNA is transcribed from an HLA-A*02:01:01:01 allele. In some embodiments, the mRNA comprises a coding sequence. In some embodiments, the mRNA sequence comprises an untranslated region.

Gene sequences for the target genes described herein are known in the art. The sequences can be found at public databases, such as NCBI GenBank or the NCBI nucleotide database. Sequences may be found using gene identifiers, for example, the HLA-A gene has NCBI Gene ID: 3105, the HLA-B gene has NCBI Gene ID: 3106, and the HLA-C gene has NCBI Gene ID: 3107. Gene sequences may also be found by searching public databases using keywords. For example, HLA-A alleles may be found in the NCBI nucleotide database by searching keywords, “HLA-A*02”, “HLA-A*02:01”, “HLA-A*02:01:01”, or “HLA-A*02:01:01:01.” These sequences can be used for targeting in various gene editing techniques known in the art. Non-limiting illustrative sequences for the target HLA-A allele gene sequences targeted for modification as described herein are set forth in SEQ ID NOs: 8346-8349 and 16871.

In some embodiments, the target gene or target gene sequence is a B2M gene or B2M gene sequence. In some embodiments, the target sequence is the coding sequence (CDS) of the B2M gene. In some embodiments, the target sequence is a promoter sequence. In some embodiments, the sequence is the B2M promoter.

In some embodiments, modifying the B2M gene comprises deleting all or a portion of the B2M gene. In some embodiments, modifying the B2M gene comprises introducing a mutation in the B2M gene. In some embodiments, the mutation comprises a deletion, insertion, substitution, or frameshift mutation. In some embodiments, modifying the gene comprises using a nucleic acid guided endonuclease.

Gene sequences for B2M and transcribed RNA will be known to persons of ordinary skill in the art. The B2M gene sequences and transcribed regions can be found at public databases, such as NCBI GenBank or the NCBI nucleotide database. Sequences may be found using gene identifiers, for example, the B2Mgene has NCBI Gene ID: 567 and NCBI Reference Sequence: NC 000015.10. Gene sequences may also be found by searching public databases using keywords. For example, B2M may be found in the NCBI nucleotide database by searching keywords, “B2M” or “beta-2-microglobulin.” Gene sequences include sequence elements such as, but not limited to, exons, coding sequences (CDS), introns, pre-cursor RNAs, mRNAs, and the like. Transcriptional regulatory elements controlling B2M expression, such as promoters and enhancers, are also considered part of the gene and can be targeted using the methods described herein. Any B2M gene transcribed sequence may be targeted by any gene editing methods known in the art or the interfering RNAs described herein (e.g. mRNA). Non-limiting illustrative sequences for B2M gene sequences and transcribed RNA sequences, of which the RNA sequences that can be targeted for modification or degradation by interfering RNAs as described herein are set forth in SEQ ID NOs: 8471, 8472, and 21897.

siRNA, miRNA, and shRNA to Inhibit Expression of a Target Protein

In some embodiments, expression of a target protein can be inhibited using an interfering RNA (RNA interference, or RNAi). In some embodiments, the interfering RNA is short interfering RNA (siRNA), microRNA (miRNA), or short hairpin RNA (shRNA) that targets a nucleic acid encoding the target protein in an allogeneic immune cell described herein. Expression of siRNA, miRNAs, and shRNAs in an allogeneic immune cell can be achieved using any expression system known in the art, e.g., a lentiviral expression system.

RNA interference (RNAi) is a process by which double-stranded RNA (dsRNA) is used to silence gene expression. While not wanting to be bound by theory, RNAi begins with the cleavage of longer dsRNAs into small interfering RNAs (siRNAs) by an RNaseIII-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the antisense strand) can favor incorporation of the antisense strand into RISC.

RISC-mediated cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein via translational repression without cleavage of the target mRNA. Other RNA molecules and RNA-like molecules can also interact with RISC and silence gene expression. Examples of other RNA molecules that can interact with RISC include short hairpin RNAs (shRNAs), single-stranded siRNAs, microRNAs (miRNAs), and dicer-substrate 27-mer duplexes. The term “siRNA” as used herein refers to a double-stranded interfering RNA unless otherwise noted. Examples of RNA-like molecules that can interact with RISC include RNA molecules containing one or more chemically modified nucleotides, one or more deoxyribonucleotides, and/or one or more non-phosphodiester linkages. For purposes of the present discussion, all RNA or RNA-like molecules that can interact with RISC and participate in RISC-mediated changes in gene expression will be referred to as “interfering RNAs.” SiRNAs, shRNAs, miRNAs, and dicer-substrate 27-mer duplexes are, therefore, subsets of “interfering RNAs.”

Interfering RNA of embodiments of the invention appear to act in a catalytic manner for cleavage of target mRNA, i.e., interfering RNA is able to effect inhibition of target mRNA in substoichiometric amounts. As compared to antisense therapies, significantly less interfering RNA is required to provide a therapeutic effect under such cleavage conditions.

Interfering RNAs can be designed for a target gene using methods known in the art. In embodiments of the present invention, interfering RNA target sequences (e.g., siRNA target sequences) within a target mRNA sequence are selected using available design tools. Techniques for selecting target sequences for siRNAs are provided by Tuschl, T. et al., “The siRNA User Guide.” revised May 6, 2004, available on the Rockefeller University web site; by Technical Bulletin #506, “siRNA Design Guidelines.” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 18 and 27 nucleotides. The target sequence may be located in the coding region or in the 5′ or 3′ untranslated regions of the mRNA.

Interfering RNAs are delivered to the cell using a construct made using methods known in the art. Constructs are commonly made by synthesizing the interfering RNA, annealing, and ligating two complementary oligonucleotides into an expression vector. Another known method uses PCR, whereby a promoter sequence serves as a template and the interfering sequence is contained in the reverse primer, and PCR results in an amplified cloning cassette comprising both promoter and interfering RNA. The amplified cassette is purified from truncated oligos by polyacrylamide gel electrophoresis prior to delivery to the cell. Another approach an interfering RNA template formed from two long partially complementary oligos of approximately equal length, overlapping at their 3′ ends. Each oligo serves as both template (for extending the opposite oligo) and primer (to copy the opposite oligo). Extension and repeated cycling generates a double-stranded product, akin to that generated in the annealed oligo method. The product can be further amplified by PCR with addition of another short primer binding the extended strand. Other methods for making and preparing interfering RNA constructs for cellular delivery and expression are known in the art (Timmons L. Methods Mol Biol. 351:109-117 (2006); Paul C et al. Nature Biotechnology. 20:505-508 (2002); Gupta S et al. PNAS 7:1927-1932 (2004)).

Exemplary shRNAs that downregulate expression of a target gene, such as components of the TCR are described, e.g., in US Publication No.: 2012/0321667. Exemplary siRNA and shRNA that downregulate expression of a target gene, as described herein, are described, e.g., in U.S. publication No.: US 2007/0036773.

### Illustrative Interfering RNAs

The disclosure provides interfering RNAs. The double stranded RNA molecule of the invention may be in the form of any type of RNA interference molecule known in the art. In some embodiments, the double stranded RNA molecule is a small interfering RNA (siRNA). In other embodiments, the double stranded RNA molecule is a short hairpin RNA (shRNA) molecule. In other embodiments, the double stranded RNA molecule is a Dicer substrate that is processed in a cell to produce an siRNA. In other embodiments the double stranded RNA molecule is part of a microRNA precursor molecule.

In some embodiments, the shRNA is a length to be suitable as a Dicer substrate, which can be processed to produce a RISC active siRNA molecule. See, e.g., Rossi et al., US2005/0244858.

A Dicer substrate double stranded RNA (e.g. a shRNA) can be of a length sufficient that it is processed by Dicer to produce an active siRNA, and may further include one or more of the following properties: (i) the Dicer substrate shRNA can be asymmetric, for example, having a 3′ overhang on the anti-sense strand, (ii) the Dicer substrate shRNA can have a modified 3′ end on the sense strand to direct orientation of Dicer binding and processing of the dsRNA to an active siRNA, for example the incorporation of one or more DNA nucleotides, and (iii) the first and second strands of the Dicer substrate ds RNA can be from 21-30 bp in length.

shRNAs of the disclosure may be generated exogenously by chemical synthesis, by in vitro transcription, or by cleavage of longer double-stranded RNA with Dicer or another appropriate nuclease with similar activity. Chemically synthesized siRNAs, produced from protected ribonucleoside phosphoramidites using a conventional DNA/RNA synthesizer, may be obtained from commercial suppliers such as Millipore Sigma (Houston, Tex.), Ambion Inc. (Austin, Tex.). Invitrogen (Carlsbad, Calif.), or Dharmacon (Lafayette, Colo.). siRNAs can be purified by extraction with a solvent or resin, precipitation, electrophoresis, chromatography, or a combination thereof, for example. Alternatively, siRNAs may be used with little if any purification to avoid losses due to sample processing.

In some embodiments, shRNAs of the disclosure can be produced using an expression vector into which a nucleic acid encoding the double stranded RNA has been cloned, for example under control of a suitable promoter.

In some embodiments, the interfering RNAs comprise a sequence complementary to a sequence of a HLA-A*02 mRNA. In some embodiments, the interfering RNA is capable of inducing RNAi-mediated degradation of the HLA-A*02 mRNA. In some embodiments, the HLA-A*02 mRNA sequence comprises a coding sequence. In some embodiments, the HLA-A*02 mRNA sequence comprises an untranslated region.

In some embodiments, the interfering RNA (is a short hairpin RNA (shRNA). In some embodiments, the shRNA comprises a first sequence, having from 5′ to 3′ end a sequence complementary to the HLA-A*02 mRNA; and a second sequence, having from 5′ to 3′ end a sequence complementary to the first sequence, wherein the first sequence and second sequence form the shRNA.

In some embodiments, the first sequence is 18, 19, 20, 21, or 22 nucleotides. In some embodiments, the first sequence is complementary to a sequence selected from SEQ ID NOs: 8476-15870. In some embodiments, the first sequence has GC content greater than or equal to 25% and less than 60%. In some embodiments, first sequence is complementary to a sequence selected from SEQ ID NOs: 8476-12066. In some embodiments, the first sequence does not comprise four nucleotides of the same base or a run of seven C or G nucleotide bases. In some embodiments, the first sequence is complementary to a sequence selected from SEQ ID NOs: 8476-11584. In some embodiments, the first sequence is complementary to a sequence selected from SEQ ID NO: 8476-8754. In some embodiments, the first sequence is complementary to a sequence selected from SEQ ID NOs: 8476-8561. Illustrative target HLA sequences complementary to the first sequence are set forth in SEQ ID NOs: 8476-8561. Target HLA sequences set forth herein may be presented as DNA sequences. In all target HLA sequences, thymine (T) may be replaced by uracil (U) to arrive at the sequence of the target mRNA sequence.

In some embodiments, the interfering RNAs comprise a sequence complementary to a sequence of a B2M mRNA. In some embodiments, the interfering RNA is capable of inducing RNAi-mediated degradation of the B2M mRNA. In some embodiments, the B2M mRNA sequence comprises a coding sequence. In some embodiments, the B2M mRNA sequence comprises an untranslated region.

In some embodiments, the shRNA comprises a first sequence, having from 5′ to 3′ end a sequence complementary to the B2M mRNA; and a second sequence, having from 5′ to 3′ end a sequence complementary to the first sequence, wherein the first sequence and second sequence form the shRNA.

In some embodiments, the first sequence is 18, 19, 20, 21, or 22 nucleotides. In some embodiments, the first sequence is complementary to a sequence selected from SEQ ID NOs: 16897-21508, 847-8474, and 8368-8370. In some embodiments, the first sequence has GC content greater than or equal to 25% and less than 60%. In some embodiments, the first sequence is complementary to a sequence selected from SEQ ID NOs: 16897-20484, 847-8474, and 8368-8370. In some embodiments, the first sequence does not comprise four nucleotides of the same base or a run of seven C or G nucleotide bases. In some embodiments, the first sequence is complementary to a sequence selected from SEQ ID NOs: 16897-19888, 847-8474, and 8368-8370. In some embodiments, the first sequence is 21 nucleotides. In some embodiments, the first sequence is complementary to a sequence selected from SEQ ID NOs: 16897-17478. In some embodiments, the first sequence is complementary to a sequence selected from SEQ ID NOs: 16897-17178. In some embodiments, the first sequence is complementary to a sequence selected from SEQ ID NOs: 16897-17034. Illustrative target B2M sequences complementary to the first sequence are set forth in SEQ ID NOs: 16897-17034.

In some cases, the first sequence may have 100% identity, i.e. complete identity, homology, complementarity to the target nucleic acid sequence. In other cases, there may be one or more mismatches between the first sequence and the target nucleic acid sequence. For example, there may be 1, 2, 3, 4, 5, 6, or 7 mismatches between the sense region and the target nucleic acid sequence.

In some embodiments, the first and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA. In some embodiments, the loop region comprises a sequence selected from SEQ ID NOs: 16872-16884, and 16895.

In some embodiments, the first sequence and second sequence are separated by a linker, sometimes referred to as a loop. In some embodiments, both the first sequence and the second sequence are encoded by one single-stranded RNA or DNA vector. In some embodiments, the loop is between the first and second sequences. In these embodiments, and the first sequence and the second sequence hybridize to form a duplex region. The first sequence and second sequence are joined by a linker sequence, forming a “hairpin” or “stem-loop” structure. The shRNA can have complementary first sequences and second sequences at opposing ends of a single stranded molecule, so that the molecule can form a duplex region with the complementary sequence portions, and the strands are linked at one end of the duplex region by a linker (i.e. loop sequence). The linker, or loop sequence, can be either a nucleotide or non-nucleotide linker. The linker can interact with the first sequence, and optionally, second sequence through covalent bonds or non-covalent interactions.

Any suitable nucleotide loop sequence is envisaged as within the scope of the disclosure. An shRNA of this disclosure may include a nucleotide, non-nucleotide, or mixed nucleotide/non-nucleotide linker that joins the first sequence of the shRNA to the second sequence of the shRNA. A nucleotide loop sequence can be >2 nucleotides in length, for example about 4, 5, 6, 7, 8, 9, 10, 11, 12, 13, 14, or 15 nucleotides in length.

Examples of a non-nucleotide linker include an abasic nucleotide, polyether, polyamine, polyamide, peptide, carbohydrate, lipid, polyhydrocarbon, or other polymeric agents, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc, 1991, Vol. 113, pp. 6324-6326; Jaeschke et al., Tetrahedron Lett., 1993, Vol. 34, pp. 301; Arnold et al., WO 1989/002439; Usman et al., WO 1995/006731; Dudycz et al., WO 1995/011910, and Ferentz et al., J. Am. Chem. Soc, 1991, Vol. 113, pp. 4000-4002.

In some embodiments, the shRNA further comprises a 5′ flank sequence and a 3′ flank sequence. In some embodiments, wherein the 5′ flank sequence is joined to the 5′ end of the first sequence, and wherein the 3′ flank sequence is joined to the 3′ end of the second sequence.

In some embodiments, the shRNA is capable of inducing RNAi-mediated degradation of the B2M mRNA. In some embodiments, the shRNA is encoded by a sequence comprising a sequence of GCACTCAAAGCTTGTTAAGATCGAAATCTTAACAAGCTTTGAGTGC (SEQ ID NO: 21900) or GTTAACTTCCAATTTACATACCGAAGTATGTAAATTGGAAGTTAAC (SEQ ID NO: 21901), or a sequence having at least 80%, at least 90%, or at least 95% identity thereto.

Without wishing to be bound by theory, it is thought that flanking shRNA stem loop sequence with 5′ and 3′ sequences similar to those found in microRNAs can target the shRNA for processing by the endogenous microRNA processing machinery, increasing the effectiveness of shRNA processing. Alternatively, or in addition, flanking sequences may increase shRNA compatibility with polymerase II or polymerase III promoters, leading to more effective regulation of shRNA expression.

In some embodiments, the 5′ flank sequence is selected from SEQ ID NO: 16885-16887. In some embodiments, the 3′ flank sequence is selected from SEQ ID NO: 16888, 16889, and 16896.

### Targeted Gene Editing Using Nucleic Acid Guided Endonuclease Systems

In some embodiments, a target gene (or target sequence) is edited in the allogeneic immune cells described herein using a nucleic acid guided endonuclease. Exemplary nucleic acid guided endonucleases include Class 2 endonucleases, such as CRISPR/Cas9.

“CRISPR” or “CRISPR gene editing” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence, knock out, or mutate a target gene.

Naturally-occurring CRISPR/Cas systems are found in approximately 40% of sequenced eubacteria genomes and 90% of sequenced archaea. This system is a type of prokaryotic immune system that confers resistance to foreign genetic elements such as plasmids and phages and provides a form of acquired immunity. The CRISPR/Cas system has been modified for use in gene editing (silencing, knock out, enhancing or changing specific genes) in eukaryotes. Wiedenheft et al. (2012) Nature 482: 331-8. This is accomplished by introducing into the eukaryotic cell a one or more specifically designed guide nucleic acids (gNAs), typically guide RNAs (gRNAs), and an appropriate Cas endonuclease which forms a ribonucleoprotein complex with the gNA. The gNA guides the gNA-endonuclease protein complex to a target genomic location, and the endonuclease introduces a double strand break at the target genomic location. This double strand break can be repaired by cellular mechanisms such non-homologous end joining (leading to deletions) or homologous repair (which can generate insertions), thereby introducing genetic modifications into the host cell genome.

CRISPR/Cas systems are classified by class and by type. Class 2 systems currently represent a single interference protein that is categorized into three distinct types (types II, V and VI). Any class 2 CRISPR/Cas system suitable for gene editing, for example a type II, a type V or a type VI system, is envisaged as within the scope of the instant disclosure. Exemplary Class 2 type II CRISPR systems include Cas9, Csn2 and Cas4. Exemplary Class 2, type V CRISPR systems include, Cas12, Cas12a (Cpf1), Cas12b (C2c1), Cas12c (C2c3), Cas12d (CasY), Cas12e (CasX), Cas12f, Cas12g, Cas12h, Cas12i and Cas12k (C2c5). Exemplary Class 2 Type VI systems include Cas13, Cas13a (C2c2) Cas13b, Cas13c and Cas13d.

The CRISPR sequence, sometimes called a CRISPR locus, comprises alternating repeats and spacers. In a naturally-occurring CRISPR, the spacers usually comprise sequences foreign to the bacterium such as a plasmid or phage sequence. As described herein, spacer sequences may also be referred to as “targeting sequences.” In CRISPR/Cas systems for a genetic engineering, the spacers are derived from the target gene sequence (the gNA).

As these CRISPR/Cas occurs naturally in many different types of bacteria, the exact arrangements of the CRISPR and structure, function and number of Cas genes and their product differ somewhat from species to species. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cm′ or Cas2. The Cmr (Cas RAMP module) proteins in Pyrococcus furiosus and other prokaryotes form a functional complex with small CRISPR RNAs that recognizes and cleaves complementary target RNAs.

An exemplary Class 2 type II CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836. In some embodiments, the Cas protein used to modify the allogeneic immune cells is Cas9.

The CRISPR/Cas system can thus be used to edit a target gene, such as a gene targeted for editing in the allogeneic immune cells described herein, by adding or deleting a base pair, or introducing a premature stop which thus decreases expression of the target. The CRISPR/Cas system can alternatively be used like RNA interference, turning off a target gene in a reversible fashion. In a mammalian cell, for example, the RNA can guide the Cas protein to a target gene promoter, sterically blocking RNA polymerases.

A Cas protein may be derived from any bacterial or archaeal Cas protein. Any suitable CRISPR/Cas system is envisaged as within the scope of the instant disclosure. In other aspects, Cas protein comprises one or more of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. In some embodiments, the Cas protein is a Cas9 protein, a Cpf1 protein, a C2c1 protein, a C2c2 protein, a C2c3 protein, Cas3, Cas3-HD, Cas5, Cas7, Cas8, Cas10, or combinations or complexes of these. In some embodiments, the Cas protein is a Cas9 protein. In some embodiments, the Cas9 protein shares at least 90%, at least 95%, at least 96%, at least 97%, at least 98%, or at least 99% sequence identity to SEQ ID NO: 8350.

Artificial CRISPR/Cas systems can be generated which inhibit a target gene, using technology known in the art, e.g., that described in U.S. Publication No. 20140068797, and Cong (2013) Science 339: 819-823. Other artificial CRISPR/Cas systems that are known in the art may also be generated which inhibit a target gene, e.g., that described in Tsai (2014) Nature Biotechnol., 32:6 569-576, U.S. Pat. Nos. 8,871,445; 8,865,406; 8,795,965; 8,771,945; and 8,697,359. Methods of designing suitable gNAs for a particular Cas protein will be known by persons of ordinary skill in the art.

### Guide Nucleic Acids

The present disclosure provides gene-targeting guide nucleic acids (gNAs) that can direct the activities of an associated polypeptide (e.g., nucleic acid guided endonuclease) to a specific target gene sequence within a target nucleic acid genome. The genome-targeting nucleic acid can be an RNA. A genome-targeting RNA is referred to as a “guide RNA” or “gRNA” herein. A guide RNA can comprise at least a targeting sequence that hybridizes to a target nucleic acid sequence of interest, and a CRISPR repeat sequence. In some Type II systems, the gRNA also comprises a second RNA called the tracrRNA sequence, also referred to herein as a “scaffold” sequence. In the Type II guide RNA (gRNA), the CRISPR repeat sequence and scaffold sequence hybridize to each other to form a duplex. In the Type V guide RNA (gRNA), the crRNA forms a duplex. In both systems, the duplex can bind a site-directed polypeptide, such that the guide RNA and site-directed polypeptide form a complex. The gene-targeting nucleic acid can provide target specificity to the complex by virtue of its association with the site-directed polypeptide. The gene-targeting nucleic acid thus can direct the activity of the site-directed polypeptide.

In some embodiments, the disclosure provides a guide RNA comprising a targeting sequence and a guide RNA scaffold sequence, wherein the targeting sequence is complementary to the sequence of a target gene.

Exemplary guide RNAs include the targeting sequences of about 15-20 bases. As is understood by the person of ordinary skill in the art, each gRNA can be designed to include a targeting sequence complementary to its genomic target sequence. For example, each of the targeting sequences, e.g., the RNA version of the DNA sequences presented in SEQ ID NOs: 390-8344, can be put into a single RNA chimera or a crRNA (along with a corresponding scaffold sequence). See Jinek et al., Science, 337, 816-821 (2012) and Deltcheva et al., Nature, 471, 602-607 (2011).

The gene targeting nucleic acid can be a double-molecule guide RNA. The gene targeting nucleic acid can be a single-molecule guide RNA. The gene targeting nucleic acid can be any known configuration of guide RNA known in the art, such as, for example, including paired gRNA, or multiple gRNAs used in a single step. Although it is clear from genomic sequences where the coding sequences and splice junctions are, other features required for gene expression may be idiosyncratic and unclear.

A double-molecule guide RNA can comprise two strands of RNA. The first strand comprises a sequence in the 5′ to 3′ direction, an optional spacer extension sequence, a targeting sequence and a minimum CRISPR repeat sequence. The second strand can comprise a minimum tracrRNA sequence (complementary to the minimum CRISPR repeat sequence), a 3′ tracrRNA sequence and an optional tracrRNA extension sequence.

A single-molecule guide RNA (sgRNA) in a Type II system can comprise, in the 5′ to 3′ direction, an optional spacer extension sequence, a targeting sequence, a minimum CRISPR repeat sequence, a single-molecule guide linker, a minimum tracrRNA sequence, a 3′ tracrRNA sequence and an optional tracrRNA extension sequence. The optional tracrRNA extension can comprise elements that contribute additional functionality (e.g., stability) to the guide RNA. The single-molecule guide linker can link the minimum CRISPR repeat and the minimum tracrRNA sequence to form a hairpin structure. The optional tracrRNA extension can comprise one or more hairpins.

In some embodiments, guide RNA or single-molecule guide RNA (sgRNA) can comprise a targeting sequence and a scaffold sequence. In some embodiments, the scaffold sequence is a Cas9 gRNA sequence. In some embodiments, the scaffold sequence is encoded by a DNA sequence that comprises a sequence that shares at least 90%, at least 95%, at least 96%, at least 97%, at least 98%, or at least 99% identity to GTTTTAGAGCTAGAAATAGCAAGTTAAAATAAGGCTAGTCCGTTATCAACTTGAA AAAGTGGCACCGAGTCGGTGCTTTTTTT (SEQ ID NO: 8345). In some embodiments, the scaffold sequence is encoded by a DNA sequence that comprises GTTTTAGAGCTAGA AATAGCAAGTTAAAATAAGGCTAGTCCGTTATCAACTTGAAAAAGTGGCACCGA GTCGGTGCTTTTTTT (SEQ ID NO: 8345). In some embodiments, the scaffold sequence is encoded by a RNA sequence that comprises a sequence that shares at least 90%, at least 95%, at least 96%, at least 97%, at least 98%, or at least 99% identity to GUUUUAGAGCUAGAAAUAGCAAGUUAAAAUAAGGCUAGUCCGUUAUCAACUU GAAAAAGUGGCACCGAGUCGGUGCUUUUUUU (SEQ ID NO: 8473). In some embodiments, the scaffold sequence is encoded by a RNA sequence that comprises GUUUUAGAGCUAGAA AUAGCAAGUUAAAAUAAGGCUAGUCCGUUAUCAACUUGAAAAAGUGGCACCG AGUCGGUGCUUUUUUU (SEQ ID NO: 8473).

In some embodiments, for example those embodiments where the CRISPR/Cas system is a Cas9 system, the sgRNA can comprise a 20 nucleotide targeting sequence at the 5′ end of the sgRNA sequence. The sgRNA can comprise a less than a 20 nucleotide targeting sequence at the 5′ end of the sgRNA sequence. The sgRNA can comprise a more than 20 nucleotide targeting sequence at the 5′ end of the sgRNA sequence. The sgRNA can comprise a variable length targeting sequence with 17-30 nucleotides at the 5′ end of the sgRNA sequence.

In some embodiments, the sgRNA can comprise no uracil at the 3′ end of the sgRNA sequence. The sgRNA can comprise one or more uracils at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence.

The sgRNA can be unmodified or modified. For example, modified sgRNAs can comprise one or more 2′-O-methyl phosphorothioate nucleotides.

A single-molecule guide RNA (sgRNA) in a Type II system, e.g. Cas9, can comprise, in the 5′ to 3′ direction, a minimum CRISPR repeat sequence and a targeting sequence.

By way of illustration, guide RNAs used in the CRISPR/Cas9 or CRISPR/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

### Spacer Extension Sequence

In some examples of genome-targeting nucleic acids, a spacer extension sequence can modify activity, provide stability and/or provide a location for modifications of a genome-targeting nucleic acid. A spacer extension sequence can modify on- or off-target activity or specificity. In some examples, a spacer extension sequence can be provided. The spacer extension sequence can have a length of more than 1, 5, 10, 15, 20, 25, 30, 35, 40, 45, 50, 60, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. The spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. The spacer extension sequence can be less than 10 nucleotides in length. The spacer extension sequence can be between 10-30 nucleotides in length. The spacer extension sequence can be between 30-70 nucleotides in length.

The spacer extension sequence can comprise another moiety (e.g., a stability control sequence, an endoribonuclease binding sequence, a ribozyme). The moiety can decrease or increase the stability of a nucleic acid targeting nucleic acid. The moiety can be a transcriptional terminator segment (i.e., a transcription termination sequence). The moiety can function in a eukaryotic cell. The moiety can function in a prokaryotic cell. The moiety can function in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

### Targeting Sequence

The targeting sequence of a gRNA hybridizes to a sequence in a target nucleic acid of interest. The targeting sequence of a genome-targeting nucleic acid can interact with a target nucleic acid (or target sequence) in a sequence-specific manner via hybridization (i.e., base pairing). The nucleotide sequence of the targeting sequence can vary depending on the sequence of the target nucleic acid of interest.

In a Cas9 system described herein, the targeting sequence can be designed to hybridize to a target nucleic acid that is located 5′ of the reverse complement of a PAM of the Cas9 enzyme used in the system. The targeting sequence may perfectly match the target sequence or may have mismatches. Each CRISPR/Cas system protein may have a particular PAM sequence, in a particular orientation and position, that it recognizes in a target DNA. For example, S. pyogenes Cas9 recognizes in a target nucleic acid a PAM that comprises the sequence 5′-NRG-3′, where R comprises either A or G, where N is any nucleotide and N is immediately 3′ of the target nucleic acid sequence targeted by the targeting sequence. Selection of appropriate PAM sequences will be apparent to the person of ordinary skill in the art.

The target sequence is complementary to, and hybridizes with, the targeting sequence of the gRNA. The target nucleic acid sequence can comprise 20 nucleotides. The target nucleic acid can comprise less than 20 nucleotides. The target nucleic acid can comprise more than 20 nucleotides. The target nucleic acid can comprise at least: 5, 10, 15, 16, 17, 18, 19, 20, 21, 22, 23, 24, 25, 30 or more nucleotides. The target nucleic acid can comprise at most: 5, 10, 15, 16, 17, 18, 19, 20, 21, 22, 23, 24, 25, 30 or more nucleotides. In some embodiments, for example those embodiments where the CRISPR/Cas system is a Cas9 system, the target nucleic acid sequence can comprise 20 nucleotides immediately 5′ of the first nucleotide of the reverse complement of the PAM sequence. This target nucleic acid sequence is often referred to as the PAM strand or a target strand, and the complementary nucleic acid sequence is often referred to the non-PAM strand or non-target strand. One of skill in the art would recognize that the targeting sequence hybridizes to the non-PAM strand of the target nucleic acid, see e.g., US20190185849A1.

The targeting sequence that hybridizes to the target nucleic acid can have a length of at least about 6 nucleotides (nt). The targeting sequence can be at least about 6 nt, at least about nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about nt, or from about 20 nt to about 60 nt. In some examples, the targeting sequence can comprise 20 nucleotides. In some examples, the targeting can comprise 19 nucleotides. In some examples, the targeting can comprise 18 nucleotides. In some examples, the targeting can comprise 22 nucleotides.

In some examples, the percent complementarity between the targeting sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the targeting sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the targeting sequence and the target nucleic acid is 100% over the six contiguous 5′-most nucleotides of the target sequence of the complementary strand of the target nucleic acid. The percent complementarity between the targeting sequence and the target nucleic acid can be at least 60% over about 20 contiguous nucleotides. The length of the targeting sequence and the target nucleic acid can differ by 1 to 6 nucleotides, which may be thought of as a bulge or bulges.

The targeting sequence can be designed or chosen using computer programs known to persons of ordinary skill in the art. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like. Available computer programs can take as input NCBI gene IDs, official gene symbols, Ensembl Gene IDs, genomic coordinates, or DNA sequences, and create an output file containing sgRNAs targeting the appropriate genomic regions designated as input. The computer program may also provide a summary of statistics and scores indicating on- and off-target binding of the sgRNA for the target gene (Doench et al. Nat Biotechnol. 34:184-191 (2016)).

### Illustrative Targeting Sequences

The disclosure provides guide RNAs comprising a targeting sequence. In some embodiments, the guide RNA further comprises a guide RNA scaffold sequence. In some embodiments, the targeting sequence is complementary to the sequence of a target gene selected from the group consisting of HLA-A, HLA-B, HLA-C, or an allele thereof. In some embodiments, the target gene is a HLA-A gene. In some embodiments, the target gene is a HLA-B gene. In some embodiments, the target gene is a HLA-C gene. In some embodiments the target gene is HLA-A, HLA-B, HLA-C, or a combination thereof. Exemplary gRNA sequences targeting HLA-A are set forth in SEQ ID NOs: 390-509. Further exemplary gRNA sequences targeting HLA-A are set forth as SEQ ID NOs: 390-8344.

In some embodiments, the target gene is a HLA-A allele. In some embodiments, the HLA-A allele comprises HLA-A*02, HLA-A*02:01, HLA-A*02:01:01, and/or HLA-A*02:01:01:01. In some embodiments, the HLA-A allele is HLA-A*02. In some embodiments, the HLA-A*02 allele comprises a sequence that shares about 90%, about 95%, about 96%, about 97%, about 98%, about 99% identity to SEQ ID NO: 8346. In some embodiments, the HLA-A*02 allele comprises SEQ ID NO: 8346. In some embodiments, the HLA-A allele is HLA-A*02:01. In some embodiments, the HLA-A*02:01 allele comprises a sequence that shares about 90%, about 95%, about 96%, about 97%, about 98%, about 99% identity to SEQ ID NO: 8347. In some embodiments, the HLA-A*02:01 allele comprises SEQ ID NO: 8347. In some embodiments, the HLA-A allele is HLA-A*02:01:01. In some embodiments, the HLA-A*02:01:01 allele comprises a sequence that shares about 90%, about 95%, about 96%, about 97%, about 98%, about 99% identity to SEQ ID NO: 8348. In some embodiments, the HLA-A*02:01:01 allele comprises SEQ ID NO: 8348. In some embodiments, the HLA-A allele is HLA-A*02:01:01:01. In some embodiments, the HLA-A*02:01:01:01 allele comprises a sequence that shares about 90%, about 95%, about 96%, about 97%, about 98%, about 99% identity to SEQ ID NO: 8349. In some embodiments, HLA-A*02:01:01:01 allele comprises SEQ ID NO: 8349.

In some embodiments, the gNAs specifically target the sequence of an HLA-A locus. In some embodiments, the gNAs that specifically target the sequence of an HLA-A locus comprise a sequence that shares about 90%, about 95%, about 96%, about 97%, about 98%, or about 99% identity to a sequence selected from SEQ ID NOs: 390-3276. In some embodiments, the gNAs that specifically target the sequence of an HLA-A locus comprise a sequence selected from SEQ ID NOs: 390-3276. The sequences disclosed as SEQ ID NOs: 390-3276 include the corresponding genomic sequences, inclusive of the PAM sequence. The skilled artisan will understand that the targeting sequence of the gRNA does not include three 3′ terminal nucleotides of these sequences, which represent the corresponding PAM site for the gRNA.

In some embodiments, the gNA specifically targets a sequence of HLA-A*02 alleles. For example, the gRNA specifically targets, and hybridize to, a sequence shared by all HLA-A*02 alleles, but that is not shared by HLA-A*02 and HLA-A*03 alleles. In some embodiments, the gNA specifically targets a sequence of HLA-A*02 alleles comprising a sequence that shares about 90%, about 95%, about 96%, about 97%, about 98%, or about 99% identity to a sequence selected from SEQ ID NOs: 390-1585. In some embodiments, the gNA specifically targets a sequence of HLA-A*02 alleles comprising a sequence selected from SEQ ID NOs: 390-1585.

In some embodiments, the gNA specifically targets a sequence of HLA-A*02:01 alleles. In some embodiments, the gNA specifically targets a sequence of HLA-A*02:01 alleles comprising a sequence that shares about 90%, about 95%, about 96%, about 97%, about 98%, or about 99% identity to a sequence selected from SEQ ID NOs: 390-1174. In some embodiments, the gNA specifically targets a sequence of HLA-A*02:01 alleles comprising a sequence selected from SEQ ID NOs: 390-1174.

In some embodiments, the gNA specifically targets a sequence of HLA-A*02:01:01 alleles. In some embodiments, the gNA specifically targets a sequence of HLA-A*02:01:01 alleles comprising a sequence that shares about 90%, about 95%, about 96%, about 97%, about 98%, or about 99% identity to a sequence selected from SEQ ID NOs: 390-1166. In some embodiments, the gNA specifically targets a sequence of HLA-A*02:01:01 alleles comprising a sequence selected from SEQ ID NOs: 390-1166.

In some embodiments, the gNA specifically targets a sequence of HLA-A*02:01:01:01 alleles. In some embodiments, the gNA specifically targets a sequence of HLA-A*02:01:01:01 alleles comprising a sequence that shares about 90%, about 95%, about 96%, about 97%, about 98%, or about 99% identity to a sequence selected from SEQ ID NOs: 390-1126. In some embodiments, the gNA specifically targets a sequence of HLA-A*02:01:01:01 alleles comprising a sequence selected from SEQ ID NOs: 390-1126.

In some embodiments, the gNA specifically targets a coding DNA sequence of HLA-A*02. In some embodiments, the gNA specifically targets a coding DNA sequence of the HLA-A*02 comprising a sequence that shares about 90%, about 95%, about 96%, about 97%, about 98%, or about 99% identity to a sequence selected from SEQ ID NOs: 390-509. In some embodiments, the gNA specifically targets a coding DNA sequence of the HLA-A*02 comprising a sequence selected from SEQ ID NOs: 390-509.

In some embodiments, the gNA specifically targets a coding DNA sequence that is shared by more than 1000 HLA-A*02 alleles. In some embodiments, the gNA specifically targets a coding DNA sequence in greater than 1000 HLA-A*02 alleles comprising a sequence that shares about 90%, about 95%, about 96%, about 97%, about 98%, or about 99% identity to a sequence selected from SEQ ID NOs: 390-455. In some embodiments, the gNA specifically targets a coding DNA sequence in greater than 1000 HLA-A*02 alleles comprising a sequence selected from SEQ ID NOs: 390-455.

In some embodiments, the gNA target sequence comprises a sequence that shares about 90%, about 95%, about 96%, about 97%, about 98%, or about 99% identity to a sequence selected from SEQ ID NOs: 394, 407, 408, 414, 421, 423, 426, 429, 4333, 435, 438, 440, 448, 451, 454. In some embodiments, the gNAs comprise a sequence selected from SEQ ID NOs: 394, 407, 408, 414, 421, 423, 426, 429, 433, 435, 438, 440, 448, 451, and 454.

In some embodiments, the gNA target comprises a sequence that shares about 90%, about 95%, about 96%, about 97%, about 98%, or about 99% identity to SEQ ID NO: 394. In some embodiments, the gNA target sequence comprises SEQ ID NO: 394.

In some embodiments, the gNA target sequence comprises a sequence that shares about 90%, about 95%, about 96%, about 97%, about 98%, or about 99% identity to SEQ ID NO: 423. In some embodiments, the gNA target sequence comprises SEQ ID NO: 423.

In some embodiments, the gNA target sequence comprises a sequence that targets multiple alleles of the HLA-A, B, and C loci. In some embodiments, the gNA target sequence comprises a sequence that shares about 90%, about 95%, about 96%, about 97%, about 98%, or about 99% identity to SEQ ID NO: 408. In some embodiments, the gNA target sequence comprises SEQ ID NO: 408.

The disclosure provides gNAs comprising a targeting sequence specific to the B2M gene and B2M gene regulatory elements. In some embodiments the gNA comprise a targeting sequence and a gNA scaffold sequence. In some embodiments, the targeting sequence is complementary to a sequence of the B2Mgene. In some embodiments, the B2Mgene comprises a sequence that shares about 90%, about 95%, about 96%, about 97%, about 98%, about 99% identity to SEQ ID NO: 8471. In some embodiments, the B2M gene comprises SEQ ID NO: 8471. Exemplary gRNA sequences targeting B2M are set forth in SEQ ID NOs: 8345, 8350, and 8357-8470.

In some embodiments, the gNA specifically target a sequence of the B2Mgene. In some embodiments, the gNA comprises a targeting sequence that shares about 80%, about 85%, about 90%, about 95%, about 96%, about 97%, about 98%, or about 99% identity to a sequence selected from SEQ ID NOs: 8357-8470. In some embodiments, the gNA comprises a sequence that shares about 80%, about 85%, about 90%, about 95%, about 96%, about 97%, about 98%, or about 99% identity to a sequence selected from SEQ ID NOs: 8357-8470. In some embodiments, the gNA that specifically targets the sequence of the B2M gene comprise a sequence selected from SEQ ID NOs: 8357-8470.

In some embodiments, the gNA specifically targets the coding sequence (CDS) sequence of the B2M gene. In some embodiments, the gNA that specifically targets the CDS sequence of the B2Mgene comprise a sequence that shares about 80%, about 85%, about 90%, about 95%, about 96%, about 97%, about 98%, or about 99% identity to a sequence selected from SEQ ID NOs: 8357-8397. In some embodiments, the gNA that specifically targets the sequence of an HLA-A locus comprise a sequence selected from SEQ ID NOs: 8357-8397.

In some embodiments, the gNA comprises a sequence that targets the B2M gene promoter sequence. In some embodiments, the gNA comprises a sequence that shares about 80%, about 85%, about 90%, about 95%, about 96%, about 97%, about 98%, or about 99% identity to SEQ ID NO: 8398-8470. In some embodiments, the gNA comprises SEQ ID NO: 8398-8470.

In some embodiments, the gNA comprises a sequence that targets the B2M gene sequence. In some embodiments, the gNA comprises a sequence that shares about 80%, about 85%, about 90%, about 95%, about 96%, about 97%, about 98%, or about 99% identity to SEQ ID NOs: 8357-8365 and 8398-8444. In some embodiments, the gNA comprises SEQ ID NO: 8357-8365 and 8398-8444.

The illustrative sequences targeting B2M and HLA presented herein may presented as DNA sequences. In these sequences, thymine (T) may be replaced by uracil (U) to arrive at the sequence of the gRNA targeting sequence in those embodiments wherein the gRNA is a gRNA.

### TALEN for Targeted Gene Editing

In some embodiments, the allogeneic immune cells described herein are edited using TALEN gene editing.

“TALEN” or “TALEN gene editing” refers to a transcription activator-like effector nuclease, which is an artificial nuclease used to edit a target gene.

TALENs are produced artificially by fusing a TAL effector DNA binding domain to a DNA cleavage domain. Transcription activator-like effectors (TALEs) can be engineered to bind any desired DNA sequence, including a portion of target genes such as TCR subunits, MHC class I complex components, or CD52. By combining an engineered TALE with a DNA cleavage domain, a restriction enzyme can be produced which is specific to any desired DNA sequence, including a target gene sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is a wild-type or mutated Fold endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A target gene TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in a target gene or introduce such a defect into a wild type target gene, thus decreasing expression of the target gene.

TALENs specific to sequences in a target gene can be constructed using any method known in the art, including various schemes using modular components. Zhang et al. (2011) Nature Biotech. 29: 149-53; Geibler et al. (2011) PLoS ONE 6: e19509.

### Zinc Finger Nucleases for Targeted Gene Editing

In some embodiments, a target gene is edited in the allogeneic immune cells described herein using ZFN gene editing.

“ZFN” or “Zinc Finger Nuclease” or “ZFN gene editing” refer to a zinc finger nuclease, an artificial nuclease which can be used to edit a target gene.

Like a TALEN, a ZFN comprises a Fold nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression and amount of a target gene or gene product in a cell. ZFNs can also be used with homologous recombination to mutate in a target gene.

ZFNs specific to sequences in a target gene can be constructed using any method known in the art. See, e.g., Provasi (2011) Nature Med. 18: 807-815; Torikai (2013) Blood 122: 1341-1349; Cathomen et al. (2008) Mol. Ther. 16: 1200-7; Guo et al. (2010) J. Mol. Biol. 400: 96; U.S. Patent Publication 2011/0158957; and U.S. Patent Publication 2012/0060230.

### Activators

The disclosure provides a first ligand, an activator, and a first engineered receptor comprising the first ligand binding domain that binds to the first activator ligand.

The disclosure provides a first engineered receptor comprising an extracellular region, the extracellular region comprising a first ligand binding domain capable of specifically binding a first ligand that activates or promotes activation of the receptor, which promotes activation of effector cells expressing the receptor. The disclosure further provides a second engineered receptor comprising a second ligand binding domain capable of binding a second ligand, wherein binding of the second ligand by the second ligand binding domain inhibits or reduces activation of effector cells even in the presence of the first receptor bound to the first ligand.

As used herein, an “activator” or “activator ligand” refers to a first ligand that binds to a first, activator ligand binding domain (LBD) of an engineered receptor of the disclosure, such as a CAR or TCR, thereby mediating activation of a T cell expressing the engineered receptor. The activator is expressed by target cells, for example cancer cells, and may also be expressed more broadly than just the target cells. For example the activator can be expressed on some, or all types of normal, non-target cells.

In some embodiments, the first ligand is a peptide ligand from any of the activator targets disclosed herein. In some embodiments, the first ligand is a peptide antigen complexed with a major histocompatibility (MHC) class I complex (peptide MHC, or pMHC), for example an MHC complex comprising human leukocyte antigen A*02 allele (HLA-A*02).

Target cell-specific first activator ligands comprising peptide antigens complexed with pMHC comprising any of human leukocyte antigen (HLA) HLA-A, HLA-B, HLA-C, HLA-E, HLA-F, and HLA-G are envisaged as within the scope of the disclosure. In some embodiments, the first ligand comprises a pMHC comprising HLA-A. HLA-A receptors are heterodimers comprising a heavy α chain and smaller β chain. The α chain is encoded by a variant of HLA-A, while the β chain (β2-microglobulin) is an invariant. There are several thousand HLA-A gene variants, all of which fall within the scope of the instant disclosure. In some embodiments, the MHC-I comprises a human leukocyte antigen A*02 allele (HLA-A*02).

In some embodiments, the first activator ligand comprises a pMHC comprising HLA-B. Hundreds of versions (alleles) of the HLA-B gene are known, each of which is given a particular number (such as HLA-B*27).

In some embodiments, the first activator ligand comprises a pMHC comprising HLA-C. HLA-C belongs to the HLA class I heavy chain paralogues. This class I molecule is a heterodimer consisting of a heavy chain and a light chain (beta-2 microglobulin). Over one hundred HLA-C alleles are known in the art.

In some embodiments, the first activator ligand comprises a pMHC comprising HLA-A. In some embodiments, the first activator ligand comprises a pMHC comprising HLA-B. In some embodiments, the first activator ligand comprises a pMHC comprising HLA-C. In some embodiments, the first activator ligand comprises a pMHC comprising HLA-E. In some embodiments, the first activator ligand comprises a pMHC comprising HLA-F. In some embodiments, the first activator ligand comprises a pMHC comprising HLA-G.

In some embodiments, the first activator ligand comprises HLA-A. In some embodiments, the first activator ligand comprises HLA-B. In some embodiments, the first activator ligand comprises HLA-C. In some embodiments, the first activator ligand comprises HLA-E. In some embodiments, the first activator ligand comprises HLA-F. In some embodiments, the first activator ligand comprises HLA-G. In some embodiments, the first activator ligand comprises HLA-A, HLA-B, HLA-C, HLA-E, HLA-F or HLA-G.

In some embodiments, the first, activator ligand binding domain comprises an ScFv domain.

In some embodiments, the first, activator ligand binding domain comprises a VP-only ligand binding domain.

In some embodiments, the first, activator ligand binding domain comprises an antigen binding domain isolated or derived from a T cell receptor (TCR). For example, the first, activator ligand binding domain comprises TCR α and β chain variable domains.

In some embodiments, the first, activator ligand and the second, inhibitor ligand are not the same.

In some embodiments, the first, activator ligand is expressed by target cells and is not expressed by non-target cells (i.e. normal cells not targeted by the adoptive cell therapy). In some embodiments, the target cells are cancer cells and the non-target cells are non-cancerous cells.

In some embodiments, the activator ligand has high cell surface expression on the target cells. This high cell surface expression confers the ability to deliver large activation signals. Methods of measuring cell surface expression will be known to the person of ordinary skill in the art and include, but are not limited to, immunohistochemistry using an appropriate antibody against the activator ligand, followed by microscopy or fluorescence activated cell sorting (FACS).

In some embodiments, the activator ligand is encoded by a gene with an essential cellular function. Essential cellular functions are functions required for a cell to live, and include protein and lipid synthesis, cell division, replication, respiration, metabolism, ion transport, and providing structural support for tissues. Selecting activator ligands encoded by genes with essential cellular functions prevents loss of the activator ligand due to aneuploidy in cancer cells, and makes gene encoding the activator ligand less likely to undergo mutagenesis during the evolution of the cancer. In some embodiments, the activator ligand is encoded by a gene that is haploinsufficient, i.e. loss of copies of the gene encoding the activator ligand are not tolerated by the cell and lead to cell death or a disadvantageous mutant phenotype.

In some embodiments, the activator ligand is present on all target cells. In some embodiments, the target cells are cancer cells.

In some embodiments, the activator ligand is present on a plurality of target cells. In some embodiments, the target cells are cancer cells. In some embodiments, the activator ligand is present on at least 50%, at least 60%, at least 70%, at least 80%, at least 85%, at least 90%, at least 95%, at least 96%, at least 97%, at least 98%, at least 99%, at least 99.5% or at least 99.9% of target cells. In some embodiments, the activator ligand is present on at least 95% target cells. In some embodiments, the activator ligand is present on at least 99% target cells.

In some embodiments, the activator ligand is present on all cells (ubiquitous activator ligands). Activator ligands can be expressed on all cells, if, for example, the second inhibitor ligand is also expressed on all cells except the target cells.

In some embodiments, the first, activator ligand is expressed by a plurality of target cells and a plurality of non-target cells. In some embodiments, the plurality of non-target cells expresses both the first, activator ligand and the second inhibitor ligand.

In some embodiments, and the first, activator ligand and second, inhibitor ligand are present on the plurality of non-target target cells at a ratio of about 1:100 to about 100:1 of the first ligand to the second ligand. In some embodiments, and the first, activator ligand and second, inhibitor ligand are present on the plurality of non-target target cells at a ratio of about 1:50 to about 50:1 of the first ligand to the second ligand. In some embodiments, and the first, activator ligand and second, inhibitor ligand are present on the plurality of non-target target cells at a ratio of about 1:25 to about 25:1 of the first ligand to the second ligand. In some embodiments, and the first, activator ligand and second, inhibitor ligand are present on the plurality of non-target target cells at a ratio of about 1:10 to about 10:1 of the first ligand to the second ligand. In some embodiments, and the first, activator ligand and second, inhibitor ligand are present on the plurality of non-target target cells at a ratio of about 1:5 to about 5:1 of the first ligand to the second ligand. In some embodiments, and the first, activator ligand and second, inhibitor ligand are present on the plurality of non-target target cells at a ratio of about 1:3 to about 3:1 of the first ligand to the second ligand. In some embodiments, and the first, activator ligand and second, inhibitor ligand are present on the plurality of non-target target cells at a ratio of about 1:2 to about 2:1 of the first ligand to the second ligand. In some embodiments, and the first, activator ligand and second, inhibitor ligand are present on the plurality of non-target target cells at a ratio of about 1:1.

The first, activator ligand is recognized by a first ligand binding domain (sometimes referred to herein as the activator LBD).

Exemplary activator ligands include ligands selected from the group consisting of cell adhesion molecules, cell-cell signaling molecules, extracellular domains, molecule involved in chemotaxis, glycoproteins, G protein-coupled receptors, transmembrane proteins, receptors for neurotransmitters and voltage gated ion channels. In some embodiments, the first, activator ligand is transferrin receptor (TFRC) or a peptide antigen thereof. Human transferrin receptor is described in NCBI record No. AAA61153.1, the contents of which are incorporated herein by reference. In some embodiments, TFRC is encoded by a sequence of SEQ ID NO: 18.

In some embodiments, the activator ligand is a tumor specific antigen (TSA). In some embodiments, the tumor specific antigen is mesothelin (MSLN), CEA cell adhesion molecule (CEACAMS, or CEA), epidermal growth factor receptor (EGFR) or a peptide antigen thereof. In some embodiments, the TSA is MSLN, CEA, EGFR, delta like canonical Notch ligand 4 (DLL4), mucin 16, cell surface associated (MUC 16 also known as CA125), ganglioside GD2 (GD2), receptor tyrosine kinase like orphan receptor 1 (ROR1), erb-b2 receptor tyrosine kinase 2 (HER2/NEU) or a peptide antigen thereof. Exemplary mouse and humanized scFv antigen binding domains targeting TSAs are set forth in SEQ ID NOs: 83, 85, 87, 89, 91, 93, 95, 97, 99, 101, 103, 105, 107, 109, 111, 113, 115, 273, 275, 266, 268, 277, and 381. Exemplary polynucleotide sequences encoding mouse and humanized scFv antigen binding regions targeting TSA are set forth in SEQ ID NOs: 84, 86, 88, 90, 92, 94, 96, 98, 100, 102, 104, 106, 108, 110, 112, 114, 116, 267, 269, 274, 276, and 278.

In some embodiments, the activator ligand is MSLN or a peptide antigen thereof, and the activator ligand binding domain comprises a MSLN binding domain. In some embodiments, the MSLN ligand binding domain comprises an scFv domain. In some embodiments, the MSLN ligand binding domain comprises a sequence of SEQ ID NO: 83, SEQ ID NO: 85, SEQ ID NO: 87 or SEQ ID NO: 89. In some embodiments, the MSLN ligand binding domain comprises a sequence at least 90%, at least 95% or at least 99% identical to SEQ ID NO: 83, SEQ ID NO: 85, SEQ ID NO: 87 or SEQ ID NO: 89. In some embodiments, the MSLN ligand binding domain is encoded by a sequence comprising SEQ ID NO: 84, SEQ ID NO: 86, SEQ ID NO: 88 or SEQ ID NO: 90. In some embodiments, the MSLN ligand binding domain is encoded by a sequence having at least 80% identity, at least 85% identity, at least 90% identity, at least 95% identity or at least 99% identity to a sequence of SEQ ID NO: 84, SEQ ID NO: 86, SEQ ID NO: 88 or SEQ ID NO: 90.

In some embodiments, the activator ligand is CEA or a peptide antigen thereof, and the activator ligand binding domain comprises a CEA binding domain. In some embodiments, the CEA ligand binding domain comprises an ScFv domain. In some embodiments, the CEA ligand binding domain comprises a sequence of SEQ ID NO: 91, SEQ ID NO: 93, SEQ ID NO: 95, SEQ ID NO: 97, SEQ ID NO: 273, SEQ ID NO: 275 or SEQ ID NO: 277. In some embodiments, the CEA ligand binding domain comprises a sequence at least 90%, at least 95% or at least 99% identical to SEQ ID NO: 94, SEQ ID NO: 96, SEQ ID NO: 98, SEQ ID NO: 97, SEQ ID NO: 273, SEQ ID NO: 275 or SEQ ID NO: 277. In some embodiments, the CEA ligand binding domain is encoded by a sequence comprising SEQ ID NO: 92, SEQ ID NO: 94, SEQ ID NO: 96, SEQ ID NO: 98, SEQ ID NO: 274, SEQ ID NO: 276 or SEQ ID NO: 278. In some embodiments, the CEA ligand binding domain is encoded by a sequence having at least 80% identity, at least 85% identity, at least 90% identity, at least 95% identity or at least 99% identity to a sequence of SEQ ID NO: 92, SEQ ID NO: 94, SEQ ID NO: 96, SEQ ID NO: 98, SEQ ID NO: 274, SEQ ID NO: 276 or SEQ ID NO: 278.

In some embodiments, the activator ligand is CEA or a peptide antigen thereof, and the activator ligand binding domain comprises a CEA binding domain. In some embodiments, the CEA ligand binding domain comprises a CDR-H1 of EFGMN (SEQ ID NO: 285), a CDR-H2 of WINTKTGEATYVEEFKG (SEQ ID NO: 286), a CDR-H3 of WDFAYYVEAMDY (SEQ ID NO: 287) or WDFAHYFQTMDY (SEQ ID NO: 288), a CDR-L1 of KASQNVGTNVA (SEQ ID NO: 289) or KASAAVGTYVA (SEQ ID NO: 290), a CDR-L2 of SASYRYS (SEQ ID NO: 291) or SASYRKR (SEQ ID NO: 292), and a CDR-L3 of HQYYTYPLFT (SEQ ID NO: 293) or sequences having at least 85% or at least 95% identity thereto. In some embodiments, a CEA ScFv comprises a CDR-H1 of EFGMN (SEQ ID NO: 285), a CDR-H2 of WINTKTGEATYVEEFKG (SEQ ID NO: 286), a CDR-H3 of WDFAYYVEAMDY (SEQ ID NO: 287) or WDFAHYFQTMDY (SEQ ID NO: 288), a CDR-L1 of KASQNVGTNVA (SEQ ID NO: 289) or KASAAVGTYVA (SEQ ID NO: 290), a CDR-L2 of SASYRYS (SEQ ID NO: 291) or SASYRKR (SEQ ID NO: 292) and a CDR-L3 of HQYYTYPLFT (SEQ ID NO: 293). In some embodiments, a CEA binding domain comprises a CDR-H1 of EFGMN (SEQ ID NO: 285), a CDR-H2 of WINTKTGEATYVEEFKG (SEQ ID NO: 286), a CDR-H3 of WDFAYYVEAMDY (SEQ ID NO: 287), a CDR-L1 of KASQNVGTNVA (SEQ ID NO: 289), a CDR-L2 of SASYRYS (SEQ ID NO: 290) and a CDR-L3 of HQYYTYPLFT (SEQ ID NO: 293). In some embodiments, a CEA ScFv comprises a CDR-H1 of EFGMN (SEQ ID NO: 285), a CDR-H2 of WINTKTGEATYVEEFKG (SEQ ID NO: 286), a CDR-H3 of WDFAYYVEAMDY (SEQ ID NO: 287), a CDR-L1 of KASAAVGTYVA (SEQ ID NO: 290), a CDR-L2 of SASYRKR, and a CDR-L3 of HQYYTYPLFT (SEQ ID NO: 293). In some embodiments, a CEA binding domain comprises a CDR-H1 of EFGMN (SEQ ID NO: 285), a CDR-H2 of WINTKTGEATYVEEFKG (SEQ IDNO: 286), a CDR-H3 of WDFAHYFQTMDY (SEQ ID NO: 288), a CDR-L1 of KASAAVGTYVA (SEQ ID NO: 290), a CDR-L2 of SASYRKR, and a CDR-L3 of HQYYTYPLFT (SEQ ID NO: 293).

In some embodiments, the activator ligand is CEA or a peptide antigen thereof, and the activator receptor is a CEA CAR. In some embodiments, the CEA CAR comprises sequence at least 90%, at least 95% or at least 99% identical to SEQ ID NO: 279, SEQ ID NO: 281 or SEQ ID NO: 283. In some embodiments, the CEA CAR comprises or consists essentially of SEQ ID NO: 279, SEQ ID NO: 281 or SEQ ID NO: 283. In some embodiments, the CEA CAR is encoded by a sequence comprising or consisting essentially of SEQ ID NO: 280, SEQ ID NO: 282 or SEQ ID NO: 284. In some embodiments, the CEA CAR is encoded by a sequence having at least 80% identity, at least 85% identity, at least 90% identity, at least 95% identity or at least 99% identity to SEQ ID NO: 280, SEQ ID NO: 282 or SEQ ID NO: 284.

In some embodiments, the activator ligand is EGFR or a peptide antigen thereof, and the activator ligand binding domain comprises an EGFR binding domain. In some embodiments, the EGFR ligand binding domain comprises an ScFv domain. In some embodiments, the EGFR ligand binding domain comprises a sequence of SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115 or SEQ ID NO: 381. In some embodiments, the EGFR ligand binding domain comprises a sequence at least 90%, at least 95% or at least 99% identical to SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115 or SEQ ID NO: 381. In some embodiments, the EGFR ligand binding domain is encoded by a sequence comprising SEQ ID NO: 100, SEQ ID NO: 101, SEQ ID NO: 104, SEQ ID NO: 106, SEQ ID NO: 108, SEQ ID NO: 110, SEQ ID NO: 112, SEQ ID NO: 114 or SEQ ID NO: 116. In some embodiments, the EGFR ligand binding domain is encoded by a sequence having at least 80% identity, at least 85% identity, at least 90% identity, at least 95% identity or at least 99% identity to a sequence of SEQ ID NO: 100, SEQ ID NO: 101, SEQ ID NO: 104, SEQ ID NO: 106, SEQ ID NO: 108, SEQ ID NO: 110, SEQ ID NO: 112, SEQ ID NO: 114 or SEQ ID NO: 116.

In some embodiments, the activator ligand is EGFR or a peptide antigen thereof, and the activator ligand binding domain comprises an EGFR ligand binding domain. In some embodiments, the EGFR ligand binding domain comprises a VH domain selected from the group consisting of SEQ ID NO: 117, SEQ ID NO: 119, SEQ ID NO: 121, SEQ ID NO: 123, SEQ ID NO: 125 and SEQ ID NO: 127. In some embodiments, the EGFR ligand binding domain comprises a VH selected from the group consisting of SEQ ID NO: 117, SEQ ID NO: 119, SEQ ID NO: 121, SEQ ID NO: 123, SEQ ID NO: 125 and SEQ ID NO: 127 or a sequence having at least 90%, at least 95% or at least 99% identity thereto. In some embodiments, the EGFR ligand binding domain comprises a VL domain selected from the group consisting of SEQ ID NO: 118, SEQ ID NO: 120, SEQ ID NO: 122, SEQ ID NO: 124, SEQ ID NO: 126 and SEQ ID NO: 128. In some embodiments, the EGFR ligand binding domain comprises a VH selected from the group consisting of SEQ ID NO: 118, SEQ ID NO: 120, SEQ ID NO: 122, SEQ ID NO: 124, SEQ ID NO: 126 and SEQ ID NO: 128 or a sequence having at least 90%, at least 95% or at least 99% identity thereto.

In some embodiments, the activator ligand is EGFR or a peptide antigen thereof, and the activator ligand binding domain is an EGFR ligand binding domain. In some embodiments, the EGFR ligand binding domain comprises CDRs selected from SEQ ID NOs: 129-162. In some embodiments, the EGFR ligand binding domain comprises a heavy chain CDR 1 (CDR H1) selected from the group consisting of SEQ ID NOs: 129-134. In some embodiments, the EGFR ligand binding domain comprises a heavy chain CDR 2 (CDR H2) selected from the group consisting of SEQ ID NOs: 135-140. In some embodiments, the EGFR ligand binding domain comprises a heavy chain CDR 3 (CDR H3) selected from the group consisting of SEQ ID NOs: 141-146. In some embodiments, the EGFR ligand binding domain comprises a light chain CDR 1 (CDR L1) selected from the group consisting of SEQ ID NOs: 147-151. In some embodiments, the EGFR ligand binding domain comprises a light chain CDR 2 (CDR L2) selected from the group consisting of SEQ ID NOs: 152-156. In some embodiments, the EGFR ligand binding domain comprises a light chain CDR 3 (CDR L3) selected from the group consisting of SEQ ID NOs: 157-162. In some embodiments, the EGFR ligand binding domain comprises a CDR H1 selected from SEQ ID NOs: 129-134, a CDR H2 selected from SEQ ID NOs: 135-140, a CDR H3 selected from SEQ ID NOs: 141-146, a CDR L1 selected from SEQ ID NOs: 147-151, a CDR L2 selected from SEQ ID NOs: 152-156, and a CDR L3 selected from SEQ ID NOs: 152-156.

In some embodiments, the activator ligand is mesothelin (MSLN) or a peptide antigen thereof, and the activator ligand binding domain is an mesothelin ligand binding domain. Examples of mesothelin ligand binding domains are described, for example, in International Patent Application No. PCT/US2021/046751 which is incorporated herein in its entirety for examples of mesothelin ligand binging domains that may be used in the compositions and methods described herein. In some embodiments, the mesothelin ligand binding domain comprises an scFv. In some embodiments the mesothelin ligand binding domain is an scFv comprising a sequence that is least 90%, at least 95%, at least 98%, or at least 99% identical to the sequence set forth in any one of SEQ ID NOs: 21510-21572. In some embodiments the mesothelin ligand binding domain is an scFv comprising the sequence set forth in any one of SEQ ID NOs: 21510-21572.

In some embodiments, the mesothelin ligand binding domain comprises a VH CDR 1 selected from the group consisting of SEQ ID NOs: 21573-21593. In some embodiments, the mesothelin ligand binding domain comprises a VH CDR2 selected from the group consisting of SEQ ID NOs: 21594-21614. In some embodiments, the mesothelin ligand binding domain comprises a VH CDR3 selected from the group consisting of SEQ ID NOs: 21615-21678. In some embodiments, the mesothelin ligand binding domain comprises a VL CDR1 selected from the group consisting of SEQ ID NOs: 21679-21683. In some embodiments, the mesothelin ligand binding domain comprises a VL CDR2 selected from the group consisting of SEQ ID NOs: 152, 21684 and 21685. In some embodiments, the mesothelin ligand binding domain comprises a VL CDR3 selected from the group consisting of SEQ ID NOs: 157, 21686, 21687, and 21688.

In some embodiments, the mesothelin ligand binding domain comprises a VH CDR 1 selected from the group consisting of SEQ ID NOs: 21573-21593; a VH CDR2 selected from the group consisting of SEQ ID NOs: 21594-21614; a VH CDR3 selected from the group consisting of SEQ ID NOs: 21615-21678; a VL CDR1 selected from the group consisting of SEQ ID NOs: 21679-21683; a VL CDR2 selected from the group consisting of SEQ ID NOs: 152, 21684 and 21685; and a VL CDR3 selected from the group consisting of SEQ ID NOs: 157, 21686, 21687, and 21688.

In some embodiments, the activator ligand is a pan-HLA ligand, and the activator binding domain is a pan-HLA binding domain, i.e. a binding domain that binds to and recognizes an antigenic determinant shared among products of the HLA A, B and C loci. Various single variable domains known in the art or disclosed herein are suitable for use in embodiments. Such scFvs include, for example and without limitation, the following mouse and humanized pan-HLA scFv antibodies. An exemplary pan-HLA ligand is W6/32, which recognizes a conformational epitope, reacting with HLA class I alpha3 and alpha2 domains. Illustrative pan-HLA scFv binding domains derived from W6/32 are set forth in SEQ ID NOs: 163, 165, 167, 169, 171, and 173. Illustrative polynucleotide sequences encoding an-HLA scFv binding domains derived from W6/32 are set forth in SEQ ID NOs: 164, 166, 168, 170, 172, and 174.

In some embodiments, the activator ligand is pan-HLA ligand, and the activator ligand binding domain comprises a pan-HLA ligand binding domain. In some embodiments, the pan-HLA ligand binding domain comprises an ScFv domain. In some embodiments, the pan-HLA ligand binding domain comprises a sequence of SEQ ID NO: 163, SEQ ID NO: 165, SEQ ID NO: 167, SEQ ID NO: 169, SEQ ID NO: 171, or SEQ ID NO: 173. In some embodiments, the pan-HLA ligand binding domain comprises a sequence at least 90%, at least 95% or at least 99% identical to SEQ ID NO: 163, SEQ ID NO: 165, SEQ ID NO: 167, SEQ ID NO: 169, SEQ ID NO: 171, or SEQ ID NO: 173. In some embodiments, the pan-HLA ligand binding domain is encoded by a sequence comprising SEQ ID NO: 164, SEQ ID NO: 166, SEQ ID NO: 168, SEQ ID NO: 170, SEQ ID NO: 172, or SEQ ID NO: 174. In some embodiments, the pan-HLA ligand binding domain is encoded by a sequence having at least 80% identity, at least 85% identity, at least 90% identity, at least 95% identity or at least 99% identity to a sequence of SEQ ID NO: 164, SEQ ID NO: 166, SEQ ID NO: 168, SEQ ID NO: 170, SEQ ID NO: 172, or SEQ ID NO: 174.

In some embodiments, the activator ligand is CD19 molecule (CD19) or a peptide antigen thereof, and the activator ligand binding domain comprises a CD19 ligand binding domain. In some embodiments, the CD19 ligand binding domain comprises an ScFv domain. In some embodiments, the CD19 ligand binding domain comprises a sequence at least 90%, at least 95% or at least 99% identical to SEQ ID NO: 266 or SEQ ID NO: 268. In some embodiments, the CD-19 ligand binding domain comprises a sequence of SEQ ID NO: 266 or SEQ ID NO: 268. In some embodiments, the CD19 ligand binding domain is encoded by a sequence comprising SEQ ID NO: 267, or SEQ ID NO: 269. In some embodiments, the CD19 ligand binding domain is encoded by a sequence having at least 80% identity, at least 85% identity, at least 90% identity, at least 95% identity or at least 99% identity to a sequence of SEQ ID NO: 267 or SEQ ID NO: 269.

In some embodiments, activator ligand is CD19 molecule (CD19) or a peptide antigen thereof, and the activator receptor is a CAR. In some embodiments, the CD19 CAR comprises a sequence at least 90%, at least 95% or at least 99% identical to SEQ ID NO: 270 or SEQ ID NO: 272. In some embodiments, the CD19 CAR comprises or consists essentially of SEQ ID NO: 270 or SEQ ID NO: 272. In some embodiments, the CD19 CAR is encoded by a sequence having at least 80% identity, at least 85% identity, at least 90% identity, at least 95% identity or at least 99% identity to a sequence of SEQ ID NO: 271 or SEQ ID NO: 380. In some embodiments, the CD19 CAR is encoded by a sequence comprising or consisting essentially of SEQ ID NO: 271 or SEQ ID NO: 380. It will be appreciated by the person of ordinary skill that first, activator ligand binding domains for the first receptor may be isolated or derived from any source known in the art, including, but not limited to, art recognized T cell receptors, chimeric antigen receptors and antibody binding domains. For example, the first ligand binding domain may be derived from any of the antibodies disclosed in Table 1, and bind to a first ligand selected from the antigens described in Table 1. Accordingly, the immune cells comprising the two receptor system described can be used to treat any of the diseases or disorders described in Table 1. Selection of an appropriate first, activator receptor ligand binding domain to treat any the cancers described herein will be apparent to those of skill in the art.

TABLE 1
Exemplary Antibodies

| | | Exemplary Diseases and |
|---|---|---|
| Antigen | Antibody | Disorders |
| TNF receptor superfamily | Urelumab, Utomilumab | cancer, diffuse large B- |
| member 9 (4-1BB, CD137) | | cell lymphoma |
| 5′-nucleotidase | Oleclumab | pancreatic and colorectal |
| | | cancer |
| trophoblast glycoprotein | Naptumomab | non-small cell lung |
| (5T4) | | carcinoma, renal cell |
| | | carcinoma |
| activin receptor-like kinase 1 | Ascrinvacumab | cancer |
| alpha-fetoprotein | Tacatuzumab | cancer |
| angiopoietin 2 | Nesvacumab, Vanucizumab | cancer |
| TNF superfamily member | Belimumab, Tabalumab, | cancers and autoimmune |
| 13b (BAFF) | Tibulizumab | disorders |
| TNF receptor superfamily | Belantamab | multiple myeloma |
| member 17 (BCMA) | | |
| mucin 16, cell surface | Igovomab, Oregovomab, | ovarian cancer |
| associated (CA-125) | Sofituzumab | |
| C-C motif chemokine | Mogamulizumab | adult T-cell |
| receptor 4 (CCR4) | | leukemia/lymphoma |
| interleukin 3 receptor | Talacotuzumab | leukemia |
| subunit alpha (CD123) | | |
| TNF receptor superfamily | Tavolimab, Vonlerolizumab | cancer |
| member 4 (CD134) | | |
| cytotoxic T-lymphocyte | Ipilimumab | melanoma |
| associated protein 4 | | |
| (CD152) | | |
| CD19 molecule (CD19) | Duvortuxizumab, Blinatumomab, | cancer |
| | Coltuximab, Denintuzumab, | |
| | Inebilizumab, Loncastuximab, | |
| | Taplitumomab | |
| membrane spanning 4- | Ibritumomab, Obinutuzumab, | cancers, multiple |
| domains Al (CD20) | Ocaratuzumab, Ocrelizumab, | sclerosis, autoimmune |
| | Ofatumumab, Rituximab, | disorders |
| | Tositumomab, Veltuzumab | |
| CD200 molecule (CD200) | Samalizumab | cancer |
| CD22 molecule (CD22) | Bectumomab, Epratuzumab, | cancer |
| | Inotuzumab, Moxetumomab, | |
| | Pinatuzumab | |
| Fc fragment of IgE | Gomiliximab, Lumiliximab | chronic lymphocytic |
| receptor II (CD23, IgE | | leukemia |
| receptor) | | |
| interleukin 2 receptor | Camidanlumab, Basiliximab, | leukemias and lymphomas |
| subunit alpha (CD25) | Inolimomab, Daclizumab | |
| CD27 molecule (CD27) | Varlilumab | solid tumors and |
| | | hematologic malignancies |
| CD276 molecule (CD276) | Enoblituzumab, Omburtamab | cancer |
| TNF receptor superfamily | Brentuximab, Iratumumab | Hodgkin's lymphoma |
| member 8 (CD30, | | |
| TNFRSF8) | | |
| CD33 molecule (CD33) | Gemtuzumab, Lintuzumab, | acute myelogenous |
| | Vadastuximab | leukemia |
| CD37 molecule (CD37) | Lilotomab, Otlertuzumab, | cancer |
| | Tetulomab | |
| CD38 molecule (CD38) | Daratumumab, Isatuximab | multiple myeloma |
| CD44 molecule v6 (CD44 | Bivatuzumab | squamous cell carcinoma |
| v6) | | |
| integrin subunit alpha V | Abituzumab, Intetumumab | cancer |
| (CD51) | | |
| neural cell adhesion | Lorvotuzumab | cancer |
| molecule 1 (CD56) | | |
| CD6 molecule (CD6) | Itolizumab | psoriasis |
| CD70 molecule (CD70) | Cusatuzumab, Vorsetuzumab | cancer |
| CD74 molecule (CD74) | Milatuzumab | hematological |
| | | malignancies |
| CD79B molecule (CD79B) | Polatuzumab, Iladatuzumab | Hematological cancers |
| CD80 molecule (CD80) | Galiximab | B-cell lymphoma |
| CEA cell adhesion | Altumomab, Arcitumomab, | cancer, colorectal cancer |
| molecule 5 (CEA) | Labetuzumab, Cibisatamab | |
| Claudin 18 Isoform 2 | Zolbetuximab | gastric cancer |
| Colony stimulating factor 1 | Lacnotuzumab | cancer |
| (CSF1) | | |
| colony stimulating factor 1 | Cabiralizumab, Emactuzumab | cancer |
| receptor (CSF1R) | | |
| Colony stimulating factor 2 | Gimsilumab,Lenzilumab, Otilimab, | leukemias |
| (CSF2) | Mavrilimumab | |
| cytotoxic T-lymphocyte | Tremelimumab | non-small cell lung, head |
| associated protein 4 | | & neck, urothelial cancer |
| (CTLA-4) | | |
| CXCR4 (CD184) | Ulocuplumab | hematologic malignancies |
| dendritic cell-associated | Tepoditamab | cancer |
| lectin 2 | | |
| delta like canonical Notch | Rovalpituzumab | small cell lung cancer |
| ligand 3 (DLL3) | | |
| delta like canonical Notch | Demcizumab | cancer |
| ligand 4 (DLL4) | | |
| TNF receptor superfamily | Drozitumab | cancer |
| member 10b (DR5) | | |
| EGF like domain multiple | Parsatuzumab | cancer |
| 7 (EGFL7) | | |
| epidermal growth factor | Cetuximab, Depatuxizumab, | cancer |
| receptor (EGFR) | Futuximab, Imgatuzumab, | |
| | Laprituximab, Matuzumab, | |
| | Necitumumab, Nimotuzumab, | |
| | Panitumumab, Zalutumumab, | |
| | Modotuximab, Amivantamab, | |
| | Tomuzotuximab, Losatuxizumab | |
| epithelial cell adhesion | Adecatumumab, Citatuzumab, | cancer |
| molecule (EpCAM) | Edrecolomab, Oportuzumab, | |
| | Solitomab, Tucotuzumab, | |
| | Catumaxomab | |
| EPH receptor A3 (EPHA3) | Ifabotuzumab | glioblastoma multiforme |
| erb-b2 receptor tyrosine | Duligotuzumab, Elgemtumab, | cancer |
| kinase 3 (ERBB3, HER3) | Lumretuzumab, Patritumab, | |
| | Seribantumab, Zenocutuzumab | |
| fibroblast growth factor | Aprutumab, Bemarituzumab | cancer |
| receptor (FGFR2) | | |
| Frizzled receptor | Vantictumab | cancer |
| GD2 ganglioside | Dinutuximab | neuroblastoma |
| GD3 ganglioside | Ecromeximab | malignant melanoma |
| GD3 ganglioside | Mitumomab | small cell lung carcinoma |
| glypican 3 | Codrituzumab | cancer |
| glycoprotein nmb | Glembatumumab | melanoma, breast cancer |
| (GPNMB) | | |
| epidermal growth factor | Zatuximab | cancer |
| receptor (HER1) | | |
| erb-b2 receptor tyrosine | Ertumaxomab, Margetuximab, | cancer, breast cancer |
| kinase 2 (HER2) | Timigutuzumab, Gancotamab, | |
| | Pertuzumab, Trastuzumab | |
| hepatocyte growth factor | Ficlatuzumab, Rilotumumab | cancer |
| (HGF) | | |
| MET proto-oncogene, | Telisotuzumab, Emibetuzumab | cancer |
| receptor tyrosine kinase | | |
| (HGFR) | | |
| IGF-1 receptor (CD221) | Cixutumumab, Dalotuzumab, | cancer |
| | Figitumumab, Ganitumab, | |
| | Robatumumab, Teprotumumab | |
| Interleukin 3 receptor | Flotetuzumab | hematological |
| | | malignancies |
| Interleukin 1 alpha (IL1A) | Bermekimab | colorectal cancer |
| Interleukin 2 (IL2) | Cergutuzumab | cancer |
| integrin a5ß1 | Volociximab | solid tumors |
| integrin avß3 | Etaracizumab | melanoma, prostate |
| | | cancer, ovarian cancer |
| lymphocyte activating 3 | Relatlimab | melanoma |
| (LAG3) | | |
| C-C motif chemokine | Carlumab | cancer |
| ligand 2 (MCP-1) | | |
| mesothelin | Amatuximab | cancer |
| Mucin 1 | Clivatuzumab, Gatipotuzumab, | cancer |
| | Pemtumomab, Cantuzumab, | |
| | Pankomab | |
| NGNA ganglioside | Racotumomab | non-small cell lung cancer |
| Notch 1 | Brontictuzumab | cancer |
| Notch receptor | Tarextumab | cancer |
| neuropilin 1 (NRP1) | Vesencumab | cancer |
| programmed cell death 1 | Camrelizumab, Cetrelimab, | cancer |
| (PD-1) | Nivolumab,Pembrolizumab, | |
| | Pidilizumab, Cemiplimab, | |
| | Spartalizumab | |
| CD274 molecule (PD-L1) | Atezolizumab, Avelumab, | cancer |
| | Durvalumab | |
| receptor tyrosine kinase | Cirmtuzumab | leukemia |
| like orphan receptor 1 | | |
| (ROR1) | | |
| tenascin C | Tenatumomab | cancer |
| transforming growth factor | Fresolimumab | cancer |
| beta 1 (TGF-ß) | | |
| VEGF-A | Brolucizumab, Bevacizumab, | cancer |
| | Ranibizumab, Varisacumab, | |
| | Faricimab | |
| VEGFR-1 | Icrucumab | cancer |
| VEGFR2 | Alacizumab, Ramucirumab | cancer |

### Inhibitors

The disclosure provides a second ligand, an inhibitor, and a second engineered receptor comprising a second ligand binding domain that binds to the inhibitor ligand.

The disclosure provides a second engineered receptor comprising an extracellular region, the extracellular region comprising a second ligand binding domain capable of specifically binding to a second ligand that inhibits activation of effector cells expressing the first and second receptors, wherein the effector cells are activated by binding of the first ligand to the first engineered receptor.

As used herein an “inhibitor” or “inhibitor ligand,” sometimes called a “blocker,” refers to a second ligand that binds to a second, ligand binding domain (inhibitor LBD) of an engineered receptor of the disclosure, but inhibits activation of an immune cell expressing the engineered receptor. The inhibitor is not expressed by the target cells. The inhibitor ligand is also expressed in a plurality of normal, non-target cells, including normal, non-target cells that express the activator ligand, thereby protecting these cells from the cytotoxic effects of the adoptive cell therapy. Without wishing to be bound by theory, inhibitor ligands can block activation of the effector cells through a variety of mechanisms. For example, binding of the inhibitor ligand to the inhibitor LBD can block transmission of a signal that occurs upon binding of the activator ligand to the activator LBD that would, in the absence of the inhibitor, lead to activation of the immune cell expressing the engineered receptors described herein.

Alternatively, or in addition, binding of the inhibitor ligand to the second engineered receptor can cause loss of cell surface expression the first, activator receptor from the surface of the immune cells comprising the two receptor system described herein. Without wishing to be bound by theory, it is thought that immune cell engagement of activator and inhibitor ligands on normal cells causes the inhibitor receptor to cause removal of nearby activator receptor molecules from the immune cell surface. This process locally desensitizes the immune cell, reversibly raising its activation threshold. Immune cells that engage only the activator ligand on a target cell cause local activation signals which are unimpeded by signals from the second, inhibitory receptor. This local activation increases until release of cytotoxic granules leads to target cell selective cell death. However, modulation of surface receptor expression levels may not be the only mechanism by which blocker receptors inhibit activation of immune cells by the first activator receptor. Without wishing to be bound by theory, other mechanisms may come into play, including, but not limited to, cross-talk between activator and blocker receptor signaling pathways.

In some embodiments, the second ligand is not expressed by the target cells, and is expressed by the non-target cells. In some embodiments, the target cells are cancer cells and the non-target cells are non-cancerous cells.

In some embodiments, the second, inhibitor ligand binding domain comprises an ScFv domain.

In some embodiments, the second, inhibitor ligand binding domain comprises a VP-only ligand binding domain.

In some embodiments, the second, inhibitor ligand binding domain comprises an antigen binding domain isolated or derived from a T cell receptor (TCR). For example, the second, inhibitor ligand binding domain comprises TCR α and β chain variable domains.

### Inhibitor Targets

In some embodiments, the inhibitor ligand comprises a gene with high, homogeneous surface expression across tissues, or a peptide antigen thereof. Without wishing to be bound by theory, high, homogeneous surface expression across tissues allows the inhibitor ligand to deliver a large, even inhibitory signal. Alternatively, or in addition, expression of activator and inhibitor targets may be correlated, i.e. the two are expressed at similar levels on non-target cells.

In some embodiments, the second, inhibitor ligand is a peptide ligand. In some embodiments, the second, inhibitor ligand is a peptide antigen complexed with a major histocompatibility (MHC) class I complex (peptide MHC, or pMHC). Inhibitor ligands comprising peptide antigens complexed with pMHC comprising any of HLA-A, HLA-B or HLA-C are envisaged as within the scope of the disclosure.

In some embodiment, the inhibitor ligand is encoded by a gene that is absent or polymorphic in many tumors.

Methods of distinguishing the differential expression of inhibitor ligands between target and non-target cells will be readily apparent to the person or ordinary skill in the art. For example, the presence or absence of inhibitor ligands in non-target and target cells can be assayed by immunohistochemistry with an antibody that binds to the inhibitor ligand, followed by microscopy or FACS, RNA expression profiling of target cells and non-target cells, or DNA sequencing of non-target and target cells to determine if the genomic locus of the inhibitor ligand comprises mutations in either the target or non-target cells.

### Alleles Lost Due to Loss of Heterozygosity (LOH)

Homozygous deletions in primary tumors are rare and small, and therefore unlikely to yield target B candidates. For example, in an analysis of 2218 primary tumors across 21 human cancer types, the top four candidates were cyclin dependent kinase inhibitor 2A (CDKN2A), RB transcriptional corepressor 1 (RBI), phosphatase and tensin homolog (PTEN) and N3PB2. However, CDKN2A (P16) was deleted in only 5% homozygous deletion across all cancers. Homozygous HLA-A deletions were found in less than 0.2% of cancers (Cheng et al., Nature Comm. 8:1221 (2017)). In contrast, deletion of a single copy of a gene in cancer cells due to loss of hemizygosity occurs far more frequently.

In some embodiments, the second, inhibitor ligand comprises an allele of a gene that is lost in target cells due to loss of heterozygosity. In some embodiments, the target cells comprises cancer cells. Cancer cells undergo frequent genome rearrangements, including duplication and deletions. These deletions can lead to the deletion of one copy of one or more genes in the cancer cells.

As used herein, “loss of heterozygosity (LOH)” refers to a genetic change that occurs at high frequency in cancers, whereby one of the two alleles is deleted, leaving a single mono-allelic (hemizygous) locus.

### HLA Class I Alleles

In some embodiments, the second, inhibitor ligand comprises an HLA class I allele. The major histocompatibility complex (MHC) class I is a protein complex that displays antigens to cells of the immune system, triggering immune response. The Human Leukocyte Antigens (HLAs) corresponding to MHC class I are HLA-A, HLA-B and HLA-C.

In some embodiments, the second, inhibitor ligand comprises an HLA class I allele. In some embodiments, the second, inhibitor ligand comprises an allele of HLA class I that is lost in a target cell through LOH. HLA-A is a group of human leukocyte antigens (HLA) of the major histocompatibility complex (MHC) that are encoded by the HLA-A locus. HLA-A is one of three major types of human MHC class I cell surface receptors. The receptor is a heterodimer comprising a heavy α chain and smaller β chain. The α chain is encoded by a variant of HLA-A, while the β chain (β2-microglobulin) is invariant. There are several thousand HLA-A variants, all of which fall within the scope of the instant disclosure.

In some embodiments, the second, inhibitor ligand comprises an HLA-B allele. The HLA-B gene has many possible variations (alleles). Hundreds of versions (alleles) of the HLA-B gene are known, each of which is given a particular number (such as HLA-B27).

In some embodiments, the second, inhibitor ligand comprises an HLA-C allele. HLA-C belongs to the HLA class I heavy chain paralogues. This class I molecule is a heterodimer consisting of a heavy chain and a light chain (beta-2 microglobulin). Over one hundred HLA-C alleles have been described.

In some embodiments, the HLA class I allele has broad or ubiquitous RNA expression.

In some embodiments, the HLA class I allele has a known, or generally high minor allele frequency.

In some embodiments, the HLA class I allele does not require a peptide-MHC antigen, for example when the HLA class I allele is recognized by a pan-HLA ligand binding domain.

In some embodiments, the second inhibitor ligand comprises an HLA-A allele. In some embodiments the HLA-A allele comprises HLA-A*02. Various single variable domains known in the art or disclosed herein that bind to and recognize HLA-A*02 are suitable for use in embodiments. Such scFvs include, for example and without limitation, the following mouse and humanized scFv that bind HLA-A*02 in a peptide-independent which are set forth in SEQ ID NOs: 50-61. Illustrative polynucleotide sequences encoding scFvs that bind HLA-A*02 are set forth in SEQ ID NOs: 175-186.

In some embodiments, the scFv comprises the complementarity determined regions (CDRs) of any one of SEQ ID NOS: 39-49. In some embodiments, the scFv comprises a sequence at least 95% identical to any one of SEQ ID NOS: 39-49. In some embodiments, the scFv comprises a sequence identical to any one of SEQ ID NOS: 39-49. In some embodiments, the heavy chain of the antibody or scFv comprises the heavy chain CDRs of any one of SEQ ID NOS: 50-61, and wherein the light chain of the antibody or scFv comprises the light chain CDRs of any one of SEQ ID NOS: 50-61. In some embodiments, the heavy chain of the antibody or scFv comprises a sequence at least 95% identical to the heavy chain portion of any one of SEQ ID NOS: 50-61, and wherein the light chain of the antibody or scFv comprises a sequence at least 95% identical to the light chain portion of any one of SEQ ID NOS: 50-61.

In some embodiments, the heavy chain of the antibody or scFv comprises a sequence identical to the heavy chain portion of any one of SEQ ID NOS: 50-61, and wherein the light chain of the antibody or scFv comprises a sequence identical to the light chain portion of any one of SEQ ID NOS: 50-61.

In some embodiments, the scFv comprises a sequence at least 95% identical, at least 96% identical, at least 97% identical, at least 98% identical, at least 99% identical or identical to any one of SEQ ID NOS: 50-61.

In some embodiments, the second, inhibitory ligand is HLA-A*02, and the inhibitory ligand binding domain comprises an HLA-A*02 ligand binding domain. In some embodiments, the second ligand binding domain binds HLA-A*02 independent of the peptide in a pMHC complex comprising HLA-A*02. In some embodiments, the HLA-A*02 ligand binding domain comprises an ScFv domain. In some embodiments, the HLA-A*02 ligand binding domain comprises a sequence of any one of SEQ ID NOs: 50-61. In some embodiments, the HLA-A*02 ligand binding domain comprises a sequence at least 90%, at least 95% or at least 99% identical to a sequence of any one of SEQ ID NOs: 50-61. In some embodiments, the HLA-A*02 ligand binding domain is encoded by a sequence comprising any one of SEQ ID NOs: 175-186. In some embodiments, the HLA-A*02 ligand binding domain is encoded by a sequence having at least 80% identity, at least 85% identity, at least 90% identity, at least 95% identity or at least 99% identity to a sequence of any one of SEQ ID NOs: 175-186.

In some embodiments, the non-target antigen comprises HLA-A*01, and the ligand binding domain of the second receptor comprises an HLA-A*01 ligand binding domain. Exemplary HLA-A*01 ligand binding domains are described in International Patent Application No. PCT/US2021/046733, which is incorporated herein by reference in its entirety for examples of sequences of HLA-A*01 ligand binding domains. In some embodiments, the ligand binding domain binds HLA-A*01 independent of the peptide in a pMHC complex comprising HLA-A*01. In some embodiments, the HLA-A*01 ligand binding domain comprises an scFv domain. In some embodiments, the HLA-A*01 ligand binding domain comprises a sequence of any one of SEQ ID NOs: 21767-21775. In some embodiments, the HLA-A*01 ligand binding domain comprises a sequence at least 90%, at least 95% or at least 99% identical to a sequence of any one of SEQ ID NO: 21767-21775.

In some embodiments, the HLA-A*01 scFv comprises (a) a VH comprising a complementarity determined region (CDR) 1 comprising the sequence of any one of SEQ ID NOs: 21585, 21578, 21576, 21583, 21794, 21581, and 21575; a VH CDR2 comprising the sequence of any one of SEQ ID NOs: 21608, 21600, 21598, 21606, 21605, and 21596; and a VH CDR3 comprising the sequence of any one of SEQ ID NOs: 21870, 21871, 21872, 21873, 21874, 21875, 21876, 21877, and 21878 and (b) a VL comprising a VL CDR1 comprising the sequence of any one of SEQ ID NOs: 21679, 21776, 21777, and 21681; a VL CDR2 comprising the sequence of any one of SEQ ID NOs: 21684, 21779, and 21781; and a VL CDR3 comprising the sequence of any one of SEQ ID NOs: 21686, 21783, 21785, and 21687.

In some embodiments, the non-target antigen comprises HLA-A*03, and the ligand binding domain of the second receptor comprises an HLA-A*03 ligand binding domain. Exemplary HLA-A*03 ligand binding domains are described in International Patent Application No. PCT/US2021/046733, which is incorporated herein by reference in its entirety for examples of sequences of HLA-A*03 ligand binding domains. In some embodiments, the ligand binding domain binds HLA-A*03 independent of the peptide in a pMHC complex comprising HLA-A*03. In some embodiments, the HLA-A*03 ligand binding domain comprises an scFv domain. In some embodiments, the HLA-A*03 ligand binding domain comprises a sequence of any one of SEQ ID NOs: 21753-21757. In some embodiments, the HLA-A*03 ligand binding domain comprises a sequence at least 90%, at least 95% or at least 99% identical to a sequence of any one of SEQ ID NOs: 21753-21757.

In some embodiments, the HLA-A*03 scFv comprises (a) a VH comprising a CDR1 comprising the sequence of any one of SEQ ID NOs: 21576-21579, 21585, 21586, 21590, 21788; a VH CDR2 comprising the sequence of any one of SEQ ID NOs: 21598-21600, 21603, 21608, 21609, 21795; and a VH CDR3 comprising the sequence of any one of SEQ ID NOs: 21801-21814 and (b) a VL comprising a VL CDR1 comprising the sequence of any one of SEQ ID NOs: 21679, 21680, 21776, and 21777; a VL CDR2 comprising the sequence of any one of SEQ ID NOs: 21684, and 21779-21781 and a VL CDR3 comprising the sequence of any one of SEQ ID NOs: 21686, and-21783-21785.

In some embodiments, the non-target antigen comprises HLA-A*11, and the ligand binding domain of the second receptor comprises an HLA-A*11 ligand binding domain. Exemplary HLA-A*11 ligand binding domains are described in International Patent Application No. PCT/US2021/046733, which is incorporated herein by reference in its entirety for examples of sequences of HLA-A*11 ligand binding domains. In some embodiments, the ligand binding domain binds HLA-A*11 independent of the peptide in a pMHC complex comprising HLA-A*11. In some embodiments, the HLA-A*11 ligand binding domain comprises an scFv domain. In some embodiments, the HLA-A*11 ligand binding domain comprises a sequence of any one of SEQ ID NOs: 21699-21707. In some embodiments, the HLA-A*11 ligand binding domain comprises a sequence at least 90%, at least 95% or at least 99% identical to a sequence of any one of SEQ ID NOs: 21699-21707.

In some embodiments, the HLA-A*11 scFv comprises (a) a VH comprising a CDR1 comprising the sequence of any one of SEQ ID NOs: 21576, 21790, 21593, 21791, and 21574; a VH CDR2 comprising the sequence of any one of SEQ ID NOs: 21598, 21797, 21597, 21798, and 21602; and a VH CDR3 comprising the sequence of any one of SEQ ID NOs: 21816-21824 and (b) a VL comprising a VL CDR1 comprising the sequence of SEQ ID NO: 21680; a VL CDR2 comprising the sequence of SEQ ID NO: 21684; and a VL CDR3 comprising the sequence of SEQ ID NO: 21686.

In some embodiments, the non-target antigen comprises HLA-B*07, and the ligand binding domain of the second receptor comprises an HLA-B*07 ligand binding domain. Exemplary HLA-B*07 ligand binding domains are described in International Patent Application No. PCT/US2021/046733, which is incorporated herein by reference in its entirety for examples of sequences of HLA-B*07 ligand binding domains. In some embodiments, the ligand binding domain binds HLA-B*07 independent of the peptide in a pMHC complex comprising HLA-B*07. In some embodiments, the HLA-B*07 ligand binding domain comprises an scFv domain. In some embodiments, the HLA-B*07 ligand binding domain comprises a sequence of any one of SEQ ID NOs: 21689-21698. In some embodiments, the HLA-B*07 ligand binding domain comprises a sequence at least 90%, at least 95% or at least 99% identical to a sequence of any one of SEQ ID NOs: 21689-21698.

In some embodiments, the HLA-B*07 scFv comprises (a) a VH comprising a CDR1 comprising the sequence of SEQ ID NO: 21789; a VH CDR2 comprising the sequence of SEQ ID NO: 21796; and a VH CDR3 comprising the sequence of SEQ ID NO: 21815 and (b) a VL comprising a VL CDR1 comprising the sequence of SEQ ID NO: 21778; a VL CDR2 comprising the sequence of SEQ ID NO: 21782; and a VL CDR3 comprising the sequence of SEQ ID NO: 21786.

In some embodiments, the non-target antigen comprises HLA-C*07, and the ligand binding domain of the second receptor comprises an HLA-C*07 ligand binding domain. Exemplary HLA-C*07 ligand binding domains are described in International Patent Application No. PCT/US2021/046733, which is incorporated herein by reference in its entirety for examples of sequences of HLA-C*07 ligand binding domains. In some embodiments, the ligand binding domain binds HLA-C*07 independent of the peptide in a pMHC complex comprising HLA-C*07. In some embodiments, the HLA-C*07 ligand binding domain comprises an scFv domain. In some embodiments, the HLA-C*07 ligand binding domain comprises a sequence of any one of SEQ ID NOs: 21708-21752. In some embodiments, the HLA-C*07 ligand binding domain comprises a sequence at least 90%, at least 95% or at least 99% identical to a sequence of any one of SEQ ID NOs: 21708-21752.

In some embodiments, the HLA-C*07 scFv comprises (a) a VH comprising a CDR1 comprising the sequence of any one of SEQ ID NOs: 21585, 21578, 21577, 21576, 21593, 21591, 21589, 21583, 21792, and 21793; a VH CDR2 comprising the sequence of any one of SEQ ID NOs: 21608, 21600, 21599, 21598, 21597, 21613, 21612, 21606, 21799, and 21800; and a VH CDR3 comprising the sequence of any one of SEQ ID NOs: 21825-21869 and (b) a VL comprising a VL CDR1 comprising the sequence of any one of SEQ ID NOs: 21679, 21680, 21777, 21682, and 21681; a VL CDR2 comprising the sequence of any one of SEQ ID NOs: 152, 21684, 21780, and 21781; and a VL CDR3 comprising the sequence of any one of SEQ ID NOs: 157, 21686, 21784, 21785, 21787, and 21687.

### Minor Histocompatibility Antigens

In some embodiments, the second, inhibitor ligand comprises a minor histocompatibility antigen (MiHA). In some embodiments, the second, inhibitor ligand comprises an allele of a MiHA that is lost in a target cell through LOH.

MiHAs are peptides derived from proteins that contain nonsynonymous differences between alleles and are displayed by common HLA alleles. The non-synonymous differences can arise from SNPs, deletions, frameshift mutations or insertions in the coding sequence of the gene encoding the MiHA. Exemplary MiHAs can be about 9-12 amino acids in length and can bind to MHC class I and MHC class II proteins. Binding of the TCR to the MHC complex displaying the MiHA can activate T cells. The genetic and immunological properties of MiHAs will be known to the person of ordinary skill in the art. Candidate MiHAs are known peptides presented by known HLA class I alleles, are known to elicit T cell responses in the clinic (for example, in graft versus host disease, or transplant rejection, and allow for patient selection by simple SNP genotyping.

In some embodiments, the MiHA has broad or ubiquitous RNA expression.

In some embodiments, the MiHA has high minor allele frequency.

In some embodiments, the MiHA comprises a peptide derived from a Y chromosome gene.

In some embodiments, the second inhibitor ligand comprises a MiHA selected from the group of MiHAs disclosed in Table 2 and Table 3.

Exemplary, but non-limiting, examples of MiHAs that are envisaged as within the scope of the instant invention are disclosed in Table 2 below. Columns in 2 indicate, from left to right, the name of the MiHA, the gene which from which it is derived, MHC class I variant which can display the MiHA and the sequences of the peptide variants [A/B variants indicated in brackets).

TABLE 2
HLA Class I Autosomal MiHAs.

| MiHA | Gene | HLA | Peptide A/B |
|---|---|---|---|
| LB-CYBA- | cytochrome b-245 alpha chain | A*01:01 | STMERWGQK[Y/H] (SEQ ID |
| 1Y | (CYBA) | | NO: 303) |
| LB-OAS1- | 2′-5′-oligoadenylate synthetase | A*01:01 | ETDDPR[R/T]YQKY (SEQ ID |
| 1R | 1 (OAS1) | | NO: 304) |
| HA-1/A2 | Rho GTPase activating protein | A*02:01 | VL[H/R]DDLLEA (SEQ ID NO: |
| | 45 (HMHA1) | | 273) |
| HA-2 | myosin IG (MYO1G) | A*02:01 | YIGEVLVS[V/M] (SEQ ID NO: |
| | | | 305) |
| HA-8 | pumilio RNA binding family | A*02:01 | [R/P]TLDKVLEV (SEQ ID NO: |
| | member 3 (KIAA0020, PUM3) | | 306) |
| HA-3 | A-kinase anchoring protein 13 | A*01:01 | V[T/M]EPGTAQY (SEQ ID NO: |
| | (AKAP13) | | 307) |
| HwA11-S | chromosome 19 open reading | A*02:01 | CIPPD[S/T]LLFPA (SEQ ID |
| | frame 48 (C19ORF48) | | NO: 308) |
| LB-ADIR- | torsin family 3 member A | A*02:01 | SVAPALAL[F/S]PA (SEQ ID |
| 1F | (TOR3A) | | NO: 309) |
| LB- | HIVEP zinc finger 1 (HIVEP1) | A*02:01 | SLPKH[S/N]VTI (SEQ ID NO: |
| HIVEP1-1S | | | 310) |
| LB-NISCH- | nischarin (NISCH) | A*02:01 | ALAPAP[A/V]EV (SEQ ID NO: |
| 1A | | | 311) |
| LB-SSR1- | signal sequence receptor | A*02:01 | [S/L]LAVAQDLT (SEQ ID |
| 1S | subunit 1 (SSR1) | | NO:312) |
| LB-WNK1- | WNK lysine deficient protein | A*02:01 | RTLSPE[I/M]ITV (SEQ ID NO: |
| 1I | kinase 1 (WNK1) | | 313) |
| T4A | tripartite motif containing 4 | A*02:01 | GLYTYWSAG[A/E] (SEQ ID |
| | (TRIM42) | | NO: 314) |
| UTA2-1 | retroelement silencing factor 1 | A*02:01 | QL[L/P]NSVLTL (SEQ ID NO: |
| | (KIAA1551) | | 315) |
| LB- | citramalyl-CoA lyase | A*02:01 | SLAA(Y/D)IPRL (SEQ ID NO: |
| CLYBL-1Y | (CLYBL) | | 316) |
| TRIM22 | tripartite motif containing 22 | A*02:01 | MAVPPC[C/R]IGV (SEQ ID |
| | (TRIM22) | | NO: 317) |
| PARP10-1L | poly(ADP-ribose) polymerase | A*02:01 | GL[L/P]GQEGLVEI (SEQ ID |
| | family member 10 (PARP10) | | NO: 318) |
| FAM119A- | methyltransferase like 21A | A*02:01 | AMLERQF[T/I]V (SEQ ID NO: |
| 1T | (FAM119A) | | 319) |
| GLRX3-1S | glutaredoxin 3 (GLRX3) | A*02:01 | FL[S/P]SANEHL (SEQ ID NO: |
| | | | 320) |
| HNF4G-1M | hepatocyte nuclear factor 4 | A*02:01 | M[M/I]YKDILLL (SEQ ID NO: |
| | gamma (HNF4G) | | 321) |
| HMMR-1V | hyaluronan mediated motility | A*02:01 | SLQEK[V/A]AKA (SEQ ID NO: |
| | receptor (HMMR) | | 322) |
| BCL2A1 | BCL2 related protein Al | A*02:01 | VLQ[N/K]VAFSV (SEQ ID NO: |
| | (BCL2A1) | | 323) |
| CDC26-1F | cell division cycle 26 (CDC26) | A*02:01 | [F/S]VAGTQEVFV (SEQ ID |
| | | | NO: 324) |
| APOBEC3F- | apolipoprotein B mRNA | A*02:01 | FL[S/A]EHPNVTL (SEQ ID |
| 1S/A | editing enzyme catalytic | | NO: 325) |
| | subunit 3F (APOBEC3F) | | |
| LB-PRCP- | prolylcarboxypeptidase | A*02:01 | FMWDVAE[D/E]L (9 mer) (SEQ |
| 1D | (PRCP) | | ID NO: 326), |
| | | | FMWDVAE[D/E]LKA (11 mer) |
| | | | (SEQ ID NO: 327) |
| LB-CCL4- | C-C motif chemokine ligand 4 | A*02:01 | CADPSE[T/S]WV (SEQ ID NO: |
| 1T | (CCL4) | | 328) |
| LB- | non-SMC condensin II | A*02:01 | WL[Q/R]GVVPVV (SEQ ID |
| NCAPD3- | complex subunit D (NCAPD3) | | NO: 329) |
| 1Q | | | |
| LB-NDC80- | NDC80 kinetochore complex | A*02:01 | HLEEQI[P/A]KV (SEQ ID NO: |
| 1P | component (NDC80) | | 330) |
| LB-TTK- | TTK protein kinase (TTK) | A*02:01 | RLH[D/E]GRVFV (SEQ ID NO: |
| 1D | | | 331) |
| WDR27-1L | WD repeat domain 27 | A*02:01 | S[L/P]DDHVVAV (SEQ ID NO: |
| | (WDR27) | | 332) |
| MIIP | migration and invasion | A*02:01 | SEESAVP[K/E]RSW (11 mer) |
| | inhibitory protein (MIIP) | | (SEQ ID NO: 333), |
| | | | EESAVP[K/E]RSW (10 mer) |
| | | | (SEQ ID NO: 334) |
| HER- | E erb-b2 receptor tyrosine | A*02:01 | not reported |
| 2/NEU | kinase 2 (RBB2) | | |
| LB- | DEAH-box helicase 33 | A*02:01, | YLYEGGIS[C/R] (SEQ ID NO: |
| DHX33-1C | (DHX33) | C*03:03 | 335) |
| PANE1 | centromere protein M | A*03:01 | RVWDLPGVLK (SEQ ID NO: |
| | (CENPM) | | 336) |
| SP110 | SP110 nuclear body protein | A*03:01 | SLP[R/G]GTSTPK (SEQ ID NO: |
| | (SP110) | | 337) |
| ACC-1C/Y | BCL2 related protein Al | A*24:02 | DYLQ[Y/C]VLQI (SEQ ID NO: |
| | (BCL2A1) | | 338) |
| P2RX7 | purinergic receptor P2X 7 | A*29:02 | WFHHC[H/R]PKY (SEQ ID |
| | (P2RX7) | | NO: 339) |
| ACC-4 | cathepsin H (CTSH) | A*31:01 | ATLPLLCA[R/G] (SEQ ID NO: |
| | | | 340) |
| ACC-5 | CTSH | A*33:03 | WATLPLLCA[R/G] (SEQ ID |
| | | | NO: 341) |
| AKAP13 | A-kinase anchoring protein 13 | B*07:02 | APAGVREV[M/T] (SEQ ID NO: |
| | (AKAP13) | | 342) |
| LB- | apolipoprotein B mRNA | B*07:02, | [K/E]PQYHAEMCF (SEQ ID |
| APOBEC3B- | editing enzyme catalytic | B*08:01 | NO: 343) |
| 1K | subunit 3B (APOBEC3B) | | |
| APOBEC3H | apolipoprotein B mRNA | B*07:02 | KPQQ[K/E]GLRL (SEQ ID NO: |
| | editing enzyme catalytic | | 344) |
| | subunit 3H (APOBEC3H) | | |
| LB- | Rho GDP dissociation inhibitor | B*07:02 | LPRACW[R/P]EA (SEQ ID NO: |
| ARHGDIB- | beta (ARHGDIB) | | 345) |
| 1R | | | |
| LB- | BCAT2 - branched chain | B*07:02 | QP[R/T]RALLFVIL (SEQ ID |
| BCAT2-1R | amino acid transaminase 2 | | NO: 346) |
| | (BCAT2) | | |
| BFAR | bifunctional apoptosis regulator | B*07:02 | APNTGRANQQ[M/R] (SEQ ID |
| | (BFAR) | | NO: 347) |
| C14orf169 | ribosomal oxygenase 1 | B*07:02 | RPR[A/V]PTEELAL (SEQ ID |
| | (C14orf169 or RIOX1) | | NO: 348) |
| LB- | C16ORF | B*07:02 | [R/W]PCPSVGLSFL (SEQ ID |
| C16ORF- | | | NO: 349) |
| 1R | | | |
| C18orf21 | chromosome 18 open reading | B*07:02 | NPATP[A/T]SKL (SEQ ID NO: |
| | frame 21 (C18orf21) | | 350) |
| LB-EBI3-1I | Epstein-Barr virus induced 3 | B*07:02 | RPRARYY[I/V]QV (SEQ ID |
| | (EBI3) | | NO: 351) |
| POP1 | POP1 homolog, ribonuclease | B*07:02 | LPQKKS[N/K]AL (SEQ ID NO: |
| | P/MRP subunit (POP1) | | 352) |
| SCRIB | scribble planar cell polarity | B*07:02 | LPQQPP[L/P]SL (SEQ ID NO: |
| | protein (SCRIB) | | 353) |
| MTRR | 5-methyltetrahydrofolate- | B*07:02 | SPAS[S/L]RTDL (SEQ ID NO: |
| | homocysteine | | 354) |
| | methyltransferase reductase | | |
| | (MTRR) | | |
| LLGL2 | LLGL scribble cell polarity | B*07:02 | SPSL[R/H]ILAI (SEQ ID |
| | complex component 2 | | NO: 355) |
| | (LLGL2) | | |
| LB-ECGF- | thymidine phosphorylase | B*07:02 | RP[H/R]AIRRPLAL (SEQ ID |
| 1H | (TYMP) | | NO: 356) |
| LB-ERAP1- | endoplasmic reticulum | B*07:02 | HP[R/P]QEQIALLA (11 mer) |
| 1R | aminopeptidase 1 (ERAP1) | | (SEQ ID NO: 357), |
| | | | HP[R/P]QEQIAL (9 mer) (SEQ |
| | | | ID NO: 358) |
| LB- | alpha-L-fucosidase 2 (FUCA2) | B*07:02 | RLRQ[V/M]GSWL (SEQ ID |
| FUCA2-1V | | | NO: 359) |
| LB- | gem nuclear organelle | B*07:02, | FPALRFVE[V/E] (SEQ ID NO: |
| GEMIN4- | associated protein 4 (GEMIN4) | B*08:01 | 360) |
| 1V | | | |
| HDGF | heparin binding growth factor | B*07:02 | LPMEVEKNST[L/P] (SEQ ID |
| | (HDGF) | | NO: 361) |
| LB- | programmed cell death 11 | B*07:02 | GPDSSKT[F/L]LCL (SEQ ID |
| PDCD11-1F | (PDCD11) | | NO: 362) |
| LB-PFAS- | phosphoribosylformylglycina | B*07:02 | A[P/S]GHTRRKL (SEQ ID NO: |
| 1P | midine synthase (PFAS) | | 363) |
| LB-TEP1- | telomerase associated protein 1 | B*07:02 | APDGAKVA[S/P]L (SEQ ID |
| 1S | (TEP1) | | NO: 364) |
| LB- | post- | B*07:02 | RPRSVT[I/V]QPLL (SEQ ID |
| TMEM8A- | glycosylphosphatidylinositol | | NO: 365) |
| 1I | attachment to proteins 6 | | |
| | (TMEM8A or PGAP6) | | |
| LB-USP15- | ubiquitin specific peptidase 15 | B*07:02 | MPSHLRN[I/T]LL (SEQ ID NO: |
| 1I | (USP15) | | 366) |
| LRH-1 | purinergic receptor P2X 5 | B*07:02 | TPNQRQNVC (SEQ ID NO: |
| | (P2RX5) | | 367) |
| LB- | MOB kinase activator 3A | B*07:02 | [C/S]PRPGTWTC (SEQ ID NO: |
| MOB3A-1C | (MOB3A) | | 368) |
| LB- | zinc finger DHHC-type | B*07:02 | RPR[Y/H]WILLVKI (SEQ ID |
| ZDHHC6- | palmitoyltransferase 6 | | NO: 369) |
| 1Y | (ZDHHC6) | | |
| ZAPHIR | zinc finger protein 419 | B*07:02 | IPRDSWWVEL (SEQ ID NO: |
| | (ZNF419) | | 370) |
| HEATR1 | HEAT repeat containing 1 | B*08:01 | ISKERA[E/G]AL (SEQ ID NO: |
| | (HEATR1) | | 371) |
| LB-GSTP1- | glutathione S-transferase pi 1 | B*08:01 | DLRCKY[V/I]SL (SEQ ID NO: |
| 1V | (GSTP1) | | 372) |
| HA-1/B60 | Rho GTPase activating protein | B*40:01 | KECVL[H/R]DDL (SEQ ID NO: |
| | 45 (HMHA1) | | 373) |
| LB-SON- | SON DNA and RNA binding | B*40:01 | SETKQ[R/C]TVL (SEQ ID NO: |
| 1R | protein (SON) | | 374) |
| LB- | switching B cell complex | B*40:01 | MEQLE[Q/E]LEL (SEQ ID NO: |
| SWAP70- | subunit SWAP70 (SWAP70) | | 375) |
| 1Q | | | |
| LB- | thyroid hormone receptor | B*40:01 | G[E/G][P/S]QDL[C/G]TL (SEQ |
| TRIP10- | interactor 10 (TRIP10) | | ID NO: 376) |
| 1EPC | | | |
| LB- | nucleoporin 133 (NUP133) | B*40:01 | SEDLILC[R/Q]L (SEQ ID NO: |
| NUP133-1R | | | 377) |
| LB-ZNFX1- | zinc finger NFX1-type | B*40:01 | NEIEDVW[Q/H]LDL (SEQ ID |
| 1Q | containing 1 (ZNFX1) | | NO: 378) |
| SLC1A5 | solute carrier family 1 member | B*40:02 | AE[A/P]TANGGLAL (SEQ ID |
| | 5 (SLC1A5) | | NO: 379) |
| ACC-2 | BCL2A1 | B*44:02, | KEFED[D/G]IINW (SEQ ID |
| | | B*44:03 | NO: 380) |
| ACC-6 | histocompatibility minor serpin | B*44:03 | MEIFIEVFSHF (SEQ ID NO: |
| | domain containing (HMSD) | | 381) |
| HB-1H/Y | histocompatibility minor HB-1 | B*44:03 | EEKRGSL[H/Y]VW (SEQ ID |
| | (HMHB1) | | NO: 382) |
| DPH1 | diphthamide biosynthesis 1 | B*57:01 | S[V/L]LPEVDVW (SEQ ID NO: |
| | (DPH1) | | 383) |
| UGT2B17/ | UDP glucuronosyltransferase | A*02:06 | CVATMIFMI (SEQ ID NO: 384) |
| A02 | family 2 member B17 | | |
| | (UGT2B17) | | |
| UGT2B17/ | UGT2B17 | A*29:02 | AELLNIPFLY (SEQ ID NO: |
| A29 | | | 385) |
| UGT2B17/ | UGT2B17 | B*44:03 | AELLNIPFLY (SEQ ID NO: |
| B44 | | | 386) |

Exemplary, but non-limiting, examples of MiHAs that are envisaged as within the scope of the instant invention are disclosed in Table 9 below. Columns in Table 3 indicate, from left to right, the name of the MiHA, the gene which from which it is derived, MHC class I variant which can display the MiHA and the sequences of the peptide variants [A/B variants indicated in brackets).

TABLE 3
HLA Class I Y linked MiHAs.

| MiHA | Gene | HLA | Peptide A/B |
|---|---|---|---|
| DFFRY | ubiquitin specific peptidase 9 | A*01:01 | IVD[C/S]LTEMY (SEQ ID NO: |
| | Y-linked (DFFRY) | | 387) |
| SMCY | lysine demethylase 5 (SMCY) | A*02:01 | FIDSYICQV (SEQ ID NO: 388) |
| TMSB4Y | thymosin beta 4 Y-linked | A*33:03 | EVLLRPGLHFR (SEQ ID NO: |
| | (TMSB4Y) | | 389) |
| SMCY | SMCY | B*07:02 | SP[S/A]VDKA[R/Q]AEL (SEQ |
| | | | ID NO: 34) |
| UTY | ubiquitously transcribed | B*08:01 | LPHN[H/R]T[D/N]L (SEQ ID |
| | tetratricopeptide repeat | | NO: 25) |
| | containing, Y-linked (UTY) | | |
| RPS4Y | ribosomal protein S4 Y-linked 1 | B*52:01 | TIRYPDP[V/L]I (SEQ ID NO: |
| | (RPS4Y) | | 24) |
| UTY | UTY | B*60:01 | [R/G]ESEE[E/A]S[V/P]SL |
| | | | (SEQ ID NO: 23) |

In some embodiments, the MiHA comprises HA-1. HA-1 is a peptide antigen having a sequence of VL[H/R]DDLLEA (SEQ ID NO: 264), and is derived from the Rho GTPase activating protein 45 (HA-1) gene.

Exemplary ligand binding domains that selectively bind to HA-1 variant H peptide (VLHDDLLEA (SEQ ID NO: 187)) are set forth in SEQ ID NOs: 189, 191, 193, 195, and 196. Illustrative nucleic acid sequences encoding ligand binding domains that selectively bind to HA-1 variant H peptide (VLHDDLLEA (SEQ ID NO: 187)) are set forth in SEQ ID NOs: 190, 192, 194, 197, and 198.

TCR alpha and TCR beta sequences in SEQ ID NO: 189 are separated by a P2A self-cleaving polypeptide of sequence ATNFSLLKQAGDVEENPGP (SEQ ID NO: 188) with an N terminal GSG linker.

In some embodiments, the second, inhibitory ligand comprises HA-1(H). In some embodiments, the second, inhibitory ligand binding is isolated or derived from a TCR. In some embodiments, the second, inhibitory ligand binding domain comprises TCR alpha and TCR beta variable domains. In some embodiments, the TCR alpha and TCR beta variable domains are separated by a self cleaving polypeptide sequence. In some embodiments, the TCR alpha and TCR beta variable domains separated by a self cleaving polypeptide sequence comprise SEQ ID NO: 189. In some embodiments, the TCR alpha and TCR beta variable domains separated by a self cleaving polypeptide sequence comprise SEQ ID NO: 189, or a sequence having at least 90%, at least 95%, or at least 99% identity thereto. In some embodiments, the TCR alpha and TCR beta variable domains are encoded by a sequence of SEQ ID NO: 190, or a sequence having at least 80% identity, at least 90%, at least 95%, or at least 99% identity thereto. In some embodiments, the TCR alpha variable domain comprises SEQ ID NO: 195 or a sequence having at least 90%, at least 95%, or at least 99% identity thereto. In some embodiments, the TCR beta variable domain comprises SEQ ID NO: 196 or a sequence having at least 90%, at least 95%, or at least 99% identity thereto.

### Loss of Y Chromosome Antigens

In some embodiments, the second, inhibitor ligand comprises a Y chromosome gene, i.e. peptide encoded by a gene on the Y chromosome. In some embodiments, the second, inhibitor ligand comprises a peptide encoded by a Y chromosome gene that is lost in target cells through loss of Y chromosome (LoY). For example, about a third of the characterized MiHAs come from the Y chromosome. The Y chromosome contains over 200 protein coding genes, all of which are envisaged as within the scope of the instant disclosure.

As used herein, “loss of Y”, or “LoY” refers a genetic change that occurs at high frequency in tumors whereby one copy of part or all of the Y chromosome is deleted, leading to a loss of Y chromosome encoded gene(s).

Loss of Y chromosome is known to occur in certain cancers. For example, there is a reported 40% somatic loss of Y chromosome in renal clear cell cancers (Arseneault et al., Sci. Rep. 7: 44876 (2017)). Similarly, clonal loss of the Y chromosome was reported in 5 out of 31 in male breast cancer subjects (Wong et al., Oncotarget 6(42):44927-40 (2015)). Loss of the Y chromosome in tumors from male patients has been described as a “consistent feature” of head and neck cancer patients (el-Naggar et al., Am J Clin Pathol 105(1):102-8 (1996)). Further, Y chromosome loss was associated with X chromosome disomy in four of seven male patients with gastric cancer (Saal et al., Virchows Arch B Cell Pathol (1993)). Thus, Y chromosome genes can be lost in a variety of cancers, and can be used as inhibitor ligands with the engineered receptors of the instant disclosure targeting cancer cells.

### Antigen Binding Domains

The disclosure provides a first ligand binding domain that activates a first engineered receptor, thereby activating immune cells expressing the first engineered receptor, and a second ligand binding domain that activates a second engineered receptor that inhibits activation of immune cells expressing the second engineered receptor, even in the presence of the first engineered receptor bound to the first ligand.

Any type of ligand binding domain that can regulate the activity of a receptor in a ligand dependent manner is envisaged as within the scope of the instant disclosure. In some embodiments, the ligand binding domain is an antigen binding domain. Exemplary antigen binding domains include, inter alia, ScFv, SdAb, VP-only domains, and TCR antigen binding domains derived from the TCR α and β chain variable domains.

In some embodiments, the first, activator LBD comprises an antigen binding domain. In some embodiments, the second, inhibitor LBD comprises an antigen binding domain. Any type of antigen binding domain is envisaged as within the scope of the instant disclosure.

For example, the first, activator LBD and/or the second, inhibitor LBD can comprise an antigen binding domain that can be expressed as part of a contiguous polypeptide chain including, for example, a single domain antibody fragment (sdAb) or heavy chain antibodies HCAb, a single chain antibody (scFv) derived from a murine, humanized or human antibodies (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some aspects, the first, activator LBD and/or the second, inhibitor LBD comprises an antigen binding domain that comprises an antibody fragment. In further aspects, the activator LBD comprises an antibody fragment that comprises a scFv or an sdAb. In further aspects, the inhibitor LBD comprises an antibody fragment that comprises a scFv or an sdAb.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)2, and FAT fragments, single-chain (sc)Fv (“scFv”) antibody fragments, linear antibodies, single domain antibodies (abbreviated “sdAb”) (either VL or VH), camelid VHH domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “VH” (or, in the case of single domain antibodies, e.g., nanobodies, “VHH”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

The term “antibody light chain,” refers to the smaller of the two types of polypeptide chains present in antibody molecules in their naturally occurring conformations. Kappa (“K”) and lambda (“λ”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “Vβ domain”, “Vβ-only domain”, “β chain variable domain” or “single variable domain TCR (svd-TCR)” refers to an antigen binding domain that consists essentially of a single T Cell Receptor (TCR) beta variable domain that specifically binds to an antigen in the absence of a second TCR variable domain. In some embodiments, the first, activator LBD comprises or consists essentially of a Vβ-only domain. In some embodiments, the second, inhibitor LBD comprises or consists essentially of a Vβ-only domain.

In some embodiments, the Vβ-only domain may include additional elements besides the TCR variable domain, including additional amino acid sequences, additional protein domains (covalently associated, non-covalently associated or covalently and non-covalently associated with the TCR variable domain), fusion or non-covalent association of the TCR variable domain with other types of macromolecules (for example polynucleotides, polysaccharides, lipids, or a combination thereof), fusion or non-covalent association of the TCR variable domain with one or more small molecules, compounds, or ligands, or a combination thereof. Any additional element, as described, may be combined provided that the TCR variable domain is configured to specifically bind the epitope in the absence of a second TCR variable domain.

In other embodiments, the Vβ-only domain as described herein functions independently of an α chain that lacks a V α segment. For example, in some embodiments the one or more Vβ-only domains are fused to transmembrane (e.g., CD3ξ and CD28) and intracellular domain proteins (e.g., CD3, CD28, and/or 4-1BB) that are capable of activating T cells in response to antigen.

In some embodiments, the Vβ-only domain engages antigen using complementarity-determining regions (CDRs). Each s Vβ-only domain contains three complement determining regions (CDR1, CDR2, and CDR3).

In some embodiments, the first Vβ-only domain comprises a TCR Vβ domain or an antigen-binding fragment thereof.

In humans, the TCR variable regions of the α and γ chains are each encoded by a V and a J segment, whereas the variable region of β and δ chains are each additionally encoded by a D segment. There are multiple Variable (V), Diversity (D) and Joining (J) gene segments (e.g. 52 Vβ gene segments, 2 Dβ gene segments and 13 Jβ gene segments) (Janeway et al. (eds.), 2001, Immunobiology: The Immune System in Health and Disease. 5th Edition, New York, FIG. 4.13) which can be recombined in different V(D)J arrangements using the enzymes RAG-1 and RAG-2, which recognize recombination signal sequences (RSSs) adjacent to the coding sequences of the V, D and J gene segments. The RSSs consist of conserved heptamers and nonamers separated by spacers of 12 or 23 bp. The RSSs are found at the 3′ side of each V segment, on both the 5′ and 3′ sides of each D segment, and at the 5′ of each J segment. During recombination, RAG-1 and RAG-2 cause the formation of DNA hairpins at the coding ends of the joint (the coding joint) and removal of the RSSs and intervening sequence between them (the signal joint). The variable regions are further diversified at the junctions by deletion of a variable number of coding end nucleotides, the random addition of nucleotides by terminal deoxynucleotidyl transferase (TdT), and palindromic nucleotides that arise due to template-mediated fill-in of the asymmetrically cleaved coding hairpins.

Patent applications WO 2009/129247 (herein incorporated by reference in its entirety) discloses an in vitro system, referred to as the HuTarg system, which utilizes V(D)J recombination to generate de novo antibodies in vitro. This same system was used to generate the variable regions of the Vβ-only domain as in patent application WO 2017/091905 (herein incorporated by reference in its entirety) by using TCR-specific V, D and J elements. In natural in vivo systems, the nucleic acid sequences which encode CDR1 and CDR2 are contained within the V (α, β, γ or δ) gene segment and the sequence encoding CDR3 is made up from portions of V and J segments (for Vα or Vγ) or a portion of the V segment, the entire D segment and a portion of the J segment (for Vβ or Vδ), but with random insertions and deletions of nucleotides at the V-J and V-D-J recombination junctions due to action of TdT and other recombination and DNA repair enzymes. The recombined T-cell receptor gene comprises alternating framework (FR) and CDR sequences, as does the resulting T-cell receptor expressed therefrom (i.e. FR1-CDR1-FR2-CDR2-FR3-CDR3-FR4). Using in vitro V(D)J recombination (i.e. V-J or V-D-J recombination), randomized insertions and deletions may be added in or adjacent to CDR1, CDR2 and/or CDR3 (i.e. not just CDR3), additional insertions may be added using flanking sequences in recombination substrates before and/or after CDR1, CDR2 and/or CDR3, and additional deletions may be made by deleting sequences in recombination substrates in or adjacent to CDR1, CDR2 and/or CDR3.

In some embodiments, TCR Vβ chains were identified that specifically bind epitopes in the absence of TCR Vα chains. Exemplary CDR3 amino acid sequences that bind epitopes in the absence of TCR Vα chains are set forth in SEQ ID NOs: 199-205.

In some embodiments, the Vβ-only domain specifically binds to an epitope in the absence of a second TCR variable domain, and consists of optional N-terminal and/or C-terminal amino acid sequences (of any length or sequence) flanking a variable domain defined by FR1-CDR1-FR2-CDR2-FR3-CDR3-FR4 regions. FR1, FR2, FR3 and FR4 may be obtained from a natural Vα, Vβ, Vγ or Vδ domain or encoded by natural Vα, Vγ or Vδ gene segments, but optionally include deletions or insertions of (e.g. 0, 1, 2, 3, 4, 5 or more than 5 amino acids) amino acids independently at one or more of the C-terminus of FR1, the N-terminus of FR2, the C-terminus of FR2, the N-terminus of FR3, the C-terminus of FR3 and the N-terminus of FR4. CDR1, CDR2 and CDR3 may be obtained from a natural Vα, Vγ or Vδ domain, or encoded by natural Vα, Vγ or Vδ gene segments, but wherein one or more of CDR1, CDR2 and CDR3 independently contains an insertion (e.g. 0, 1, 2, 3, 4, 5, 6, 7, 8, 9, 10 or more than 10 amino acids) and/or a deletion (e.g. 0, 1, 2, 3, 4, 5 or more than 5 amino acids) at the C-terminus, the N-terminus or anywhere within the CDR sequence. In some embodiments, the CDR1 contains an insertion or deletion of amino acids N-terminally, C-terminally or internally, wherein at least 50% (or optionally 60%, 70% or 80%) of natural CDR amino acid residues are retained. In some embodiments, the CDR2 contains an insertion or deletion of amino acids N-terminally, C-terminally or internally, wherein at least 50% (or optionally 60%, 70% or 80%) of natural CDR amino acid residues are retained. In some embodiments, the CDR3 contains an insertion or deletion of amino acids N-terminally, C-terminally or internally, wherein at least 50% (or optionally 60%, 70% or 80%) of natural CDR amino acid residues are retained. Insertions and/or deletions may be produced as a result of in vitro V(D)J recombination methods or from the in-vitro action of TdT and recombination and DNA repair enzymes (e.g. one or more of Artemis nuclease, NDA-dependent protein kinase (DNA-PK), X-ray repair cross-complementing protein 4 (XRCC4), DNA ligase IV, non-homologous end-joining factor 1 (NHEJ1), PAXX, and DNA polymerases λ and μ). Insertion and/or deletion (which includes substitution) may further result from insertions and/or deletions to CDR nucleic acid sequences of the in vitro V(D)J recombination substrates. The Vβ-only domain may further comprise a TCR constant region or portion thereof. The Vβ-only domain may be fused to and/or complexed with additional protein domains. A double stranded break in DNA may be introduced prior to in vitro use of the above recombination and DNA repair enzymes. The Vβ-only domain may be (or may be incorporated into) a fusion protein. As used herein, the term “fusion protein” means a protein encoded by at least one nucleic acid coding sequence that is comprised of a fusion of two or more coding sequences from separate genes, regardless of whether the organism source of those genes is the same or different.

In some embodiments, the first, activator LBD comprises an ScFv domain and the second, inhibitor LBD comprises a Vβ-only domain. In some embodiments, the first, activator LBD comprises a Vβ-only domain and the second, inhibitor LBD comprises an ScFv domain. In some embodiments, both the first, activator LBD and the second, inhibitor LBD are ScFv domains. In some embodiments, both the first, activator LBD and the second, inhibitor LBD are Vβ-only domains.

Additional antigen binding domains used with the activator and/or inhibitor receptors of the disclosure are set forth in SEQ ID NOs: 206, 208, 210,212, 214, 216, 218, and 220. Exemplary nucleic acid sequences encoding these antigen binding domains are set forth in SEQ ID NOs: 207, 209, 211, 213, 215, 217, 219, and 221. In some embodiments, the first or second ligand binding domain comprises a sequence of any one of SEQ ID NO: 206, SEQ ID NO: 208, SEQ ID NO: 210, SEQ ID NO: 212, SEQ ID NO: 214, SEQ ID NO: 216, SEQ ID NO: 218 or SEQ ID NO: 220, or a sequence having at least 90%, at least 95% or at least 99% identity thereto.

### Engineered Receptors

The disclosure provides a first engineered receptor comprising a first activator ligand binding domain and a second engineered receptor comprising a second inhibitor ligand binding domain described herein.

### Chimeric Antigen Receptors (CARs)

In some embodiments, the either the first or the second engineered receptor is a chimeric antigen receptor (CAR). In some embodiments, the first and second engineered receptors are chimeric antigen receptors. All CAR architectures are envisaged as within the scope of the instant disclosure.

### Extracellular Domains

In some embodiments, the first or second ligand binding domain is fused to the extracellular domain of the CAR.

### Hinge Region

In some embodiments, the CARs of the present disclosure comprise an extracellular hinge region. Incorporation of a hinge region can affect cytokine production from CAR-T cells and improve expansion of CAR-T cells in vivo. Exemplary hinges can be isolated or derived from IgD and CD8 domains, for example IgG1.

In some embodiments, the hinge is isolated or derived from CD8a or CD28. In some embodiments, the CD8a hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 1). In some embodiments, the CD8α hinge comprises SEQ ID NO: 1. In some embodiments, the CD8α hinge consists essentially of SEQ ID NO: 1. In some embodiments, the CD8α hinge is encoded by a nucleotide sequence having at least 80% identity, at least 90% identity, at least 95% identity, at least 99% identity or is identical to a sequence of

(SEQ ID NO: 2)
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT.

In some embodiments, the CD8α hinge is encoded by SEQ ID NO: 2.

In some embodiments, the CD28 hinge comprises an amino acid sequence having at least 80% identity, at least 90% identity, at least 95% identity, at least 99% identity or is identical to a sequence of CTIEVMYPPPYLDNEKSNGTIIHVKGKHLCPSPLFPGPSKP (SEQ ID NO: 3). In some embodiments, the CD28 hinge comprises or consists essentially of SEQ ID NO: 3. In some embodiments, the CD28 hinge is encoded by a nucleotide sequence having at least 80% identity, at least 90% identity, at least 95% identity, at least 99% identity or is identical to a sequence of

(SEQ ID NO: 4)
TGTACCATTGAAGTTATGTATCCTCCTCCTTACCTAGACAATGAGAAGA
GCAATGGAACCATTATCCATGTGAAAGGGAAACACCTTTGTCCAAGTCC
CCTATTTCCCGGACCTTCTAAGCCC.

In some embodiments, the CD28 hinge is encoded by SEQ ID NO: 4.

### Transmembrane Domain

The CARs of the present disclosure can be designed to comprise a transmembrane domain that is fused to the extracellular domain of the CAR. In some embodiments, the transmembrane domain that naturally is associated with one of the domains in the CAR is used. For example, a CAR comprising a CD28 co-stimulatory domain might also use a CD28 transmembrane domain. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions may be isolated or derived from (i.e. comprise at least the transmembrane region(s) of) the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137, CD154, or from an immunoglobulin such as IgG4. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In some embodiments of the CARs of the disclosure, the CARs comprise a CD28 transmembrane domain. In some embodiments, the CD28 transmembrane domain comprises an amino acid sequence having at least 80% identity, at least 90% identity, at least 95% identity, at least 99% identity or is identical to a sequence of FWVLVVVGGVLACYSLLVTVAFIIFWV (SEQ ID NO: 5). In some embodiments, the CD28 transmembrane domain comprises or consists essentially of SEQ ID NO: 5. In some embodiments, the CD28 transmembrane domain is encoded by a nucleotide sequence having at least 80% identity, at least 90% identity, at least 95% identity, at least 99% identity or is identical to a sequence of SEQ ID NO: 6.

In some embodiments, the CD28 transmembrane domain is encoded by SEQ ID NO: 6.

In some embodiments of the CARs of the disclosure, the CARs comprise an IL-2Rbeta transmembrane domain. In some embodiments, the IL-2Rbeta transmembrane domain comprises an amino acid sequence having at least 80% identity, at least 90% identity, at least 95% identity, at least 99% identity or is identical to a sequence of IPWLGHLLVGLSGAFGFIILVYLLI (SEQ ID NO: 7). In some embodiments, the IL-2Rbeta transmembrane domain comprises or consists essentially of SEQ ID NO: 7. In some embodiments, the IL-2Rbeta transmembrane domain is encoded by a nucleotide sequence having at least 80% identity, at least 90% identity, at least 95% identity, at least 99% identity or is identical to a sequence of SEQ ID NO: 8. In some embodiments, the IL-2Rbeta transmembrane domain is encoded by SEQ ID NO: 8.

### Cytoplasmic Domain

The cytoplasmic domain or otherwise the intracellular signaling domain of the CARs of the instant invention is responsible for activation of at least one of the normal effector functions of the immune cell in which the CAR has been placed. The term “effector function” refers to a specialized function of a cell. Effector functions of a regulatory T cell, for example, include the suppression or downregulation of induction or proliferation of effector T cells. Thus the term “intracellular signaling domain” refers to the portion of a protein which transduces the effector function signal and directs the cell to perform a specialized function. While usually the entire intracellular signaling domain can be employed, in many cases it is not necessary to use the entire domain. To the extent that a truncated portion of the intracellular signaling domain is used, such truncated portion may be used in place of the intact chain as long as it transduces the effector function signal. In some cases, multiple intracellular domains can be combined to achieve the desired functions of the CAR-T cells of the instant disclosure. The term intracellular signaling domain is thus meant to include any truncated portion of one or more intracellular signaling domains sufficient to transduce the effector function signal.

Examples of intracellular signaling domains for use in the CARs of the instant disclosure include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Accordingly, the intracellular domain of CARs of the instant disclosure comprises at least one cytoplasmic activation domain. In some embodiments, the intracellular activation domain ensures that there is T-cell receptor (TCR) signaling necessary to activate the effector functions of the CAR T-cell. In some embodiments, the at least one cytoplasmic activation is a CD247 molecule (CD3ξ) activation domain, a stimulatory killer immunoglobulin-like receptor (KIR) KIR2DS2 activation domain, or a DNAX-activating protein of 12 kDa (DAP12) activation domain. In some embodiments, the CD3ξ activation domain comprises an amino acid sequence having at least 80% identity, at least 90% identity, at least 95% identity, at least 99% identity or is identical to a sequence of

(SEQ ID NO: 9)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

In some embodiments, the CD3ξ activation domain comprises or consists essentially of SEQ ID NO: 9. In some embodiments, the CD3ξ activation domain is encoded by a nucleotide sequence having at least 80% identity, at least 90% identity, at least 95% identity, at least 99% identity or is identical to a sequence of SEQ ID NO: 10. In some embodiments, the CD3ξ activation domain is encoded by SEQ ID NO: 10.

It is known that signals generated through the TCR alone are often insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. In some embodiments, the ITAM contains a tyrosine separated from a leucine or an isoleucine by any two other amino acids (YxxL) (SEQ ID NO: 21).

In some embodiments, the cytoplasmic domain contains 1, 2, or 3 ITAMs. In some embodiments, the cytoplasmic domain contains 1 ITAM. In some embodiments, the cytoplasmic domain contains 2 ITAMs. In some embodiments, the cytoplasmic domain contains 3 ITAMs. In some embodiments, the cytoplasmic domain contains 4 ITAMs. In some embodiments, the cytoplasmic domain contains 5 ITAMs.

In some embodiments, the cytoplasmic domain is a CD3ξ activation domain. In some embodiments, CD3ξ activation domain comprises a single ITAM. In some embodiments, CD3ξ activation domain comprises two ITAMs. In some embodiments, CD3ξ activation domain comprises three ITAMs.

In some embodiments, the CD3ξ activation domain comprising a single ITAM comprises an amino acid sequence having at least 80% identity, at least 90% identity, at least 95% identity, at least 99% identity or is identical to a sequence of RVKFSRSADAPAYQQGQNQLYNELNLGRREEYDVLHMQALPPR (SEQ ID NO: 11). In some embodiments, the CD3ξ activation domain comprises SEQ ID NO: 11. In some embodiments, the CD3ξ activation domain comprising a single ITAM consists essentially of an amino acid sequence of SEQ ID NO: 11. In some embodiments, the CD3ξ activation domain comprising a single ITAM is encoded by a nucleotide sequence having at least 80% identity, at least 90% identity, at least 95% identity, at least 99% identity or is identical to a sequence of SEQ ID NO: 12. In some embodiments, the CD3ξ activation domain is encoded by SEQ ID NO: 12.

Further examples of ITAM containing primary cytoplasmic signaling sequences that can be used in the CARs of the instant disclosure include those derived from TCRξ, FcRγ, FcRβ, CD3γ, CD3δ, CD3ε, CD3ξ, CD5, CD22, CD79a, CD79b, and CD66d. It is particularly preferred that cytoplasmic signaling molecule in the CAR of the instant invention comprises a cytoplasmic signaling sequence derived from CD3ξ.

### Co-Stimulatory Domain

In some embodiments, the cytoplasmic domain of the CAR can be designed to comprise the CD3 signaling domain by itself or combined with any other desired cytoplasmic domain(s) useful in the context of the CAR of the instant disclosure. For example, the cytoplasmic domain of the CAR can comprise a CD3 chain portion and a co-stimulatory domain. The co-stimulatory domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include the co-stimulatory domain is selected from the group consisting of IL-21V, Fc Receptor gamma (FcRγ), Fc Receptor beta (FcRβ), CD3g molecule gamma (CD3γ), CD36, CD3ε, CD5 molecule (CD5), CD22 molecule (CD22), CD79a molecule (CD79a), CD79b molecule (CD79b), carcinoembryonic antigen related cell adhesion molecule 3 (CD66d), CD27 molecule (CD27), CD28 molecule (CD28), TNF receptor superfamily member 9 (4-1BB), TNF receptor superfamily member 4 (OX40), TNF receptor superfamily member 8 (CD30), CD40 molecule (CD40), programmed cell death 1 (PD-1), inducible T cell costimulatory (ICOS), lymphocyte function-associated antigen-1 (LFA-1), CD2 molecule (CD2), CD7 molecule (CD7), TNF superfamily member 14 (LIGHT), killer cell lectin like receptor C2 (NKG2C) and CD276 molecule (B7-H3) c-stimulatory domains, or functional fragments thereof.

The cytoplasmic domains within the cytoplasmic signaling portion of the CARs of the instant disclosure may be linked to each other in a random or specified order. Optionally, a short oligo- or polypeptide linker, for example between 2 and 10 amino acids in length may form the linkage. A glycine-serine doublet provides an example of a suitable linker.

In some embodiments, the intracellular domains of CARs of the instant disclosure comprise at least one co-stimulatory domain. In some embodiments, the co-stimulatory domain is isolated or derived from CD28. In some embodiments, the CD28 co-stimulatory domain comprises an amino acid sequence having at least 80% identity, at least 90% identity, at least 95% identity, at least 99% identity or is identical to a sequence of

(SEQ ID NO: 13)
RSKRSRLLHSDYMNMTPRRPGPTRKHYQPYAPPRDFAAYRS.

In some embodiments, the CD28 co-stimulatory domain comprises or consists essentially of SEQ ID NO: 13. In some embodiments, the CD28 co-stimulatory domain is encoded by a nucleotide sequence having at least 80% identity, at least 90% identity, at least 95% identity, at least 99% identity or is identical to a sequence of SEQ ID NO: 14. In some embodiments, the CD28 co-stimulatory domain is encoded by SEQ ID NO: 14.

In some embodiments, the intracellular domain of the CARs of the instant disclosure comprises an interleukin-2 receptor beta-chain (IL-2Rbeta or IL-2R-beta) cytoplasmic domain. In some embodiments, the IL-2Rbeta domain is truncated. In some embodiments, the IL-2Rbeta cytoplasmic domain comprises one or more STATS-recruitment motifs. In some embodiments, the CAR comprises one or more STATS-recruitment motifs outside the IL-2Rbeta cytoplasmic domain.

In some embodiments, the IL-2-Rbeta intracellular domain comprises an amino acid sequence having at least 80% identity, at least 90% identity, at least 95% identity, at least 99% identity or is identical to a sequence of

(SEQ ID NO: 15)
NCRNTGPWLKKVLKCNTPDPSKFFSQLSSEHGGDVQKWLSSPFPSSSFS
PGGLAPEISPLEVLERDKVTQLLPLNTDAYLSLQELQGQDPTHLV.

In some embodiments, the IL2R-beta intracellular domain comprises or consists essentially of SEQ ID NO: 15. In some embodiments, the IL-2R-beta intracellular domain is encoded by a nucleotide sequence having at least 80% identity, at least 90% identity, at least 95% identity, at least 99% identity or is identical to a sequence of SEQ ID NO: 16.

In some embodiments, the IL-2R-beta intracellular domain is encoded by SEQ ID NO: 16.

In an embodiment, the IL-2R-beta cytoplasmic domain comprises one or more STATS-recruitment motifs. Exemplary STATS-recruitment motifs are provided by Passerini et al. (2008) STATS-signaling cytokines regulate the expression of FOXP3 in CD4+CD25+ regulatory T cells and CD4+CD25+ effector T cells. International Immunology, Vol. 20, No. 3, pp. 421-431, and by Kagoya et al. (2018) A novel chimeric antigen receptor containing a JAK—STAT signaling domain mediates superior antitumor effects. Nature Medicine doi: 10.1038/nm. 4478.

In some embodiments, the STATS-recruitment motif(s) consists of the sequence Tyr-Leu-Ser-Leu (SEQ ID NO: 17).

In some embodiments, the CAR comprises an intracellular domain isolated or derived from CD28, 4-1BB and/or CD3z, or a combination thereof. In some embodiments, the intracellular domain of the CAR comprises a CD28 co-stimulatory domain, a 4-1BB costimulatory domain, and a CD3ξ activation domain. In some embodiments, the intracellular domain of the CAR comprises a sequence of RSKRSRLLHSDYMNMTPRRPGPTRKHYQPYAPPRDFAAYRSKRGRKKLLYIFKQPF MRPVQTTQEEDGCSCRFPEEEEGGCELRVKFSRSADAPAYKQGQNQLYNELNLGRR EEYDVLDKRRGRDPEMGGKPRRKNPQEGLYNELQKDKMAEAYSEIGMKGERRRGK GHDGLYQGLSTATKDTYDALHMQALPPR (SEQ ID NO: 21903), or a sequence having at least 80% identity, at least 90% identity, at least 95% identity, at least 99% identity thereto. In some embodiments, the intracellular domain of the CAR is encoded by SEQ ID NO: 21904) or a sequence having at least 80% identity, at least 90% identity, at least 95% identity, at least 99% identity thereto. In some embodiments, the intracellular domain of the CAR is encoded by SEQ ID NO: 21904.

### Inhibitory Domains

In some embodiments, for example in the second engineered receptors of the disclosure which provide an inhibitory signal, the inhibitory signal is transmitted through the intracellular domain of the receptor. In some embodiments, the engineered receptor comprises an inhibitory intracellular domain. In some embodiments, the second engineered receptor is a CAR comprising an inhibitory intracellular domain (an inhibitory CAR).

In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

Inhibitory domains can be isolated from human tumor necrosis factor related apoptosis inducing ligand (TRAIL) receptor and CD200 receptor 1.

In some embodiments, the inhibitory domain comprises an intracellular domain, a transmembrane or a combination thereof. In some embodiments, the inhibitory domain comprises an intracellular domain, a transmembrane domain, a hinge region or a combination thereof. In some embodiments, the inhibitory domain comprises an immunoreceptor tyrosine-based inhibitory motif (ITIM). In some embodiments, the inhibitory domain comprising an ITIM can be isolated or derived from an immune checkpoint inhibitor such as CTLA-4 and PD-1.

Inhibitory domains can be isolated from human tumor necrosis factor related apoptosis inducing ligand (TRAIL) receptor and CD200 receptor 1. In some embodiments, the inhibitory domain is isolated or derived from a human protein, for example a human TRAIL receptor, CTLA-4, or PD-1 protein. In some embodiments, the TRAIL receptor comprises TR10A, TR10B or TR10D.

Endogenous TRAIL is expressed as a 281-amino acid type II trans-membrane protein, which is anchored to the plasma membrane and presented on the cell surface. TRAIL is expressed by natural killer cells, which, following the establishment of cell-cell contacts, can induce TRAIL-dependent apoptosis in target cells. Physiologically, the TRAIL-signaling system was shown to be essential for immune surveillance, for shaping the immune system through regulating T-helper cell 1 versus T-helper cell 2 as well as “helpless” CD8+ T-cell numbers, and for the suppression of spontaneous tumor formation.

In some embodiments, the inhibitory domain comprises an intracellular domain isolated or derived from a CD200 receptor. The cell surface glycoprotein CD200 receptor 1 (Uniprot ref: Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the engineered receptor comprises an inhibitory domain isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1, also called LIR-1 and LILRB1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A (kinase inactive ZAP70).

In some embodiments, the inhibitory domain is isolated or derived from a human protein.

In some embodiments, the second, inhibitory receptor comprises a cytoplasmic domain and transmembrane domain isolated or derived from the same protein, for example an ITIM containing protein. In some embodiments, the second, inhibitory receptor comprises a cytoplasmic domain, a transmembrane domain, and an extracellular domain or a portion thereof isolated or derived isolated or derived from the same protein, for example an ITIM containing protein. In some embodiments, the second, inhibitory receptor comprises a hinge region isolated or derived from isolated or derived from the same protein as the intracellular domain and/or transmembrane domain, for example an ITIM containing protein.

In some embodiments, the second, inhibitory engineered receptor comprises an inhibitory domain. In some embodiments, the second, inhibitory engineered receptor comprises an inhibitory intracellular domain and/or an inhibitory transmembrane domain. In some embodiments, the second engineered receptor is a CAR comprising an inhibitory domain (an inhibitory CAR). In some embodiments, the inhibitory intracellular domain is fused to the intracellular domain of a CAR. In some embodiments, the inhibitory intracellular domain is fused to the transmembrane domain of a CAR.

### T Cell Receptors (TCRs)

In some embodiments, the first or second engineered receptor is a T Cell Receptor (TCR). In some embodiments, the first and second engineered receptors are a T Cell Receptors (TCR).

As used herein, a “TCR”, sometimes also called a “TCR complex” or “TCR/CD3 complex” refers to a protein complex comprising a TCR alpha chain, a TCR beta chain, and one or more of the invariant CD3 chains (zeta, gamma, delta and epsilon), sometimes referred to as subunits. The TCR alpha and beta chains can be disulfide-linked to function as a heterodimer to bind to peptide-MHC complexes. Once the TCR alpha/beta heterodimer engages peptide-MHC, conformational changes in the TCR complex in the associated invariant CD3 subunits are induced, which leads to their phosphorylation and association with downstream proteins, thereby transducing a primary stimulatory signal. In an exemplary TCR complex, the TCR alpha and TCR beta polypeptides form a heterodimer, CD3 epsilon and CD3 delta form a heterodimer, CD3 epsilon and CD3 gamma for a heterodimer, and two CD3 zeta form a homodimer.

### Extracellular Domains

The disclosure provides a first engineered receptor comprising a first extracellular ligand binding domain and a second engineered receptor comprising a second extracellular ligand binding domain. Either the first engineered receptor, the second engineered receptor, or both, may be a TCR. Any suitable ligand binding domain may be fused to an extracellular domain, hinge domain or transmembrane of the engineered TCRs described herein.

In some embodiments, the first and/or second ligand binding domain is fused to an extracellular domain of a TCR subunit. The TCR subunit can be TCR alpha, TCR beta, CD3 delta, CD3 epsilon or CD3 gamma. In some embodiments, both the first and second ligand binding domains are fused to the same TCR subunit in different TCR receptors. In some embodiments, the first and second ligand binding domains are fused to different TCR subunits in different TCR receptors. In some embodiments, the first, activator ligand binding domain is fused to a first TCR subunit in a first engineered receptor and the second, inhibitor ligand binding domain is fused to a second TCR subunit in a second engineered receptor. In some embodiments, the first and second TCR subunits are not the same subunit. In some embodiments, the first and second TCR subunits are the same subunit. For example, the first ligand binding domain can be fused to TCR alpha, and the second ligand binding domain can be fused to TCR beta. As a further example, the first ligand binding is fused to TCR beta and the second ligand binding domain used fused to TCR alpha.

In some embodiments, the first, activator LBD comprises an ScFv domain and the second, inhibitor LBD comprises a Vβ-only domain. In some embodiments, the first, activator LBD comprises a Vβ-only domain and the second, inhibitor LBD comprises an ScFv domain. In some embodiments, both the first, activator LBD and the second, inhibitor LBD are ScFv domains. In some embodiments, both the first, activator LBD and the second, inhibitor LBD are Vβ-only domains.

In some embodiments, the first engineered TCR of the disclosure comprises an extracellular domain comprising a Vβ-only domain, a transmembrane domain and an intracellular domain. In some embodiments, the intracellular domain comprises one or more exogenous domains.

In some embodiments, the first engineered TCR of the disclosure comprises an extracellular domain comprising an ScFv domain, a transmembrane domain and an intracellular domain. In some embodiments, the intracellular domain comprises one or more exogenous domains.

In some embodiments, the second engineered TCR of the disclosure comprises an extracellular domain comprising a Vβ-only domain, a transmembrane domain and an inhibitory intracellular domain.

In some embodiments, the second engineered TCR of the disclosure comprises an extracellular domain comprising an ScFv domain, a transmembrane domain and an inhibitory intracellular domain.

TCR subunits include TCR alpha, TCR beta, CD3 zeta, CD3 delta, CD3 gamma and CD3 epsilon. Any one or more of TCR alpha, TCR beta chain, CD3 gamma, CD3 delta or CD3 epsilon, or fragments or derivative thereof, can be fused to one or more domains capable of providing a stimulatory signal of the disclosure, thereby enhancing TCR function and activity. Any one or more of TCR alpha, TCR beta chain, CD3 gamma, CD3 delta or CD3 epsilon, or fragments or derivative thereof, can be fused to an inhibitory intracellular domain of the disclosure.

In some embodiments, for example those embodiments wherein the first engineered receptor or second engineered receptor comprises a first and a second polypeptide, the antigen binding domain is isolated or derived from a T cell receptor (TCR) extracellular domain or an antibody.

In some embodiments, the first engineered receptor and second engineered receptor comprise a first antigen binding domain and a second antigen binding domain. The antigen-binding domain or domains of the engineered receptor may be provided on the same or a different polypeptide as the intracellular domain.

In some embodiments, the antigen-binding domain of the first and/or second engineered receptor comprises a single chain variable fragment (scFv).

In some embodiments, the first and/or second engineered receptor comprises a second polypeptide. The disclosure provides receptors having two polypeptides each having a part of a ligand-binding domain (e.g. cognates of a heterodimeric LDB, such as a TCRα/β- or Fab-based LBD). The disclosure further provides receptors having two polypeptides, each having a part of a ligand-binding domain (e.g. cognates of a heterodimeric LDB, such as a TCRα/β- or Fab-based LBD) and one part of the ligand binding domain is fused to a hinge or transmembrane domain, while the other part of the ligand binding domain has no intracellular domain. Further variations include receptors where each polypeptide has a hinge domain, and where each polypeptide has a hinge and transmembrane domain. In some embodiments, the hinge domain is absent. In other embodiments, the hinge domain is a membrane proximal extracellular region (MPER), such as the LILRB1 D3D4 domain.

In some embodiments, for example those embodiments where the first and/or second engineered receptor comprises at least two polypeptides, the first polypeptide comprises a first chain of an antibody and the second polypeptide comprise a second chain of said antibody.

In some embodiments, the receptor comprises a Fab fragment of an antibody. In embodiments, a first polypeptide comprises an antigen-binding fragment of the heavy chain of the antibody and an intracellular domain, and a second polypeptide comprises an antigen-binding fragment of the light chain of the antibody. In some embodiments, the first polypeptide comprises an antigen-binding fragment of the light chain of the antibody and the intracellular domain, and the second polypeptide comprises an antigen-binding fragment of the heavy chain of the antibody.

In some embodiments, the first and/or second engineered receptor comprises an extracellular fragment of a T cell receptor (TCR). In some embodiments, a first polypeptide comprises an antigen-binding fragment of the alpha chain of the TCR and the intracellular domain, and a second polypeptide comprises an antigen-binding fragment of the beta chain of the TCR. In some embodiments, a first polypeptide comprises an antigen-binding fragment of the beta chain of the TCR and the intracellular domain, and the second polypeptide comprises an antigen-binding fragment of the alpha chain of the TCR.

### TCRs Comprising Vβ-Only Domains

Certain embodiments of present disclosure relate to engineered TCRs comprising a TCR variable domain, the TCR variable domain specifically binding to an antigen in the absence of a second TCR variable domain (a Vβ-only domain).

In some embodiments, the engineered TCR comprises additional elements besides the TCR variable domain, including additional amino acid sequences, additional protein domains (covalently associated, non-covalently associated or covalently and non-covalently associated with the TCR variable domain), fusion or non-covalent association of the TCR variable domain with other types of macromolecules (for example polynucleotides, polysaccharides, lipids, or a combination thereof), fusion or non-covalent association of the TCR variable domain with one or more small molecules, compounds, or ligands, or a combination thereof. Any additional element, as described, may be combined provided that the TCR variable domain is configured to specifically bind the epitope in the absence of a second TCR variable domain.

An engineered TCR comprising a Vβ-only domain as described herein may comprise a single TCR chain (e.g. α, β, γ, or δ chain), or it may comprise a single TCR variable domain (e.g. of α, β, γ, or δ chain). If the engineered TCR is a single TCR chain, then the TCR chain comprises a transmembrane domain, a constant (or C domain) and a variable (or V domain), and does not comprise a second TCR variable domain. The engineered TCR may therefore comprise or consist of a TCR α chain, a TCR β chain, a TCR γ chain or a TCR δ chain. The engineered TCR may be a membrane bound protein. The engineered TCR may alternatively be a membrane-associated protein.

In some embodiments, the engineered TCR as described herein utilizes a surrogate a chain that lacks a V α segment, which forms activation-competent TCRs complexed with the six CD3 subunits.

In other embodiments, the engineered TCR as described herein functions independently of a surrogate a chain that lacks a Vα segment. For example, in some embodiments the one or more engineered TCRs are fused to transmembrane (e.g., CD3ξ and CD28) and intracellular domain proteins (e.g., CD3ξ, CD28, and/or 4-1BB) that are capable of activating T cells in response to antigen.

In some embodiments, the engineered TCR comprises one or more single TCR chains fused to the Vβ-only domain described herein. For example, the engineered TCR may comprise, or consist essentially of single α TCR chain, a single β TCR chain, a single γ TCR chain, or a single δ TCR chain fused to one or more Vβ-only domains.

In some embodiments, the engineered TCR engages antigen using complementarity-determining regions (CDRs). Each engineered TCR contains three complement determining regions (CDR1, CDR2, and CDR3).

The first and/or second ligand binding Vβ-only domain may be a human TCR variable domain. Alternatively, the first and/or second Vβ-only domain may be a non-human TCR variable domain. The first and/or second Vβ-only domain may be a mammalian TCR variable domain. The first and/or second Vβ-only domain may be a vertebrate TCR variable domain.

In embodiments where Vβ-only domain is incorporated into a fusion protein, for example a fusion protein comprising a TCR subunit, and optionally, an additional stimulatory intracellular domain. The fusion protein may comprise a Vβ-only domain and any other protein domain or domains.

### Transmembrane Domains

The disclosure provide a first fusion protein comprising a first, activator LBD and a second fusion protein comprising a second, inhibitor LBD and an inhibitor intracellular domain. In some embodiments, the first and second fusion proteins comprise transmembrane domains.

The disclosure provides polypeptides comprising a transmembrane domain, and an intracellular domain capable of providing a stimulatory signal or an inhibitory signal. In some embodiments, the engineered TCR comprises multiple intracellular domains capable of providing a stimulatory signal.

A “transmembrane domain”, as used herein, refers to a domain of a protein that spans membrane of the cell. Transmembrane domains typically consist predominantly of non-polar amino acids, and may traverse the lipid bilayer once or several times. Transmembrane domains usually comprise alpha helices, a configuration which maximizes internal hydrogen bonding.

Transmembrane domains isolated or derived from any source are envisaged as within the scope of the fusion proteins of the disclosure.

In some embodiments, the transmembrane domain is one that is associated with one of the other domains of the fusion protein, or isolated or derived from the same protein as one of the other domains of the fusion protein. In some embodiments, the transmembrane domain and the second intracellular domain are from the same protein, for example a TCR complex subunit such as TCR alpha, TCR beta, CD3 delta, CD3 epsilon or CD3 gamma. In some embodiments, the extracellular domain (svd-TCR), the transmembrane domain and the second intracellular domain are from the same protein, for example a TCR complex subunit such as TCR alpha, TCR beta, CD3 delta, CD3 epsilon or CD3 gamma. In other embodiments, the extracellular domain (comprising one or more ligand binding domains, such as Vβ-only domain and ScFv domains), the transmembrane domain and the intracellular domain(s) are from different proteins. For example, in some embodiments the engineered svd-TCR comprises a CD28 transmembrane domain with a CD28, 4-1BB and CD3ξ intracellular domain.

The transmembrane domain may be derived either from a natural or from a recombinant source. Where the source is natural, the domain may be derived from any membrane-bound or transmembrane protein.

In some embodiments, the transmembrane domain is capable of signaling to the intracellular domain(s) whenever the TCR complex has bound to a target. A transmembrane domain of particular use in this invention may include at least the transmembrane region(s) of e.g., the alpha, beta or zeta chain of the TCR, CD3 delta, CD3 epsilon or CD3 gamma, CD28, CD3 epsilon, CD45, CD4, CDS, CD8, CD9, CD16, CD22, CD33, CD37, CD64, CD80, CD86, CD134, CD137, CD154.

In some embodiments, the transmembrane domain can be attached to the extracellular region of the fusion protein, e.g., the antigen binding domain of the TCR alpha or beta chain, via a hinge, e.g., a hinge from a human protein. For example, in one embodiment, the hinge can be a human immunoglobulin (Ig) hinge, e.g., an IgG4 hinge, or a CD8α hinge.

In some embodiments, the hinge is isolated or derived from CD8α or CD28. In some embodiments, the CD8α hinge comprises an amino acid sequence having at least 80% identity, at least 90% identity, at least 95% identity, at least 99% identity or is identical to a sequence of TTTPAPRPPTPAPTIASQPLSLRPEACRPAAGGAVHTRGLDFACD (SEQ ID NO: 1). In some embodiments, the CD8α hinge comprises SEQ ID NO: 1. In some embodiments, the CD8α hinge consists essentially of SEQ ID NO: 1. In some embodiments, the CD8α hinge is encoded by a nucleotide sequence having at least 80% identity, at least 90% identity, at least 95% identity, at least 99% identity or is identical to a sequence of:

(SEQ ID NO: 2)
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT.

In some embodiments, the CD8α hinge is encoded by SEQ ID NO: 2.

In some embodiments, the CD28 hinge comprises an amino acid sequence having at least 80% identity, at least 90% identity, at least 95% identity, at least 99% identity or is identical to a sequence of CTIEVMYPPPYLDNEKSNGTIIHVKGKHLCPSPLFPGPSKP (SEQ ID NO: 3. In some embodiments, the CD28 hinge comprises or consists essentially of SEQ ID NO: 3. In some embodiments, the CD28 hinge is encoded by a nucleotide sequence having at least 80% identity, at least 90% identity, at least 95% identity, at least 99% identity or is identical to a sequence of

(SEQ ID NO: 4)
TGTACCATTGAAGTTATGTATCCTCCTCCTTACCTAGACAATGAGAAGA
GCAATGGAACCATTATCCATGTGAAAGGGAAACACCTTTGTCCAAGTCC
CCTATTTCCCGGACCTTCTAAGCCC.

In some embodiments, the CD28 hinge is encoded by SEQ ID NO: 4.

In some embodiments, the transmembrane domain comprises a TCR alpha transmembrane domain. In some embodiments, the TCR alpha transmembrane domain comprises an amino acid sequence having at least 85% identity, at least 90% identity, at least 95% identity, at least 96% identity, at least 97% identity, at least 98% identity, at least 99% identity or is identical to a sequence of: VIGFRILLLKVAGFNLLMTLRLW (SEQ ID NO: 26). In some embodiments, the TCR alpha transmembrane domain comprises, or consists essentially of, SEQ ID NO: 26. In some embodiments, the TCR alpha transmembrane domain is encoded by a sequence of

(SEQ ID NO: 27)
GTGATTGGGTTCCGAATCCTCCTCCTGAAAGTGGCCGGGTTTAATCTGC
TCATGACGCTGCGGCTGTGG.

In some embodiments, the transmembrane domain comprises a TCR beta transmembrane domain. In some embodiments, the TCR beta transmembrane domain comprises an amino acid sequence having at least 85% identity, at least 90% identity, at least 95% identity, at least 96% identity, at least 97% identity, at least 98% identity, at least 99% identity or is identical to a sequence of: TILYEILLGKATLYAVLVSALVL (SEQ ID NO: 28). In some embodiments, the TCR beta transmembrane domain comprises, or consists essentially of, SEQ ID NO: 28. In some embodiments, the TCR beta transmembrane domain is encoded by a sequence of

(SEQ ID NO: 20)
ACCATCCTCTATGAGATCTTGCTAGGGAAGGCCACCTTGTATGCCGTGC
TGGTCAGTGCCCTCGTGCTG.

In some embodiments, the transmembrane comprises a CD3 zeta transmembrane domain. In some embodiments, the CD3 zeta transmembrane domain comprises an amino acid sequence having at least 85% identity, at least 90% identity, at least 95% identity, at least 96% identity, at least 97% identity, at least 98% identity, at least 99% identity or is identical to a sequence of: LCYLLDGILFIYGVILTALFL (SEQ ID NO: 29). In some embodiments, the CD3 zeta transmembrane domain comprises, or consists essentially of, SEQ ID NO: 29.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region).

In some embodiments, the transmembrane domain can be selected or modified by amino acid substitution to avoid binding of such domains to the transmembrane domains of the same or different surface membrane proteins, e.g., to minimize interactions with other members of the receptor complex.

When present, the transmembrane domain may be a natural TCR transmembrane domain, a natural transmembrane domain from a heterologous membrane protein, or an artificial transmembrane domain. The transmembrane domain may be a membrane anchor domain. Without limitation, a natural or artificial transmembrane domain may comprise a hydrophobic a-helix of about 20 amino acids, often with positive charges flanking the transmembrane segment. The transmembrane domain may have one transmembrane segment or more than one transmembrane segment. Prediction of transmembrane domains/segments may be made using publicly available prediction tools (e.g. TMHMM, Krogh et al. Journal of Molecular Biology 2001; 305(3):567-580; or TMpred, Hofmann & Stoffel Biol. Chem. Hoppe-Seyler 1993; 347: 166). Non-limiting examples of membrane anchor systems include platelet derived growth factor receptor (PDGFR) transmembrane domain, glycosylphosphatidylinositol (GPI) anchor (added post-translationally to a signal sequence) and the like.

### Intracellular Domain

The disclosure provides fusion proteins comprising an intracellular domain. An “intracellular domain,” as the term is used herein, refers to an intracellular portion of a protein.

In some embodiments, the intracellular domain comprises one or more domains capable of providing a stimulatory signal to a transmembrane domain. In some embodiments, the intracellular domain comprises a first intracellular domain capable of providing a stimulatory signal and a second intracellular domain capable of providing a stimulatory signal. In other embodiments, the intracellular domain comprises a first, second and third intracellular domain capable of providing a stimulatory signal. The intracellular domains capable of providing a stimulatory signal are selected from the group consisting of a CD28 molecule (CD28) domain, a LCK proto-oncogene, Src family tyrosine kinase (Lck) domain, a TNF receptor superfamily member 9 (4-1BB) domain, a TNF receptor superfamily member 18 (GITR) domain, a CD4 molecule (CD4) domain, a CD8a molecule (CD8a) domain, a FYN proto-oncogene, Src family tyrosine kinase (Fyn) domain, a zeta chain of T cell receptor associated protein kinase 70 (ZAP70) domain, a linker for activation of T cells (LAT) domain, lymphocyte cytosolic protein 2 (SLP76) domain, (TCR) alpha, TCR beta, CD3 delta, CD3 gamma and CD3 epsilon intracellular domains.

In some embodiments, an intracellular domain comprises at least one intracellular signaling domain. An intracellular signaling domain generates a signal that promotes a function a cell, for example an immune effector function of a TCR containing cell, e.g., a TCR-expressing T-cell. In some embodiments, the intracellular domain of the fusion proteins of the disclosure includes at least one intracellular signaling domain. For example, the intracellular domains of CD3 gamma, delta or epsilon comprise signaling domains.

In some embodiments, the extracellular domain, transmembrane domain and intracellular domain are isolated or derived from the same protein, for example T-cell receptor (TCR) alpha, TCR beta, CD3 delta, CD3 gamma or CD3 epsilon.

Examples of intracellular domains for use in the fusion proteins of the disclosure include the cytoplasmic sequences of the TCR alpha, TCR beta, CD3 zeta, and 4-1BB, and the intracellular signaling co-receptors that act in concert to initiate signal transduction following antigen receptor engagement, as well as any derivative or variant of these sequences and any recombinant sequence that has the same functional capability.

In some embodiments, the intracellular signaling domain comprises a primary intracellular signaling domain. Exemplary primary intracellular signaling domains include those derived from the proteins responsible for primary stimulation, or antigen dependent stimulation.

An intracellular signaling domain is generally responsible for activation of at least one of the normal effector functions of the immune cell in which the fusion protein has been introduced. The term “effector function” refers to a specialized function of a cell. Effector function of a T-cell, for example, may be cytolytic activity or helper activity including the secretion of cytokines. Thus the term “intracellular signaling domain” refers to the portion of a protein which transduces the effector function signal and directs the cell to perform a specialized function.

While in some cases the entire intracellular signaling domain can be employed, in many cases it is not necessary to use the entire intracellular signaling domain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular domain comprises a CD3 delta intracellular domain. In some embodiments, the CD3 delta intracellular domain comprises an amino acid sequence having at least 85% identity, at least 90% identity, at least 95% identity, at least 96% identity, at least 97% identity, at least 98% identity, at least 99% identity or is identical to a sequence of

(SEQ ID NO: 30)
GHETGRLSGAADTQALLRNDQVYQPLRDRDDAQYSHLGGNWARNKGGSR
SKRSRLLHSDYMNMTPRRPGPTRKHYQPYAPPRDFAAYRS.

In some embodiments, the CD3 delta intracellular domain comprises or consists essentially of, SEQ ID NO: 30. In some embodiments, the CD3 delta intracellular domain is encoded by a sequence of SEQ ID NO: 31.

In some embodiments, the intracellular domain comprises a CD3 epsilon intracellular domain. In some embodiments, the CD3 epsilon intracellular domain comprises an amino acid sequence having at least 85% identity, at least 90% identity, at least 95% identity, at least 96% identity, at least 97% identity, at least 98% identity, at least 99% identity or is identical to a sequence of: KNRKAKAKPVTRGAGAGGRQRGQNKERPPPVPNPDYEPIRKGQRDLYSGLNQRRIG GSRSKRSRLLHSDYMNMTPRRPGPTRKHYQPYAPPRDFAAYRS (SEQ ID NO: 32). In some embodiments, the CD3 epsilon intracellular domain comprises or consists essentially of, SEQ ID NO: 32. In some embodiments, the CD3 epsilon intracellular domain is encoded by a sequence of SEQ ID NO: 19.

In some embodiments, the intracellular domain comprises a CD3 gamma intracellular domain. In some embodiments, the CD3 gamma intracellular domain comprises an amino acid sequence having at least 85% identity, at least 90% identity, at least 95% identity, at least 96% identity, at least 97% identity, at least 98% identity, at least 99% identity or is identical to a sequence of

(SEQ ID NO: 33)
GQDGVRQSRASDKQTLLPNDQLYQPLKDREDDQYSHLQGNQLRRNGGSR
SKRSRLLHSDYMNMTPRRPGPTRKHYQPYAPPRDFAAYRS.

In some embodiments, the CD3 gamma intracellular domain comprises, or consists essentially of, SEQ ID NO: 33. In some embodiments, the CD3 gamma intracellular domain is encoded by a sequence of SEQ ID NO: 22.

In some embodiments, the intracellular domain comprises a CD3 zeta intracellular domain. In some embodiments, the CD3 zeta intracellular domain comprises an amino acid sequence having at least 85% identity, at least 90% identity, at least 95% identity, at least 96% identity, at least 97% identity, at least 98% identity, at least 99% identity or is identical to a sequence of RVKFSRSADAPAYKQGQNQLYNELNLGRREEYDVLDKRRGRDPEMGGKPRRKNPQ EGLYNELQKDKMAEAYSEIGMKGERRRGKGHDGLYQGLSTATKDTYDALHMQALP PR (SEQ ID NO: 9) or a subsequence thereof.

In some embodiments, the CD3 zeta intracellular domain comprises, or consists essentially of, SEQ ID NO: 9.

In some embodiments, the intracellular domain comprises a TCR alpha intracellular domain. In some embodiments, a TCR alpha intracellular domain comprises Ser-Ser. In some embodiments, a TCR alpha intracellular domain is encoded by a sequence of TCCAGC (SEQ ID NO: 21885).

In some embodiments, the intracellular domain comprises a TCR beta intracellular domain. In some embodiments, the TCR beta intracellular domain comprises an amino acid sequence having at least 80% identity, at least 90% identity, or is identical to a sequence of: MAMVKRKDSR (SEQ ID NO: 35). In some embodiments, the TCR beta intracellular domain comprises, or consists essentially of SEQ ID NO: 35. In some embodiments, the TCR beta intracellular domain is encoded by a sequence of (SEQ ID NO: 36.

In some embodiments, the intracellular signaling domain comprises at least one stimulatory intracellular domain. In some embodiments, the intracellular signaling domain comprises a primary intracellular signaling domain, such as a CD3 delta, CD3 gamma and CD3 epsilon intracellular domain, and one additional stimulatory intracellular domain, for example a co-stimulatory domain. In some embodiments, the intracellular signaling domain comprises a primary intracellular signaling domain, such as a CD3 delta, CD3 gamma and CD3 epsilon intracellular domain, and two additional stimulatory intracellular domains.

Exemplary co-stimulatory intracellular signaling domains include those derived from proteins responsible for co-stimulatory signals, or antigen independent stimulation.

The term “co-stimulatory molecule” refers to the cognate binding partner on a T-cell that specifically binds with a co-stimulatory ligand, thereby mediating a co-stimulatory response by the T-cell, such as, but not limited to, proliferation. Co-stimulatory molecules are cell surface molecules other than antigen receptors. Co-stimulatory molecules and their ligands are required for an efficient immune response. Co-stimulatory molecules include, but are not limited to an MHC class I molecule, BTLA, a Toll ligand receptor, as well as DAP10, DAP12, CD30, LIGHT, OX40, CD2, CD27, CDS, ICAM-1, LFA-1 (CD11a/CD18) 4-1BB (CD137, TNF receptor superfamily member 9), and CD28 molecule (CD28).

A “co-stimulatory domain”, sometimes referred to as “a co-stimulatory intracellular signaling domain” can be the intracellular portion of a co-stimulatory protein. A co-stimulatory domain can be a domain of a co-stimulatory protein that transduces the co-stimulatory signal. A co-stimulatory protein can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, a ligand that specifically binds with CD83, CD4, and the like. The co-stimulatory domain can comprise the entire intracellular portion, or the entire native intracellular signaling domain, of the molecule from which it is derived, or a functional fragment thereof.

In some embodiments, the stimulatory domain comprises a co-stimulatory domain. In some embodiments, the co-stimulatory domain comprises a CD28 or 4-1BB co-stimulatory domain. CD28 and 4-1BB are well characterized co-stimulatory molecules required for full T cell activation and known to enhance T cell effector function. For example, CD28 and 4-1BB have been utilized in chimeric antigen receptors (CARs) to boost cytokine release, cytolytic function, and persistence over the first-generation CAR containing only the CD3 zeta signaling domain. Likewise, inclusion of co-stimulatory domains, for example CD28 and 4-1BB domains, in engineered TCR can increase T cell effector function and specifically allow co-stimulation in the absence of co-stimulatory ligand, which is typically down-regulated on the surface of tumor cells.

In some embodiments, the stimulatory domain comprises a CD28 intracellular domain. In some embodiments, the CD28 intracellular domain comprises an amino acid sequence having at least 85% identity, at least 90% identity, at least 95% identity, at least 96% identity, at least 97% identity, at least 98% identity, at least 99% identity or is identical to a sequence of: RSKRSRLLHSDYMNMTPRRPGPTRKHYQPYAPPRDFAAYRS (SEQ ID NO: 13). In some embodiments, the CD28 intracellular domain comprises, or consists essentially of, RSKRSRLLHSDYMNMTPRRPGPTRKHYQPYAPPRDFAAYRS (SEQ ID NO: 13). In some embodiments, a CD28 intracellular domain is encoded by a nucleotide sequence comprising SEQ ID NO: 14.

In some embodiments, the stimulatory domain comprises a 4-1BB intracellular domain. In some embodiments, the 4-1BB intracellular domain comprises an amino acid sequence having at least 85% identity, at least 90% identity, at least 95% identity, at least 96% identity, at least 97% identity, at least 98% identity, at least 99% identity or is identical to a sequence of: KRGRKKLLYIFKQPFMRPVQTTQEEDGCSCRFPEEEEGGCEL (SEQ ID NO: 37). In some embodiments, the 4-1BB intracellular domain comprises, or consists essentially of, KRGRKKLLYIFKQPFMRPVQTTQEEDGCSCRFPEEEEGGCEL (SEQ ID NO: 37). In some embodiments, a 4-1BB intracellular domain is encoded by a nucleotide sequence comprising SEQ ID NO: 38.

### Inhibitory Domains

The disclosure provides inhibitory intracellular domains which can be fused to the transmembrane or intracellular domain of any of the TCR subunits to generate an inhibitory TCR. In those embodiments where the inhibitory receptor is an inhibitory CAR, the same intracellular domains described below to generate an inhibitory TCR can also be used to generate an inhibitory CAR.

In some embodiments, the inhibitory intracellular domain comprises an immunoreceptor tyrosine-based inhibitory motif (ITIM). In some embodiments, the inhibitory intracellular domain comprising an ITIM can be isolated or derived from an immune checkpoint inhibitor such as CTLA-4 and PD-1. CTLA-4 and PD-1 are immune inhibitory receptors expressed on the surface of T cells, and play a pivotal role in attenuating or terminating T cell responses.

Inhibitory domains can be isolated from human tumor necrosis factor related apoptosis inducing ligand (TRAIL) receptor and CD200 receptor 1.

In some embodiments, the inhibitory domain comprises an intracellular domain, a transmembrane or a combination thereof. In some embodiments, the inhibitory domain comprises an intracellular domain, a transmembrane domain, a hinge region or a combination thereof. In some embodiments, the inhibitory domain comprises an immunoreceptor tyrosine-based inhibitory motif (ITIM). In some embodiments, the inhibitory domain comprising an ITIM can be isolated or derived from an immune checkpoint inhibitor such as CTLA-4 and PD-1.

Inhibitory domains can be isolated from human tumor necrosis factor related apoptosis inducing ligand (TRAIL) receptor and CD200 receptor 1. In some embodiments, the inhibitory domain is isolated or derived from a human protein, for example a human TRAIL receptor, CTLA-4, or PD-1 protein. In some embodiments, the TRAIL receptor comprises TR10A, TR10B or TR10D.

Endogenous TRAIL is expressed as a 281-amino acid type II trans-membrane protein, which is anchored to the plasma membrane and presented on the cell surface. TRAIL is expressed by natural killer cells, which, following the establishment of cell-cell contacts, can induce TRAIL-dependent apoptosis in target cells. Physiologically, the TRAIL-signaling system was shown to be essential for immune surveillance, for shaping the immune system through regulating T-helper cell 1 versus T-helper cell 2 as well as “helpless” CD8+ T-cell numbers, and for the suppression of spontaneous tumor formation.

In some embodiments, the inhibitory domain comprises an intracellular domain isolated or derived from a CD200 receptor. The cell surface glycoprotein CD200 receptor 1 (Uniprot ref: Q8TD46) represents another example of an inhibitory intracellular domain of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli.

In some embodiments, the engineered receptor comprises an inhibitory domain isolated or derived from killer cell immunoglobulin like receptor, three Ig domains and long cytoplasmic tail 2 (KIR3DL2), killer cell immunoglobulin like receptor, three Ig domains and long cytoplasmic tail 3 (KIR3DL3), leukocyte immunoglobulin like receptor B1 (LIR1), programmed cell death 1 (PD-1), Fc gamma receptor IIB (FcgRIIB), killer cell lectin like receptor K1 (NKG2D), CTLA-4, a domain containing a synthetic consensus ITIM, a ZAP70 SH2 domain (e.g., one or both of the N and C terminal SH2 domains), or ZAP70 KI_K369A(kinas e inactive ZAP70).

In some embodiments, the inhibitory domain is isolated or derived from a human protein.

In some embodiments, the second, inhibitory receptor comprises a cytoplasmic domain and transmembrane domain isolated or derived from the same protein, for example an ITIM containing protein. In some embodiments, the second, inhibitory receptor comprises a cytoplasmic domain, a transmembrane domain, and an extracellular domain or a portion thereof isolated or derived isolated or derived from the same protein, for example an ITIM containing protein. In some embodiments, the second, inhibitory receptor comprises a hinge region isolated or derived from isolated or derived from the same protein as the intracellular domain and/or transmembrane domain, for example an ITIM containing protein.

In some embodiments, the second engineered receptor is a TCR comprising an inhibitory domain (an inhibitory TCR). In some embodiments, the inhibitory TCR comprises an inhibitory intracellular domain and/or an inhibitory transmembrane domain. In some embodiments, the inhibitory intracellular domain is fused to the intracellular domain of TCR alpha, TCR beta, CD3 delta, CD3 gamma or CD3 epsilon or a portion thereof a TCR. In some embodiments, the inhibitory intracellular domain is fused to the transmembrane domain of TCR alpha, TCR beta, CD3 delta, CD3 gamma or CD3 epsilon.

In some embodiments, the second engineered receptor is a TCR comprising an inhibitory domain (an inhibitory TCR). In some embodiments, the inhibitory domain is isolated or derived from LILRB1.

### LILRB1 Inhibitory Receptors

The disclosure provides a second, inhibitory receptor comprising a LILRB1 inhibitory domain, and optionally, a LILRB1 transmembrane and/or hinge domain, or functional variants thereof. The second, inhibitory receptor can be a CAR or TCR. The inclusion of the LILRB1 transmembrane domain and/or the LILRB1 hinge domain in the inhibitory receptor may increase the inhibitory signal generated by the inhibitory receptor compared to a reference inhibitory receptor having another transmembrane domain or another hinge domains. The second, inhibitory receptor comprising the LILRB1 inhibitory domain may be a CAR or TCR, as described herein. Any suitable ligand binding domain, as described herein, may be fused to the LILRB1-based second, inhibitory receptors.

Leukocyte immunoglobulin-like receptor subfamily B member 1 (LILRB1), also known as Leukocyte immunoglobulin-like receptor B1, as well as ILT2, LIR1, MIR7, PIRB, CD85J, ILT-2 LIR-1, MIR-7 and PIR-B, is a member of the leukocyte immunoglobulin-like receptor (LIR) family. The LILRB1 protein belongs to the subfamily B class of LIR receptors. These receptors contain two to four extracellular immunoglobulin domains, a transmembrane domain, and two to four cytoplasmic immunoreceptor tyrosine-based inhibitory motifs (ITIMs). The LILRB1 receptor is expressed on immune cells, where it binds to MHC class I molecules on antigen-presenting cells and transduces a negative signal that inhibits stimulation of an immune response. LILRB1 is thought to regulate inflammatory responses, as well as cytotoxicity, and to play a role in limiting auto-reactivity. Multiple transcript variants encoding different isoforms of LILRB1 exist, all of which are contemplated as within the scope of the instant disclosure.

In some embodiments of the inhibitory receptors described herein, the inhibitory receptor comprises one or more domains isolated or derived from LILRB1. In some embodiments of the receptors having one or more domains isolated or derived from LILRB1, the one or more domains of LILRB1 comprise an amino acid sequence that is at least 80%, at least 90%, at least 95%, at least 96%, at least 97%, at least 98%, at least 99% or is identical to a sequence or subsequence of SEQ ID NO: 62. In some embodiments, the one or more domains of LILRB1 comprise an amino acid sequence that is identical to a sequence or subsequence of SEQ ID NO: 65. In some embodiments, the one or more domains of LILRB1 consist of an amino acid sequence that is at least 80%, at least 90%, at least 95%, at least 96%, at least 97%, at least 98%, at least 99% or is identical to a sequence or subsequence of SEQ ID NO: 62. In some embodiments, the one or more domains of LILRB1 consist of an amino acid sequence that is identical to a sequence or subsequence of SEQ ID NO: 62.

In some embodiments of the receptors having one or more domains isolated or derived from LILRB1, the one or more domains of LILRB1 are encoded by a polynucleotide sequence that is at least 80%, at least 90%, at least 95%, at least 96%, at least 97%, at least 98%, at least 99% or is identical to a sequence or subsequence of SEQ ID NO: 62.

In some embodiments of the receptors having one or more domains of LILRB1, the one or more domains of LILRB1 are encoded by a polynucleotide sequence that is identical to a sequence or subsequence of SEQ ID NO: 62.

In various embodiments, an inhibitory receptor is provided, comprising a polypeptide, wherein the polypeptide comprises one or more of: an LILRB1 hinge domain or functional fragment or variant thereof; an LILRB1 transmembrane domain or a functional variant thereof; and an LILRB1 intracellular domain or an intracellular domain comprising at least one, or at least two immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

As used herein an “immunoreceptor tyrosine-based inhibitory motif” or “ITIM” refers to a conserved sequence of amino acids with a consensus sequence of S/I/V/LxYxxI/V/L (SEQ ID NO: 265), or the like, that is found in the cytoplasmic tails of many inhibitory receptors of the immune system. After ITIM-possessing inhibitory receptors interact with their ligand, the ITIM motif is phosphorylated, allowing the inhibitory receptor to recruit other enzymes, such as the phosphotyrosine phosphatases SHP-1 and SHP-2, or the inositol-phosphatase called SHIP.

In some embodiments, the polypeptide comprises an intracellular domain comprising at least one immunoreceptor tyrosine-based inhibitory motif (ITIM), at least two ITIMs, at least 3 ITIMs, at least 4 ITIMs, at least 5 ITIMs or at least 6 ITIMs. In some embodiments, the intracellular domain has 1, 2, 3, 4, 5, or 6 ITIMs.

In some embodiments, the polypeptide comprises an intracellular domain comprising at least one ITIM selected from the group of ITIMs consisting of NLYAAV (SEQ ID NO: 64), VTYAEV (SEQ ID NO: 65), VTYAQL (SEQ ID NO: 66), and SIYATL (SEQ ID NO: 67).

In further particular embodiments, the polypeptide comprises an intracellular domain comprising at least two immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In some embodiments, the intracellular domain comprises both ITIMs NLYAAV (SEQ ID NO: 64) and VTYAEV (SEQ ID NO: 65). In some embodiments, the intracellular domain comprises a sequence at least 95% identical to SEQ ID NO: 68. In some embodiments, the intracellular domain comprises or consists essentially of a sequence identical to SEQ ID NO: 68.

In some embodiments, the intracellular domain comprises both ITIMs VTYAEV (SEQ ID NO: 65) and VTYAQL (SEQ ID NO: 66). In some embodiments, the intracellular domain comprises a sequence at least 95% identical to SEQ ID NO: 69. In some embodiments, the intracellular domain comprises or consists essentially of a sequence identical to SEQ ID NO: 69.

In some embodiments, the intracellular domain comprises both ITIMs VTYAQL (SEQ ID NO: 66) and SIYATL (SEQ ID NO: 67). In some embodiments, the intracellular domain comprises a sequence at least 95% identical to SEQ ID NO: 70. In some embodiments, the intracellular domain comprises or consists essentially of a sequence identical to SEQ ID NO:

In some embodiments, the intracellular domain comprises the ITIMs NLYAAV (SEQ ID NO: 64), VTYAEV (SEQ ID NO: 65), and VTYAQL (SEQ ID NO: 66). In some embodiments, the intracellular domain comprises a sequence at least 95% identical to SEQ ID NO: 71. In some embodiments, the intracellular domain comprises or consists essentially of a sequence identical to SEQ ID NO: 71.

In some embodiments, the intracellular domain comprises the ITIMs VTYAEV (SEQ ID NO: 65), VTYAQL (SEQ ID NO: 66), and SIYATL (SEQ ID NO: 67). In some embodiments, the intracellular domain comprises a sequence at least 95% identical to SEQ ID NO: 72. In some embodiments, the intracellular domain comprises or consists essentially of a sequence identical to SEQ ID NO: 72.

In some embodiments, the intracellular domain comprises the ITIMs NLYAAV (SEQ ID NO: 64), VTYAEV (SEQ ID NO: 65), VTYAQL (SEQ ID NO: 66), and SIYATL (SEQ ID NO: 67). In embodiments, the intracellular domain comprises a sequence at least 95% identical to SEQ ID NO: 73. In some embodiments, the intracellular domain comprises or consists essentially of a sequence identical to SEQ ID NO: 73.

In some embodiments, the intracellular domain comprises a sequence at least 95% identical to the LILRB1 intracellular domain (SEQ ID NO: 78). In some embodiments, the intracellular domain comprises or consists essentially of a sequence identical to the LILRB1 intracellular domain (SEQ ID NO: 78).

LILRB1 intracellular domains or functional variants thereof of the disclosure can have at least 1, at least 2, at least 4, at least 4, at least 5, at least 6, at least 7, or at least 8 ITIMs. In some embodiments, the LILRB1 intracellular domain or functional variant thereof has 2, 3, 4, 5, or 6 ITIMs.

In particular embodiments, the intracellular domain comprises two, three, four, five, or six immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In particular embodiments, the intracellular domain comprises at least three immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In particular embodiments, the intracellular domain comprises three immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In particular embodiments, the intracellular domain comprises four immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In particular embodiments, the intracellular domain comprises five immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In particular embodiments, the intracellular domain comprises six immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In particular embodiments, the intracellular domain comprises at least seven immunoreceptor tyrosine-based inhibitory motifs (ITIMs), wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

The LILRB1 protein has four immunoglobulin (Ig) like domains termed D1, D2, D3 and D4. In some embodiments, the LILRB1 hinge domain comprises an LILRB1 D3D4 domain or a functional variant thereof. In some embodiments, the LILRB1 D3D4 domain comprises a sequence at least 95%, at least 96%, at least 97%, at least 98%, at least 99% or identical to SEQ ID NO: 74. In some embodiments, the LILRB1 D3D4 domain comprises or consists essentially of SEQ ID NO: 74.

In some embodiments, the polypeptide comprises the LILRB1 hinge domain or functional fragment or variant thereof. In embodiments, the LILRB1 hinge domain or functional fragment or variant thereof comprises a sequence at least 95%, at least 96%, at least 97%, at least 98%, at least 99% identical or identical to SEQ ID NO: 81, SEQ ID NO: 74, or SEQ ID NO: 75. In embodiments, the LILRB1 hinge domain or functional fragment or variant thereof comprises a sequence at least 95% identical to SEQ ID NO: 81, SEQ ID NO: 74, or SEQ ID NO: 75.

In some embodiments, the LILRB1 hinge domain comprises a sequence identical to SEQ ID NO: 81, SEQ ID NO: 74, or SEQ ID NO: 75.

In some embodiments, the LILRB1 hinge domain consists essentially of a sequence identical to SEQ ID NO: 81, SEQ ID NO: 74, or SEQ ID NO: 75.

In some embodiments, the transmembrane domain is a LILRB1 transmembrane domain or a functional variant thereof. In some embodiments, the LILRB1 transmembrane domain or a functional variant thereof comprises a sequence at least 95% identical, at least 96% identical, at least 97% identical, at least 98% identical or at least 99% to SEQ ID NO: 81. In some embodiments, the LILRB1 transmembrane domain or a functional variant thereof comprises a sequence at least 95% identical to SEQ ID NO: 81. In some embodiments, the LILRB1 transmembrane domain comprises a sequence identical to SEQ ID NO: 81. In embodiments, the LILRB1 transmembrane domain consists essentially of a sequence identical to SEQ ID NO: 81.

In some embodiments, the transmembrane domain can be attached to the extracellular region of the second, inhibitory receptor, e.g., the antigen binding domain or ligand binding domain, via a hinge, e.g., a hinge from a human protein. For example, in some embodiments, the hinge can be a human immunoglobulin (Ig) hinge, e.g., an IgG4 hinge, a CD8α hinge or an LILRB1 hinge.

In some embodiments, the second, inhibitory receptor comprises an inhibitory domain. In some embodiments, the second, inhibitory receptor comprises an inhibitory intracellular domain and/or an inhibitory transmembrane domain. In some embodiments, the inhibitory domain is isolated or derived from LILR1B.

### Inhibitory Receptors Comprising Combinations of LILRB1 Domains

In some embodiments, the LILRB1-based inhibitory receptors of the disclosure comprise more than one LILRB1 domain or functional equivalent thereof. For example, in some embodiments, the inhibitory receptor comprises an LILRB1 transmembrane domain and intracellular domain, or an LILRB1 hinge domain, transmembrane domain and intracellular domain.

In particular embodiments, the inhibitory receptor comprises an LILRB1 hinge domain or functional fragment or variant thereof, and the LILRB1 transmembrane domain or a functional variant thereof. In some embodiments, the polypeptide comprises a sequence at least 95% identical, at least 96% identical, at least 97% identical, at least 98% identical, at least 99% identical or identical to SEQ ID NO: 76. In some embodiments, the polypeptide comprises a sequence at least 95% identical to SEQ ID NO: 76. In some embodiments, the polypeptide comprises a sequence identical to SEQ ID NO: 76.

In further embodiments, the inhibitory receptor comprises: the LILRB1 transmembrane domain or a functional variant thereof, and an LILRB1 intracellular domain and/or an intracellular domain comprising at least one immunoreceptor tyrosine-based inhibitory motif (ITIM), wherein the ITIM is selected from NLYAAV (SEQ ID NO: 64), VTYAEV (SEQ ID NO: 65), VTYAQL (SEQ ID NO: 66), and SIYATL (SEQ ID NO: 67). In some embodiments, the polypeptide comprises the LILRB1 transmembrane domain or a functional variant thereof, and an LILRB1 intracellular domain and/or an intracellular domain comprising at least two ITIM, wherein each ITIM is independently selected from NLYAAV (SEQ ID NO: 64), VTYAEV (SEQ ID NO: 65), VTYAQL (SEQ ID NO: 66), and SIYATL (SEQ ID NO: 67).

In some embodiments, the inhibitory receptor comprises a LILRB1 transmembrane domain and intracellular domain. In some embodiments, the polypeptide comprises a sequence at least 95% identical, at least 96% identical, at least 97% identical, at least 98% identical, at least 99% identical or identical to SEQ ID NO: 77. In some embodiments, the polypeptide comprises a sequence at least 95% identical to SEQ ID NO: 77. In some embodiments, the polypeptide comprises a sequence identical to SEQ ID NO: 77. In some embodiments, the inhibitory receptor comprises the LILRB1 transmembrane domain and intracellular domain of SEQ ID NO: 77 fused to an extracellular ligand binding domain. In some embodiments, the inhibitory receptor comprises a first polypeptide comprising SEQ ID NO: 77 fused to a TCR alpha variable domain, and a second polypeptide comprising SEQ ID NO: 77 fused to a TCR beta variable domain.

In preferred embodiments, the inhibitory receptor comprises: an LILRB1 hinge domain or functional fragment or variant thereof; an LILRB1 transmembrane domain or a functional variant thereof; and an LILRB1 intracellular domain and/or an intracellular domain comprising at least two immunoreceptor tyrosine-based inhibitory motifs (ITIMs), wherein each ITIM is independently selected from LYAAV (SEQ ID NO: 64), VTYAE (SEQ ID NO: 65), VTYAQL (SEQ ID NO: 66), and SIYATL (SEQ ID NO: 11).

In some embodiments, the inhibitory receptor comprises a sequence at least 95% identical to SEQ ID NO: 79 or SEQ ID NO: 80, or at least 99% identical to SEQ ID NO: 79 or SEQ ID NO: 80, or identical to SEQ ID NO: 79 or SEQ ID NO: 80.

In some embodiments, the polypeptide comprises a sequence at least 99% identical to SEQ ID NO: 76, or at least 99% identical to SEQ ID NO: 76, or identical to SEQ ID NO: 76.

In some embodiments, the polypeptide comprises a sequence at least 99% identical to SEQ ID NO: 77, or at least 99% identical to SEQ ID NO: 77, or identical to SEQ ID NO: 77.

### Linkers

In some embodiments, the engineered receptors comprise a linker linking two domains of the engineered receptor. Provided herein are linkers that, in some embodiments, can be used to link domains of the engineered receptors described herein.

The terms “linker” and “flexible polypeptide linker” as used in the context of linking protein domains, for example intracellular domains or domains within an scFv, refers to a peptide linker that consists of amino acids such as glycine and/or serine residues used alone or in combination, to link two domains together.

Any linker may be used and many fusion protein linker formats are known. For example, the linker may be flexible or rigid. Non-limiting examples of rigid and flexible linkers are provided in Chen et al. (Adv Drug Deliv Rev. 2013; 65(10):1357-1369).

The antigen-binding domains described herein may be linked to each other in a random or specified order.

The antigen-binding domains described herein may be linked to each other in any orientation of N to C terminus.

Optionally, a short oligo- or polypeptide linker, for example, between 2 and 40 amino acids (e.g., 2, 3, 4, 5, 6, 7, 8, 9, or 10 amino acids) in length may form the linkage between the domains.

In some embodiments, the linker is a peptide of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 amino acid residues. Non-limiting examples of amino acids found in linkers include Gly, Ser, Glu, Gin, Ala, Leu, Iso, Lys, Arg, Pro, and the like. In some embodiments, the linker is [(Gly)n1 Ser]n2, where n1 and n2 may be any number (e.g. n1 and n2 may independently be 1, 2, 4, 5, 6, 7, 8, 9, 10 or more than 10). In some embodiments, n1 is 4.

In some embodiments, the flexible polypeptide linker is a Gly/Ser linker and comprises the amino acid sequence (Gly-Gly-Ser), (Gly-Gly-Gly-Ser, SEQ ID NO: 227), or (Gly-Gly-Gly-Gly-Ser, SEQ ID NO: 222) which can be repeated n times, where n is a positive integer equal to or greater than 1. For example, n=1, n=2, n=3, n=4, n=5, n=6, n=7, n=8, n=9 and n=10. In some embodiments, the flexible polypeptide linkers include, but are not limited to, GGS, GGGGS (SEQ ID NO: 222), GGGGS GGGGS (SEQ ID NO: 223), GGGGS GGGGS GGGGS (SEQ ID NO: 224), GGGGS GGGGS GGGGS GG (SEQ ID NO: 225) or GGGGS GGGGS GGGGS GGGGS (SEQ ID NO: 226).

In some embodiments, the linkers include multiple repeats of (Gly Gly Ser), (Gly Ser) or (Gly Gly Gly Ser (SEQ ID NO: 227)). Also included within the scope of the invention are linkers described in WO2012/138475 (incorporated herein by reference).

In some embodiments, the linker sequence comprises a long linker (LL) sequence. In some embodiments, the long linker sequence comprises GGGGS (SEQ ID NO: 222), repeated four times. In some embodiments, a GGGGS GGGGS GGGGS GGGGS (SEQ ID NO: 226) is used to link intracellular domains in a TCR alpha fusion protein of the disclosure.

In some embodiments, the long linker sequence comprises GGGGS (SEQ ID NO: 222), repeated three times. In some embodiments, a GGGGS GGGGS GGGGS (SEQ ID NO: 224) is used to link intracellular domains in a TCR beta fusion protein of the disclosure.

In some embodiments, the linker sequence comprises a short linker (SL) sequence. In some embodiments, the short linker sequence comprises GGGGS (SEQ ID NO: 222).

In some embodiments, a glycine-serine doublet can be used as a suitable linker.

In some embodiments, domains are fused directly to each other via peptide bonds without use of a linker.

### Assays

Provided herein are assays that can be used to measure the activity of the engineered receptors of the disclosure.

The activity of engineered receptors can be assayed using a cell line engineered to express a reporter of receptor activity such as a luciferase reporter. Exemplary cell lines include Jurkat T cells, although any suitable cell line known in the art may be used. For example, Jurkat cells expressing a luciferase reporter under the control of an NFAT promoter can be used as effector cells. Expression of luciferase by this cell line reflects TCR-mediated signaling.

The reporter cells can be transfected with each of the various fusion protein constructs, combinations of fusion protein constructs or controls described herein.

Expression of the fusion proteins in reporter cells can be confirmed by using fluorescently labeled MHC tetramers, for example Alexa Fluor 647-labeled NY-ESO-1-MHC tetramer, to detect expression of the fusion protein.

To assay the activity of engineered receptors, target cells are loaded with antigen prior to exposure to the effector cells comprising the reporter and the engineered receptor. For example, target cells can be loaded with antigen at least 12, 14, 16, 18, 20, 22 or 24 hours prior to exposure to effector cells. Exemplary target cells include A375 cells, although any suitable cells known in the art may be used. In some cases, target cells can be loaded with serially diluted concentrations of an antigen, such as NY-ESO-1 peptide. The effector cells can then be co-cultured with target cells for a suitable period of time, for example 6 hours. Luciferase is then measured by luminescence reading after co-culture. Luciferase luminescence can be normalized to maximum and minimum intensity to allow comparison of activating peptide concentrations for each engineered receptor construct.

Provided herein are methods of determining the relative EC50 of engineered receptors of the disclosure. As used herein, “EC50” refers to the concentration of an inhibitor or agent where the response (or binding) is reduced by half. EC50s of engineered receptors of the disclosure refer to concentration of antigen where binding of the engineered receptor to the antigen is reduced by half. Binding of the antigen, or probe to the engineered receptor can be measured by staining with labeled peptide or labeled peptide-MHC complex, for example MHC:NY-ESO-1 pMHC complex conjugated with fluorophore. EC50 can be obtained by nonlinear regression curve fitting of reporter signal with peptide titration. Probe binding and EC50 can be normalized to the levels of benchmark TCR without a fusion protein, e.g. NY-ESO-1 (clone 1G4).

### Polynucleotides

The disclosure provides polynucleotides encoding the sequence(s) of the engineered receptors described herein.

In some embodiments, the sequence of the first and/or second fusion protein is operably linked to a promoter. In some embodiments, the sequence encoding the first fusion protein is operably linked to a first promoter, and the sequence encoding a second fusion protein is operably linked to a second promoter.

The disclosure provides polynucleotides encoding the sequence(s) of the activator and inhibitory receptors described herein.

In some embodiments, the sequence of the first and/or second receptor, or a fusion protein of the first and/or second receptor is operably linked to a promoter. In some embodiments, the sequence encoding the activator receptor, or a polypeptide thereof, is operably linked to a first promoter, and the sequence encoding a inhibitory receptor, or a fusion protein thereof, is operably linked to a second promoter.

The disclosure provides polynucleotides comprising the sequence(s) of the interfering RNA described herein. In some embodiments, the polynucleotides comprise the shRNA described herein. In some embodiments, the shRNA comprises a first sequence, having from end to 3′ end a sequence complementary to an HLA-A*02 mRNA; and a second sequence, having from 5′ end to 3′ end a sequence complementary to the first sequence. In some embodiments, the HLA-A*02 mRNA sequence comprises a coding sequence. In some embodiments, the HLA-A*02 mRNA sequence comprises an untranslated region. In some embodiments, the first and second sequence are present on a polynucleotide, wherein the first sequence and the second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA. In some embodiments, the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence.

In some embodiments, the polynucleotide encoding an shRNA has from 5′ end to 3′ end, a 5′ flank sequence, a first sequence, a loop sequence, a second sequence, a 3′ flank sequence. In some embodiments, the polynucleotide encoding an shRNA has from 5′ end to 3′ end, a 5′ flank sequence, a second sequence, a loop sequence, a first sequence, and a 3′ flank sequence.

In some embodiments, the first sequences is 18, 19, 20, 21, or 22 nucleotides. In some embodiments, the first sequence is complementary to a sequence selected from SEQ ID NOs: 8476-16870. In some embodiments, the first sequence has GC content greater than or equal to 25% and less than 60%. In some embodiments, the first sequence is complementary to a sequence selected from SEQ ID NOs: 8476-12066. In some embodiments, the first sequence does not comprise four nucleotides of the same base or a run of seven C or G nucleotide bases. In some embodiments, the first sequence is complementary to a sequence selected from SEQ ID NOs: 8476-11584. In some embodiments, the first sequence is complementary to a sequence selected from SEQ ID NOs: 8476-8754. In some embodiments, the first sequence is complementary to a sequence selected from SEQ ID NOs: 8476-8561.

In some embodiments, the polynucleotide encoding an shRNA further comprises a promoter sequence and a terminator sequence. In some embodiments, the shRNA is operably linked to the promoter. In some embodiments, the polynucleotide has from 5′ end to 3′ end, a promoter sequence, a 5′ flank sequence, a first sequence, a loop sequence, a second sequence, a 3′ flank sequence, and a terminator. In some embodiments, the polynucleotide encodes, from end to 3′ end, a promoter sequence, a 5′ flank sequence, a second sequence, a loop sequence, a first sequence, a 3′ flank sequence, and a terminator sequence.

In some embodiments, the polynucleotides comprise a promoter operably linked to the shRNA, such as a mammalian, viral or synthetic promoter. In some embodiments, the promoter sequence is a U6 promoter sequence. In some embodiments, the promoter sequence shares at least 80%, at least 85%, at least 90%, at least 95%, at least 98%, at least 99% identity to SEQ ID NO: 16890. In some embodiments, the promoter sequence is SEQ ID NO: 16890. In some embodiments, the promoter sequence is a H1 promoter sequence. In some embodiments, the promoter sequence shares at least 80%, at least 85%, at least 90%, at least 95%, at least 98%, at least 99% identity to SEQ ID NO: 16891. In some embodiments, the promoter sequence is SEQ ID NO: 16891. In some embodiments, the promoter sequence is a 7SK promoter sequence. In some embodiments, the promoter sequence shares at least 80%, at least 85%, at least 90%, at least 95%, at least 98%, at least 99% identity to SEQ ID NO: 16892. In some embodiments, the promoter sequence is SEQ ID NO: 16892. In some embodiments, the promoter sequence is a Ef1a promoter sequence. In some embodiments, the promoter sequence shares at least 80%, at least 85%, at least 90%, at least 95%, at least 98%, at least 99% identity to SEQ ID NO: 16893. In some embodiments, the promoter sequence is SEQ ID NO: 16893.

### Vectors

The disclosure provides vectors comprising the polynucleotides described herein.

The disclosure provides vectors encoding the interfering RNA described herein. In some embodiments, the vectors encode the shRNA described herein. In some embodiments, the shRNA comprises a first sequence, having from 5′ end to 3′ end a sequence complementary to the HLA-A*02 mRNA; and a second sequence, having from 5′ end to 3′ end a sequence complementary to the first sequence. In some embodiments, the first and second sequence are present on a polynucleotide, wherein the first sequence and the second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA. In some embodiments, the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence.

In some embodiments, the vectors encoding an shRNA has from 5′ end to 3′ end, a 5′ flank sequence, a first sequence, a loop sequence, a second sequence, a 3′ flank sequence. In some embodiments, the vectors encode, from 5′ end to 3′ end, and a 3′ flank sequence. In some embodiments, the vectors encode an shRNA has from 5′ end to 3′ end, a 5′ flank sequence, a second sequence, a loop sequence, a first sequence, and a 3′ flank sequence.

In some embodiments, the vectors encoding an shRNA further comprises a promoter sequence and a terminator sequence. In some embodiments, the shRNA is operably linked to the promoter. In some embodiments, the vector has from 5′ end to 3′ end, a promoter sequence, a 5′ flank sequence, a first sequence, a loop sequence, a second sequence, a 3′ flank sequence, and a terminator. In some embodiments, the vector has from 5′ end to 3′ end, a promoter sequence, a 5′ flank sequence, a second sequence, a loop sequence, a first sequence, a 3′ flank sequence, and a terminator sequence.

In some embodiments of the vectors of the disclosure, the promoter sequence is a U6 promoter sequence. In some embodiments, the promoter sequence shares at least 80%, at least 85%, at least 90%, at least 95%, at least 98%, at least 99% identity to SEQ ID NO: 16890. In some embodiments, the promoter sequence is SEQ ID NO: 16890. In some embodiments, the promoter sequence is a H1 promoter sequence. In some embodiments, the promoter sequence shares at least 80%, at least 85%, at least 90%, at least 95%, at least 98%, at least 99% identity to SEQ ID NO: 16891. In some embodiments, the promoter sequence is SEQ ID NO: 16891. In some embodiments, the promoter sequence is a 7SK promoter sequence. In some embodiments, the promoter sequence shares at least 80%, at least 85%, at least 90%, at least 95%, at least 98%, at least 99% identity to SEQ ID NO: 16892. In some embodiments, the promoter sequence is SEQ ID NO: 16892. In some embodiments, the promoter sequence is a Ef1a promoter sequence. In some embodiments, the promoter sequence shares at least 80%, at least 85%, at least 90%, at least 95%, at least 98%, at least 99% identity to SEQ ID NO: 16893. In some embodiments, the promoter sequence is SEQ ID NO: 16893.

In some embodiments, the vector described herein is a viral vector. In some embodiments, the vector is a lentiviral vector.

The disclosure provides vectors encoding an interfering RNA described herein and an inhibitory receptor. In some embodiments, the vector comprises an shRNA described herein and a polynucleotide encoding an inhibitor receptor comprising a ligand binding domain specific to a class I major histocompatibility complex (MHC-I) molecule, or a peptide-MHC complex thereof.

The disclosure provides polynucleotides encoding the sequence(s) of the activator and inhibitory receptors described herein.

The disclosure provides vectors comprising the polynucleotides described herein.

The disclosure provides vectors encoding the coding sequence or sequences of any of the engineered receptors described herein. In some embodiments, the sequence of the first and/or second fusion protein is operably linked to a promoter. In some embodiments, the sequence encoding the first fusion protein is operably linked to a first promoter, and the sequence encoding a second fusion protein is operably linked to a second promoter.

In some embodiments, the first engineered receptor is encoded by a first vector and the second engineered receptor is encoded by second vector. In some embodiments, both engineered receptors are encoded by a single vector.

In some embodiments, the first and second receptors are encoded by a single vector. Methods of encoding multiple polypeptides using a single vector will be known to persons of ordinary skill in the art, and include, inter alia, encoding multiple polypeptides under control of different promoters, or, if a single promoter is used to control transcription of multiple polypeptides, use of sequences encoding internal ribosome entry sites (IRES) and/or self-cleaving peptides. Exemplary self-cleaving peptides include T2A, P2A, E2A and F2A self-cleaving peptides. In some embodiments, the T2A self-cleaving peptide comprises a sequence of EGRGSLLTCGDVEENPGP (SEQ ID NO: 261). In some embodiments, the P2A self-cleaving peptide comprises a sequence of ATNFSLLKQAGDVEENPGP (SEQ ID NO: 188). In some embodiments, the E2A self-cleaving peptide comprises a sequence of QCTNYALLKLAGDVESNPGP (SEQ ID NO: 262). In some embodiments, the F2A self-cleaving peptide comprises a sequence of VKQTLNFDLLKLAGDVESNPGP (SEQ ID NO: 263). In some embodiments, the P2A self-cleaving peptide comprises a sequence of VKQTLNFDLLKLAGDVESNPGP (SEQ ID NO: 21905).

The disclosure provides polynucleotides encoding the gene editing systems described herein.

In some embodiments, the vector is an expression vector, i.e. for the expression of the fusion protein in a suitable cell.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The expression of natural or synthetic nucleic acids encoding fusion proteins is typically achieved by operably linking a nucleic acid encoding the fusion protein or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The polynucleotides encoding the fusion proteins can be cloned into a number of types of vectors. For example, the polynucleotides can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to cells, such as immune cells, in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication function in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 basepairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α, Ef1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a fusion protein, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected or transduced cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). One method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Immune Cells

Provided herein are immune cells comprising the polynucleotides, vectors, fusion proteins and engineered receptors described herein.

Provided herein are immune cells edited using the gene editing systems described herein.

In some embodiments, the immune cells comprise the polynucleotides, vectors, fusion proteins or engineered receptors of the disclosure.

Provided herein are immune cells comprising the interfering RNAs, polynucleotides, vectors, fusion proteins and engineered receptors described herein.

Provided herein are immune cells comprising the interfering RNAs, e.g. shRNAS, described herein. In some embodiments, the immune cells comprise the interfering RNAs. polynucleotides, vectors, fusion proteins or engineered receptors of the disclosure.

In some embodiments, the immune cell is a T cell, B cell, or Natural Killer (NK) cell. In some embodiments, the immune cell is autologous to a subject. In some embodiments, the immune cell is allogeneic to a subject. In some embodiments, the immune cell is non-natural.

In some embodiments, the immune cell is isolated. In some embodiments, the immune cell is for use as a medicament. In some embodiments, the medicament is for the treatment of cancer in a subject in need thereof.

In some embodiments, the immune cell comprises an inhibitory receptor comprising a ligand binding domain specific to a class I major histocompatibility complex (MHC-I) molecule, or a peptide-MHC complex thereof wherein expression and/or function of a human leukocyte antigen (HLA) polypeptide, or an allele thereof, in said immune cell has been reduced or eliminated. In some embodiments, the HLA allele is an HLA-A, HLA-B, HLA-C, and/or HLA-E allele. In some embodiments, the HLA-A allele is selected from HLA-A*02, HLA-A*02:01, HLA-A*02:01:01, and HLA-A*02:01:01:01. In some embodiments, the HLA-A allele is HLA-A*02.

In some embodiments, the immune cell comprises an interfering RNA, comprising a sequence complementary to a sequence of a HLA-A*02 mRNA. In some embodiments, the interfering RNA is capable of inducing RNAi-mediated degradation of the HLA-A*02 mRNA. In some embodiments, the interfering RNA is a short hairpin RNA (shRNA).

In some embodiments, the shRNA comprises a first sequence, having from 5′ to 3′ end a sequence complementary to the HLA-A*02 mRNA; and a second sequence, having from 5′ to 3′ end a sequence complementary to the first sequence, wherein the first sequence and second sequence form the shRNA.

In some embodiments, the first sequences is 18, 19, 20, 21, or 22 nucleotides. In some embodiments, the first sequence is complementary to a sequence selected from SEQ ID NOs: 8476-16870. In some embodiments, the first sequence has GC content greater than or equal to 25% and less than 60%. In some embodiments, the first sequence is complementary to a sequence selected from SEQ ID NOs: 8476-12066. In some embodiments, the first sequence does not comprise four nucleotides of the same base or a run of seven C or G nucleotide bases. In some embodiments, the first sequence is complementary to a sequence selected from SEQ ID NOs: 8476-11584. In some embodiments, the first sequence is complementary to a sequence selected from SEQ ID NOs: 8476-8754. In some embodiments, the first sequence is complementary to a sequence selected from SEQ ID NOs: 8476-8561.

In some embodiments, the first and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA. In some embodiments, the loop region comprises a sequence selected from SEQ ID NOs: 16872-16884 and 16895.

In some embodiments, the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence. In some embodiments, the 5′ flank sequence is selected from SEQ ID NO: 16885-16887. In some embodiments, the 3′ flank sequence is selected from SEQ ID NO: 16888, 16889, and 16896.

As used herein, the term “immune cell” refers to a cell involved in the innate or adaptive (acquired) immune systems. Exemplary innate immune cells include phagocytic cells such as neutrophils, monocytes and macrophages, Natural Killer (NK) cells, polymophonuclear leukocytes such as neutrophils eosinophils and basophils and mononuclear cells such as monocytes, macrophages and mast cells. Immune cells with roles in acquired immunity include lymphocytes such as T-cells and B-cells.

As used herein, a “T-cell” refers to a type of lymphocyte that originates from a bone marrow precursor that develops in the thymus gland. There are several distinct types of T-cells which develop upon migration to the thymus, which include, helper CD4+ T-cells, cytotoxic CD8+ T cells, memory T cells, regulatory CD4+ T-cells and stem memory T-cells. Different types of T-cells can be distinguished by the ordinarily skilled artisan based on their expression of markers. Methods of distinguishing between T-cell types will be readily apparent to the ordinarily skilled artisan.

In some embodiments, the engineered immune cell expresses the first and second receptors at a ratio of about 100:1 to 1:100 of first receptor to second receptor. In some embodiments, the engineered immune cell expresses the first and second receptors at a ratio of about 50:1 to 1:50 of first receptor to second receptor. In some embodiments, the engineered immune cell expresses the first and second receptors at a ratio of about 10:1 to 1:10 of first receptor to second receptor. In some embodiments, the engineered immune cell expresses the first and second receptors at a ratio of about 5:1 to 1:5 of first receptor to second receptor. In some embodiments, the engineered immune cell expresses the first and second receptors at a ratio of about 3:1 to 1:3 of first receptor to second receptor. In some embodiments, the engineered immune cell expresses the first and second receptors at a ratio of about 2:1 to 1:2 of first receptor to second receptor. In some embodiments, the engineered immune cell expresses the first and second receptors at a ratio of about 1:1.

In some embodiments, the engineered immune cell comprising the engineered receptors of the disclosure is a T cell. In some embodiments, the T cell is an effector T cell or a regulatory T cell.

Methods transforming populations of immune cells, such as T cells, with the vectors of the instant disclosure will be readily apparent to the person of ordinary skill in the art. For example, CD3+ T cells can be isolated from PBMCs using a CD3+ T cell negative isolation kit (Miltenyi), according to manufacturer's instructions. T cells can be cultured at a density of 1×10\hat{ }6 cells/mL in X-Vivo 15 media supplemented with 5% human A/B serum and 1% Pen/strep in the presence of CD3/28 Dynabeads (1:1 cell to bead ratio) and 300 Units/mL of IL-2 (Miltenyi). After 2 days, T cells can be transduced with viral vectors, such as lentiviral vectors using methods known in the art. In some embodiments, the viral vector is transduced at a multiplicity of infection (MOI) of 5. Cells can then be cultured in IL-2 or other cytokines such as combinations of IL-7/15/21 for an additional 5 days prior to enrichment. Methods of isolating and culturing other populations of immune cells, such as B cells, or other populations of T cells, will be readily apparent to the person of ordinary skill in the art. Although this method outlines a potential approach it should be noted that these methodologies are rapidly evolving. For example excellent viral transduction of peripheral blood mononuclear cells can be achieved after 5 days of growth to generate a >99% CD3+ highly transduced cell population.

Additional methods of transforming populations of immune cells, such as T cells, include transfection. For example, immune cells, such as T cells, can be transfected with nucleic acids encoding guide RNAs (gRNAs) or shRNAs under control of a promoter, or directly with the gRNA itself. Alternatively, immune cells can be transfected with the gRNA in complex with a CRISPR/Cas protein as a ribonucleoprotein complex. As a still further alternative, immune cells can be transfected with gRNA, or a nucleic acid encoding a gRNA, and CRISPR/Cas protein or a nucleic acid encoding a CRISPR/Cas protein. Methods of immune cell transfection will be known to persons of skill in the art, and include, inter alia, electroporation methods such as nucleofection and chemical based methods such as through the use of cationic lipid or calcium phosphate reagents.

Methods of activating and culturing populations of T cells comprising the engineered TCRs, CARs, fusion proteins or vectors encoding the fusion proteins of the instant disclosure, will be readily apparent to the person of ordinary skill in the art.

Whether prior to or after genetic modification of T cells to express an engineered TCR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, 10040846; and U.S. Pat. Appl. Pub. No. 2006/0121005.

In some embodiments, T cells of the instant disclosure are expanded and activated in vitro. Generally, the T cells of the instant disclosure are expanded in vitro by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4+ T cell expansion and T cell growth is used. In some embodiments, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In some embodiments, a ratio of 1:1 cells to beads is used. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3ξ and anti-CD28 are attached to contact the T cells. In one embodiment the cells (for example, CD4+ T cells) and beads (for example, DYNABEADS CD3/CD28 T paramagnetic beads at a ratio of 1:1) are combined in a buffer. Again, those of ordinary skill in the art can readily appreciate any cell concentration may be used. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. In some embodiments, cells that are cultured at a density of 1×10⁶ cells/mL are used.

In some embodiments, the mixture may be cultured for several hours (about 3 hours) to about 14 days or any hourly integer value in between. In another embodiment, the beads and T cells are cultured together for 2-3 days. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. In some embodiments, the media comprises X-VIVO-15 media supplemented with 5% human A/B serum, 1% penicillin/streptomycin (pen/strep) and 300 Units/ml of IL-2 (Miltenyi).

The T cells are maintained under conditions necessary to support growth, for example, an appropriate temperature (e.g., 37° C.) and atmosphere (e.g., air plus 5% CO2).

In some embodiments, the T cells comprising engineered receptors of the disclosure are autologous. In some embodiments, the T cells comprising engineered receptors of the disclosure are allogeneic. Prior to expansion and genetic modification, a source of T cells is obtained from a subject. Immune cells such as T cells can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation.

In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In alternative embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In some embodiments, immune cells such as T cells are isolated from peripheral blood lymphocytes by lysing the red blood cells and depleting the monocytes, for example, by centrifugation through a PERCOLL™ gradient or by counterflow centrifugal elutriation. Specific subpopulations of immune cells, such as T cells, B cells, or CD4+ T cells can be further isolated by positive or negative selection techniques. For example, in one embodiment, T cells are isolated by incubation with anti-CD4-conjugated beads, for a time period sufficient for positive selection of the desired T cells.

Enrichment of an immune cell population, such as a T cell population, by negative selection can be accomplished with a combination of antibodies directed to surface markers unique to the negatively selected cells. One method is cell sorting and/or selection via negative magnetic immune-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD 11b, CD 16, HLA-DR, and CD8.

In some embodiments, an immune cell population, such as a T cell population, is depleted of NK cells. NK cells can be depleted through any methods known in the art. For example, NK cells can be depleted using antibodies, such as anti-CD56 antibodies, that, when bound to beads, can be used to bind to and isolate CD56 positive NK cells from a mixture of immune cell types. NK cell isolation kits are available commercially, and include, for example, the FastStep NK Cell Isolation kit from Creative Biolabs, the NK Cell Isolation kit from Miltenyi Biotec, and the EasySep Human NK Cell Isolation kit from StemCell Technologies. An exemplary NK cell depletion protocol includes incubating peripheral blood mononuclear cells (PBMCs) with microbeads coated with anti-human CD56 (available from, e.g., Miltenyi Biotec, catalog number #130-050-401, or #170-076-713 if CliniMACS CD56 GMP Microbeads) according to the manufacturer's instructions. Anti-CD56 bound cells are then separated on an AutoMACS Pro, CliniMACS Prodigy (Miltyenyi Biotec), or equivalent. The negative fraction, i.e. the fraction not containing anti-CD56 bound cells, is collected and incubated with microbeads suitable to isolate T cells. Suitable microbeads for T cells isolation include anti-human CD4 and CD8 microbeads (Miltyenyi Biotec #130-045-101 and 130-045-201 respectively) or CliniMACS CD4 GMP and CD8 MicroBeads (Miltyenyi Biotec #170-076-702 and 170-076-703). CD4 and/or CD8 positive T cells are then separated on an AutoMACS Pro or CliniMACS prodigy, or equivalent.

For isolation of a desired population of immune cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads.

In some embodiments, the cells may be incubated on a rotator for varying lengths of time at varying speeds at either 2-10° C. or at room temperature.

T cells for stimulation, or PBMCs from which immune cells such as T cells are isolated, can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

### Pharmaceutical Compositions

The disclosure provides pharmaceutical compositions comprising immune cells edited using the gene editing systems of the disclosure, and comprising the engineered receptors of the disclosure and a pharmaceutically acceptable diluent, carrier or excipient.

In some embodiments, the immune cell comprises an inhibitory receptor comprising a ligand binding domain specific to a class I major histocompatibility complex (MHC-I) molecule, or a peptide-MHC complex thereof; wherein expression and/or function of human leukocyte antigen (HLA) in said immune cell has been reduced or eliminated. In some embodiments, the immune cell comprises an interfering RNA, comprising a sequence complementary to a sequence of a HLA-A*02 mRNA. In some embodiments, the interfering RNA is capable of inducing RNAi-mediated degradation of the HLA-A*02 mRNA. In some embodiments, the interfering RNA is a short hairpin RNA (shRNA) as described herein. In some embodiments, the immune cell further comprises an activator receptor as described herein.

Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; and preservatives.

Provided herein are methods of producing an immune cell with reduced autocrine binding/signaling comprising transducing and/or transfecting the immune cell with a vector described herein. In some embodiments, the method comprises transducing the immune cell with a first vector comprising a sequence encoding an activator receptor and a second vector comprising a sequence encoding an inhibitory receptor, thereby producing an immune cell expressing the activator and inhibitory receptors. In some embodiments, the inhibitory receptor specifically binds to an HLA-A*02 pMHC antigen and the target gene comprises HLA-A*02. In some embodiments, prior to the transducing and/or transfecting steps, the immune cell comprises a polynucleotide or vector encoding interfering RNA targeting a HLA-A*02 mRNA.

### Methods of Treating Disease

Provided herein are methods of treating a subject in need thereof, comprising administering to the subject a therapeutically effective amount of a composition comprising allogeneic immune cells comprising the engineered receptors and gene editing modifications of the disclosure. The immune cells express both engineered receptors in the same cell. As used herein, the term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. For example, cells of the same species that differ genetically are allogerieic.

In some embodiments, the method of treating a subject comprises providing immune cells from a subject suffering from or at risk for cancer or a hematological malignancy; transducing the immune cell with a vector comprising a sequence encoding a nucleic acid-guided endonuclease, thereby expressing the nuclease; transfecting the immune cell with at least one guide nucleic acid (gNA) complementary to a target sequence of a target gene selected from the group consisting of HLA-A, HLA-B, HLA-C, an allele thereof, or a combination thereof, wherein the gNA bind to the target sequence and the nuclease cleaves the target sequence, thereby producing a modified target gene; and administering the immune cell to the subject.

Provided herein are methods of treating a subject in need thereof, comprising administering to the subject a therapeutically effective amount of a composition comprising immune cells comprising the engineered receptors and having reduced or eliminated expression and/or function of HLA. In some embodiments, the immune cell comprises an interfering RNA (e.g. an shRNA), polynucleotide, vector, fusion protein, engineered receptor (e.g. an inhibitory receptor) of the disclosure.

In some embodiments, the method of treating a subject in need thereof comprises providing immune cells from a subject suffering from or at risk for cancer or a hematological malignancy; transducing the immune cell with the vectors described herein; and administering the immune cell to the subject.

Provided herein are methods of manufacturing a composition comprising immune cells with reduced autocrine binding/signaling comprising providing immune cells from a subject suffering from or at risk for cancer or a hematological malignancy; and transducing or transfecting the immune cell with one or more vectors described herein. In some embodiments, the vector encodes an interfering RNA targeting HLA-A*02 mRNA. In some embodiments, the vector encodes an engineered receptor (e.g. an inhibitory receptor that specifically binds to HLA-A*02 pMHC antigen and the target gene comprises HLA-A*02. In some embodiments, prior to the transducing and/or transfecting steps, the immune cell comprises a polynucleotide encoding activator and/or inhibitory receptors. In some embodiments, the method comprises transducing the immune cell with a first vector comprising a sequence encoding the activator receptor and a second vector comprising a sequence encoding the inhibitory receptor, thereby producing an immune cell expressing the activator and inhibitory receptors.

The current method for adoptive cell therapy using autologous cells includes isolating immune cells from patient blood, performing a series of modifications on the isolated cells, and administering the cells to a patient (Papathanasiou et al. Cancer Gene Therapy. 27:799-809 (2020)). Providing immune cells from a subject suffering from or at risk for cancer or a hematological malignancy requires isolation of immune cell from the patient's blood, and can be accomplished through methods known in the art, for example, by leukapheresis. During leukapheresis, blood from a subject is extracted and the peripheral blood mononuclear cells (PBMCs) are separated, and the remainder of the blood is returned to the subject's circulation. The PBMCs are stored either frozen or cryopreserved as a sample of immune cells and provided for further processing steps, such as, e.g. the modifications described herein.

In some embodiments, the method of treating a subject described herein comprises modifications to immune cells from the subject comprising a series of modifications comprising enrichment, activation, genetic modification, expansion, formulation, and cryopreservation.

The disclosure provides enrichment steps that can be, for example, washing and fractionating methods known in the art for preparation of subject PBMCs for downstream procedures, e.g. the modifications described herein. For example, without limitation, methods can include devices to remove gross red blood cells and platelet contaminants, systems for size-based cell fractionation for the depletion of monocytes and the isolation of lymphocytes, and/or systems that allow the enrichment of specific subsets of T cells, such as, e.g. CD4+, CD8+, CD25+, or CD62L+ T cells. Following the enrichment steps, a target sub-population of immune cells will be isolated from the subject PMBCs for further processing. Those skilled in the art will appreciate that enrichment steps, as provided herein, may also encompass any newly discovered method, device, reagent or combination thereof.

The disclosure provides activation steps that can be any method known in the art to induce activation of immune cells, e.g. T cells, required for their ex vivo expansion. Immune cell activation can be achieved, for example, by culturing the subject immune cells in the presence of dendritic cells, culturing the subject immune cells in the presence of artificial antigen-presenting cells (AAPCs), or culturing the immune cells in the presence of irradiated K562-derived AAPCs. Other methods for activating subject immune cells can be, for example, culturing the immune cells in the presence of isolated activating factors and compositions, e.g. beads, surfaces, or particles functionalized with activating factors. Activating factors can include, for example, antibodies, e.g. anti-CD3 and/or anti-CD28 antibodies. Activating factors can also be, for example, cytokines, e.g. interleukin (IL)-2 or IL-21. Activating factors can also be costimulatory molecules, such as, for example, CD40, CD40L, CD70, CD80, CD83, CD86, CD137L, ICOSL, GITRL, and CD134L. Those skilled in the art will appreciate that activating factors, as provided herein, may also encompass any newly discovered activating factor, reagent, composition, or combination thereof that can activate immune cells.

The disclosure provides genetic modification steps for modifying the subject immune cells. In some embodiments, the genetic modification comprises transducing the immune cell with a vector comprising a sequence encoding a nucleic acid-guided endonuclease, thereby expressing the nuclease; transfecting the immune cell with at least one guide nucleic acid (gNA) complementary to a target sequence of a target gene selected from the group consisting of HLA-A, HLA-B, HLA-C, or an allele thereof, including all alleles of HLA-A, B, and/or C, wherein the gNA bind to the target sequence and the nuclease cleaves the target sequence, thereby producing a modified target gene or genes; and administering the immune cell to the subject. The gNA can be a guide nucleic acid described herein. The genetic modification steps can also be transduction of the immune cell with an engineered receptor. In some embodiments, the method comprises transducing the immune cell with a first vector comprising a sequence encoding the activator receptor and a second vector comprising a sequence encoding the inhibitory receptor, thereby producing an immune cell expressing the activator and inhibitory receptors.

The disclosure provides expansion steps for the genetically modified subject immune cells. Genetically modified subject immune cells can be expanded in any immune cell expansion system known in the art to generate therapeutic doses of immune cells for administration. For example, bioreactor bags for use in a system comprising controller pumps, and probes that allow for automatic feeding and waste removal can be used for immune cell expansion. Cell culture flasks with gas-permeable membranes at the base may be used for immune cell expansion. Any such system known in the art that enables expansion of immune cells for clinical use is encompassed by the expansion step provided herein. Immune cells are expanded in culture systems in media formulated specifically for expansion. Expansion can also be facilitated by culturing the immune cell of the disclosure in the presence of activation factors as described herein. Those skilled in the art will appreciate that expansion steps, as provided herein, may also encompass any newly discovered culture systems, media, or activating factors that can be used to expand immune cells.

The disclosure provides formulation and cryopreservation steps for the expanded genetically modified subject immune cells. Formulation steps provided include, for example, washing away excess components used in the preparation and expansion of immune cells of the methods of treatment described herein. Any pharmaceutically acceptable formulation medium or wash buffer compatible with immune cell known in the art may be used to wash, dilute/concentration immune cells, and prepare doses for administration. Formulation medium can be acceptable for administration of the immune cells, such as, for example crystalloid solutions for intravenous infusion. Cryopreservation can optionally be used to store immune cells long-term. Cryopreservation can be achieved using known methods in the art, including for example, storing cells in a cryopreservation medium containing cryopreservation components. Cryopreservation components can include, for example, dimethyl sulfoxide or glycerol. Immune cells stored in cryopreservation medium can be cryopreserved by reducing the storage temperature to −80° C. to −180° C.

In some embodiments, the method comprises administering an allogeneic immune cells described herein. In some embodiments, the method comprises administering a conditioning regimen prior to administering the allogeneic immune cells described herein. In some embodiments, the conditioning regimen is lymphodepletion. A lymphodepletion regimen can include, for example, administration of alemtuzumab, cyclophosphamide, benduamustin, rituximab, pentostatin, and/or fludarabine. Lymphodepletion regimen can be administered in one or more cycles until the desired outcome of reduced circulating immune cells.

In some embodiments, the conditioning regimen comprises administering an agent that specifically targets, and reduces or eliminates CD52+ cells in the subject, and the allogeneic immune cells are modified to reduce or eliminate CD52 expression.

In some embodiments, the method of treatment comprises determining the HLA germline type of the subject. In some embodiments, determining the HLA germline type comprises determining the presence of HLA-A*02:01 heterozygosity. In some embodiments, the HLA germline type is determined in bone marrow.

In some embodiments, the method of treatment comprises determining the level of expression of an activator ligand. In some embodiments, the level of expression of an activator ligand is determined in tumor tissue samples from the subject. In some embodiments, the expression level of an activator ligand is determined using next generation sequencing. In some embodiments, the expression level of an activator ligand is determined using RNA sequencing. In some embodiments, the level of an activator ligand is determined using immunohistochemistry.

In some embodiments, the method of treatment comprises administering a therapeutically effective dose of allogeneic immune cells in a subject in need thereof, wherein the subject is determined to be HLA germline HLA-A*02:01 heterozygous and have tumor tissue with activator expression and loss of HLA-A*02:01.

In some embodiments, a therapeutically effective dose of the allogeneic immune cells described herein are administered. In some embodiments, the genetically modified allogeneic immune cells of the disclosure are administered by intravenous injection. In some embodiments, the genetically modified allogeneic immune cells of the disclosure are administered by intraperitoneal injection. In some embodiments, a therapeutically effective dose comprises about 0.5×10⁶ cells, about 1×10⁶ cells, about 2×10⁶ cells, about 3×10⁶ cells, 4×10⁶ cells, about 5×10⁶ cells, about 6×10⁶ cells, about 7×10⁶ cells, about 8×10⁶ cells, about 9×10⁶ cells, about 1×10⁷, about 2×10⁷, about 3×10⁷, about 4×10⁷, about 5×10⁷, about 6×10⁷, about 7×10⁷, about 8×10⁷, about 9×10⁷, 1×10⁸ cells, about 2×10⁸ cells, about 3×10⁸ cells, about 4×10⁸ cells, about 5×10⁸ cells, or about 6×10⁸ cells. In some embodiments, a therapeutically effective dose comprises about 0.5×10⁶ cells to about 6×10⁸ cells, about 1×10⁶ cells to about 5×10⁸ cells, about 2×10⁶ cells to about 5×10⁸ cells, about 3×10⁶ cells to about 4×10⁸ cells, about 4×10⁶ cells to about 3×10⁸ cells, about 5×10⁶ cells to about 2×10⁸ cells, about 6×10⁶ cells to about 1×10⁸ cells, about 7×10⁶ cells to about 9×10⁷ cells, about 8×10⁶ cells to about 8×10⁷ cells, about 9×10⁶ cells to about 7×10⁷ cells, about 1×10⁷ cells to about 6×10⁷ cells, or about 2×10⁷ cells to about 5×10⁷ cells. In some embodiments, a therapeutically effective dose comprises about 0.5×10⁶ cells to about 6×10⁸ cells. The term “about” as referred to in a therapeutically dose, can be, for example, ±0.5×10⁶ cells, ±0.5×10⁷ cells, or ±0.5×10⁸ cells.

In some embodiments, the subject in need thereof has cancer. Cancer is a disease in which abnormal cells divide without control and spread to nearby tissue. In some embodiments, the cancer comprises a liquid tumor or a solid tumor. Exemplary liquid tumors include leukemias and lymphomas. Further cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Exemplary solid tumors include sarcomas and carcinomas. Cancers can arise in virtually an organ in the body, including blood, bone marrow, lung, breast, colon, bone, central nervous system, pancreas, prostate and ovary. Further cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

Any cancer wherein a plurality of the cancer cells express the first, activator ligand and do not express the second, inhibitor ligand is envisaged as within the scope of the instant disclosure. For example, CEA positive cancers that can be treated using the methods described herein include colorectal cancer, pancreatic cancer, esophageal cancer, gastric cancer, lung adenocarcinoma, head and neck cancer, diffuse large B cell cancer or acute myeloid leukemia cancer.

As a further example, EGFR positive cancers that can be treated using the methods described herein include lung cancer, small cell lung cancer, non-small cell lung cancer, pancreatic ductal carcinoma, colorectal cancer, head and neck cancer, esophagus and gastric adenocarcinoma, ovarian cancer, glioblastoma multiforme, cervical squamous cell carcinoma, kidney cancer, papillary kidney cancer, kidney renal clear cell carcinoma, bladder cancer, breast cancer, bile duct cancer, liver cancer, prostate cancer, sarcoma, thyroid cancer, thymus cancer, stomach cancer, or uterine cancer, and all-other EGFR target expressing tumors. The compositions and methods disclosure herein may be used to treating EGFR positive cancers that are relapsed, refractory and/or metastatic.

As a further example, MSNL positive cancers that ca be treated using the methods described herein include mesothelioma cancer, ovarian cancer, cervical cancer, colorectal cancer, esophageal cancer, head and neck cancer, kidney cancer, uterine cancer, gastric cancer, pancreatic cancer, lung cancer, lung adenocarcinomas, colorectal cancer, or cholangiocarcinoma, as well as other solid epithelial tumors. Further cancers that express MSLN include relapsed, refractory or metastatic gastric, esophageal, head and neck and kidney cancers. In some embodiments, the MSLN positive cancer comprises an epithelial tumor, for example a carcinoma.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

### Kits and Articles of Manufacture

The disclosure provides kits and articles of manufacture comprising the polynucleotides and vectors encoding the engineered receptors described herein, and immune cells comprising the engineered receptors described herein (e.g., edited using gene editing systems described herein and/or comprising the engineered receptors described herein). In some embodiments, the kit comprises articles such as vials, syringes and instructions for use.

In some embodiments, the kit comprises a polynucleotide or vector comprising a sequence encoding one or more engineered receptors of the disclosure.

In some embodiments, the kit comprises a plurality of immune cells comprising an engineered receptor as described herein. In some embodiments, the plurality of immune cells comprises a plurality of T cells.

The disclosure provides kits and articles of manufacture comprising the polynucleotides and vectors encoding the interfering RNAs and engineered receptors described herein, and immune cells with reduced or eliminated HLA expression and/or function described herein and comprising the engineered receptors described herein. In some embodiments, the kit comprises articles such as vials, syringes and instructions for use.

In some embodiments, the kit comprises a polynucleotide or vector comprising a sequence encoding the interfering RNAs and/or one or more engineered receptors of the disclosure.

In some embodiments, the kit comprises a plurality of immune cells comprising an interfering RNA and/or engineered receptor as described herein. In some embodiments, the plurality of immune cells comprises a plurality of T cells.

### EXAMPLES

### Example 1: Selection of Activator Target Ligands

The inventors surveyed the GTex gene expression database (gtexportal.org/home/) for activator ligands. Activator ligands should have the following properties: first, One type of activator ligand should have high surface expression, which confers the potential to deliver large activation signals. Alternatively, activators such as MiHAs can have low density on the cell surface. Second, activator ligands can have essential cellular functions, which prevents alleles of the activator ligands being lost due to aneuploidy in tumor cells, and makes them less likely to undergo mutagenesis during the evolution of the tumor. Lastly, activator ligands should be present on all tumor cells. Activator ligands can be expressed on all cells, if the inhibitor ligand is also expressed on all cells except the target cells. Activators should also be expressed on cancer cells. Activators, when used in combination with inhibitors can be widely expressed, for example on all cells.

FIG. 4A shows the RNA expression profile of an exemplary activator ligand, the transferrin receptor (TFRC). As seen in FIG. 4A, expression of TFRC at the RNA level is ubiquitous and relatively even. Further, TFRC is an essential gene: loss of function homozygous TFRC mutations are embryonic lethal in mice.

FIG. 4B shows the expression profiles of a candidate blocker, HLA-A, and candidate activator, HLA-B. As can be seen in FIG. 4B, candidate activator and blocker HLA class I expression tracks together, easing the challenging of optimizing activator and blocker pairs.

### Example 2: Selection of Inhibitor Target Ligands that are Lost in Cancer Cells

### Loss of Heterozygosity

One potential pool of inhibitor ligands are ligands that are lost in tumor cells due to loss of heterozygosity. In an analysis of 3131 tumor samples across 26 histological types, Beroukhim et al. found that in a typical tumor, 25% of the genome is affected by arm number single copy number alterations (duplications and deletions), and 10% of the genome is affected by focal single copy number alterations, with 2% overlap. (Beroukhim et al, Nature 463:899-905 (2010)). Further, many of the LOH regions overlap between tumor types, and only 22% of regions are unique to one tumor type. For example, Beroukhim et al. found that 80% of amplification peaks and 78% of deletion peaks were common to the 17 most represented tumor types. Thus, alleles that are lost to LOH that can be selectively bound by an inhibitor LBD are potential inhibitor targets that are not expressed by target cells.

The inventors surveyed the Cancer Genome Atlas Program (http://portals.broadinstitute.org/tcga/home) for potential inhibitor ligands that were lost in cancers through loss of heterozygosity. The dataset all_cancers dataset (all_cancers) consisted of 10,844 cancer samples from 33 cancer types. One type of inhibitor ligands should have the following properties: first, inhibitor ligands should have high, homogeneous surface expression across tissues. This confers the ability to deliver a large, even-handed inhibitory signal. Inhibitor ligands should be absent or polymorphic in many tumors. Further, it should be easy to distinguish loss of the inhibitor ligand in tumor cells via conventional methods such as antibody stains or genetic analysis. Other types of inhibitor ligands, such as MiHAs, can have low surface expression.

One pool of inhibitor ligands are major histocompatibility complex (MHC) alleles that are lost through LOH in cancer cells. Use of these alleles as inhibitor ligands does not require a peptide MHC target (pMHC), for example, a pan HLA-A*02 allele can be used.

### Loss of Y Chromosome

Adult male-expressed Y chromosome genes are potential inhibitor ligands through loss of Y chromosome. There are at least 60 protein coding genes on the Y chromosome. Several Y chromosome genes are expressed broadly in adult males and may be lost in cancers via loss of Y chromosome. Several other broadly expressed cytoplasmic proteins are pMHC inhibitor candidates (e.g., TMSB4Y, EIF1AY). NLGN4Y is a Type I integral membrane protein expressed broadly in males, and also a candidate.

### Example 3: Targeting Cells Lacking Surface Antigen with Paired A and B Receptors

We show that the targeting system for loss of heterozygosity works in vitro and in a mouse cancer model.

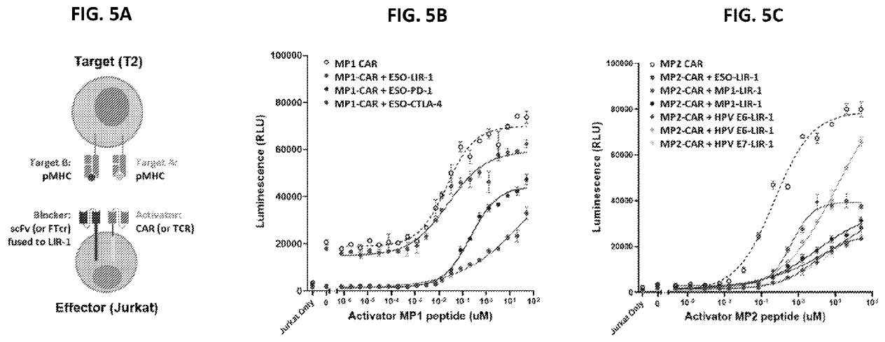

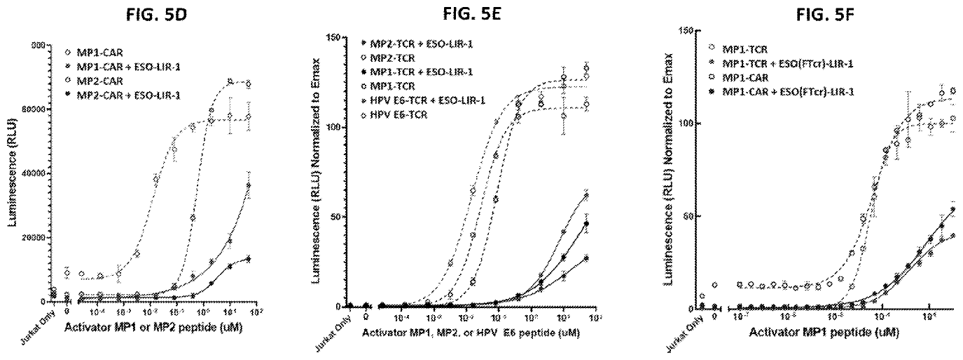

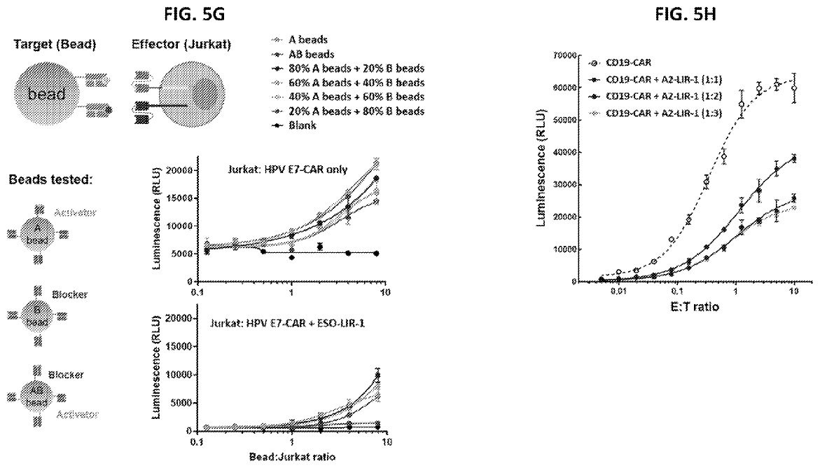

Discrimination between normal and tumor cells depends on two functions: (i) an activator (“A”) receptor that recognizes an epitope on the surface of normal cells that is also retained on the tumor; and, (ii) a blocker (“B”) receptor that recognizes a second surface epitope on an allelic product lost from the tumor cell. In this Example, we used peptide-MHC (pMHC) targets for both the A and B (see FIG. 5A):
a chimeric antigen receptor comprising an scFv against HLA-A*02-MAGE-A3 (FLWGPRALV) pMHC as the A receptor; and
a chimeric antigen receptor comprising an scFv that binds HLA-A*02-NY-ESO-1 (SLLMWITQC/V) as the B receptor and comprising a PD-1 intracellular domain (ICD), a CTLA-4 intracellular domain (ICD) or a LILRB1 (LIR1) intracellular domain (ICD).

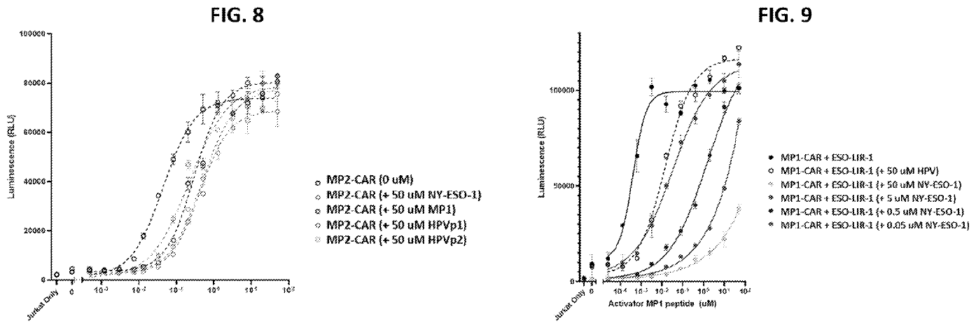

Each blocker (B) receptor, with a PD-1 ICD or a CTLA-4-ICD, mediated a shift in EC50 of activation in Jurkat cells of <10×, measured by titration of peptides loaded on T2 cells as stimulus (FIG. 5B). Surprisingly, B receptors comprising a NY-ESO-1 LBD and the intracellular, transmembrane and hinge domains of the LIR-1 (LILRB1) receptor mediated an EC50 shift of >5,000× (also FIG. 5B). Titration of unrelated control HLA-A*02-binding peptides other than SLLMWITQC/V provided an estimate of the shift caused by competition of loaded peptides on T2 cells for available HLA molecules, a contribution to the total shift typically <10× (FIG. 8). For the EC50 shift values reported here, we typically compare to EC50s of activator-only constructs.

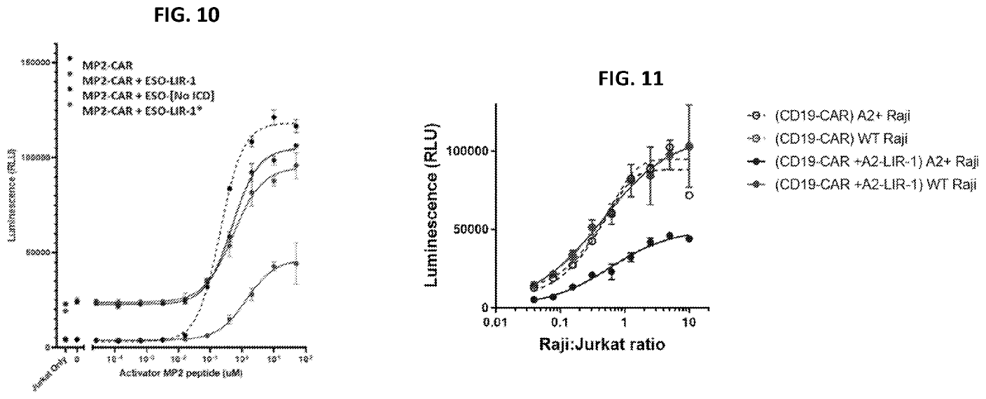

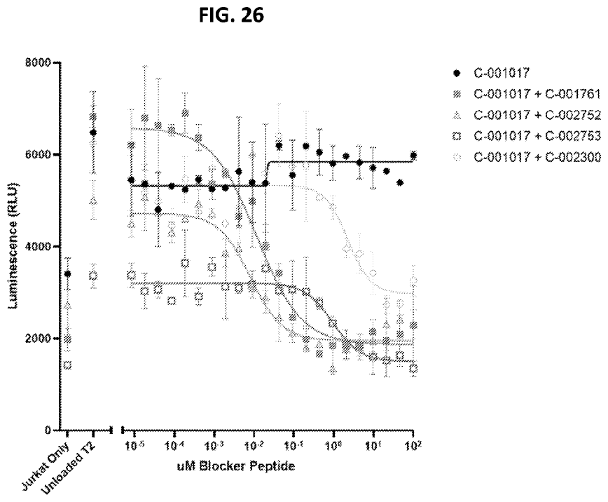

For four different pMHC targets, a total of six different scFvs grafted onto LIR-1 mediated dramatic shifts in EC50, ranging from 10 to 1,000× (FIG. 5C). The degree of EC50 shift (i.e., blocking strength) correlated with the EC50 of the scFv when fused to a standard CAR (data not shown). LIR-1B signaling blocked A signaling from multiple A targets and scFvs (FIG. 5D, FIG. 26). The blockade was ligand-dependent (FIG. 9). Control B receptors with a LBD, but which lack an ICD or contain mutations in key elements of the ICD do not block activation by A receptor signal. (FIG. 10). Engineered T cells with A receptor and B receptor function across multiple target antigens and antigen binding domains (i.e., LBD sequences).

The LIR-1 ICD also functions when fused to a T cell receptor (TCR) extracellular domain with three different pMHC targets (see Methods). TCRs against three different pMHC targets, two from MAGE-A3 and one form HPV, were assayed. In every case, a LIR-1 based B receptor shifted the activation EC50 by large amounts, ranging from 1,000 to 10,000×. LIR-1 based B receptors with an NY-ESO-1 TCR variable domain LBD “ESO (Ftcr)” fused to it were also able to block activation by a CAR or TCR. This included the following receptor pairs

1. An activator (A) TCR comprising a TCR LBD (“MP1-TCR”) that binds MAGE-A3_{FLWGPRALV} peptide:MHC complexes was blocked by a B receptor comprising an scFv NY-ESO-1 scFv LBD (“ESO”) and a LIR-1 ICD, which shifted the activation EC50 by a large amount (FIG. 5E);
2. An A TCR comprising a second TCR LBD (“MP2-TCR”) that binds the MAGE-A3_{MPKVAELVHFL} peptide:MHC complexes was blocked by a B receptor comprising an scFv NY-ESO-1 scFv LBD (“ESO”) and a LIR-1 ICD, which shifted the activation EC50 by a large amount (FIG. 5E);
3. An A TCR comprising a TCR LBD (“HPV E6-TCR”) that binds an HPV_{TIHDIILECV} peptide:MHC complex was blocked by a B receptor comprising an scFv NY-ESO-1 scFv LBD (“ESO”) and a LIR-1 ICD, which shifted the activation EC50 by large amounts (FIG. 5E);
4. An A TCR comprising a TCR LBD (“MP1-TCR”) that binds a MAGE-A3_{FLWGPRALV} peptide:MHC complexes was blocked by a B receptor comprising an NY-ESO-1 TCR LBD (“ESO(Ftcr)”), and a LIR-1 ICD, This blocker shifted the activation EC50 by large amounts (FIG. 5F);
5. An A CAR comprising a scFv LBD (“MP1-CAR”) that binds the MAGE-A3_{FLWGPRALV} peptide:MHC complexes was blocked by a B receptor comprising an TCR NY-ESO-1 TCR variable domain LBD (“ESO(Ftcr)”), and a LIR-1 ICD. This blocker shifted the activation EC50 by large amounts (FIG. 5F).

### Confirming Cis Effect

Engineered effector cells should discriminate potential target cells that are A+ only, i.e. display only the activator, from those that are dual A+ and B+. To affirm our receptor system works as intended, target-loaded beads roughly the size of cells (d˜2.8 μm) were tested with engineered effector cells (Jurkat cells) having A receptor and B receptor (FIG. 5G). Effector cells were indeed activated by a mixture of A+ and B+ beads, even when the A+ beads comprised only 20% of the total beads. This confirms effector cells are able to recognize targets having loss of heterozygosity (represented by A+ beads) in a mixed population comprising normal cells (represented by B+ beads).

### Confirming Target Concentration Independence

In patients, target density will vary depending on the expression levels of the A target and B target. We confirmed the system works with both high density and low density targets, both when A target density is varied (data not shown) and when B target density is varied (FIG. 5H) FIG. 5H, scFvs that bound either the B-cell marker CD19 or HLA-A*02 in a peptide-independent fashion were tested. These non-pMHC targets represent surface antigens that can extend into the realm of 100,000 epitopes/cell. In this case, the ratio of A to B module expression was varied using different DNA concentrations in transient transfection assays. Emax shifts of over 10× were observed. These experiments showed that the properties of the dual receptor system observed for pMHC targets were generally the same for high-density targets.

### B Receptor Function in Primary T Cells

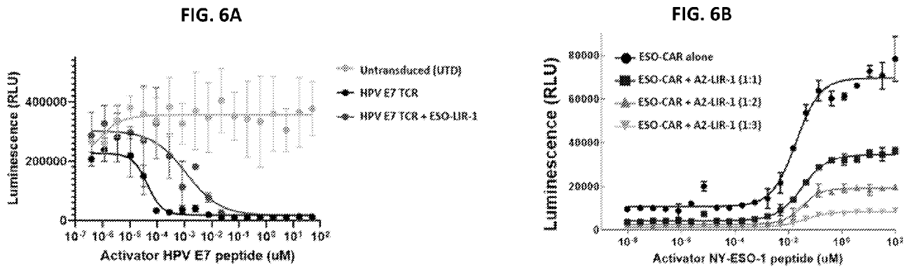

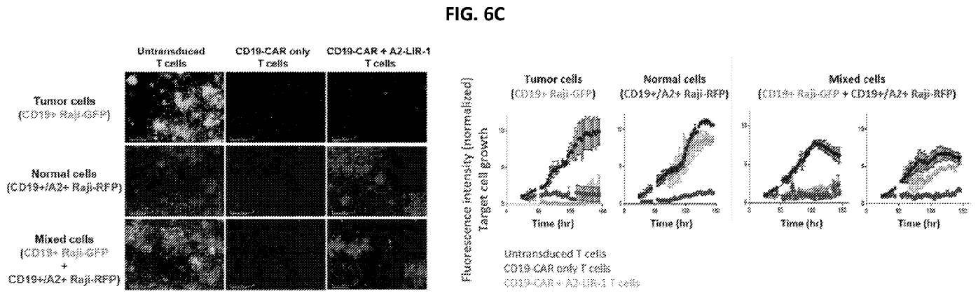

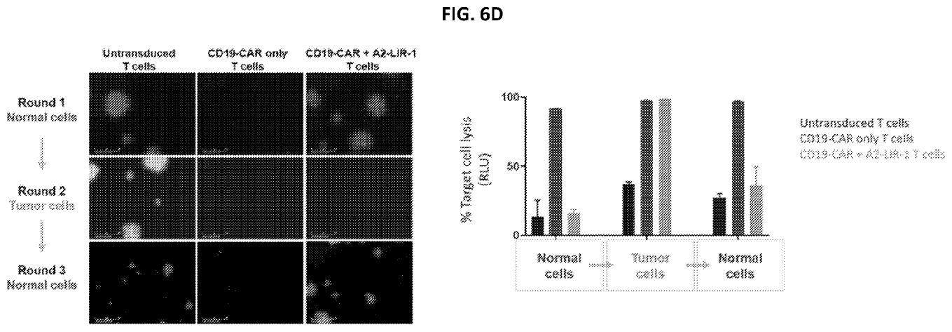

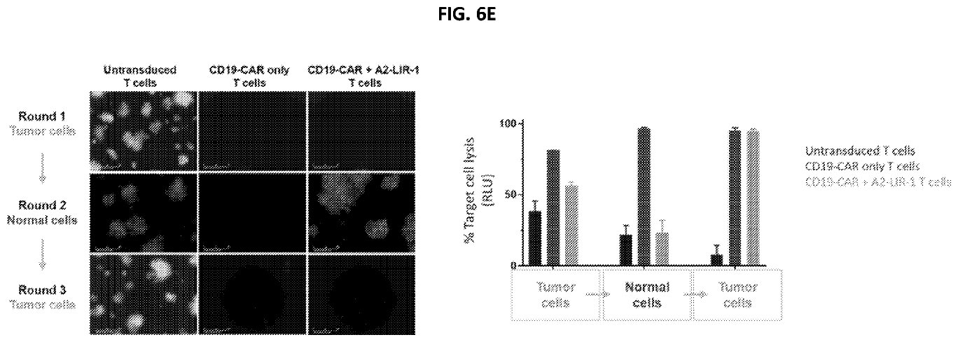

MCF7 tumor cells expressing renilla luciferase (Biosettia) loaded with a titration of target peptide were used as target cells, with the luciferase as the readout for cell viability. Primary T cells were transduced with an HPV TCR as the A receptor (“HPV E7 TCR”) and a B receptor comprising an anti-NY-ESO-1 scFv fused to a LIR-1 hinge, transmembrane domain and ICD (“ESO-LIR-1”), or not transduced (“Untransduced”). Transduced T cells were enriched via physical selection using beads coupled to HLA-A*02 tetramers that bind to the B receptor LBD. To vary target concentration, the target cells were loaded with varying amounts of HPV peptide. Primary T cells were activated in a dose dependent matter. Expression of the B receptor shifted the EC50 curve by ˜100× (FIG. 6A). A similar result was obtained for an anti-NY-ESO-1 CAR A receptor paired with B receptor comprising an anti-HLA-A*02 LBD and an LIR-1 hinge, transmembrane domain and ICD at various ratios of A receptor to B receptor (achieved by transfecting various activator:blocker DNA ratios) in Jurkat cells (FIG. 6B). This result was confirmed with a CD19 CAR activator paired with an HLA-A*02 blocker in T cells (FIG. 6C). Thus, the basic function of the activator and blocker receptor pair was reproduced in primary T cells, despite their complexity, heterogeneity and donor-to-donor variability.

### Example 4: Targeting Loss of Heterozygosity with Paired A and B Receptors

The HLA locus is polymorphic with only a subset of the population having the HLA-A*02 allele. A ligand-binding domain that binds MHC of the HLA*A02 allele independent of loaded peptide (a “pan HLA-A*02” LBD) may be used to target tumors in subjects heterozygous for HLA and having LOH of the HLA-A*02 allele in tumor cells.

An HLA-A*02-specific scFv was fused to the LIR-1 module and shown to function as a blocker in the presence of pMHC-dependent activators (ESO-CAR, FIG. 6B) in Jurkat cells. Furthermore, in primary T cells expressing both an A receptor comprising an anti-CD19 scFv and a B receptor comprising an HLA-A*02-specific scFv and a LIR-1 LBD, the B receptor blocked the A receptor as desired (FIG. 6C).

Raji target cells that are CD19+ and negative for HLA-A*02 can be used to model tumor cells that have lost HLA-A*02 through LOH. The same cell line stably expressing HLA-A*02 can be used as a model of normal cells. Raji cell lines activated Jurkat effector cells expressing a CD19 CAR and the HLA-A*02 LIR-1 blocker if the Raji target cells expressed CD19 only. When the Raji target cells were transfected with a polynucleotide encoding HLA-A*02, activation of the Jurkat effector cells was blocked (FIG. 11).

As described above, the A receptor binding CD19 and B receptor binding HLA-A*02 worked in primary T cells as well as Jurkat cells. Engineered T cells killed CD19-expressing Raji cells in the absence of HLA-A*02 expression (FIG. 6C, upper panels). Raji cells that expressed both CD19 and HLA-A*02 were killed by T cells expressing only the activating module, but blocked from both gamma-interferon (IFNg) secretion (data not shown) and cytotoxicity when co-cultured with T cells expressing both activator and blocker modules (FIG. 6C, middle panels). Primary T cells bearing the activator and blocker modules distinguished CD19+“tumor” (FIG. 6C, lower panels) from CD19+/HLA-A*02+“normal” cells (FIG. 6C, right panels) in a mixed culture.

A T cell therapeutic based on the activator and blocker mechanism should be able to function reversibly, i.e. be able to cycle from a state of blockade to activation and back to blockade. Effector cells were co-cultured with multiple rounds of Raji cells that were either CD19+ or D19+/HLA-A2*02+, which were removed from culture between rounds. As desired, effector cells exposed to normal cells were not activated when exposed to Raji target cells for both block-kill-block (FIG. 6D) and kill-block-kill programs of target cell exposure (FIG. 6E). Effector T cells were able to cycle from a state of block to cytotoxicity and back, depending on the target cells to which they were exposed.

### Example 5: In Vivo Targeting of Loss of Heterozygosity with Paired A and B Receptors

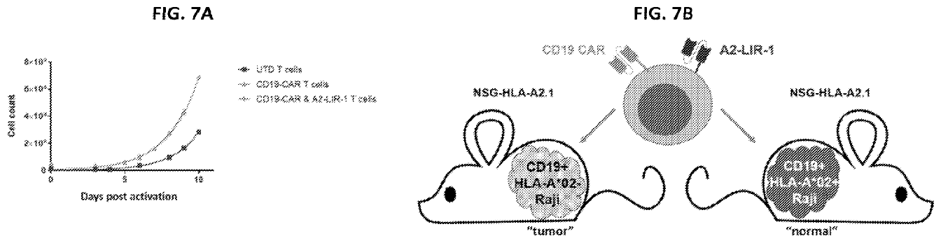

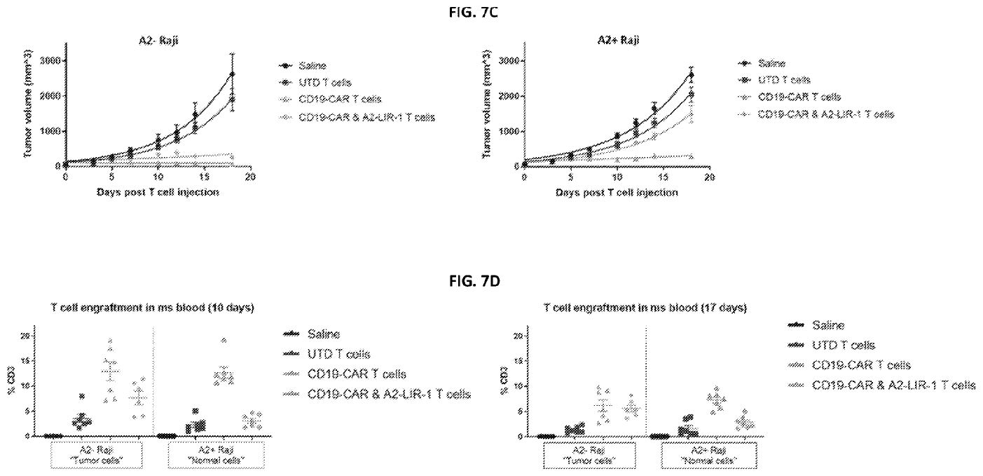

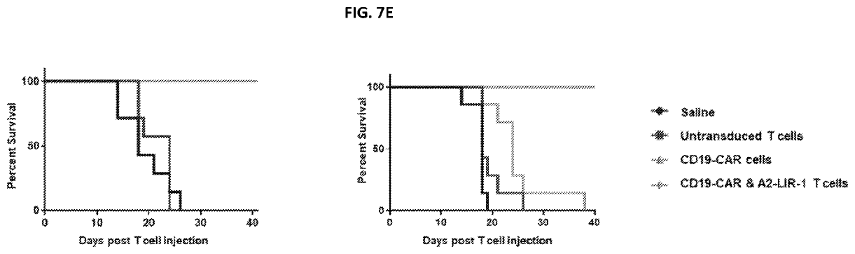

To prepare for in vivo experiments, we showed that the CD19/HLA-A*02 activator/blocker pair engineered in primary T cells allowed expansion in vitro to large numbers using standard CD3/CD28 stimulation (FIG. 7A). Thus, a cell product can be produced in sufficient quantity for use in patients as a therapeutic.

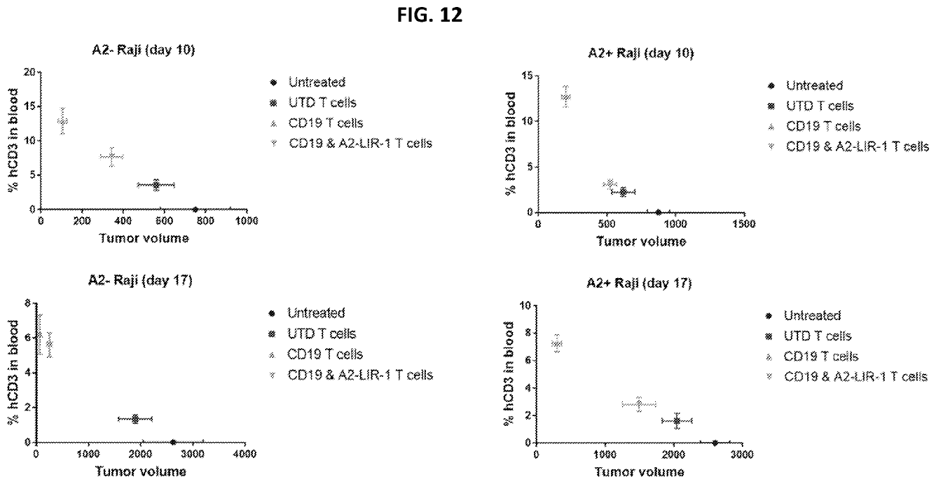

A CD19+/HLA-A*02+ or CD19+/HLA-A*02− tumor cell mouse xenograft was generated by injecting Raji target cells into the flanks of immunocompromised (NGS-HLA-A2.1) mice (FIG. 7B). Raji cells were injected at two doses, 2e6 or 1e7 T cells, and the growth of the tumor and the persistence of the implanted T cells were analyzed over time. Only CD19+/HLA-A*02− tumor cells were killed in the mouse and the tumor control tracked with transferred T cell numbers, promoting survival of the host mice (FIGS. 7C-7E and FIG. 12). Normal CD19+/HLA-A*02+ cells designed to model normal cells were unaffected by treatment.

### SUMMARY

We have developed a synthetic signal integration system that can take advantage of a large, new class of cancer targets derived from LOH. The system, without undue experimentation, meets requirements of a cell therapy for patients with LOH. The system works robustly in Jurkat cells, primary T cells, and in vivo. This system is also (i) modular and flexible, and works across CAR and TCR modalities with different target densities; (ii) silenced when the blocker and activator targets are present on one surface in cis, but not when a minority of cells expresses only the activator; and, (iii) switches states reversibly, consistent with the need to hunt tumor cells throughout the body.

### Example 6: Methods for Examples 3-5

### Cell Culture

Jurkat cells encoding an NFAT Luciferase reporter were obtained from BPS Bioscience. All other cell lines used in this study were obtained from ATCC. In culture, Jurkat cells were maintained in RPMI media supplemented with 10% FBS, 1% Pen/Strep and 0.4 mg/mL G418/Geneticin. T2, MCF7, and Raji cells were maintained as suggested by ATCC. “Normal” Raji cells were made by transducing Raji cells with HLA-A*02 lentivirus (custom lentivirus, Alstem) at a MOI of 5. HLA-A*02-positive Raji cells were sorted using a FACSMelody Cell Sorter (BD).

### Plasmid Construction

The NY-ESO-1-responsive inhibitory construct was created by fusing the NY-ESO-1 scFv LBD to domains of receptors including hinge, transmembrane region, and/or intracellular domain of leukocyte immunoglobulin-like receptor subfamily B member 1, LILRB1 (LIR-1), programmed cell death protein 1, PDCD1 (PD-1), or cytotoxic T-lymphocyte protein 4, CTLA4 (CTLA-4). Gene segments were combined using Golden Gate cloning and inserted downstream of a human EFla promoter contained in a lentiviral expression plasmid.

### Jurkat Cell Transfection

Jurkat cells were transiently transfected via 100 uL format Neon electroporation system (Thermo Fisher Scientific) according to manufacturer's protocol using the following settings: 3 pulses, 1500V, 10 msec. Cotransfection was performed with 1-3 ug of activator CAR or TCR construct and 1-3 ug of either scFv or Ftcr blocker constructs or empty vector per 1e6 cells and recovered in RPMI media supplemented with 20% heat-inactivated FBS and 0.1% Pen/Strep.

### Jurkat-NFAT-Luciferase Activation Studies

Peptides, MAGE-A3 (MP1; FLWGPRALV), MAGE-A3 (MP2; MPKVAELVHFL), HPV E6 (TIHDIILECV), HPV E7 (YMLDLQPET) and modified NY-ESO-1 ESO (ESO; SLLMWITQV), were synthesized by Genscript. Activating peptide was serially diluted starting at 50 uM. Blocker peptide, NY-ESO-1, was diluted to 50 uM (unless otherwise indicated) which was added to the activating peptide serial dilutions and subsequently loaded onto 1e4 T2 cells in 15 uL of RPMI supplemented with 1% BSA and 0.1% Pen/Strep and incubated in Corning® 384-well Low Flange White Flat Bottom Polystyrene TC-treated Microplates. The following day, 1e4 Jurkat cells were resuspended in 15 uL of RPMI supplemented with 10% heat-inactivated FBS and 0.1% Pen/Strep, added to the peptide-loaded T2 cells and cocultured for 6 hours. ONE-Step Luciferase Assay System (BPS Bioscience) was used to evaluate Jurkat luminescence. Assays were performed in technical duplicates.

### Primary T Cell Transduction, Expansion, and Enrichment

Frozen PBMCs were thawed in 37° C. water bath and cultured at 1e6 cells/mL in LymphoONE (Takara) with 1% human serum and activated using 1:100 of T cell TransAct (Miltenyi) supplemented with IL-15 (long/mL) and IL-21 (1 Ong/mL). After 24 hours, lentivirus was added to PBMCs at a MOI of 5. PBMCs were cultured for 2-3 additional days to allow cells to expand under TransAct stimulation. Post expansion, activator and blocker transduced primary T cells were enriched using anti-PE microbeads (Miltenyi) according to manufacturer's instructions. Briefly, primary T cells were incubated with CD19-Fc (R&D Systems) at 1:100 dilution for 30 minutes at 4° C. in MACS buffer (0.5% BSA+2 mM EDTA in PBS). Cells were washed 3 times in MACS buffer and incubated in secondary antibody (1:200) for 30 minutes at 4° C. in MACS buffer. Cells were then incubated in anti-PE microbeads and passed through the LS column (Miltenyi).

### Primary T Cell In Vitro Cytotoxicity Studies

For cytotoxicity studies with pMHC targets, enriched primary T cells were incubated with 2e3 MCF7 cells expressing renilla luciferase (Biosettia) loaded with a titration of target peptide as described above at an effector:target ratio of 3:1 for 48 hours. Live luciferase-expressing MCF7 cells were quantified using a Renilla Luciferase Reporter Assay System (Promega). For cytotoxicity studies with non-pMHC targets, enriched primary T cells were incubated with 2e3 WT Raji cells (“tumor” cells) or HLA-A*02 transduced Raji cells (“normal” cells) at an effector:target ratio of 3:1 for up to 6 days. WT “tumor” Raji cells stably expressing GFP and renilla luciferase (Biosettia) or HLA-A*02 “normal” Raji cells were stably expressing RFP and firefly luciferase (Biosettia) were imaged together with unlabeled primary T cells using an IncuCyte live cell imager. Fluorescence intensity of live Raji cells over time was quantified using IncuCyte imaging software. For reversibility studies, enriched primary T cells were similarly cocultured with “normal” or “tumor” Raji cells for 3 days and imaged. After 3 days, T cells were separated from remaining Raji cells using CD19 negative selection and reseeded with fresh “normal” or “tumor” Raji cells as described. In separate wells, live luciferase-expressing Raji cells were quantified using a Dual-Luciferase Reporter Assay System (Promega).

### Mouse Xenograft Study

Frozen PBMCs were thawed in 37° C. water bath and rested overnight in serum-free TexMACS Medium (Miltenyi) prior to activation. PBMCs were activated in 1.5e6 cells/mL using T cell TransAct (Miltenyi) and TexMACS Medium supplemented with IL-15 (20 ng/mL) and IL-21 (20 ng/mL). After 24 hours, lentivirus was added to PBMCs at a MOI of 5. PBMCs were cultured for 8-9 additional days to allow cells to expand under TransAct stimulation. Post expansion, T cells were enriched on A2-LIR-1 (pMHC HLA-A*02 ScFv fused to a LIR-1 hinge, TM and ICD) using anti-PE microbeads (Miltenyi) against streptavidin-PE-HLA-A*02− pMHC prior to in vivo injection.

5-6 week old female NOD.Cg-Prkdcscid I12rgtm1Wjl Tg(HLA-A/H2-D/B2M)1Dvs/SzJ (NSG-HLA-A2/HHD) mice were purchased from The Jackson Labs. Animals were acclimated to the housing environment for at least 3 days prior to the initiation of the study. Animals were injected with 2e6 WT Raji cells or HLA-A*02 transduced Raji cells in 100 uL volume subcutaneously in the right flank. When tumors reached an average of 70 mm³ (V=L×W×W/2), animals were randomized into 5 groups (n=7) and 2e6 (data not shown) or 1e7 T cells were administered via the tail vein. Post T cell injection, tumor measurements were performed 3 times per week and blood was collected 10 days and 17 days after for flow analysis. Post RBC lysis, cells were stained with anti-hCD3 antibody, anti-hCD4 antibody, anti-hCD8 antibody, anti-msCD45 antibody (Biolegend).

### Example 7

The ability of a blocker receptor with an HLA-A-A*02 antigen binding domain and a LIR-1 ICD (C1765) to block activation of Jurkat cells expressing an activator CAR with an EGFR antigen binding domain (CT479) was assayed using the NFAT-luciferase reporter system as previously described. Wild type HeLa tumor cells, which were EGFR+ and HLA-A*02−, were used as target cells. EGFR+/HLA-A*02− HeLa cells were also transduced with a polynucleotide encoding HLA-A*02+ to generate EGFR+/HLA-A*02+ HeLa cells to use as target cells expressing both activator and blocker antigens.

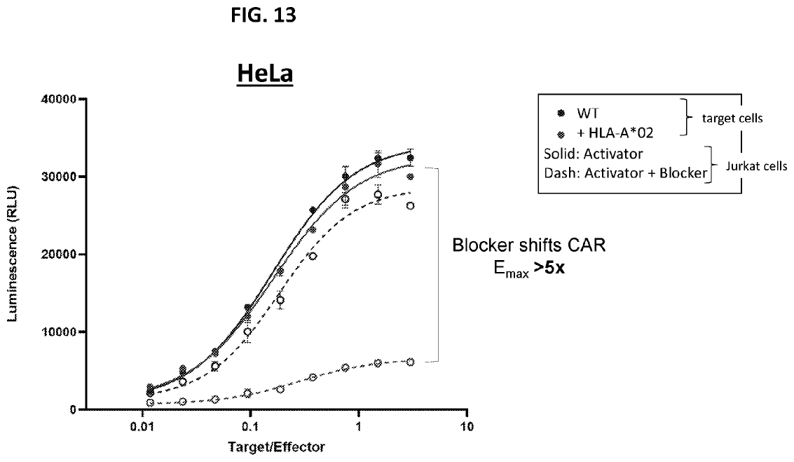

As shown in FIG. 13, expression of the HLA-A*02 LIR-1 blocker in Jurkat cells expressing the EGFR CAR shifts the CAR E_{MAX} by >5 fold compared to the CAR Emax of Jurkat cells that do not express the blocker.

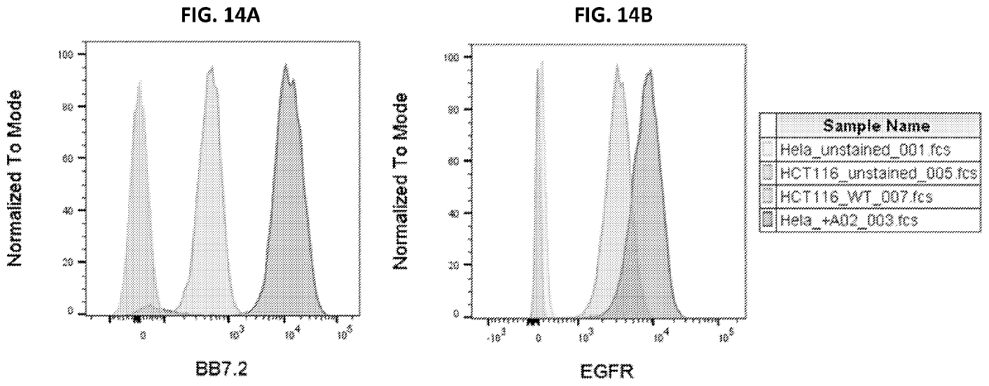

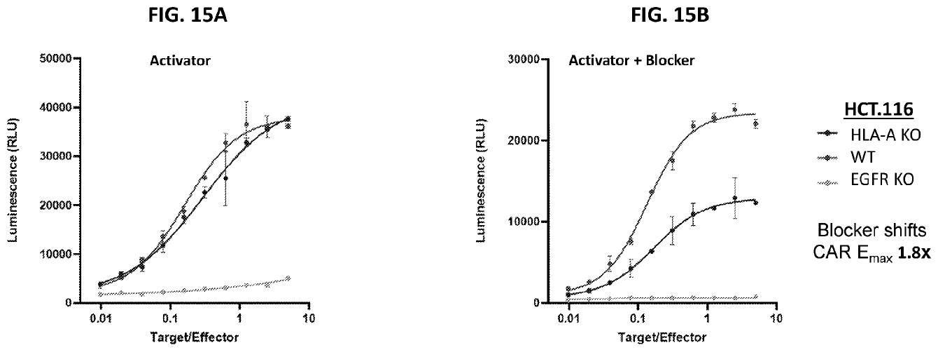

Furthermore, lower blocking was observed with lower HLA-A2 expression levels on target cells. Wild type HCT116 cells are EGFR+ and HLA-A*02. The levels of EGFR and HLA-A*02 were assayed in HCT116 cells and HeLa cells transduced with polynucleotides encoding the HLA-A*02 polynucleotides using an anti-EGFR antibody and an anti-HLA-A*02 antibody (BB7.2) followed by FACs sorting. As shown in FIGS. 14A & 14B, HCT116 cells have lower levels of blocker HLA-A*02 antigen than transduced HeLa cells. When Jurkat cells expressing the EGFR CAR and HLA-A*02 LIR-1 blocker were presented with HCT116 target cells expressing EGFR and HLA-A*02 antigens, presence of the HLA-A*02 LIR-1 blocker shifted the E_{MAX} of the EGFR CAR 1.8 fold (FIG. 15B). In contrast, transduced HeLa cells, which expressed a higher level of HLA-A*02 antigen, were able to mediate an EGFR CAR E_{MAX} shift of >5 fold (FIG. 13). As a control, there was minimal activation by EGFR knockout HCT116 cells (FIG. 15A).

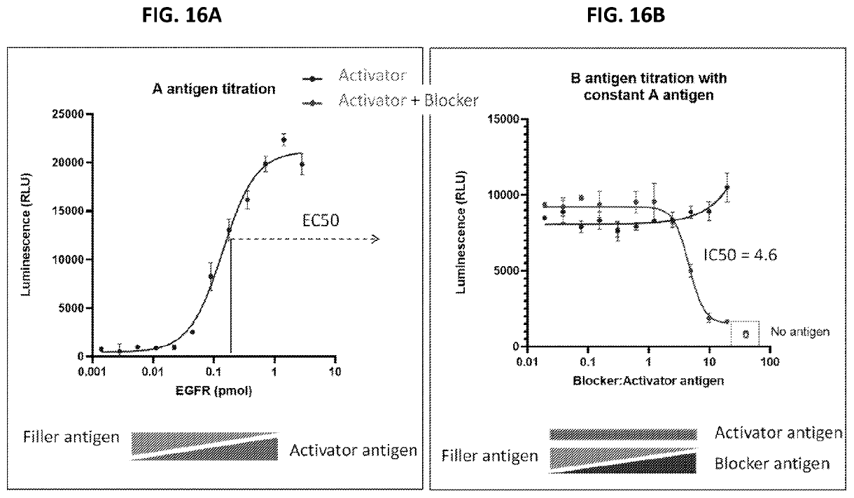

The ratio of blocker to activator necessary to achieve 50% blocking using the EGFR CAR and HLA-A*02 LIR-1 blocker was assayed using a bead based system, which is shown in FIGS. 16A and 16B.

To determine the EC50 of the activator antigen, activator beads were coated with activator antigen at different concentrations. An irrelevant protein was added to each concentration so that the total protein concentration was the same, and a constant amount of beads was added to Jurkat effector cells expressing the EGFR CAR (FIG. 16A).

To determine blocker antigen IC50, beads were coated with activator antigen at the EC50 concentration (determined in FIG. 16A), and coated with blocker antigen at different concentrations. An irrelevant protein was added at each concentration so that the total protein concentration remained the same, and a constant amount of beads was added to Jurkat effector cells expressing either the EGFR CAR or the EGFR CAR and the HLA-A*02 LIR-1 blocker (FIG. 16B).

### Example 8: LIR-1 Based Blockers can Inhibit TCR Signaling Using a Solid Tumor Cell Line

Jurkat effector cells expressing a MAGE-A3 activator TCR and a NY-ESO-1 scFv LIR-1 based inhibitory receptor (comprising a LIR-1 hinge, TM and ICD), were assayed using A375 target cells loaded with different concentrations of activator and blocker peptides. Jurkat cell activation was assayed using an NFAT luciferase assay (see Example 6).

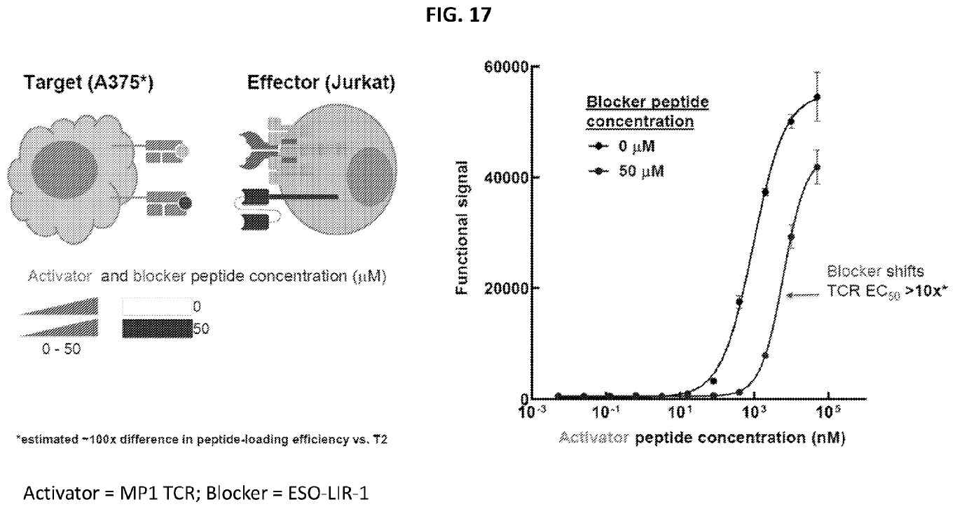

As shown in FIG. 17, loading A375 cells with 50 μM NY-ESO-1 peptide shifted the activator TCR E_{MAX} greater than 10 fold. There is an estimated ˜100× difference in peptide loading efficiency in A375 cells versus T2 target cells. Peptide loading may account for the apparent therapeutic window.

### Example 9: HLA-A*02 LIR-1 Based Blockers can Inhibit CAR Signaling Using a B Cell Leukemia Cell Line

Jurkat effector cells expressing the non-pMHC, high density CD19 specific activator (CD19 scFv CAR activator), with or without co-expression of a pMHC HLA-A*02 scFv LIR-1 based inhibitory receptor (comprising a LIR hinge, TIM and ICD), were assayed using NALM6 target cells. Jurkat cell activation was assayed using an NFAT luciferase assay (see Example 6), and the effector to target cell (E:T) ratio was varied.

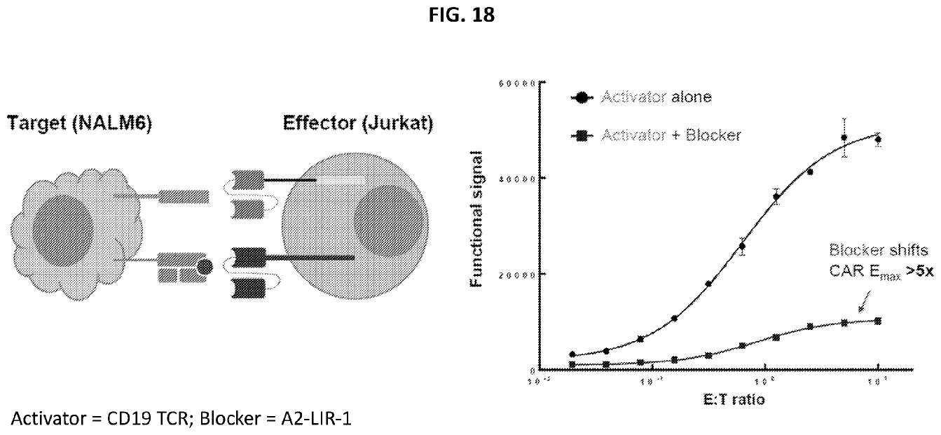

As shown in FIG. 18, expression of the blocker by Jurkat cells was able to shift the E_{MAX} of the CAR by greater than 5 fold.

### Example 10: HLA-A*02 LIR-1 Based Blockers can Inhibit CAR Signaling in a Dose Dependent Manner

Jurkat effector cells expressing an NY-ESO-1 scFv CAR, and a pMHC HLA-A*02 scFv LIR-1 based inhibitory receptor, were assayed using T2 target cells loaded with varying amounts of peptide (note, in this case the same peptide is recognized by both the activator and blocker ScFv). Jurkat cell activation was assayed using an NFAT luciferase assay (see Example 6). Jurkat cells were transfected with varying ratios of activator to blocker DNA, i.e. 1:1, 1:2 and 1:3 activator to blocker, to vary the ratios of the receptors expressed by the Jurkat cells.

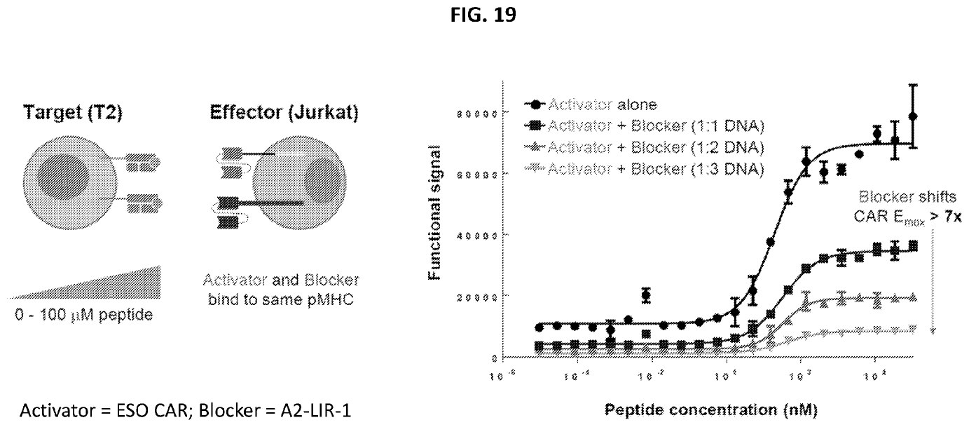

As can be seen in FIG. 19, even with Jurkat cells transfected with activator and blocker receptor DNA at a ratio of 1:1, the MHC HLA-A*02 scFv LIR-1 based inhibitory receptor (blocker) was able to inhibit activation of Jurkat cells by the activator CAR. Furthermore, the degree to which the inhibitory receptor blocked activation increased with an increased amount of inhibitory receptor DNA compared to activator receptor DNA used in Jurkat cell transfection.

### Example 11: HLA-A*02 LIR-1 Based Blockers can Inhibit a Universal (Pan HLA Class I) Activator with Tunable Strengths

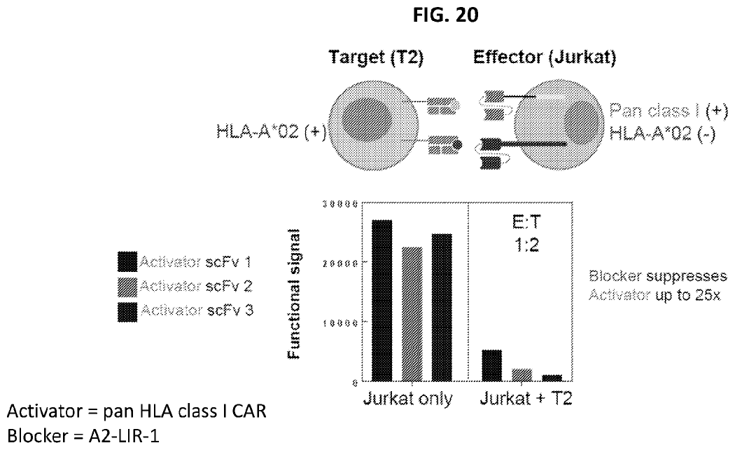

Activation of Jurkat effector cells expressing pan HLA scFv CARs with three different scFv binding domains based on the pan HLA antibody W6/32, and a pMHC HLA-A*02 scFv LIR-1 based inhibitory receptor were assayed using HLA-A*02 positive T2 cells. As can be seen from FIG. 20, each activator scFv supported a different functional signal in HLA-A*02− negative Jurkat cells. The pMHC HLA-A*02 scFv LIR-1 based inhibitory receptor was able to block functional signal from all three pan HLA scFv CARs when Jurkat cells were contacted with HLA-A*02-positive T2 target cells at an E:T ratio of 1:2. Moreover, the pMHC HLA-A*02 scFv LIR-1 based inhibitory receptor was able to suppress the activator up to 25 fold.

### Example 12: HLA-A*02 LIR-1 Based Inhibitory Receptors can Block Activation by an MSLN CAR Activator

Activation of Jurkat effector cells expressing an MSLN CAR activator and a pMHC HLA-A*02 ScFv LIR-1 based inhibitory receptor was assayed using the NFAT Luciferase assay described in Example 6.

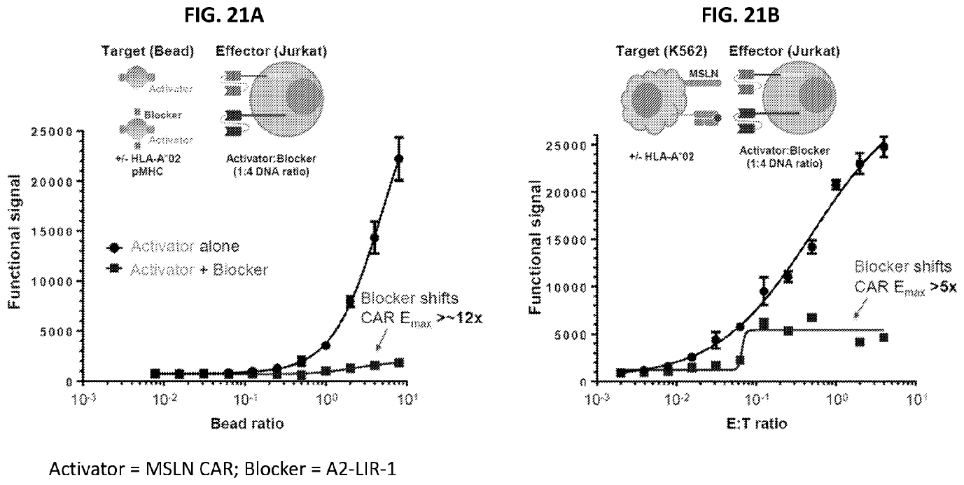

Jurkat cells were transfected with activator: blocker DNA at a ratio of 1:4, and activation was assayed in a cell-free bead based assay (FIG. 21A). Beads were loaded with either activator antigen, or activator and blocker antigens, and the ratio of beads to Jurkat cells was varied. In the cell-free bead based assay, the pMHC HLA-A*02 ScFv LIR-1 based inhibitory receptor was able to block activation of the Jurkat cells when cells were contacted with beads carrying the pMHC HLA-A*02 blocker and MSLN activator in cis. Presence of the pMHC HLA-A*02 blocker on the beads was able to shift E_{MAX} of MSLN CAR by greater than or equal to 12X (FIG. 21A).

Activation Jurkat cells transfected with the same activator and blocker at a 1:4 DNA ratio were assayed for activation using the chronic myelogenous leukemia cell line K562. K562 expresses MLSN, the activator antigen. The response of Jurkat effector cells to K562 cells transduced with HLA-A*02 to express both activator and blocker antigens (MSLN+ HLA-A*02+) and untransduced K562 (MSLN+ HLA-A*02−) that expressed the activator but not the blocker antigen was assayed. As can be seen in FIG. 21B, expression of HLA-A*02+ by the K562 cells was able to shift the MSLN CAR E_{MAX} by greater than 5X.

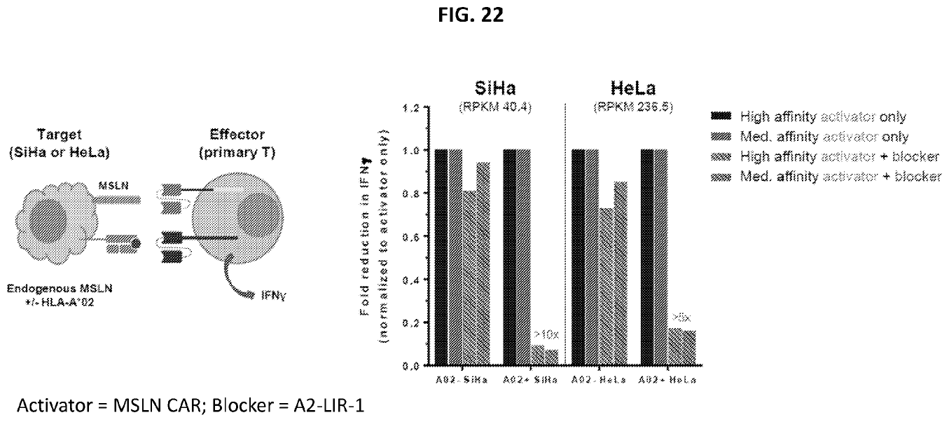

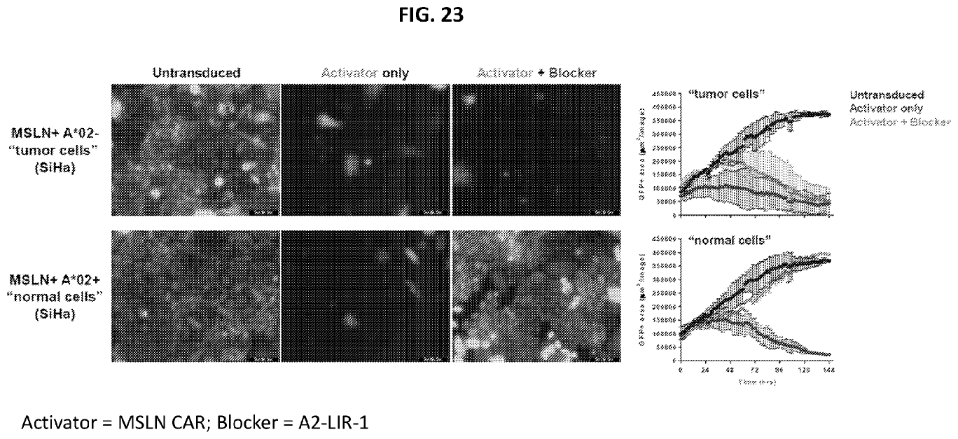

The ability of the pMHC HLA-A*02 inhibitory receptor to block activation via the MSNL ScFv CAR was also assayed using effector primary T cells and SiHa or HeLa target cells as described for Raji in Example 6. SiHa and HeLa cells endogenously express MSLN, and were transduced to express the HLA-A*02 inhibitory receptor target. Activation of primary effector T cells was assayed by looking at fold induction of IFNγ. As shown in FIG. 22, the pMHC HLA-A*02 LIR-1 inhibitory receptor was able to block activation of primary T cells when the primary T cells were presented with SiHa or HeLa target cells expressing HLA-A*02 (greater than 10X and 5X inhibition, respectively).

The pMHC HLA-A*02 inhibitory receptor was also able to inhibit killing by T cells expressing both the MSLN ScFv CAR and the pMHC HLA-A*02 LIR-1 inhibitory receptor, when the T cells were presented with SiHa cells that expressed MSLN but not HLA-A*02 (FIG. 23).

### Example 13: HLA-A*02 LIR-1 Based Inhibitory Receptors can Block Activation by an EGFR CAR Activator

Activation of Jurkat effector cells expressing an EGFR CAR activator and a pMHC HLA-A*02 ScFv LIR-1 based inhibitory receptor (comprising a LIR-1 hinge, transmembrane and ICD) was assayed using the NFAT Luciferase assay described in Example 6.

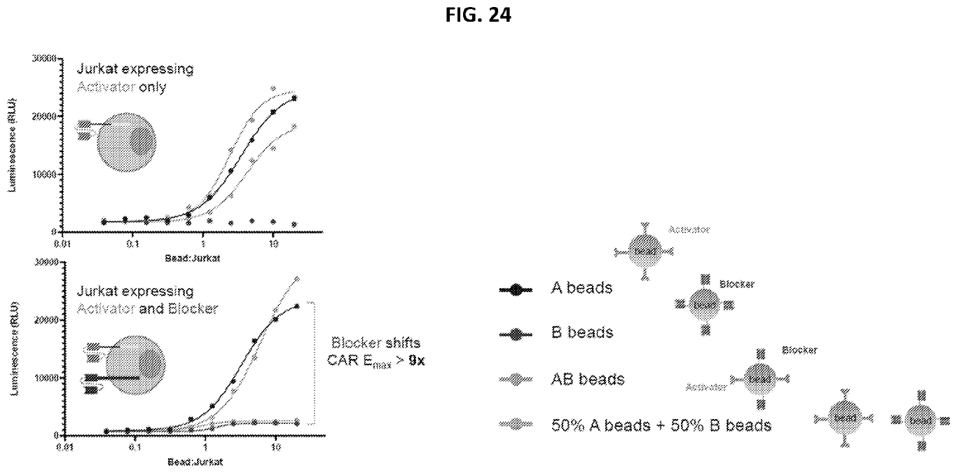

Jurkat cells were transfected with activator and blocker receptor DNA, and activation was assayed in a cell-free bead based assay (FIG. 24). Beads were loaded with either activator antigen, blocker antigen, or activator and inhibitor antigens, and the ratio of beads to Jurkat cells was varied. In the cell-free bead based assay, the HLA-A*02 ScFv LIR-1 based inhibitory receptor was able to block activation of the Jurkat cells when cells were contacted with beads carrying the HLA-A*02 blocker and EGFR activator in cis, but not when the HLA-A*02 blocker and EGFR activator were in trans (on different beads). Presence of the HLA-A*02 blocker on the beads was able to shift E_{MAX} of EGFR CAR by greater than or equal to 9X (FIG. 24).

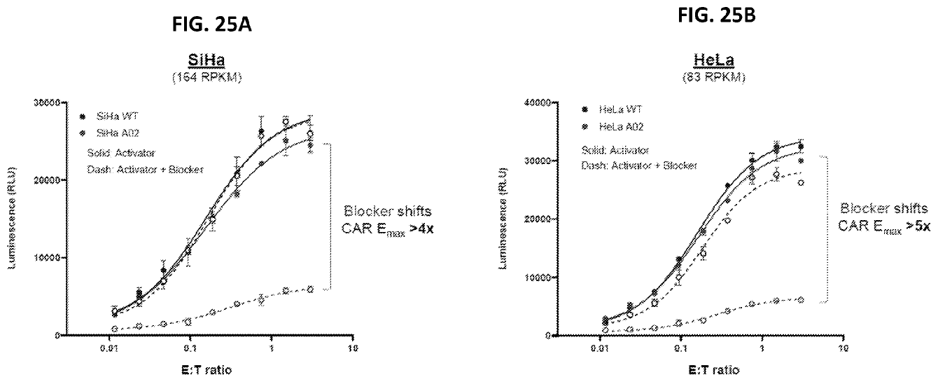

Activation of Jurkat cells expressing the EGFR CAR activator and a HLA-A*02 ScFv LIR-1 based inhibitory receptor was also assayed using HeLa and SiHa cells as target cells. Wild type HeLa and SiHa cell lines express EGFR but not HLA-A*02 (SiHa WT and HeLa WT), but were transduced to express the HLA-A*02 inhibitory receptor target (SiHa A02 and HeLa A02). As can be seen in FIGS. 25A-25B, the HLA-A*02 ScFv LIR-1 based inhibitory receptor was able to shift the EGFR E_{MAX} by greater than 4X using SiHa target cells (FIG. 25A) and greater than 5X using HeLa target cells (FIG. 25B).

### Example 14: Activator and Blocker Pairs can Discriminate Between KRAS Alleles

MiHAs are peptides derived from proteins that contain nonsynonymous differences between alleles. Using KRAS as a model for MiHAs, the activator and blocker pairs were able to discriminate and respond to different KRAS variants using antigen binding domains specific to the KRAS G12V and KRAS G12D mutations.

Using the Jurkat-NFAT-luciferase activation studies described in Example 6 and T2 target cells, the ability of KRAS ScFv or Ftcr inhibitory LIR-1 based receptors to inhibit activation mediated by an activator KRAS CAR or TCR was assayed.

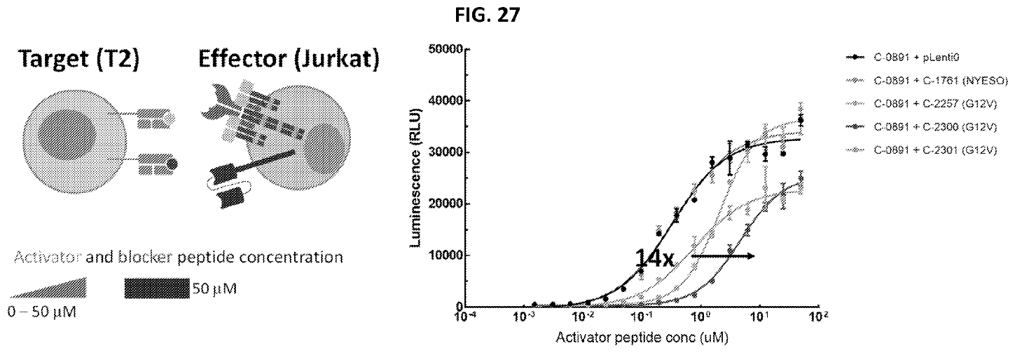

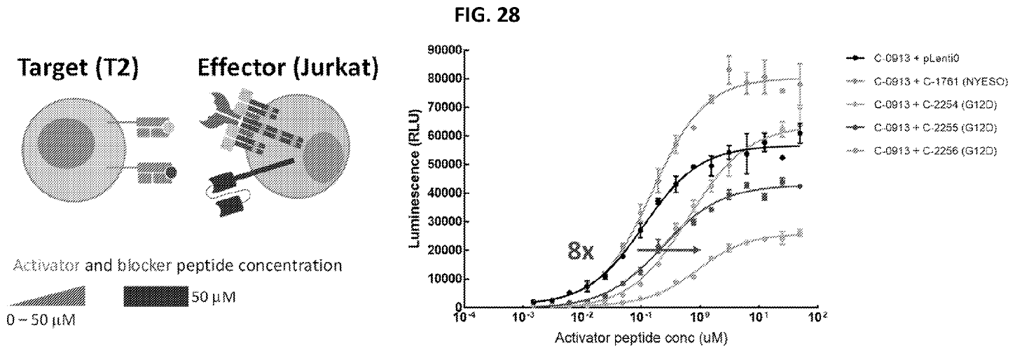

FIG. 27 shows that a KRAS G12V scFv blocker was able to inhibit activation of Jurkat cells by a KRAS G12D TCR (C-891) and shift the KRAS G12D E_{Max} by 14X. FIG. 28 shows a similar result with a reciprocal pair, a KRAS G12D ScFv blocker and a KRAS G12V TCR activator (C-913), where the inhibitor was able to shift the KRAS G12V E_{Max} by 8X.

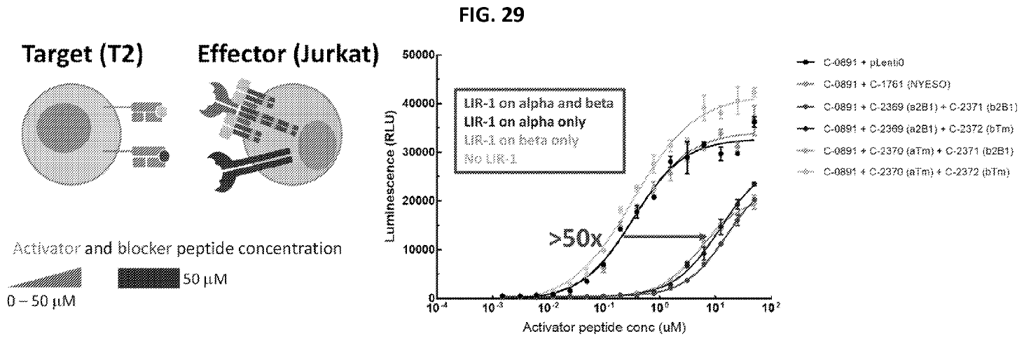

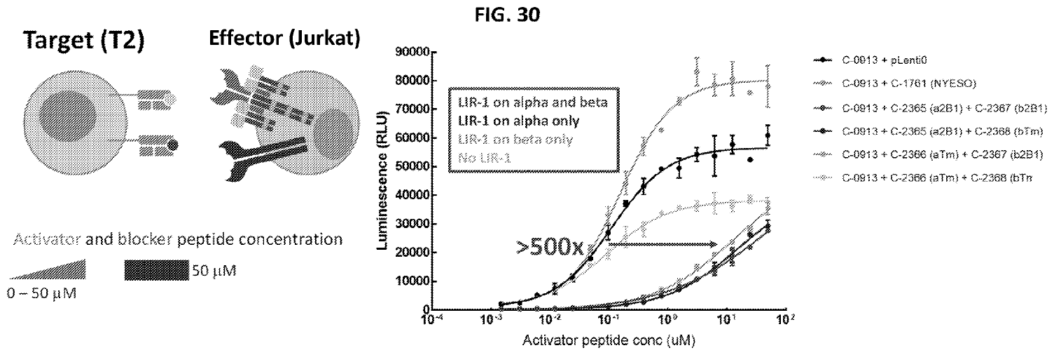

FIG. 29 shows that a KRAS G12V Ftcr blocker was able to inhibit a KRAS G12D TCR. The inhibitor was able to shift the KRAS G12D E_{Max} by greater than 50X. In this case, constructs with a LIR-1 transmembrane domain and intracellular domain were included on both the alpha and beta chains of the inhibitory TCR (LIR-1 on alpha and beta), on the TCR alpha chain only (LIR-1 on alpha only), on the TCR beta chain only (LIR-1 on beta only), and a version with no LIR-1 ICD was included as a control (no LIR-1). In a reciprocal experiment, a KRAS G12D Ftcr blocker was able to inhibit a KRAS G12V activator TCR, shifting the KRAS G12V E_{Max} by greater than 500X.

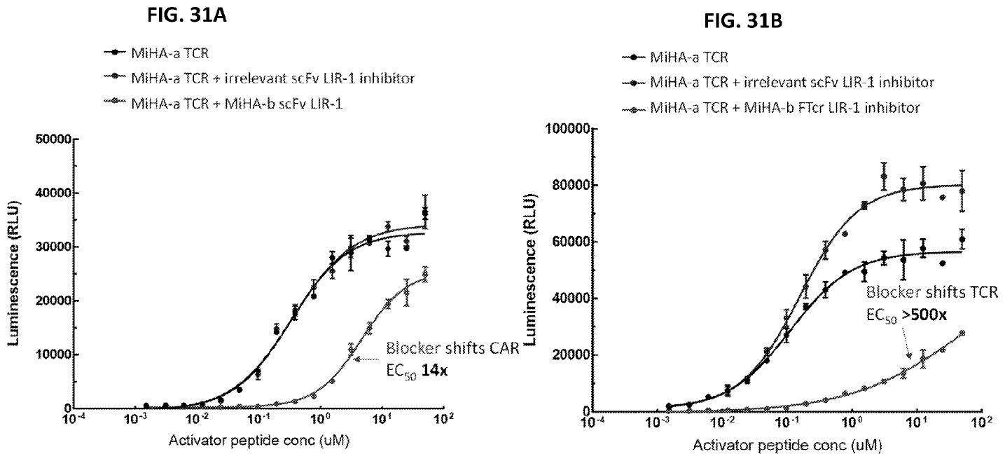

Finally, this effect was dependent on the specific ligand binding domains, as pairs with an inhibitory receptor that had an irrelevant ScFv domain had little effect on activator E_{Max} (FIGS. 31A-31B).

Table 4 lists the KRAS ScFv and Ftcr sequences. All ScFvs were fused to a LIR-1 hinge, TM and ICD. All Ftcrs were fused to a LIR-1 TM and ICD.

TABLE 4
KRAS ScFv and Ftcr Sequences.

| C-02256 (Kp33A1101_H125 scFv): | C-02256 |
|---|---|
| QVQLVESGGGLVKPGGSLRLSCAASGFTFSDYYMSWIRQAPGKGLE | (Kp33A1101_H125 |
| WVSYISSSGSTIYYADSVKGRFTISRDNAKNSLYLQMNSLRAEDTAV | scFv) DNA |
| YYCARDFTRDYYYYYYMDVWGKGTTVTVSSGGGGSGGGGSGGG | Sequence: SEQ |
| GSGGDIQMTQSPSSLSASVGDRVTITCRASQSISSYLNWYQQKPGKA | ID NO: 229 |
| PKLLIYAASSLQSGVPSRFSGSGSGTDFTLTISSLQPEDFATYYCQQS | |
| YSTPLTFGGGTKVEIK (SEQ ID NO: 228) | |
| C-02257 (K14A11:01_V001_scFv): | C-02257 |
| QVTLRESGPALVKPTQTLTLTCTFSGFSLSTSGMCVSWIRQPPGKAL | (K14A11:01_V001_ |
| EWLALIDWDDDKYYSTSLKTRLTISKDTSKNQVVLTMTNMDPVDT | scFv) DNA |
| ATYYCARSYDELYYFDYWGQGTLVTVSSGGGGSGGGGSGGGGSG | Sequence: SEQ |
| GDIQMTQSPSSLSASVGDRVTITCRASQSISSYLNWYQQKPGKAPKL | ID NO: 231 |
| LIYAASSLQSGVPSRFSGSGSGTDFTLTISSLQPEDFATYYCQQSYSTP | |
| LTFGGGTKVEIK (SEQ ID NO: 230) | |
| C-002300 (K14A11:01 H001/L004_scFv): | C-002300 |
| QVTLRESGPALVKPTQTLTLTCTFSGFSLSTSGMCVSWIRQPPGKAL | (K14A11:01 |
| EWLALIDWDDDKYYSTSLKTRLTISKDTSKNQVVLTMTNMDPVDT | H001/L004_scFv) |
| ATYYCARSYDELYYFDYWGQGTLVTVSSGGGGSGGGGSGGGGSG | DNA |
| GDIQMTQSPSSLSASVGDRVTITCRASQSIWTSYLNWYQQKPGKAP | Sequence: SEQ |
| KLLIYAASSLQSGVPSRFSGSGSGTDFTLTISSLQPEDFATYYCQQSY | ID NO: 232 |
| STPLTFGGGTKVEIK | |
| (SEQ ID NO: 21890) | |
| C-002301 (K14A11:01 H001/L010 scFv); | C-002301 |
| QVTLRESGPALVKPTQTLTLTCTFSGFSLSTSGMCVSWIRQPPGKAL | (K14A11:01 |
| EWLALIDWDDDKYYSTSLKTRLTISKDTSKNQVVLTMTNMDPVDT | H001/L010 |
| ATYYCARSYDELYYFDYWGQGTLVTVSSGGGGSGGGGSGGGGSG | scFv) DNA |
| GDIQMTQSPSSLSASVGDRVTITCRASQSISSYLNWYQQKPGKAPKL | Sequence: SEQ |
| LIYAASSLQSGVPSRFSGSGSGTDFTLTISSLQPEDFATYYCQQSYST | ID NO: 233 |
| RLTFGGGTKVEIK (SEQ ID NO: 21891) | |
| C-002365 [pLenti 1 K33A1101_V002 TCRa T48C (G12D TRAV4- | C-002365 |
| 4/DV10*01)]: | [pLenti 1 |
| MQRNLGAVLGILWVQICWVRGDQVEQSPSALSLHEGTDSALRCNF | K33A1101_V002 |
| TTTMRSVQWFRQNSRGSLISLFYLASGTKENGRLKSAFDSKERRYST | TCRa T48C |
| LHIRDAQLEDSGTYFCAADSSNTGYQNFYFGKGTSLTVIPNIQNPEP | (G12D TRAV4- |
| AVYQLKDPRSQDSTLCLFTDFDSQINVPKTMESGTFITDKCVLDMK | 4/DV10*01)] |
| AMDSKSNGAIAWSNQTSFTCQDIFKETNATYPSSDVPCDATLTEKSF | DNA Sequence: |
| ETDMNLNFQNLS (SEQ ID NO: 234) | SEQ ID NO: |
| | 235 |
| C-002367 [pLenti 1 K33A1101_V002 TCRb S51C | C-002367 |
| (TRBV12-2*01)]: | [pLenti 1 |
| MSNTAFPDPAWNTTLLSWVALFLLGTSSANSGVVQSPRYIIKGKGE | K33A1101_V002 |
| RSILKCIPISGHLSVAWYQQTQGQELKFFIQHYDKMERDKGNLPSRF | TCRb S51C |
| SVQQFDDYHSEMNMSALELEDSAVYFCASSLTDPLDSDYTFGSGTR | (TRBV12- |
| LLVIEDLRNVTPPKVSLFEPSKAEIANKQKATLVCLARGFFPDHVEL | 2*01)] DNA |
| SWWVNGKEVHSGVCTDPQAYKESNYSYCLSSRLRVSATFWHNPRN | Sequence: |
| HFRCQVQFHGLSEEDKWPEGSPKPVTQNISAEAWGRADCGITSASY | SEQ ID NO: |
| QQGVLS | 237 |
| (SEQ ID NO: 236) | |
| C-002368 [pLenti 1 K33A1101_V002 TCRb S51C | C-002368 |
| (TRBV12-2*01)]: | [pLenti 1 |
| MSNTAFPDPAWNTTLLSWVALFLLGTSSANSGVVQSPRYIIKGKGE | K33A1101_V002 |
| RSILKCIPISGHLSVAWYQQTQGQELKFFIQHYDKMERDKGNLPSRF | TCRb S51C |
| SVQQFDDYHSEMNMSALELEDSAVYFCASSLTDPLDSDYTFGSGTR | (TRBV12- |
| LLVIEDLRNVTPPKVSLFEPSKAEIANKQKATLVCLARGFFPDHVEL | 2*01)] DNA |
| SWWVNGKEVHSGVCTDPQAYKESNYSYCLSSRLRVSATFWHNPRN | Sequence: SEQ |
| HFRCQVQFHGLSEEDKWPEGSPKPVTQNISAEAWGRADCGITSASY | ID NO: 238 |
| QQGVLS (SEQ ID NO: 21892) | |
| C-002369 [pLenti 1 Kp514A1101_V001 TCRa T48C (G12 V | C-002369 |
| TRAV3-3*01)]: | [pLenti 1 |
| MKTVTGPLFLCFWLQLNCVSRGEQVEQRPPHLSVREGDSAVITCTY | Kp514A1101_ |
| TDPNSYYFFWYKQEPGASLQLLMKVFSSTEINEGQGFTVLLNKKDK | V001 TCRa |
| RLSLNLTAAHPGDSAAYFCAVSGGTNSAGNKLTFGIGTRVLVRPDI | T48C (G12 V |
| QNPEPAVYQLKDPRSQDSTLCLFTDFDSQINVPKTMESGTFITDKCV | TRAV3-3*01)] |
| LDMKAMDSKSNGAIAWSNQTSFTCQDIFKETNATYPSSDVPCDATL | DNA Sequence: |
| TEKSFETDMNLNFQNLS | SEQ ID NO: |
| (SEQ ID NO: 239) | 240 |
| C-002371 [pLenti 1 Kp514A1101_V001 TCRb S51C (TRBV4*01)]: | C-002371 |
| MGCRLLSCVAFCLLGIGPLETAVFQTPNYHVTQVGNEVSFNCKQTL | [pLenti 1 |
| GHDTMYWYKQDSKKLLKIMFSYNNKQLIVNETVPRRFSPQSSDKA | Kp514A1101_ |
| HLNLRIKSVEPEDSAVYLCASSRDWGPAEQFFGPGTRLTVLEDLRN | V001 TCRb |
| VTPPKVSLFEPSKAEIANKQKATLVCLARGFFPDHVELSWWVNGKE | S51C |
| VHSGVCTDPQAYKESNYSYCLSSRLRVSATFWHNPRNHFRCQVQF | (TRBV4*01)] |
| HGLSEEDKWPEGSPKPVTQNISAEAWGRADCGITSASYHQGVLS | DNA Sequence: |
| (SEQ ID NO: 241) | SEQ ID NO: |
| | 242 |
| C-002372 [pLenti 1 Kp514A1101_V001 TCRb S51C (TRBV4*01)]: | C-002372 |
| MGCRLLSCVAFCLLGIGPLETAVFQTPNYHVTQVGNEVSFNCKQTL | [pLenti 1 |
| GHDTMYWYKQDSKKLLKIMFSYNNKQLIVNETVPRRFSPQSSDKA | Kp514A1101_ |
| HLNLRIKSVEPEDSAVYLCASSRDWGPAEQFFGPGTRLTVLEDLRN | V001 TCRb |
| VTPPKVSLFEPSKAEIANKQKATLVCLARGFFPDHVELSWWVNGKE | S51C |
| VHSGVCTDPQAYKESNYSYCLSSRLRVSATFWHNPRNHFRCQVQF | (TRBV4*01)] |
| HGLSEEDKWPEGSPKPVTQNISAEAWGRADCGITSASYHQGVLS | DNA Sequence: |
| (SEQ ID NO: 21893) | SEQ ID NO: |
| | 243 |

TABLE 4
Ftcr sequences fused to LIR-1 TM and no ICD as controls.

| C-002366 [pLenti 1 K33A1101_V002 TCRa T48C (G12D TRAV4- | C-002366 |
|---|---|
| 4/DV10*01)]: | [pLenti 1 |
| MQRNLGAVLGILWVQICWVRGDQVEQSPSALSLHEGTDSALRCNF | K33A1101_V002 |
| TTTMRSVQWFRQNSRGSLISLFYLASGTKENGRLKSAFDSKERRYST | TCRa T48C |
| LHIRDAQLEDSGTYFCAADSSNTGYQNFYFGKGTSLTVIPNIQNPEP | (G12D TRAV4- |
| AVYQLKDPRSQDSTLCLFTDFDSQINVPKTMESGTFITDKCVLDMK | 4/DV10*01)] |
| AMDSKSNGAIAWSNQTSFTCQDIFKETNATYPSSDVPCDATLTEKSF | DNA Sequence: |
| ETDMNLNFQNLS (SEQ ID NO: 21894) | SEQ ID NO: |
| | 244 |
| C-002370 [pLenti 1 Kp514A1101_V001 TCRa T48C (G12 V | C-002370 |
| TRAV3-3*01)]: | [pLenti 1 |
| MKTVTGPLFLCFWLQLNCVSRGEQVEQRPPHLSVREGDSAVITCTY | Kp514A1101_ |
| TDPNSYYFFWYKQEPGASLQLLMKVFSSTEINEGQGFTVLLNKKDK | V001 TCRa |
| RLSLNLTAAHPGDSAAYFCAVSGGTNSAGNKLTFGIGTRVLVRPDI | T48C (G12 V |
| QNPEPAVYQLKDPRSQDSTLCLFTDFDSQINVPKTMESGTFITDKCV | TRAV3-3*01)] |
| LDMKAMDSKSNGAIAWSNQTSFTCQDIFKETNATYPSSDVPCDATL | DNA Sequence: |
| TEKSFETDMNLNFQNLS (SEQ ID NO: 21895) | SEQ ID NO: |
| | 245 |

### Example 15: Characterization of TCRs Recognizing a MiHA-Y

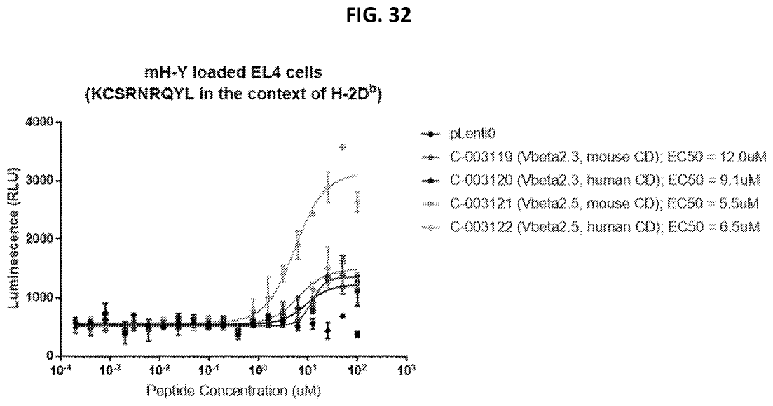

TCR alpha and beta extracellular domains from Jb2.3 and P2A mouse TCRs were cloned into activator TCR constructs. Either the native mouse or human constant regions were used. EL5 cells loaded with mH-Y H-2D^{b} peptide KCSRNRQYL (SEQ ID NO: 246) were used as target cells to assay the activation of Jurkat cells transfected with the MiHA-Y TCRs (FIG. 32). As can be seen from FIG. 32, C-003121 supports robust Jurkat cell activation.

TABLE 5
Mouse miHA-Y TCR sequences with mouse or human constant regions

| | |
|---|---|
| C-003119 (H-Y TCRalpha P2A TCRbeta Jb2.3 mouse); | C-003119 |
| MFPVTILLLSAFFSLRGNSAQSVDQPDAHVTLSEGASLELRCSYSYSAA | (H-Y |
| PYLFWYVQYPGQSLQFLLKYITGDTVVKGTKGFEAEFRKSNSSFNLKK | TCRalpha |
| SPAHWSDSAKYFCALEGQDQGGSAKLIFGEGTKLTVSSPDIQNPEPAV | P2A |
| YQLKDPRSQDSTLCLFTDFDSQINVPKTMESGTFITDKTVLDMKAMDS | TCRbeta |
| KSNGAIAWSNQTSFTCQDIFKETNATYPSSDVPCDATLTEKSFETDMN | Jb2.3 mouse) |
| LNFQNLSVMGLRILLLKVAGFNLLMTLRLWSSRAKRSGSGATNFSLLK | DNA |
| QAGDVEENPGPMSNTAFPDPAWNTTLLSWVALFLLGTKHMEAAVTQ | Sequence: |
| SPRNKVAVTGGKVTLSCNQTNNHNNMYWYRQDTGHGLRLIHYSYGA | SEQ ID NO: |
| GSTEKGDIPDGYKASRPSQENFSLILELATPSQTSVYFCASGDNSAETL | 248 |
| YFGPGTRLTVLEDLRNVTPPKVSLFEPSKAEIANKQKATLVCLARGFFP | |
| DHVELSWWVNGKEVHSGVSTDPQAYKESNYSYCLSSRLRVSATFWH | |
| NPRNHFRCQVQFHGLSEEDKWPEGSPKPVTQNISAEAWGRADCGITSA | |
| SYHQGVLSATILYEILLGKATLYAVLVSGLVLMAMVKKKNS | |
| (SEQ ID NO: 247) | |
| C-003120 (H-Y TCRalpha T48C P2A H-Y TCRbeta Jb2.3 S57C | C-003120 |
| human); | (H- |
| MFPVTILLLSAFFSLRGNSAQSVDQPDAHVTLSEGASLELRCSYSYSAA | YTCRalpha |
| PYLFWYVQYPGQSLQFLLKYITGDTVVKGTKGFEAEFRKSNSSFNLKK | T48C P2A |
| SPAHWSDSAKYFCALEGQDQGGSAKLIFGEGTKLTVSSPYIQNPDPAV | H-Y |
| YQLRDSKSSDKSVCLFTDFDSQTNVSQSKDSDVYITDKCVLDMRSMD | TCRbeta |
| FKSNSAVAWSNKSDFACANAFNNSIIPEDTFFPSPESSCDVKLVEKSFET | Jb2.3 S57C |
| DTNLNFQNLSVIGFRILLLKVAGFNLLMTLRLWSSGSGATNFSLLKQA | human) DNA |
| GDVEENPGPMSNTAFPDPAWNTTLLSWVALFLLGTKHMEAAVTQSPR | Sequence: |
| NKVAVTGGKVTLSCNQTNNHNNMYWYRQDTGHGLRLIHYSYGAGST | SEQ ID NO: |
| EKGDIPDGYKASRPSQENFSLILELATPSQTSVYFCASGDNSAETLYFGP | 250 |
| GTRLTVLEDLKNVFPPEVAVFEPSEAEISHTQKATLVCLATGFYPDHVE | |
| LSWWVNGKEVHSGVCTDPQPLKEQPALNDSRYCLSSRLRVSATFWQN | |
| PRNHFRCQVQFYGLSENDEWTQDRAKPVTQIVSAEAWGRADCGFTSE | |
| SYQQGVLSATILYEILLGKATLYAVLVSALVLMAMVKRKDSRG (SEQ | |
| ID NO: 249) | |
| C-003121 (H-Y TCRalpha P2A TCRbeta Jb2.3L mouse); | C-003121 |
| MFPVTILLLSAFFSLRGNSAQSVDQPDAHVTLSEGASLELRCSYSYSAA | (H-Y |
| PYLFWYVQYPGQSLQFLLKYITGDTVVKGTKGFEAEFRKSNSSFNLKK | TCRalpha |
| SPAHWSDSAKYFCALEGQDQGGSAKLIFGEGTKLTVSSPDIQNPEPAV | P2A |
| YQLKDPRSQDSTLCLFTDFDSQINVPKTMESGTFITDKTVLDMKAMDS | TCRbeta |
| KSNGAIAWSNQTSFTCQDIFKETNATYPSSDVPCDATLTEKSFETDMN | Jb2.3L |
| LNFQNLSVMGLRILLLKVAGFNLLMTLRLWSSRAKRSGSGATNFSLLK | mouse) DNA |
| QAGDVEENPGPMSNTAFPDPAWNTTLLSWVALFLLGTKHMEAAVTQ | Sequence: |
| SPRNKVAVTGGKVTLSCNQTNNHNNMYWYRQDTGHGLRLIHYSYGA | SEQ ID NO: |
| GSTEKGDIPDGYKASRPSQENFSLILELATPSQTSVYFCASGDNSAETL | 252 |
| YFGPGTRLLVLEDLRNVTPPKVSLFEPSKAEIANKQKATLVCLARGFFP | |
| DHVELSWWVNGKEVHSGVSTDPQAYKESNYSYCLSSRLRVSATFWH | |
| NPRNHFRCQVQFHGLSEEDKWPEGSPKPVTQNISAEAWGRADCGITSA | |
| SYHQGVLSATILYEILLGKATLYAVLVSGLVLMAMVKKKNS (SEQ ID | |
| NO: 251) | |
| C-003122 (H-Y TCRalpha T48C P2A H-Y TCRbeta Jb2.3L S57C | C-003122 |
| human); | (H-Y |
| MFPVTILLLSAFFSLRGNSAQSVDQPDAHVTLSEGASLELRCSYSYSAA | TCRalpha |
| PYLFWYVQYPGQSLQFLLKYITGDTVVKGTKGFEAEFRKSNSSFNLKK | T48C P2A |
| SPAHWSDSAKYFCALEGQDQGGSAKLIFGEGTKLTVSSPYIQNPDPAV | H-Y |
| YQLRDSKSSDKSVCLFTDFDSQTNVSQSKDSDVYITDKCVLDMRSMD | TCRbeta |
| FKSNSAVAWSNKSDFACANAFNNSIIPEDTFFPSPESSCDVKLVEKSFET | Jb2.3L S57C |
| DTNLNFQNLSVIGFRILLLKVAGFNLLMTLRLWSSGSGATNFSLLKQA | human) DNA |
| GDVEENPGPMSNTAFPDPAWNTTLLSWVALFLLGTKHMEAAVTQSPR | Sequence: |
| NKVAVTGGKVTLSCNQTNNHNNMYWYRQDTGHGLRLIHYSYGAGST | SEQ ID NO: |
| EKGDIPDGYKASRPSQENFSLILELATPSQTSVYFCASGDNSAETLYFGP | 254 |
| GTRLLVLEDLKNVFPPEVAVFEPSEAEISHTQKATLVCLATGFYPDHVE | |
| LSWWVNGKEVHSGVCTDPQPLKEQPALNDSRYCLSSRLRVSATFWQN | |
| PRNHFRCQVQFYGLSENDEWTQDRAKPVTQIVSAEAWGRADCGFTSE | |
| SYQQGVLSATILYEILLGKATLYAVLVSALVLMAMVKRKDSRG (SEQ | |
| ID NO: 253) | |

### Example 16: Minor Histocompatibility Antigen HA-1 Inhibitory Receptors

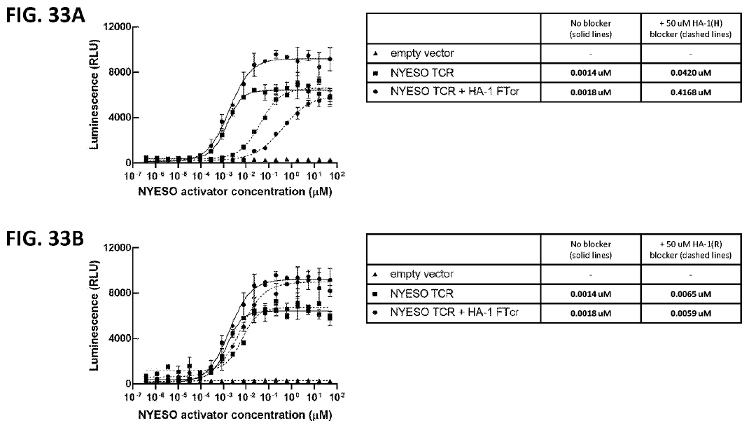

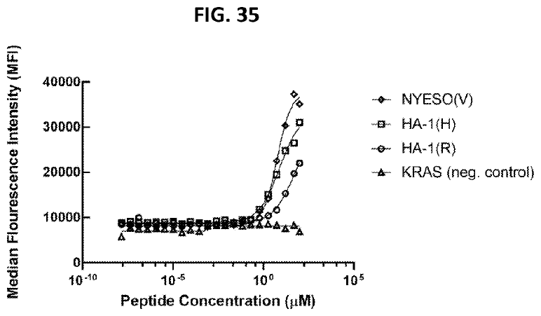

T2 cells carrying HLA-A*02 and A*11 class I alleles were loaded with a titration of A*02-specific NY-ESO-1 peptide in the absence or presence of 50 μM A*02-specific HA-1 blocker peptides. Activation of Jurkat effector cells expressing either an NY-ESO-1 TCR or an NY-ESO-1 TCR and HA-1 Ftcr was assayed as described above (see Example 6). FIG. 33A shows that in the absence of blocker peptide, sensitivity of the NY-ESO-1 TCR is not affected by the presence of HA-1 Ftcr. In the presence of HA-1(H) blocker peptide, NY-ESO-1 and HA-1(H) peptides compete for the same HLA-A*02 allele causing an EC50 right shift of ˜30× (solid squares to dashed squares). Yet, an additional right shift in activity of ˜10× is observed in the presence of HA-1 Ftcr blocker (dashed squares to dashed circles). In addition, Emax is shifted downward 1.5× (solid circles to dashed circles). FIG. 33B shows that in the presence of the non-specific, allelic variant HA-1(R) blocker peptide, essentially no blocking is observed, suggesting blocking is specific to a single amino acid. In general, since NY-ESO-1 loads into HLA-A*02 more efficiently than HA-1(R) (see FIG. 35), there is also only a 3-5× right shift observed in the presence of HA-1(R) blocker peptide (solid to dashed lines).

Peptide sequences were as follows:

NY-ESO-1 =
(SEQ ID NO: 255)
SLLMWITQV,
HA-1(H) =
(SEQ ID NO: 187)
VLHDDLLEA,
HA-1(R) =
(SEQ ID NO: 256)
VLRDDLLEA.

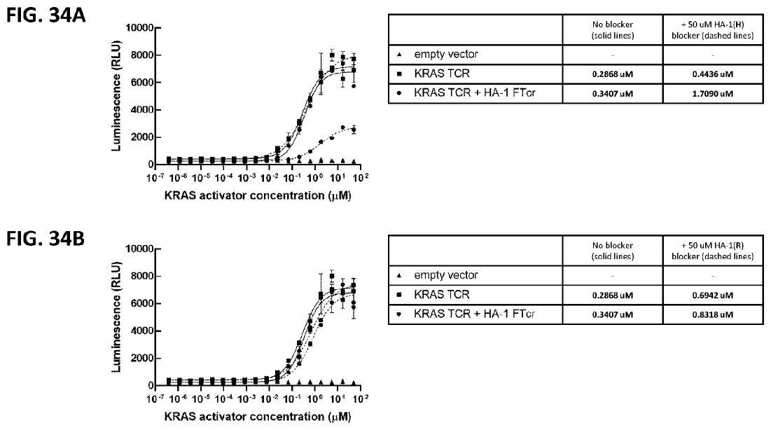

HA-1 Ftcr can also block a KRAS TCR specifically in the presence of HA-1(H) peptide. T2 cells carrying HLA-A*02 and A*11 class I alleles were loaded with a titration of A*11-specific KRAS peptide in the absence or presence of 50 μM A*02-specific HA-1 blocker peptides. Activation of Jurkat effector cells expressing either an NY-ESO-1 TCR or an NY-ESO-1 TCR and HA-1 Ftcr was assayed as described above (see Example 6). FIG. 34A shows that in the absence of blocker peptide, sensitivity of the KRAS TCR is not affected by the presence of HA-1 Ftcr. In the presence of HA-1(H) blocker peptide, HA-1 Ftcr blocks KRAS TCR by ˜5× in activity (solid circles to dashed circles). In addition, Emax is shifted downward 2.7× (solid circles to dashed circles). FIG. 34B shows that in the presence of the non-specific, allelic variant HA-1(R) blocker peptide, essentially no blocking is observed, suggesting blocking is specific to a single amino acid. In general, since KRAS and HA-1(H) or HA-1(R) do not load into the same alleles, there is no significant right shift observed in the presence of blocker peptide (solid to dashed lines).

Loading of the NY-ESO, HA-1(H) and HA-1(R) peptides by T2 cells was compared using BB7.2 staining, which specifically recognizes peptide-loaded HLA-A*02 class I allelic products, and quantified using flow cytometry. FIG. 35 shows that loading of HLA-A*02− specific NY-ESO-1 and HA-1(H) peptides is very similar in T2 cells. The allelic variant, HA-1(R), loads slightly less efficiently than HA-1(H) and NY-ESO-1 peptides.

HA-1(H) Ftcr sequences set forth in SEQ ID NOs: 189, 191, 192, 195, and 196 (amino acid) and 190, 192, 194, 197, and 198 (nucleic acid), NY-ESO-1 and KRAS TCR are shown in Table 6 below.

TABLE 6
NY-ESO-1 and KRAS TCR sequences

| | |
|---|---|
| C-000063 pLenti 1 NY-ESO1 1G4 TCRalpha T95L, S96Y, T48C | C-000063 |
| P2A TCRbeta S57C: | pLenti 1 |
| METLLGLLILWLQLQWVSSKQEVTQIPAALSVPEGENLVLNCSFTDSAI | NY-ESO1 |
| YNLQWFRQDPGKGLTSLLLIQSSQREQTSGRLNASLDKSSGRSTLYIAA | 1G4 |
| SQPGDSATYLCAVRPLYGGSYIPTFGRGTSLIVHPYIQNPDPAVYQLRDS | TCRalpha |
| KSSDKSVCLFTDFDSQTNVSQSKDSDVYITDKCVLDMRSMDFKSNSAV | T95L, S96Y, |
| AWSNKSDFACANAFNNSIIPEDTFFPSPESSCDVKLVEKSFETDTNLNFQ | T48C P2A |
| NLSVIGFRILLLKVAGFNLLMTLRLWSSGSGATNFSLLKQAGDVEENPG | TCRbeta |
| PMSIGLLCCAALSLLWAGPVNAGVTQTPKFQVLKTGQSMTLQCAQDM | S57C DNA |
| NHEYMSWYRQDPGMGLRLIHYSVGAGITDQGEVPNGYNVSRSTTEDFP | Sequence: |
| LRLLSAAPSQTSVYFCASSYVGNTGELFFGEGSRLTVLEDLKNVFPPEV | SEQ ID NO: |
| AVFEPSEAEISHTQKATLVCLATGFYPDHVELSWWVNGKEVHSGVCTD | 258 |
| PQPLKEQPALNDSRYCLSSRLRVSATFWQNPRNHFRCQVQFYGLSEND | |
| EWTQDRAKPVTQIVSAEAWGRADCGFTSESYQQGVLSATILYEILLGK | |
| ATLYAVLVSALVLMAMVKRKDSRG (SEQ ID NO: 257) | |
| C-000891 pLenti 1 K33A1101_V002 TCR (G12D TRAV4-4/ | C-000891 |
| DV10*01/BV12-2*01): | pLenti 1 |
| MQRNLGAVLGILWVQICWVRGDQVEQSPSALSLHEGTDSALRCNFTTT | K33A1101_ |
| MRSVQWFRQNSRGSLISLFYLASGTKENGRLKSAFDSKERRYSTLHIRD | V002 TCR |
| AQLEDSGTYFCAADSSNTGYQNFYFGKGTSLTVIPNIQNPEPAVYQLKD | (G12D |
| PRSQDSTLCLFTDFDSQINVPKTMESGTFITDKTVLDMKAMDSKSNGAI | TRAV4- |
| AWSNQTSFTCQDIFKETNATYPSSDVPCDATLTEKSFETDMNLNFQNLS | 4/DV10*01/ |
| VMGLRILLLKVAGFNLLMTLRLWSSRAKRSGSGATNFSLLKQAGDVEE | BV12-2*01) |
| NPGPMSNTAFPDPAWNTTLLSWVALFLLGTSSANSGVVQSPRYIIKGKG | DNA |
| ERSILKCIPISGHLSVAWYQQTQGQELKFFIQHYDKMERDKGNLPSRFS | Sequence: |
| VQQFDDYHSEMNMSALELEDSAVYFCASSLTDPLDSDYTFGSGTRLLVI | SEQ ID NO: |
| EDLRNVTPPKVSLFEPSKAEIANKQKATLVCLARGFFPDHVELSWWVN | 260 |
| GKEVHSGVSTDPQAYKESNYSYCLSSRLRVSATFWHNPRNHFRCQVQF | |
| HGLSEEDKWPEGSPKPVTQNISAEAWGRADCGITSASYQQGVLSATILY | |
| EILLGKATLYAVLVSTLVVMAMVKRKNS (SEQ ID NO: 259) | |

### Example 17: Ratios of Activator and Inhibitor Peptides

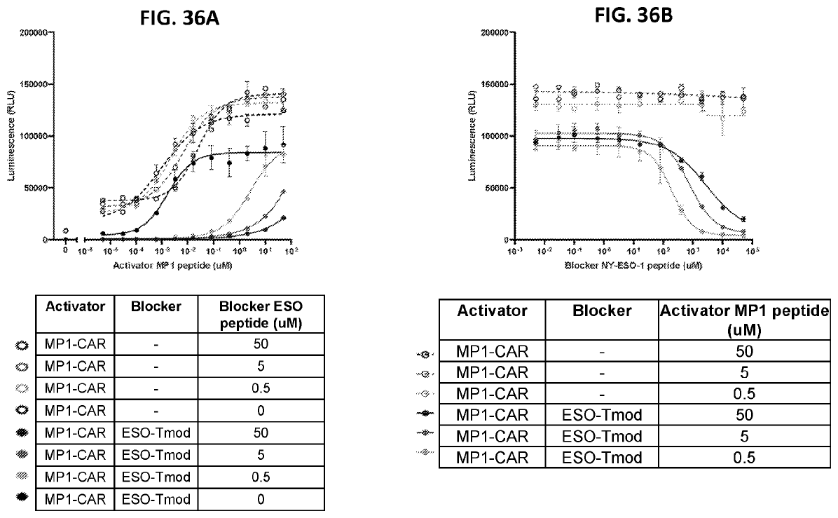

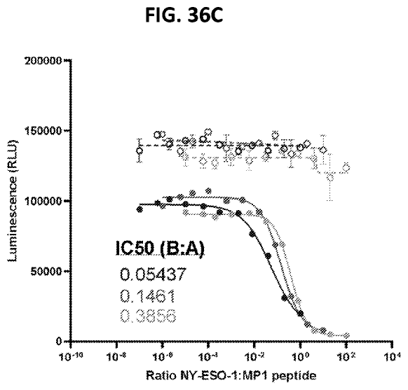

The NFAT-luciferase signal of Jurkat cells transfected with either activator MAGE-A3 CAR alone or in combination with NY-ESO-1 ScFv LIR1 blocker was measured after 6 hours of co-culture with activator and blocker peptide-loaded T2 cells. FIG. 36A shows the response of Jurkat cells co-cultured with T2 cells that were loaded with titrated amounts of activator MAGE-A3 peptide and a fixed concentration of blocker NY-ESO-1 peptide. FIG. 36B shows the response of Jurkat to T2 cells that were loaded with titrated amounts of blocker NY-ESO-1 peptide and a fixed concentration of activator MAGE-A3 peptide that was above the Emax concentration (˜0.1 μM). FIG. 36C shows x-value blocker NY-ESO-1 peptide concentrations from FIG. 36B that were normalized to the constant activator MAGE peptide concentrations used for each curve and plotted on the x-axis. The ratio of blocker peptide to activator peptide required for 50% blocking (IC50) are indicated for each curve. The B:A peptide ratio required is less than 1 indicating that, for this pair of activator CAR and blocker, similar (or fewer) blocker pMHC antigens are required on target cells to block activator pMHC antigens. Blocking is possible at pMHC antigen densities that are similar to those that generate responses in activating pMHC CARs.

### Example 18: Optimization of Specific Receptor Pairs

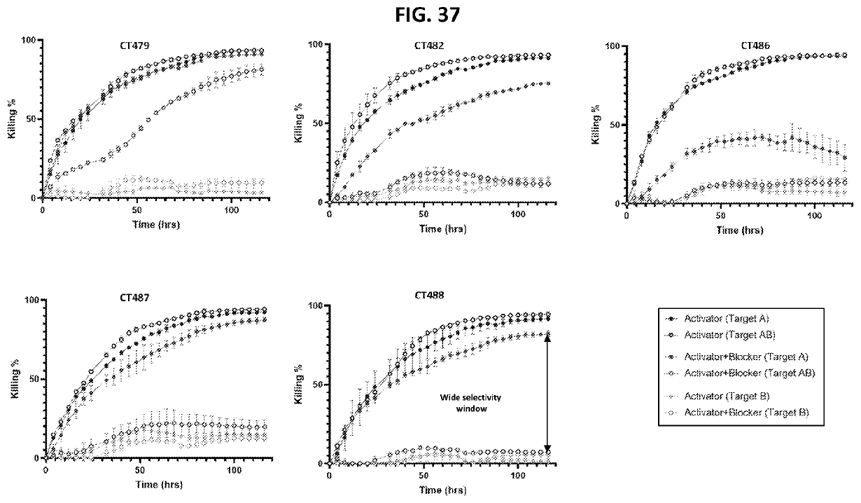

T cells transfected with either an EGFR ScFv CAR activator (CT-479, CT-482, CT-486, CT-487 or CT-488, as indicated in FIG. 37), or with EGFR ScFv CAR activator and an HLA-A*02 PA2.1 ScFv LIR1 inhibitor (C1765) were co-cultured with HeLa target cells. Wild type HeLa cell lines express EGFR but not HLA-A*02, but were transduced to express the HLA-A*02 inhibitory receptor target. Cells were co-cultured at a 1:1 ratio of effector to target (E:T). In the lower right of FIG. 37, effector cell receptor expression is indicated first, while HeLa cell expression is in paranetheses. As can be seen from FIG. 37, a different degree of blocking is observed when the same HLA-A*02 PA2.1 ScFv LIR1 inhibitor was used with different EGFR activator receptors.

### Example 19: Inhibitory Receptors Reversibly Decrease Surface Level of Activator Receptors in T Cells

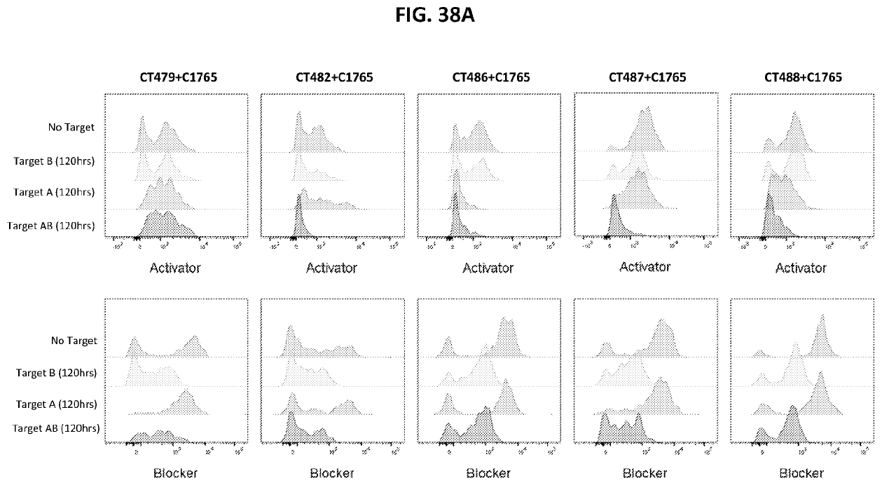

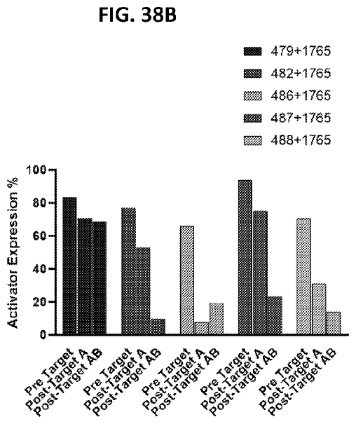

Primary T cells from two HLA-A*02 negative donors were transduced with an EGFR ScFv CAR activator (CT-479, CT-482, CT-486, CT-487 or CT-488) and an HLA-A*02 PA2.1 ScFv LIR1 inhibitor (C1765). Transduced cells were enriched by FACS sorting on the blocker and activator receptors, or by double column purification on the blocker and activator receptors. Transduced T cells were co-cultured with HeLa target cells. Wild type HeLa cell lines express EGFR but not HLA-A*02, but were transduced to express the HLA-A*02 inhibitory receptor target. Cells were co-cultured at a 1:1 ratio of effector to target (E:T). Surface expression of the EGFR CAR activator was assayed after 120 hours using labeled peptides that bound the activator and blocker receptors, and fluorescence activated cell sorting. The change in activator surface level following co-culture with HeLa cells expressing both activator and blocker ligands corresponded to the T cells' ability to kill target cells (compare FIG. 37 and FIG. 38).

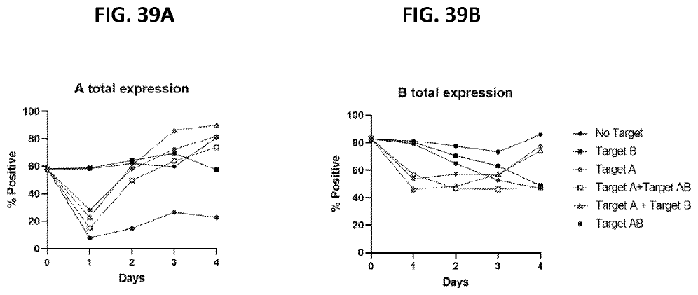

T cells expressing the CT-482 EGFR ScFv CAR activator and HLA-A*02 PA2.1 ScFv LIR1 inhibitor (C1765) combination, were co-cultured with HeLa cells expressing EGFR (Target A), HLA-A*02 (Target B), a combination of EGFR and HLA-A*02 on the same cell (Target AB), a mixed population of HeLa cells expressing Target A and Target AB on different cells, or a mixed population of HeLa cells expressing Target B and Target AB on different cells (FIGS. 39A-39B). T cells were cultured with HeLa target cells at a ratio of 1:1 effector cell to target cell. When T cells were co-cultured with a Target A plus Target AB population of HeLa cells, levels of activator decreased, then recovered (FIG. 39A). Furthermore, the activator and blocker antigens must be present together on the same cell to trigger activator surface expression loss on effector T cells. In contrast to the activator, blocker expression was largely unchanged (FIG. 39B).

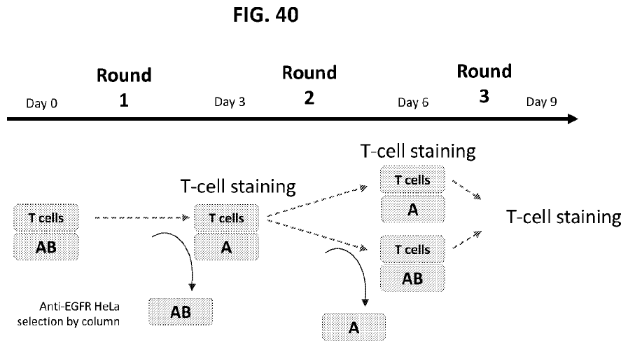

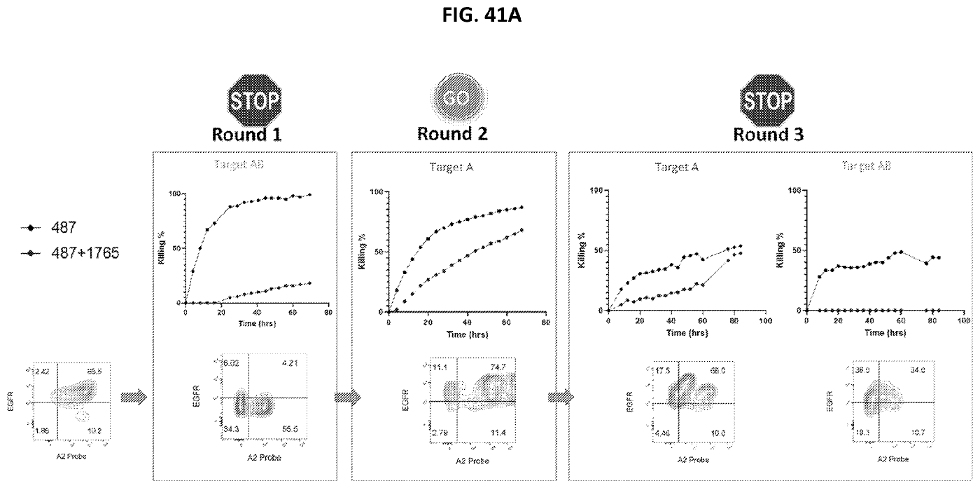

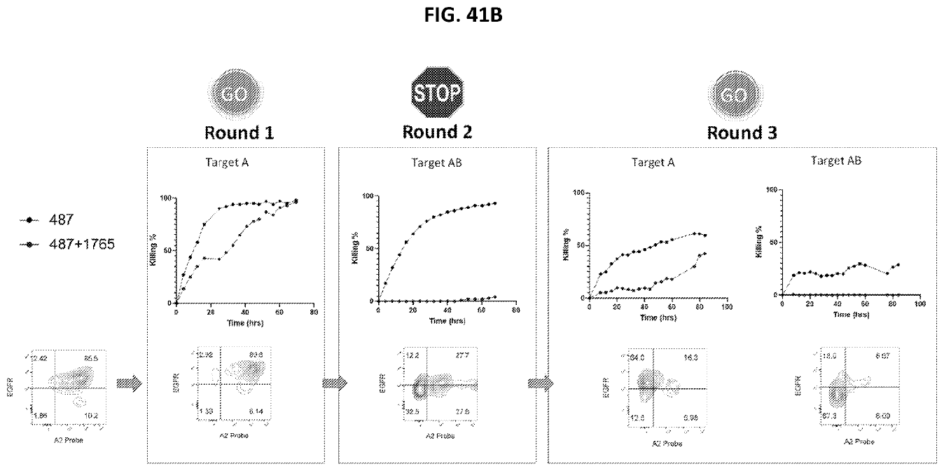

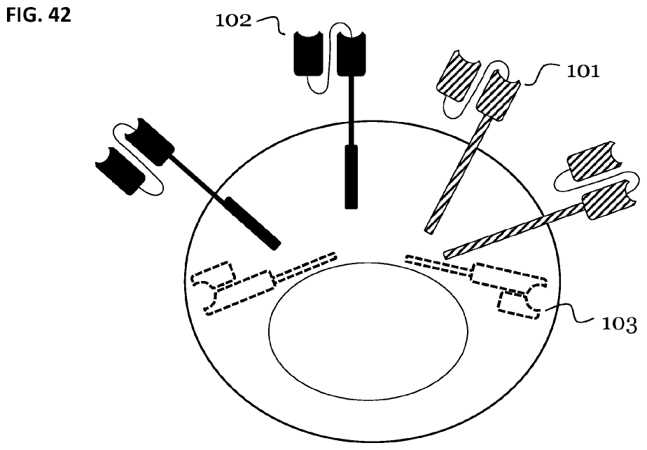

FIG. 40 shows a schematic for an experiment to determine if loss of expression of activator receptor by T cells was reversible. T cells expressing EGFR ScFv CAR activator receptor (CT-487) and HLA-A*02 PA2.1 ScFv LIR1 (C1765) inhibitor receptor were co-cultured with HeLa target cells expressing both the activator and blocker receptor targets (AB). Following 3 days co-culture, HeLa cells were removed using an anti-EGFR column, and the T cells were either stained for the activator and inhibitor receptors, or co-cultured with HeLa cells expressing EGFR activator target only for an additional 3 days. After the additional 3 days co-culture, HeLa cells were again removed using an anti-EGFR column, and the T cells were either stained for the activator and inhibitor receptors, or were co-cultured for an additional 3 days with HeLa cells expressing EGFR activator target only, or both the activator and blocker targets (AB), before staining. T cells were assayed for the presence of activator and inhibitor receptors (stained) using labeled EGFR and A2 probes, and the levels of receptor expression were quantified using fluorescence activated cell sorting. Results of the experiment are shown in FIGS. 41A-41B. As shown in FIGS. 41A-41B, co-culture of T cells with HeLa cells expressing both activator and inhibitor targets reduces EGFR activator staining (FIGS. 41A-41B, left panel). When T cells are co-cultured with HeLa cells expressing activator (Target A only) at round 2, expression of EGFR activator increases. Thus, activator surface loss is reversible and tracks with T cell cytotoxicity.

### Example 20: Reversible Repression of Graft-Versus-Host Response by an Inhibitory Receptor Specific to an MHC-I Ligand

### Methods

### Cell Culture

Raji cells were maintained as suggested by ATCC. “Normal” Raji cells were made by transducing Raji cells with HLA-A*02 lentivirus at a MOI of 5. HLA-A*02-positive Raji cells were sorted using a FACSMelody Cell Sorter (BD).

### Plasmid Construction

The CD19 CAR was created by fusing the FMC63-derived (Nicholson et al., 1997) scFv to the CD8 hinge, CD28 transmembrane region and CD28-4-1BB-CD3zeta signaling domains. The HLA-A*02-responsive inhibitory construct was created by fusing the scFv LBD of monoclonal antibody PA2.1 to the hinge, transmembrane region, and intracellular domain of LILRB1 (LIR-1). Gene segments were combined using Golden Gate cloning and inserted downstream of a human EFla promoter contained in a lentiviral expression plasmid.

### Primary T Cell Transduction, Expansion, and Enrichment

Frozen PBMCs were thawed in 37° C. water bath and cultured at 1e6 cells/mL in LymphoONE (Takara) with 1% human serum and activated using 1:100 of T cell TransAct (Miltenyi) supplemented with IL-2 (100 IU/mL). After 24 hours, lentivirus was added to PBMCs at a MOT of 5. PBMCs were cultured for 2-3 additional days to allow cells to expand under TransAct stimulation. Post expansion, activator and blocker transduced primary T cells were enriched using anti-PE microbeads (Miltenyi) according to manufacturer's instructions. Briefly, primary T cells were incubated with CD19-Fc (R&D Systems) at 1:100 dilution for 30 minutes at 4° C. in MACS buffer (0.5% BSA+2 mM EDTA in PBS). Cells were washed 3 times in MACS buffer and incubated in secondary antibody (1:200) for 30 minutes at 4° C. in MACS buffer. Cells were then incubated in anti-PE microbeads and passed through the LS column (Miltenyi).

### Primary T Cell In Vitro Cytotoxicity Studies

Enriched primary T cells were incubated with 2e3 WT Raji cells (“tumor” cells) or HLA-A*02 transduced Raji cells (“normal” cells) at an effector:target ratio of 3:1 in the presence of 100 IU/mL. WT “tumor” Raji cells stably express GFP and renilla luciferase (Biosettia), and HLA-A*02 “normal” Raji cells stably express RFP and firefly luciferase (Biosettia). For reversibility studies, enriched primary T cells were cocultured with “normal” or “tumor” Raji cells for 3 days. After 3 days, T cells were separated from remaining Raji cells using CD19 negative selection and reseeded with fresh “normal” or “tumor” Raji cells. In separate wells, live luciferase-expressing Raji cells were quantified using a Dual-Luciferase Reporter Assay System (Promega) at 72 hrs. Reversibility was assessed for 7 rounds, oscillating between exposure to “normal” or “tumor” target cells.

This example demonstrates that the graft-versus-host response is reversibly suppressed by an inhibitory receptor specific to an MHC-I. Jurkat cells engineered to express either an anti-CD19 CAR alone or an anti-CD19 CAR (“CD19-CAR”) and an inhibitory receptor specific to HLA-A*02 (“Tmod”) were generated, as summarized in Table 7.

TABLE 7

| | Activatory Receptor | Inhibitory Receptor | Activity |
|---|---|---|---|
| “UTD” | Endogenous TCRs | N/A | Gradual buildup of T cell |
| | reactive to alloantigens | | response to allogeneic HLA on |
| | | | Raji cells (mimics GvHD) |
| | | | causes increased cytoxicity as |
| | | | the rounds progress |
| “CD19- | Chimeric antigen | N/A | CD19-specific CTL response is |
| CAR” | receptor specific to | | added to the allogeneic |
| | CD19 | | response, causing high |
| | | | cytotoxicity of Raji cells at each |
| | | | round |
| “Tmod” | Chimeric antigen | Chimeric antigen | No anti-CD19 response in the |
| | receptor specific to | receptor specific to | presence of HLA-A*02-positive |
| | CD19 | HLA-A*02 with | Raji cells (Rounds 1 and 3); |
| | | intracellular domain | control/mitigation of allogeneic |
| | | from LILRBI | response during each round |

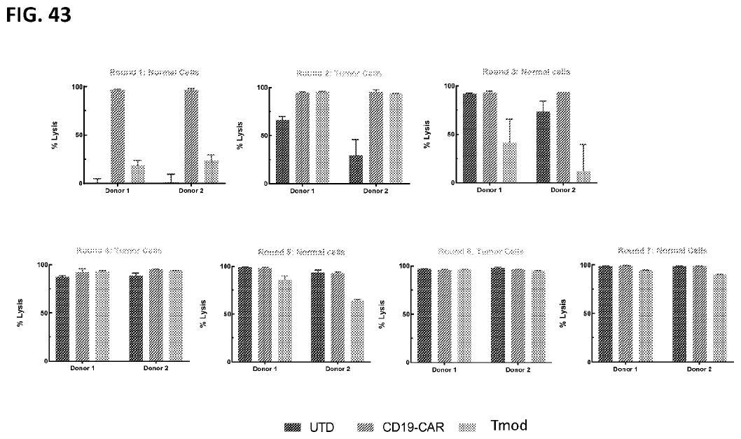

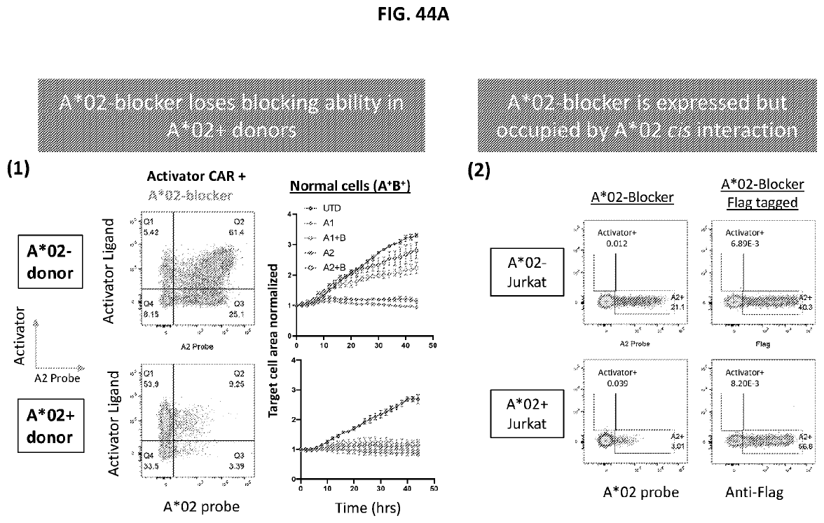

Results are shown in FIG. 43. FIG. 43 depicts the cytotoxic T-cell lymphocyte (CTL) response of engineered effector T cells over time, with multiple rounds of primary T-cell transfer. As can be seen in FIG. 43, the CTL response is suppressed by normal cells (rounds 1, 3, 5 and 7). Exposure to tumor cells (rounds 2, 4, and 6) activates the CTL response. Surprisingly, the inhibitory receptor also suppresses the allogeneic response. This is visible in the comparison between untransduced donor T cells, whose cytotoxicity is stimulated by the mismatched HLA class I haplotype of the donor T cells and the target cells (Raji). In contrast, T cell expressing the activator and inhibitor receptor pair (green bars) show an inhibited allogeneic response to Raji target cells compared to untransduced (UTD, blue bars) cells. Note that this experiment was conducted in high IL-2 (300 Units/mL), and cell groups expressing the activator and inhibitor receptor combination have up to 10% cells that do not express the inhibitory receptor. This may explain diminished control of the allogeneic response by the inhibitory receptor over time.

### Example 21: gRNA Targeting Sequences

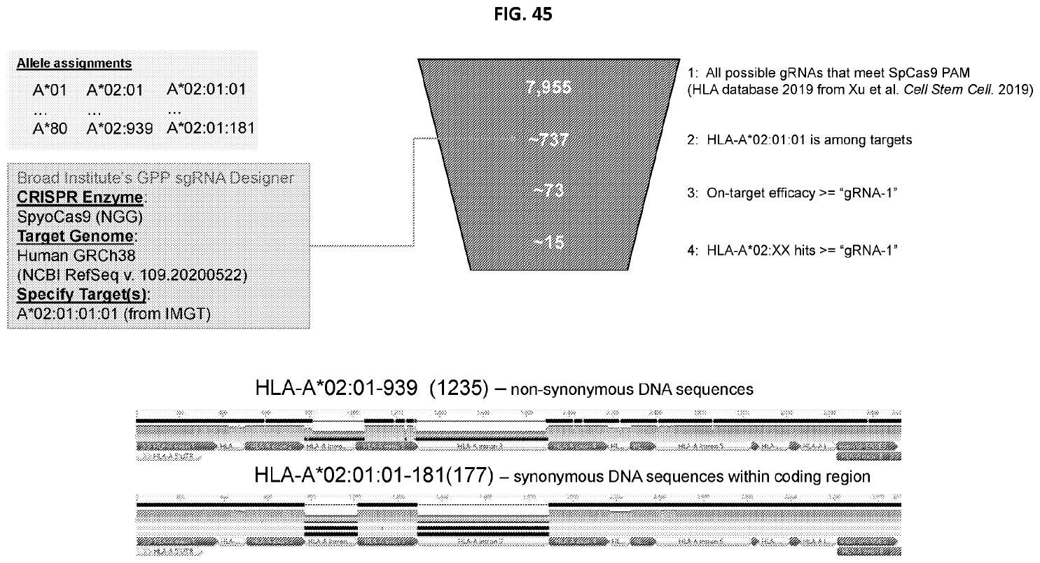

In order to select guide RNAs for targeting HLA-A alleles, the inventors retrieved all gRNAs from a published database of gRNAs that target the HLA-A and meet the requirements for use of the Streptococcus pyogenes Cas9 (SpCas9) nucleic acid guided endonuclease (Xu et al. Cell Stem Cell. 24:P566-578 (2019)). A computational approach was used to progressively filter putative gRNAs (FIG. 45). A total of 7,955 gRNA putative targeting sequences were retrieved. The gRNA candidates were analyzed and filtered to identify only those which have the HLA-A*02:01:01 allele as a target. This was accomplished using the GPP sgRNA designer algorithm publicly available from the Broad Institute, using as input the genetic locus harboring the HLA-A*02:01:01 allele retrieved from the International Immunogenetics Information System®. A total of ˜737 sequences from the initial 7,955 putative sequences were identified that target the HLA-A*02:01:01 allele. On-target efficacy scores were generated for the set of 737 targeting sequences to further reduce the number of putative targeting sequences to those with high-efficacy and bind coding DNA sequences in the HLA-A*02:01:01 allele. Efficacy scores were generated using the Azimuth 2.0 algorithm based on rules described previously (Doench et al. Nature Biotechnology. 34:184-191 (2016)). The set was reduced by removing any sequences with an on-target efficacy score of less than 0.5 and predicted to not target the coding DNA sequence of HLA-A*02 alleles. Applying this filter resulted in 120 putative sequences. Finally the putative sequences predicted to target fewer than 1000 HLA-A*02 allele variants were filtered out. Applying this filter resulted in ˜66 targeting sequences to select from for experimental characterization.

### Example 22: Putative gRNA Targeting Sequences Reduce Class I HLA Expression

gRNA-mediated knockout of HLA-A, HLA-A alleles, HLA-B, HLA-C, and HLA-G for 16 of the putative targeting sequences was experimentally determined in Jurkat cells and primary T cells. Table 8 shows the 16 targeting sequence candidates, their designation for the characterization experiment, and the percent knockout of HLA-A*02 or Class I HLA polypeptides (HLA-A, HLA-B, HLA-C, and HLA-G expression) in Jurkat cells.

TABLE 8
Targeting Sequences and percent knockout for
HLA-A*02 and Class I HLA in Jurkat cells.

| | SEQ ID | HLA-A*02 | Class I HLA |
|---|---|---|---|
| Designation | NO | % KO | % KO |
| gRNA-1 | 426 | 66.4 | 30.1 |
| gRNA-8 | 394 | 59.2 | 24.4 |
| gRNA-9 | 407 | 68.2 | 50.5 |
| gRNA-10 | 440 | 62.7 | 41.8 |
| gRNA-11 | 438 | 57.7 | 50.4 |
| gRNA-12 | 414 | 52.0 | 17.3 |
| gRNA-13 | 408 | 55.7 | 61.7 |
| gRNA-14 | 433 | 66.9 | 14.3 |
| gRNA-15 | 435 | 39.0 | 10.7 |
| gRNA-16 | 423 | 76.1 | 13.4 |
| gRNA-17 | 448 | 87.6 | 30.4 |
| gRNA-18 | 451 | 66.1 | 14.3 |
| gRNA-19 | 454 | 76.0 | 12.1 |
| gRNA-20 | 429 | 77.2 | 13.2 |
| gRNA-21 | 422 | 51.5 | 33.4 |
| gRNA-22 | 421 | 63.0 | 79.0 |

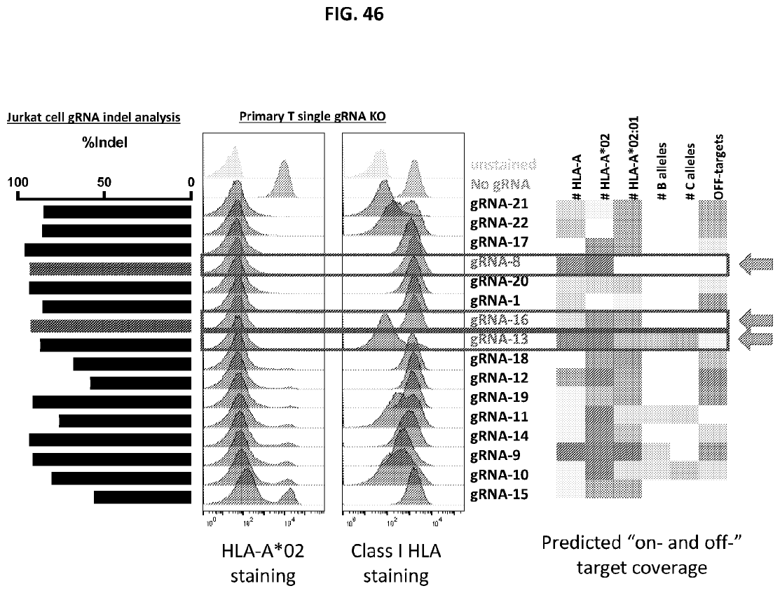

Jurkat cells expressing high levels of the HLA-A*02 allele were transfected with the putative targeting sequences and the Synthego 80 nucleotide SpCas9 scaffold. Jurkat cells were stained with fluorescently labeled anti-HLA-A*02 (HLA-A*02 staining) or a fluorescently labeled antibody that binds HLA-A/B/C (Class I HLA staining), followed by FACS (Table 8). Jurkat cells transfected with the putative targeting sequences and stained with the anti-HLA-A*02 antibody showed levels of HLA-A*02 knockout ranging from a low of 38% (gRNA-15) to a high of 87.6% (gRNA-17). Jurkat cells transfected with the putative targeting sequences and subject to Class I HLA staining showed knockout of HLA-A/B/C ranging from 10.7% gRNA-15 to 79% (gRNA-22). Indel analysis was performed on the Jurkat cells (FIG. 46), and showed that percent indel correlated with the observations of percent HLA knockout determined by FACS.

To determine the gRNAs effects on Class I HLA genes on primary T cells, primary peripheral blood mononuclear cells (PBMCs) were collected and activated. After one day, when applicable, PBMCs were transduced with polynucleotides encoding activating CAR and HLA-A*02 blocker, followed by transfection with the putative targeting sequences and the Synthego 80 nucleotide SpCas9 scaffold. Primary T cells were stained with fluorescently labeled anti-HLA-A*02 (HLA-A*02 staining) or a fluorescently labeled antibody that binds HLA-A/B/C (Class I HLA staining), followed by FACS (FIG. 46). Several HLA targeting sequences, including gRNA-8 and gRNA-16, provide robust HLA-A*02 or HLA-A knockout with wide allele coverage and low off-targets. At least one targeting sequence, gRNA-13, shows broad knockout of HLA alleles as demonstrated by decreased staining with both HLA-A*02 staining antibodies and Class I HLA staining antibodies.

### Example 23: gRNA-Mediated Knockdown of HLA-A*02 Increases Blocker Receptor Availability in HLA-A*02 Positive Donors

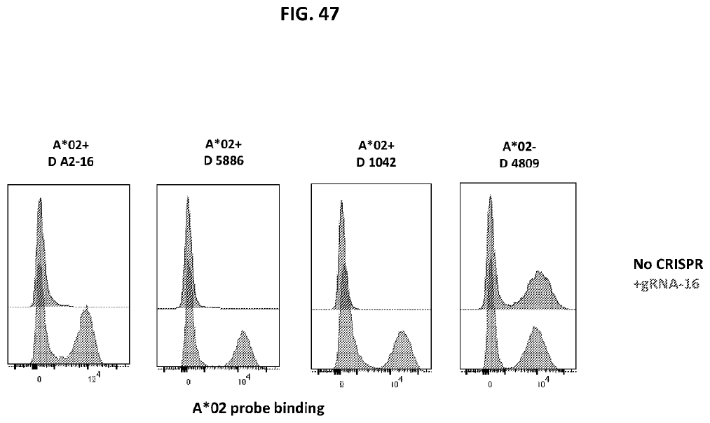

The ability of gRNA mediated knockout of HLA-A*02 alleles to increase availability of blocker ligand binding domain (LBD) was determined in T cells derived from three HLA-A*02 positive donors (FIG. 47). Primary T cells were collected from the three HLA-A*02 positive donors (D A2-16, D5886, and D 1042). One HLA-A*02 negative donor (D 4809) was used as a control. Donor T cells were transduced with CAR activator and HLA-A*02 blocker receptors. One day following transduction, T cells were either transfected with gRNA-16 to knockout HLA-A*02 or were in the No CRISPR negative control group. Cells were stained using a fluorescently labeled pMHC probe (A*02 probe binding) specific to binding HLA-A*02. Increased staining was observed in the HLA-A*02 positive cells transfected with gRNA-16, indicating that knockdown of the HLA-A*02 gene increased the capacity of blocker receptors to bind the target ligand. These results suggest that endogenous expression of HLA-A*02 interferes with the HLA-A*02-specific blocker receptor ligand binding capacity, and that knockout of endogenous HLA-A*02 reverses the effect. Thus, gRNA-mediated knockout of endogenous HLA-A*02 in immune cells expressing an HLA-A*02-specific blocker receptor can reduce autocrine interference or activation of the blocker receptor (FIG. 47).

### Example 24: Illustrative Methods of the Disclosure

### CRISPR Materials and Methods

### Cell Culture

Jurkat cells encoding an NFAT Luciferase reporter were maintained in RPMI media supplemented with 10% FBS, 1% Pen/Strep and 0.4 mg/mL G418/Geneticin. A2+ Jurkat were made by transducing Jurkat NFAT luciferase cells with HLA-A*02 lentivirus (custom lentivirus, Alstem) at a MOI of 5. HLA-A*02-positive cells were sorted using a FACSMelody Cell Sorter (BD).

### Jurkat Cell Transfection

Jurkat cells were transfected with Cas9/sgRNA complexes using 100 uL format Neon electroporation system (Thermo Fisher) according to manufacturer's protocol using the following settings: 3 pulses, 1500V, 10 msec. Transfections were performed with 20 pmol Cas9 2NLS nuclease (Synthego) and 130 pmol sgRNA (Synthego) per 1 million cells which were pre-incubated for 15 minutes at room temperature prior to transfection. Transfected cells were recovered RPMI media supplemented with 20% heat-inactivated FBS and 0.1% Pen/Strep.

Primary T cell transduction and Transfection

Frozen PBMCs were thawed in 37° C. water bath and cultured at 1e6 cells/mL in LymphoONE (Takara) with 1% human serum and activated using 1:100 of T cell TransAct (Miltenyi) supplemented with IL-2 (300 IU/mL). After 24 h, if applicable, lentivirus was added to PBMCs at MOI=5. A and B modules were simultaneously co-transduced at a MOI=5 for each lentivirus. 24 h later, PBMCs were transfected with Cas9/sgRNA complexes using 4D-Nucleofector (Lonza) according to the manufacturer's protocol using the EO-115 program. Transfections were performed with 20 pmol Cas9 2NLS nuclease (Synthego) and 130 pmol sgRNA (Synthego) per 1 million cells which were pre-incubated for 15 minutes at room temperature prior to transfection. Transfected cells were recovered in LymphoONE (Takara) with 1% human serum and IL-2 (300 IU/ml)

For gRNA screening studies, Jurkat or primary T cells were stained 4 days after Cas9/sgRNA transfection with BB7.2 antibody (Biolegend) and/or W6/32 antibody (BD Biosciences) for 30 minutes at 4° C. to assess knockout efficiency. To evaluate blocker availability to bind HLA-A*02 antigen Jurkat cells were stained with 10 ug/mL streptavidin-PE-HLA-A*02-pMHC or streptavidin-APC-HLA-A*02-pMHC tetramer for 30 minutes at 4° C. in PBS with 1% BSA and characterized by flow cytometry (BD FACSCanto II).

### Example 25: Selection of Interfering RNA Sequences Targeting the HLA Gene

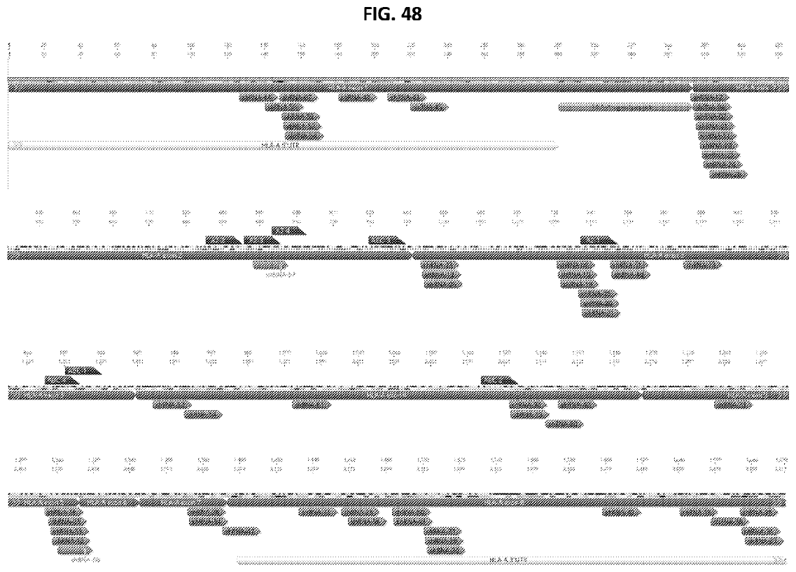

In order to select guide nucleic acids for targeting the HLA-A*02:01:01:01 mRNA, all possible 18 bp, 19 bp, 20 bp, 21 bp, and 22 bp transcribed sequences corresponding to HLA-A*02:01:01:01 mRNA were determined, yielding 8397 sequences. From this set of potential target sequences, those containing GC content less than 25% or greater than or equal to 60% were excluded, resulting in 3586 potential target sequences. From this set of potential target sequences, those with runs of 4 or more of the same base in a row or a run of 7 C or G bases in a row were removed, resulting in 3101 potential target sequences. Next, the sequences were analyzed by identifying favorable characteristics identified by computational tools, such as the GPP RNAi Designer (Broad Institute), and similar favorable characteristics. The final set of potential targets included 278 sequences that can be used to design interfering RNAs that target the HLA-A*02:01:01:01 mRNA transcript for degradation through RNA interference. Illustrative alignments between shRNAs with a complementary sequence to the identified targets of the analysis are shown in FIG. 48.

### Example 26: shRNA-Mediated Reduction of HLA-A*02 Cell Surface Expression

The effect of shRNA targeting the coding sequence (CDS) of the HLA-A*02:01:01:01 mRNA transcript on HLA-A*02 surface protein expression was assessed.

Jurkat cells expressing HLA-A*02 were transfected using 100 uL format Neon electroporation system (Thermo Fisher) according to manufacturer's protocol using the following settings: 3 pulses, 1500V, 10 msec. Co-transfections were performed with 200 ng of U6-shRNA plasmids and 200 ng of plasmid expressing EGFR as a transfection marker per 1 million cells. Transfected cells were recovered in RPMI media supplemented with 20% heat-inactivated FBS and 0.1% Pen/Strep.

4 days following transfection, cells were stained for HLA-A*02 expression with BB7.2 antibody (Biolegend) and anti-EGFR antibody (Biolegend) for 30 minutes at 4° C. to assess knockdown efficiency.

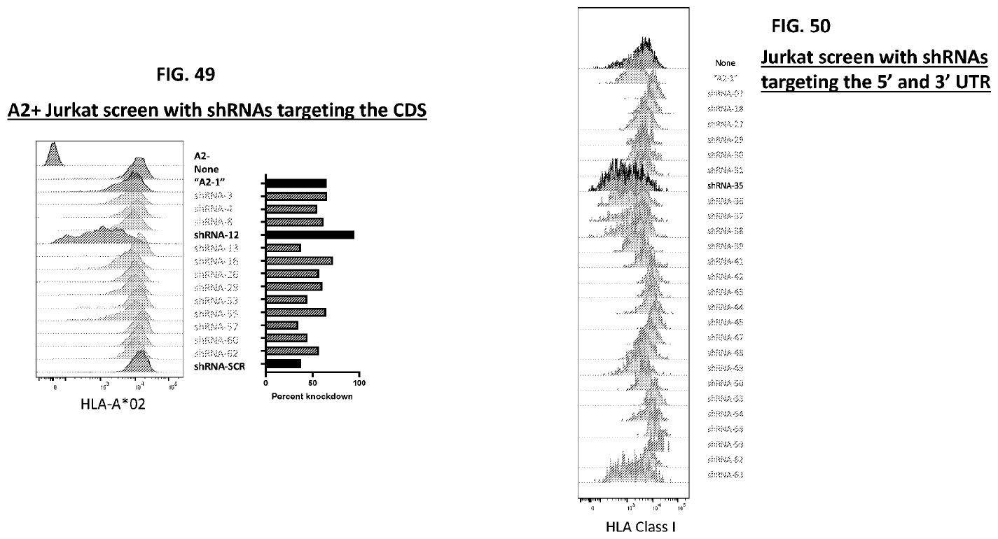

The results are shown in FIG. 49. The histograms show staining for either HLA-A*02, and corresponding quantification as % expression in control cells. The results indicate that several of the shRNA candidates were able to reduce expression of HLA-A*02 by over 50% compared to control cells. In particular, shRNA-12 showed a substantial decrease exceeding 90% knockdown compared to control cells.

These results indicate the identified shRNA targeting the CDS of the HLA-A*02:01:01:01 mRNA transcript reduces expression of HLA-A*02 surface protein in immune cells.

### Example 27: shRNA-Mediated Reduction of HLA Class I Cell Surface Expression

The effect of shRNA targeting the 5′ or 3′ untranslated region (UTR) of the HLA-A*02:01:01:01 mRNA transcript on HLA Class I surface protein expression was assessed.

Jurkat cells were transfected using 100 uL format Neon electroporation system (Thermo Fisher) according to manufacturer's protocol using the following settings: 3 pulses, 1500V, 10 msec. Co-transfections were performed with 1 μg of U6-shRNA plasmids and 100 ng of plasmid expressing green fluorescent protein (GFP) as a transfection marker per 1 million cells. Transfected cells were recovered in RPMI media supplemented with 20% heat-inactivated FBS and 0.1% Pen/Strep.

4 days following transfection, cells were stained for HLA Class I expression with W6/32 antibody (BD Biosciences) for 30 minutes at 4° C. to assess knockdown efficiency.

The results are shown in FIG. 50. The histograms show staining for HLA Class I expression. The results indicate that several of the shRNA candidates were able to reduce expression of HLA Class I compared to control cells. In particular, shRNA-35 and shRNA-63 showed a substantial decrease exceeding 90% knockdown compared to control cells.

These results indicate the identified shRNA targeting the UTRs of the HLA-A*02:01:01:01 mRNA transcript reduce expression of HLA Class I surface protein in immune cells.

### Example 28: shRNA-Mediated Reduction of HLA-A*02 Expression Increases Blocker Receptor Availability and Restores Blocker Receptor Function in HLA-A*02 Positive Jurkat Cells

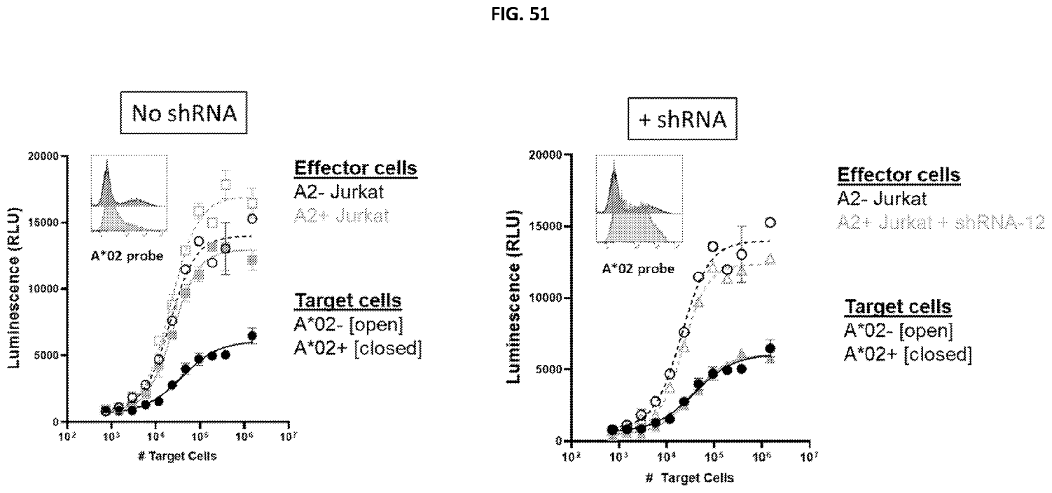

The effect of shRNA targeting the CDS of HLA-A*02:01:01:01 mRNA on the expression on blocker ligand binding receptors was determined in Jurkat cells expressing HLA-A*02. (FIG. 51).

Jurkat cells that either express HLA-A*02 (A2+ Jurkat) or do not express HLA-A*02 (A2− Jurkat) were transfected using 100 uL format Neon electroporation system (Thermo Fisher) according to manufacturer's protocol using the following settings: 3 pulses, 1500V, 10 msec. Co-transfections were performed with 2 ug plasmids encoding activator and inhibitory receptors, and 2 ug of either U6-shRNA/A*02-blocker or A*02-blocker alone per million cells. Transfected cells were recovered in RPMI media supplemented with 20% heat-inactivated FBS and 0.1% Pen/Strep.

2 days following transfection, 10⁴ Jurkat cells were co-cultured with 1.2×10⁴ HeLa target cells and incubated in Corning® 384-well Low Flange White Flat Bottom Polystyrene TC-treated Microplates for 6 hours. ONE-Step Luciferase Assay System (BPS Bioscience) was used to evaluate Jurkat luminescence. To evaluate blocker availability to bind HLA-A*02 antigen, Jurkat cells were stained with 10 ug/mL streptavidin-PE-HLA-A*02-pMHC or streptavidin-APC-HLA-A*02-pMHC tetramer for 30 minutes at 4° C. in PBS with 1% BSA and characterized by flow cytometry (BD FACSCanto II). Histograms show all cells.

Results are shown in FIG. 51. In A2+ Jurkat effector cells in the absence of shRNA targeting the CDS of HLA-A*02:01:01:01 mRNA, activation of the CAR/TCR receptor occurred in the presence of target cells expressing the activator ligand and the HLA-A*02 blocker ligand, as well as in the presence of target cells expressing the activator ligand only. In contrast, A2− Jurkat effector cells showed significantly less CAR/TCR activation in the presence of target cells expressing the activator ligand and the HLA-A*02 blocker ligand. The A*02 blocker functions to inhibit T cell activation, which was observed in A2− Jurkat effector cells co-cultured with target cells expressing HLA-A*02 but not in A2+ Jurkat effector cells co-cultured with target cells expression HLA-A*02. This result indicates that expression of HLA-A*02 in Jurkat effector cells expressing an activator CAR/TCR and A*02 blocker interferes with A*02 inhibitory receptor function. In A2+ Jurkat effector cells transfected with shRNA targeting the CDS of HLA-A*02:01:01:01 mRNA, CAR/TCR activation was inhibited in the presence of target cells expressing the HLA-A*02 blocker ligand, but not in the presence of target cells lacking expression of the HLA-A*02 blocker ligand. This result indicates that shRNA targeting the CDS of HLA-A*02:01:01:01 mRNA can restore A*02 blocker function in the A2+ Jurkat cells.

In Jurkat cells stained with the fluorescently labeled probe that binds the A*02 blocker, FACS analysis shows the availability, or binding capacity, of the A*02 blocker receptor (inhibitory receptor). In A2− Jurkat cells, histograms from the FACS analysis shows staining of a subset of cells, indicating availability of the A*02 blocker receptor. In contrast, in A2+ Jurkat cells, there is a shift in the histogram, showing the population of cells has little to no A*02 blocker receptor availability and another ill-defined population with incomplete A*02 blocker receptor availability. However, transfection in A2+ Jurkat cells transfected with shRNA targeting the CDS of HLA-A*02:01:01:01 mRNA, the histogram is shifted and resembles the A2− population, indicating near complete availability of the A*02 blocker receptor.

Taken together, the results of these experiments show that in Jurkat cells expressing 1) a CAR or TCR that binds an activator ligand and 2) an engineered receptor that binds an HLA-A*02 blocker ligand (A*02 blocker), expression of HLA-A*02 interferes with A*02 blocker receptor function and A*02 blocker binding capacity. Further, the results show that shRNA mediated reduction of HLA-A*02 expression restores A*02 blocker function and binding capacity.

### Example 29: Selection of gNA Sequences Targeting the B2M Gene

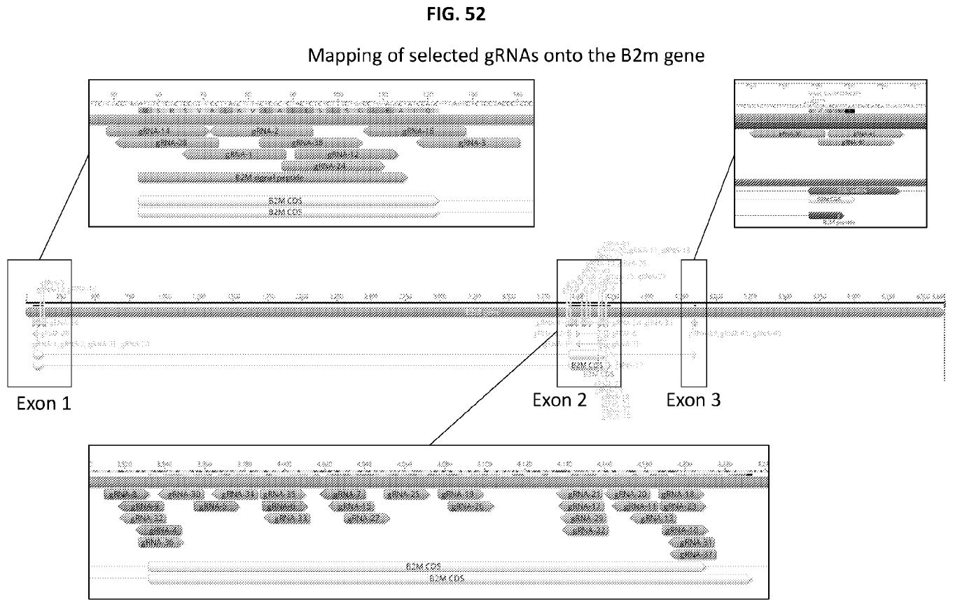

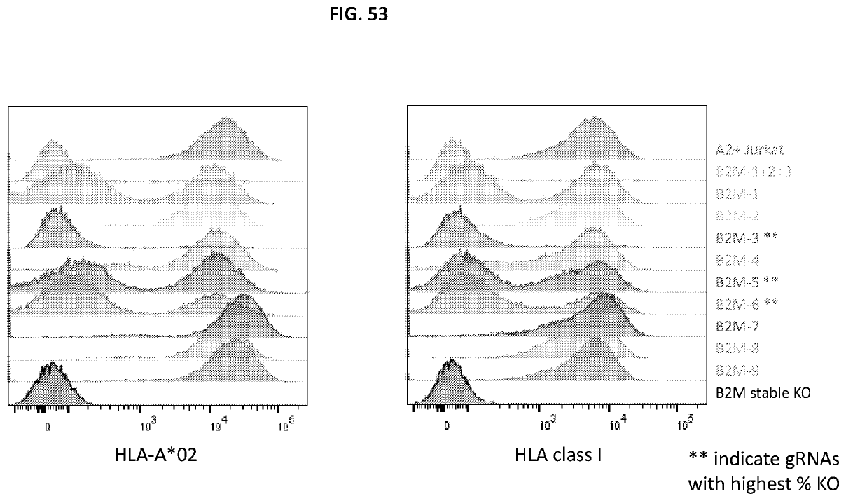

In order to select guide nucleic acids for targeting the B2M gene, all possible gNA sequences that target a 20 nucleotide sequences in the B2M gene adjacent to an NGG protoadjacent motif (PAM) were identified, resulting in a set of 741 putative gNA sequences. This set of sequences is compatible with the Steptococcus pyogenes Cas9 nucleic acid guided endonuclease. The putative gNA sequences were mapped to the B2M gene, and 41 gNA sequences were identified that target, at least partially, the coding sequence (CDS) of the B2M gene (FIG. 52). From the CDS targeting gNA sequences, nine were selected for experimental validation (Table 9).

TABLE 9
gNA sequences targeting the CDS of the B2M gene

| Designation | SEQ ID NO |
|---|---|
| B2M-1 | 8357 |
| B2M-2 | 8358 |
| B2M-3 | 8359 |
| B2M-4 | 8360 |
| B2M-5 | 8361 |
| B2M-6 | 8362 |
| B2M-7 | 8363 |
| B2M-8 | 8364 |
| B2M-9 | 8365 |

### Example 30: gNA Sequences Targeting the B2M Gene Reduce Class I HLA Expression

gNA-mediated knockout of B2M for the nine gNA sequences in Table 9 was experimentally determined in Jurkat cells and primary T cells. Table 9 shows the nine gRNA targeting sequence candidates and their designation for the characterization experiments.

B2M CDS-targeting gRNAs were tested individually or in combination for the ability to knockout HLA-A*02 or Class I HLA polypeptides (HLA-A, HLA-B, HLA-C, and HLA-G expression) in Jurkat cells transduced with HLA-A*02 (A2+ Jurkat). The A2+ Jurkat cells were transfected with pre-complexed Cas9:sgRNA complexes from Synthego. A2+ Jurkat cells without gRNA transfection and B2M stable knockout cells severed as positive and negative controls, respectively. Four days after transfection, cells were stained for either HLA-A*02 expression or expression of Class I HLA using fluorescently labeled antibodies and subject to FACS analysis. FIG. 53 shows histograms from the FACS analyses indicating the extent of knockout mediated by an individual gRNA or combination of gRNAs. Efficacy of the gRNA targeting sequences varied, with several targeting sequences achieving up to 99% knockout of HLA-A*02 and HLA Class I expression. The targeting sequences in B2M-3, B2M-5, and B2M-6 achieved the highest percent knockout of both HLA-A*02 and HLA Class I expression in the A2+ Jurkat cells.

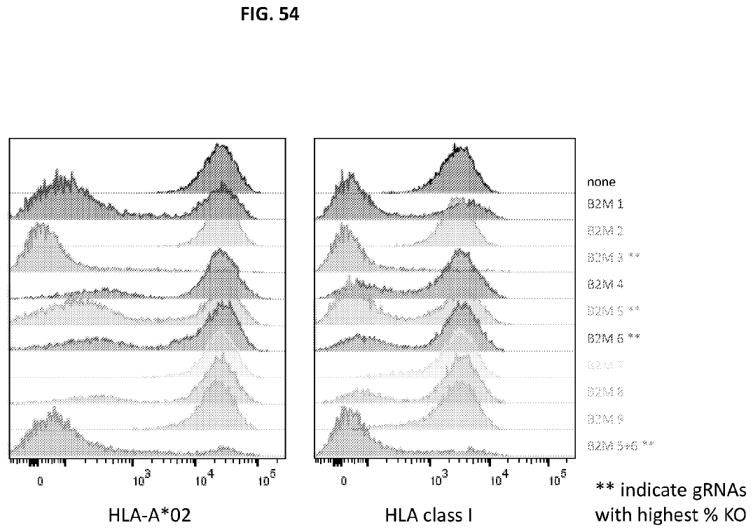

B2M CDS targeting gRNAs were tested individually or in combination for the ability to knockout HLA-A*02 or Class I HLA polypeptides (HLA-A, HLA-B, HLA-C, and HLA-G expression) in HLA-A*02 positive (HLA-A*02+) primary T cells. The primary T cells were transfected with pre-complexed Cas9:sgRNA from Synthego. Four days after transfections, cells were stained for either HLA-A*02 expression or expression of Class I HLA using fluorescently labeled antibodies followed by FACS analysis. FIG. 54 shows histograms from the FACS analyses indicating the extent of knockout mediated by an individual gRNA or combination of gRNAs. Efficacy of the gRNA targeting sequences varied, from little to no effect on HLA-A*02 and HLA Class I expression to achieving near complete knockout of HLA-A*02 and HLA Class I expression. The combination of targeting sequences in B2M-5 and B2M-6 gRNAs achieved near complete knockout of both HLA-A*02 and HLA Class I expression in the HLA-A*02+ primary T cells.

### Example 31: gRNA-mediated knockout of B2M increases blocker receptor availability and restores blocker receptor function in HLA-A*02 positive Jurkat cells

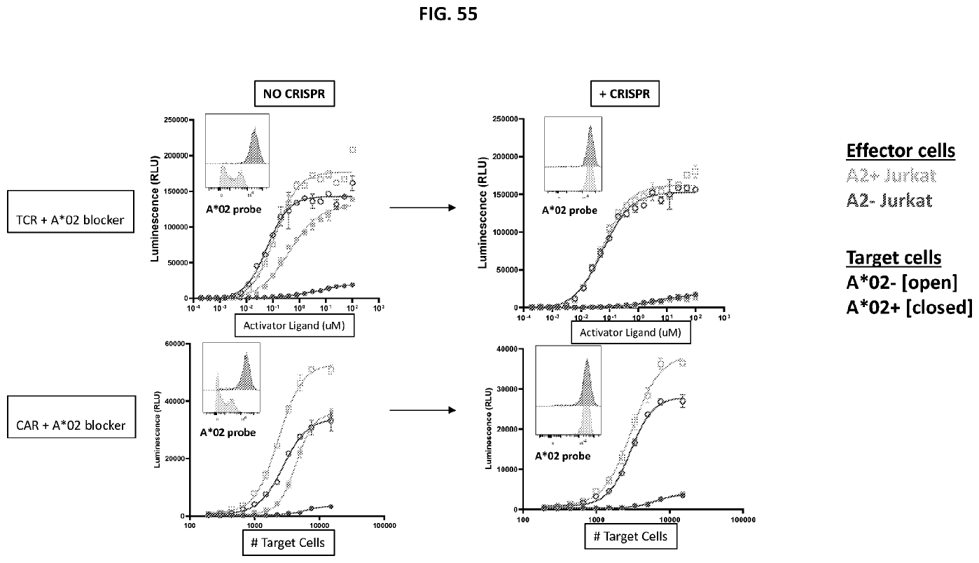

The effect of CRISPR/Cas9 mediated knockout of B2M on blocker ligand binding receptors was determined in Jurkat cells expressing HLA-A*02. (FIG. 55).

Jurkat cells that either express HLA-A*02 (A2+ Jurkat) or do not express HLA-A*02 (A2− Jurkat) were stably transduced with 1) a CAR or TCR that binds an activator ligand and 2) an engineered receptor that binds an HLA-A*02 blocker ligand (A*02 blocker). Cell lines were transfected with pre-complexed Cas9:sgRNA, where the sgRNAs contained a combination of the B2M-1, B2M-2, and B2M-3 targeting sequences in Table 9. Cells were co-cultured with target cells expressing the activator ligand. Target cells expressing the activator ligand either also expressed the HLA-A*02 blocker (inhibitory) ligand (A*02+) or did not express the HLA-A*02 blocker ligand (A*02−). Jurkat and target cells were co-cultured for 4 days, after which CAR/TCR activation was assessed with the Jurkat-NFAT-luciferase activation assay and blocker ligand binding capacity was assessed by FACS analysis following staining with a fluorescently labeled probe that binds the A*02 blocker.

Results are shown in FIG. 55. In A2+ Jurkat cells without B2M knockout (NO CRISPR), activation of the CAR/TCR receptor occurred in the presence of target cells expressing the activator ligand and the HLA-A*02 blocker ligand, as well as in the presence of target cells expressing the activator ligand only. In contrast, A2− Jurkat cells showed little CAR/TCR activation in the presence of target cells expressing the activator ligand and the HLA-A*02 blocker ligand. The A*02 blocker functions to inhibit T cell activation, which was observed in A2− Jurkat cells co-cultured with target cells expressing HLA-A*02 but not in A2+ Jurkat cells co-cultured with target cells expression HLA-A*02. This result indicates that expression of HLA-A*02 in Jurkat cells expressing an activator CAR/TCR and A*02 blocker interferes with A*02 blocker function. In A2+ Jurkat cells transfected with pre-complexed Cas9:sgRNAs targeting the B2M gene, CAR/TCR activation was inhibited in the presence of target cells expressing the HLA-A*02 blocker ligand, but not in the presence of target cells lacking expression of the HLA-A*02 blocker ligand. This result indicates that CRISPR/Cas9 mediated knockout of B2M can restore A*02 blocker function in the A2+ Jurkat cells.

In Jurkat cells stained with the fluorescently labeled probe that binds the A*02 blocker, FACS analysis shows the availability, or binding capacity, of the A*02 blocker receptor (inhibitory receptor). In A2− Jurkat cells, histograms from the FACS analysis shows near complete staining of the cells, indicating near complete availability of the A*02 blocker receptor. In contract, in A2+ Jurkat cells, there is a shift in the histogram, showing a population of cells with little to no A*02 blocker receptor availability and another population with incomplete A*02 blocker receptor availability. However, transfection in A2+ Jurkat cells transfected with pre-complexed Cas9:sgRNAs targeting the B2Mgene shifted the histogram to resemble the A2− population, indicating near complete availability of the A*02 blocker receptor.

Taken together, the results of these experiments show that in Jurkat cells expressing 1) a CAR or TCR that binds an activator ligand and 2) an engineered receptor that binds an HLA-A*02 blocker ligand (A*02 blocker), expression of HLA-A*02 interferes with A*02 blocker receptor function and A*02 blocker binding capacity. Further, the results show that CRISPR/Cas9 mediated knockout of B2M restores A*02 blocker function and binding capacity.

### Materials and Methods for Examples 30 and 31

### Cell Culture

Jurkat cells encoding an NFAT Luciferase reporter were maintained in RPMI media supplemented with 10% FBS, 1% Pen/Strep and 0.4 mg/mL G418/Geneticin. A2+ Jurkat were made by transducing Jurkat NFAT luciferase cells with HLA-A*02 lentivirus (custom lentivirus, Alstem) at a MOI of 5. HLA-A*02-positive cells were sorted using a FACSMelody Cell Sorter (BD).

### Jurkat Cell Transfection

Jurkat cells were transfected with Cas9:sgRNA complexes using a 100 uL format Neon electroporation system (Thermo Fisher) according to manufacturer's protocol using the following settings: 3 pulses, 1500V, 10 msec. Transfections were performed with 20 pmol Cas9 2NLS nuclease (Synthego) and 130 pmol sgRNA (Synthego) per 1 million cells which were pre-incubated for 15 minutes at room temperature prior to transfection. Transfected cells were recovered in RPMI media supplemented with 20% heat-inactivated FBS and 0.1% Pen/Strep.

### Primary T Cell Transduction and Transfection

Frozen PBMCs were thawed in 37° C. water bath and cultured at 1e6 cells/mL in LymphoONE (Takara) with 1% human serum and activated using 1:100 of T cell TransAct (Miltenyi) supplemented with IL-2 (300 IU/mL). After 24 h, if applicable, lentivirus was added to PBMCs at MOI=5. Activator and inhibitory receptor constructs were simultaneously co-transduced at a MOI=5 for each lentivirus. 24 h later, PBMCs were transfected with Cas9/sgRNA complexes using 4D-Nucleofector (Lonza) according to the manufacturer's protocol using the EO-115 program. Transfections were performed with 20 pmol Cas9 2NLS nuclease (Synthego) and 130 pmol sgRNA (Synthego) per 1 million cells which were pre-incubated for 15 minutes at room temperature prior to transfection. Transfected cells were recovered in LymphoONE (Takara) with 1% human serum and IL-2 (300 IU/ml).

### Cell Staining

For gRNA screening studies, Jurkat or primary T cells were stained 4 days after Cas9/sgRNA transfection with BB7.2 antibody (Biolegend) and/or W6/32 antibody (BD Biosciences) for 30 minutes at 4° C. to assess knockout efficiency. To evaluate inhibitor receptor availability to bind HLA-A*02 antigen, Jurkat cells were stained with 10 ug/mL streptavidin-PE-HLA-A*02-pMHC or streptavidin-APC-HLA-A*02-pMHC tetramer for 30 minutes at 4° C. in PBS with 1% BSA and characterized by flow cytometry (BD FACSCanto II).

### Jurkat-NFAT-Luciferase Activation Studies

Jurkat cell activation was determined using the ONE-Step Luciferase Assay System (BPS Bioscience) to evaluate Jurkat luminescence.

### Example 32: gRNA-Mediated Knockout of B2M Increases Blocker Receptor Availability and Restores Blocker Receptor Binding Capacity in HLA-A*02 Primary T Cells

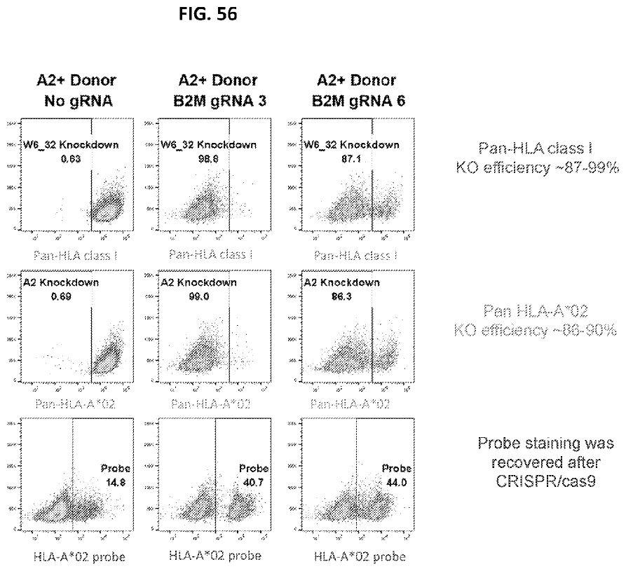

Primary T cells were from an HLA-A*02 positive donor were transduced with CAR activator and HLA-A*02 blocker (A*02 blocker) receptors. Following transduction, the cells were sorted using immunoglobulin-binding protein L and a probe that binds the A*02 blocker. Sorting based on both protein L and the A*02 blocker was necessary to capture all T cells expressing the A*02 blocker because previous experiments showed that expression of HLA-A*02 reduced blocker binding capacity. Primary T cells expressing the CAR and A*02 blocker were transfected with B2M targeting sgRNA sequences, either B2M-3 or B2M-6. Cells were subsequently stained for HLA-A*02 expression, Class I HLA expression, or A*02 blocker probe followed by FACS analysis (FIG. 56). In HLA-A*02 positive donor cells expressing the CAR activator and A*02 blocker receptors, staining and FACS analysis prior to transfection with B2M targeting sgRNA sequences shows the cells were positive for Class I HLA and the HLA-A*02 allele, while over 85% negative for the A*02 blocker. Transfection with B2M targeting sgRNA resulted in efficient knockout of Class I HLA (98.8% and 87.1% for B2M-3 and B2M-6, respectively) and the HLA-A*02 (99.0% and 86.3% for B2M-3 and B2M-6, respectively). In the cell populations with sgRNA mediated knockout of B2M, the A*02 blocker staining increased from 14.8% of the cell population to 40.7% and 44.0% for B2M-3 and B2M-6, respectively. These results corroborate the observation in Example 3 that CRISPR/Cas9 mediated knockout of B2M in HLA-A*02 positive T cells expressing CAR activator and A*02 blocker receptors increases binding capacity of the A*02 blocker. These results suggest that endogenous expression of HLA-A*02 interferes with the HLA-A*02− specific blocker receptor ligand binding capacity, and that knockout of endogenous B2M expression reverses the effect. Thus, gRNA-mediated knockout of endogenous B2M in immune cells expressing an HLA-A*02-specific blocker receptor can reduce autocrine interference or activation of the blocker receptor.

### Example 33: Design of gRNA Sequences Targeting the B2M Promoter

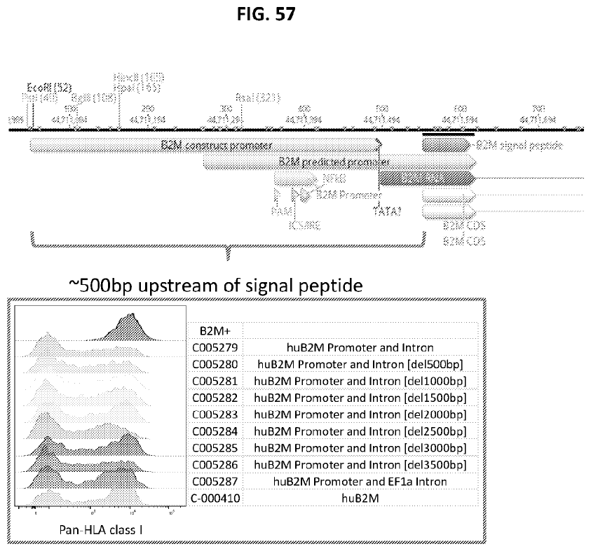

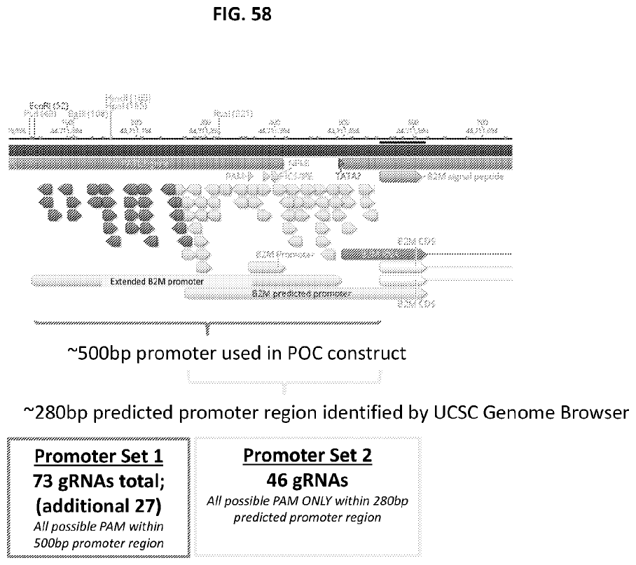

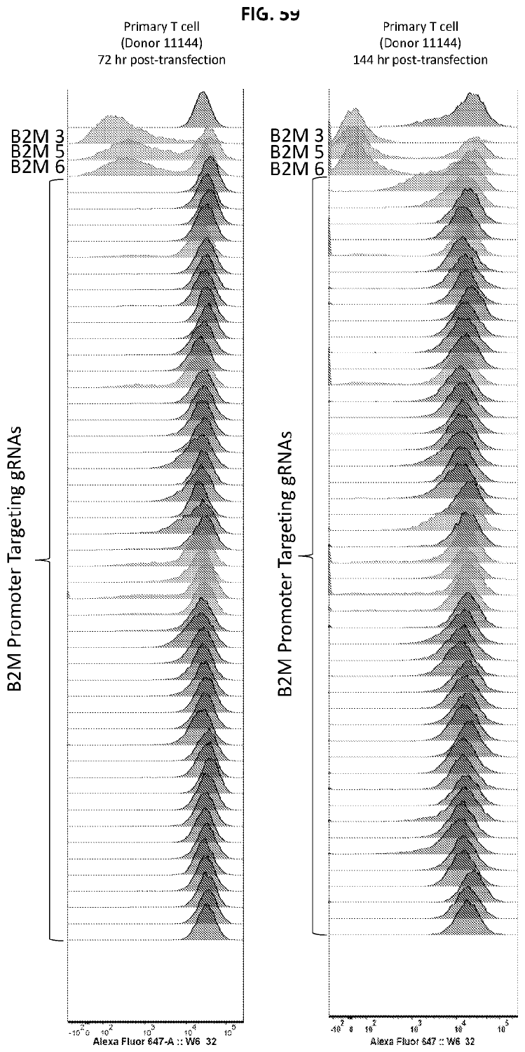

Inactivating expression of the B2M gene by targeting the B2M promoter with gRNA sequences can potentially reduce expression of Class I HLA complexes. In order to identify B2M promoter sequences for targeting with gRNAs, 500 bp truncations of the sequence upstream of the B2M gene were carried out in Jurkat cells and the effect on expression of Class I HLA complexes was determined by staining cells for Class I HLA expression followed by FACS analysis. The analysis shows that the minimum region sufficient for expression of Class I HLA is the 500 bp region upstream of the sequence encoding the signal peptide. This region may be considered the minimum promoter for the B2M gene and is a putative candidate region for gRNA targeting with gRNAs (FIG. 57). A set of gRNA sequences targeting the region 500 bp region upstream of the sequence encoding the B2M signal peptide was developed. The total of 73 gRNA sequences were designed to be complementary to a 20 nt sequence adjacent to a Cas9 compatible PAM (NGG) in the 500 bp region (FIG. 58). A subset of 46 gRNA sequences target the predicted region predicted to be the B2M promoter identified by the UCSC Genome Browser (Lee et al. Nucleic Acids Research. 48:D756-D761 (2020)). These gRNA sequences are characterized using methods as described in Examples 2-4 for validating their ability to reduce expression of Class I HLA complexes (FIG. 59). The promoter targeting gRNA sequences showed little efficacy in reducing Class I HLA expression.

### Example 34: Selection of Interfering RNA Sequences Targeting the B2M Gene

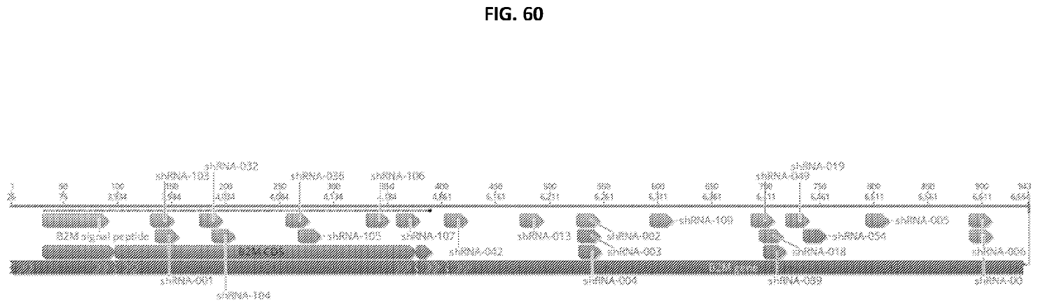

In order to select guide nucleic acids for targeting the B2M gene, all possible 18 bp, 19 bp, 20 bp, 21 bp, and 22 bp transcribed sequences corresponding to B2M mRNA were determined, yielding 4620 sequences. From this set of putative target sequences, those containing GC content less than 25% or greater than or equal to 60% were excluded, resulting in 3596 potential target sequences. From this set of potential target sequences, those with runs of 4 or more of the same base in a row or a run of 7 C or G bases in a row were removed, resulting in 3000 potential target sequences. The set was further reduced by removing all potential sequence that were not 21 bp long, resulting in 582 potential target sequences. Next, the sequences were analyzed by identifying favorable characteristics identified by computational tools, such as the GPP RNAi Designer (Broad Institute), and similar favorable characteristics. The final set of potential targets included 282 sequences that can be used to design interfering RNAs that target the B2M mRNA transcript for degradation through RNA interference. Illustrative alignments between shRNAs with a complementary sequence to the identified targets of the analysis are shown in FIG. 60.

### Example 35: shRNA-Mediated Reduction of B2M Expression Reduces Class I HLA Expression

The effect of shRNA mediated reduction of B2M expression on Class I HLA expression was assessed.

Jurkat cells expressing HLA-A*02 were transfected using 100 uL format Neon electroporation system (Thermo Fisher) according to manufacturer's protocol using the following settings: 3 pulses, 1500V, 10 msec. Co-transfections were performed with 300 ng of U6-shRNA plasmids and 1.2 ug of plasmid expressing EGFR as a transfection marker per 1 million cells. Transfected cells were recovered in RPMI media supplemented with 20% heat-inactivated FBS and 0.1% Pen/Strep.

4 days following transfection, cells were stained for HLA-A*02 expression with BB7.2 antibody (Biolegend) and/or HLA Class I expression W6/32 antibody (BD Biosciences) and anti-EGFR antibody (Biolegend) for 30 minutes at 4° C. to assess knockdown efficiency.

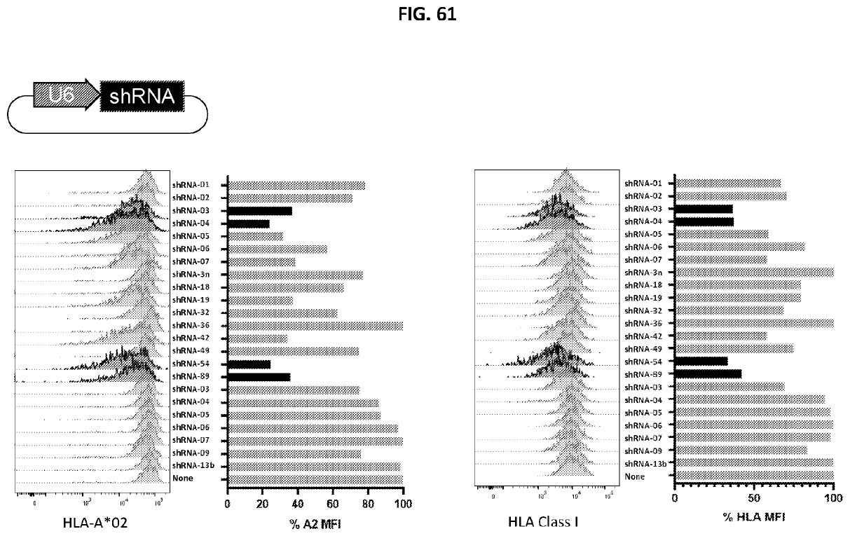

The results are shown in FIG. 61. The histograms show staining for either HLA-A*02 or HLA Class I, and corresponding quantification as % expression in control cells. The results indicate that several of the shRNA candidates were able to reduce expression of HLA-A*02 and HLA Class I. In particular, shRNA-03, shRNA-04, shRNA-54, and shRNA-89 showed greater than 50% decrease compared to control cells.

These results indicate shRNA mediated reduced expression of B2M results in reduced expression of HLA-A*02 and HLA Class I molecules in immune cells.

### Example 36: shRNA-Mediated Reduction of B2M Expression Increases Blocker Receptor Availability and Restores Blocker Receptor Function in HLA-A*02 Positive Jurkat Cells

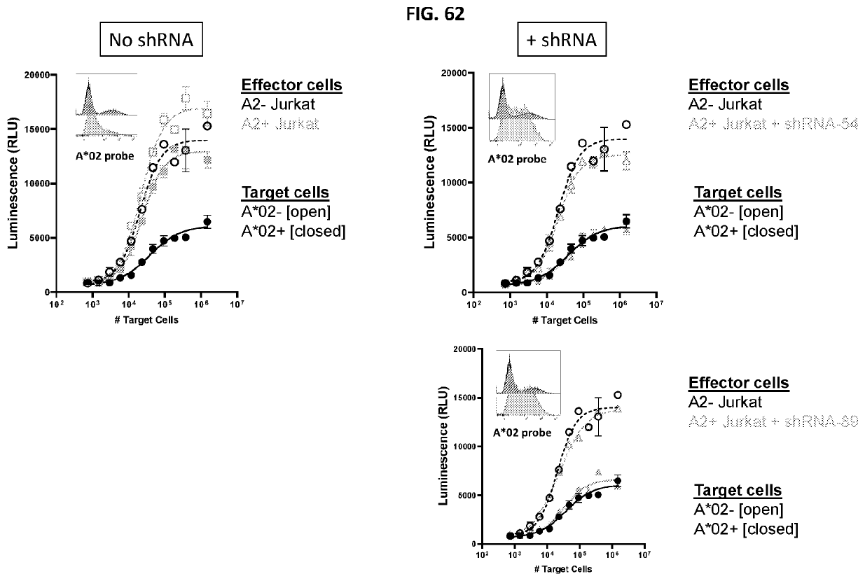

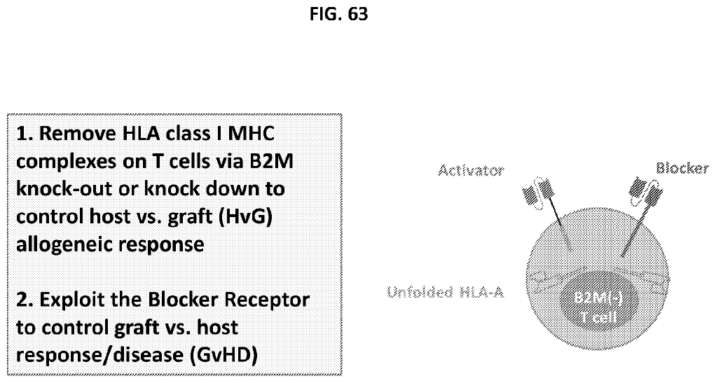

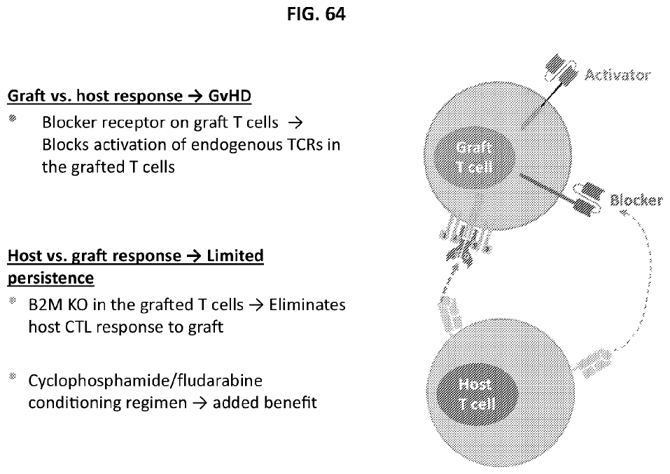

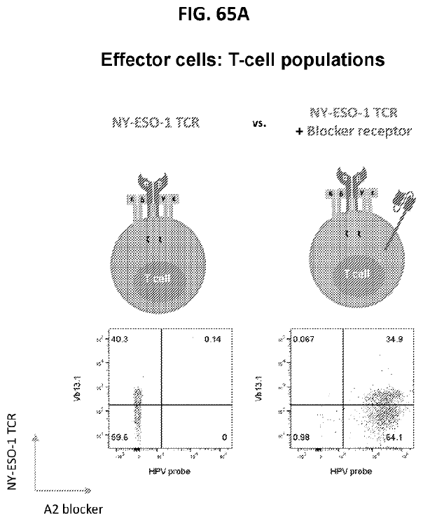

The effect of shRNA mediated reduction of B2M expression on blocker ligand binding receptors was determined in Jurkat cells expressing HLA-A*02. (FIG. 62).

A2− and A2+ transduced Jurkat cells were transfected using 100 uL format Neon electroporation system (Thermo Fisher) according to manufacturer's protocol using the following settings: 3 pulses, 1500V, 10 msec. Co-transfections were performed with 2 ug CAR plasmids and 2 ug of either U6-shRNA/A*02-blocker or A*02-blocker alone per million cells. Transfected cells were recovered in RPMI media supplemented with 20% heat-inactivated FBS and 0.1% Pen/Strep.

Jurkat cells that either express HLA-A*02 (A2+ Jurkat) or do not express HLA-A*02 (A2− Jurkat) were transfected using 100 uL format Neon electroporation system (Thermo Fisher) according to manufacturer's protocol using the following settings: 3 pulses, 1500V, 10 msec. Co-transfections were performed with 2 ug plasmids encoding activator and inhibitory receptors, and 2 ug of either U6-shRNA/A*02-blocker or A*02-blocker alone per million cells. Transfected cells were recovered RPMI media supplemented with 20% heat-inactivated FBS and 0.1% Pen/Strep.

2 days following transfection, 10⁴ Jurkat cells were co-cultured with 1.2×10⁴ HeLa target cells and incubated in Corning® 384-well Low Flange White Flat Bottom Polystyrene TC-treated Microplates for 6 hours. ONE-Step Luciferase Assay System (BPS Bioscience) was used to evaluate Jurkat luminescence. To evaluate blocker availability to bind HLA-A*02 antigen, Jurkat cells were stained with 10 ug/mL streptavidin-PE-HLA-A*02-pMHC or streptavidin-APC-HLA-A*02-pMHC tetramer for 30 minutes at 4° C. in PBS with 1% BSA and characterized by flow cytometry (BD FACSCanto II). Histograms show all cells.

Results are shown in FIG. 62. In A2+ Jurkat effector cells in the absence of shRNA targeting B2M, activation of the CAR/TCR receptor occurred in the presence of target cells expressing the activator ligand and the HLA-A*02 blocker ligand, as well as in the presence of target cells expressing the activator ligand only. In contrast, A2− Jurkat effector cells showed significantly less CAR/TCR activation in the presence of target cells expressing the activator ligand and the HLA-A*02 blocker ligand. The A*02 blocker functions to inhibit T cell activation, which was observed in A2− Jurkat effector cells co-cultured with target cells expressing HLA-A*02 but not in A2+ Jurkat effector cells co-cultured with target cells expression HLA-A*02. This result indicates that expression of HLA-A*02 in Jurkat effector cells expressing an activator CAR/TCR and A*02 blocker interferes with A*02 inhibitory receptor function. In A2+ Jurkat effector cells transfected with shRNA targeting the B2M expression, CAR/TCR activation was inhibited in the presence of target cells expressing the HLA-A*02 blocker ligand, but not in the presence of target cells lacking expression of the HLA-A*02 blocker ligand. This result indicates that shRNA mediated reduction of B2M expression can restore A*02 blocker function in the A2+ Jurkat cells.

In Jurkat cells stained with the fluorescently labeled probe that binds the A*02 blocker, FACS analysis shows the availability, or binding capacity, of the A*02 blocker receptor (inhibitory receptor). In A2− Jurkat cells, histograms from the FACS analysis shows staining of a subset of cells, indicating availability of the A*02 blocker receptor. In contrast, in A2+ Jurkat cells, there is a shift in the histogram, showing the population of cells has little to no A*02 blocker receptor availability and another ill-defined population with incomplete A*02 blocker receptor availability. However, transfection in A2+ Jurkat cells with shRNA targeting the B2M expression shifted the histogram to resemble the A2− population, indicating near complete availability of the A*02 blocker receptor.

Taken together, the results of these experiments show that in Jurkat cells expressing 1) a CAR or TCR that binds an activator ligand and 2) an engineered receptor that binds an HLA-A*02 blocker ligand (A*02 blocker), expression of HLA-A*02 interferes with A*02 blocker receptor function and A*02 blocker binding capacity. Further, the results show that shRNA mediated reduction of B2M expression restores A*02 blocker function and binding capacity.

### Example 37: Expression of a Blocker Receptor by T Cells Blocks Killing Mediated by TCR Receptors on the T Cells

The ability of the blocker receptor, when expressed by T cells, to block killing mediated by T cell TCR expression was assayed using primary T cells and the NY-ESO-1 and KRAS TCRs.

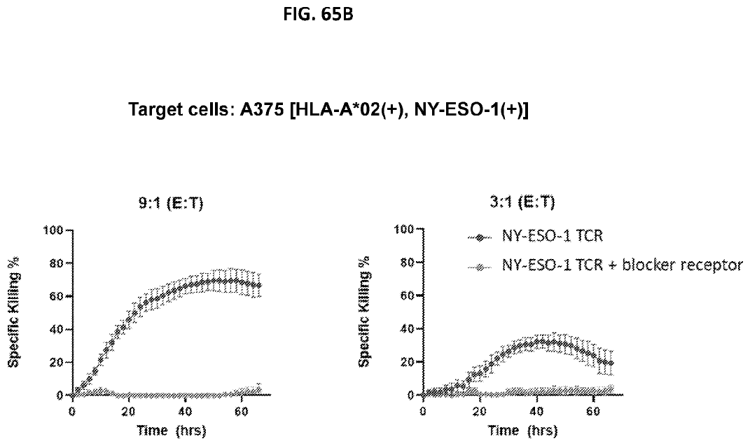

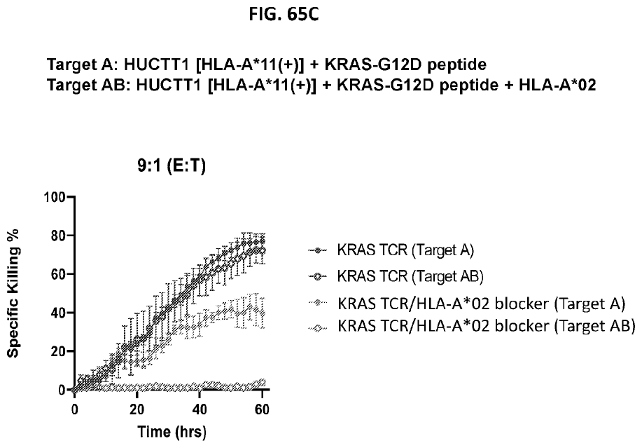

Primary T cells were transduced with a vector encoding an NY-ESO-1 or KRAS TCR receptor as a control, or co-transduced with either the NY-ESO-1 or KRAS TCR receptor in combination with an HLA-A*02 LIR1 inhibitory (blocker) receptor. Expression of the TCR and the blocker receptor were confirmed by flow cytometry (FIG. 65A, NY-ESO-1 TCR alone shown at left, NY-ESO-1 TCR and the blocker pair shown on the right; equivalent KRAS TCR and KRAS and blocker pair are not shown). T cells expressing the TCR only or the TCR the blocker receptor in combination were co-cultured with target cells as indicated in FIGS. 65B and 65C. In FIG. 65B, A375 target cells, which are NY-ESO+/HLA-A2+, were used as target cells for effector cells expressing the NY-ESO-1 TCR, or the NY-ESO-1 TCR and blocker receptor pair. As shown in FIG. 65B, significant killing was observed at 9:1 and 3:1 E:T ratio with the NY-ESO-1 TCR alone, but not when the T cells expressed both the NY-ESO-1 TCR and the HLA-A*02 LIR1 blocker receptor.

To assay the effect of the blocker receptor on effector cells expressing the KRAS TCR, HuCTT1 target cells expressing the KRAS-G12D peptide and HLA-A*11 (KRAS-G12D/A11 or KRAS G12D/A11, referred to in FIG. 65C as Target A) were co-cultured with effector T cells expressing the KRAS TCR, or T cells expressing both the KRAS TCR and the HLA-A*02 LIR1 blocker receptor. HuCTT1 target cells that did, and did not, co-express HLA-A*02 (referred to as target B in FIG. 65C), were also used in this assay. As shown in FIG. 65C, the KRAS TCR showed significant cytotoxicity on both target cell types, those expressing both target A and both targets A and B (target AB). KRAS TCR and HLA-A*02 LIR1 blocker receptor expressing T cells only showed killing of KRAS-G12D/A11 (activator antigen) expressing cells, and did not show killing of KRAS-G12D/A11 and A2 (activator antigen and blocker antigen) expressing target cells.

In summary, the blocker receptor silences an extremely potent TCR directed at NY-ESO-1 which has a sensitivity of ˜10 molecules/cell. Blocker receptor activity was not limited to HLA-A*02-restricted TCRs. As shown in FIG. 65C, the HLA-A*02 LIR1 blocker receptor was also able to silence an HLA-A*11-restricted KRAS-target TCR.

The blocker receptor is capable of controlling the graft versus host allogeneic response similarly to what is seen in T cells in which TRAC has been knocked out.

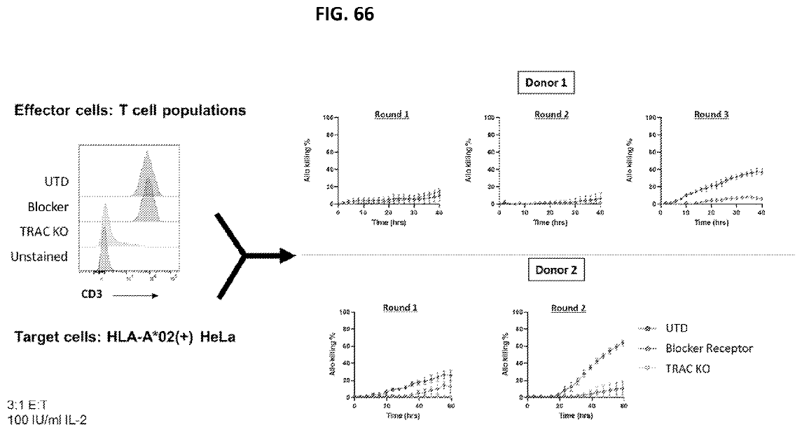

Untransduced T cells, T cells transduced with HLA-A*02 LIR1 blocker receptor, or in which TRAC (also known as TRCA) had been knocked out using CRISPR/Cas9 to induce indels in the TRAC locus, were co-cultured with HLA-A*02+ HeLa target cells to determine the cytotoxicity caused by allogeneic reactivity. T cells were purified from two donors (donors 1 and 2 in FIG. 66). Allogeneic reactivity was triggered by multiple rounds of exposure to HeLa target cells at a 3:1 effector to target ratio, in the presence of 100 IU/mL of IL-2. In round 3 for donor 1, and in round 2 for donor 2, allogeneic reactivity can be seen in the untransduced TRAC+ control cells (UTD), but was blocked to a similar degree by T cells expressing the blocker receptor, or in which TRAC had been knocked out.

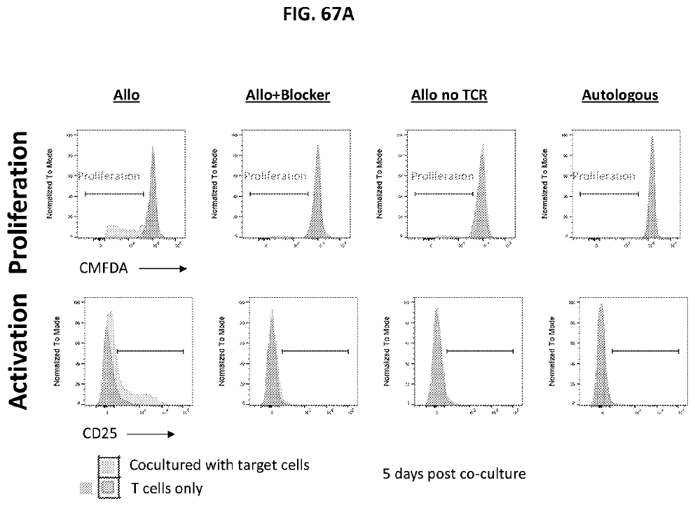

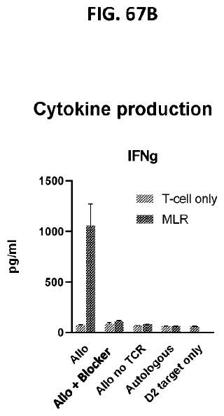

The blocker receptor is also able to minimize allogeneic response in mixed lymphocyte reactions. T cells were isolated from the peripheral blood mononuclear cells (PMBC) of two donors. The TRAC locus was knocked out in a portion of these T cells using CRISPR/Cas9 (“Allo no TCR” cells in FIG. 67A), and HLA-A*02 LIR1 blocker receptor was expressed in another portion of the isolated T cells (Allo+Blocker in FIG. 67A). T cells alone, with blocker receptor expression, or with TRAC KO from donor 1 were loaded with CMFDA dye and co-cultured with T-cell depleted PBMCs from donor 2 (Allo, Allo+Blocker, Allo no TCR, respectively in FIG. 67A). T cells alone from donor 1 were also co-cultured with T-cell depleted PBMCs from donor 1 (Autologous in FIG. 67A). Proliferation of T cells from donor 1 was measured by monitoring the decreasing of intensity of CMFDA dye, which is diluted as the cells divide, upper series of panels in FIG. 67A. T cell activation was monitored by CD25 expression, as seen in the lower panels in FIG. 67A. Cytokine (interferon gamma, IFN-g or IFNG) secretion was also measured in T cell only wells and co-cultured wells (FIG. 67B). T cells from donor 1 co-cultured with PBMC from donor 2 triggered T cell proliferation, activation and cytokine secretion. These activities were reduced in T cells with TRAC knock out, or which expressed the blocker receptor. The reduction was similar to the level seen in autologous co-culture (FIGS. 67A-67B).

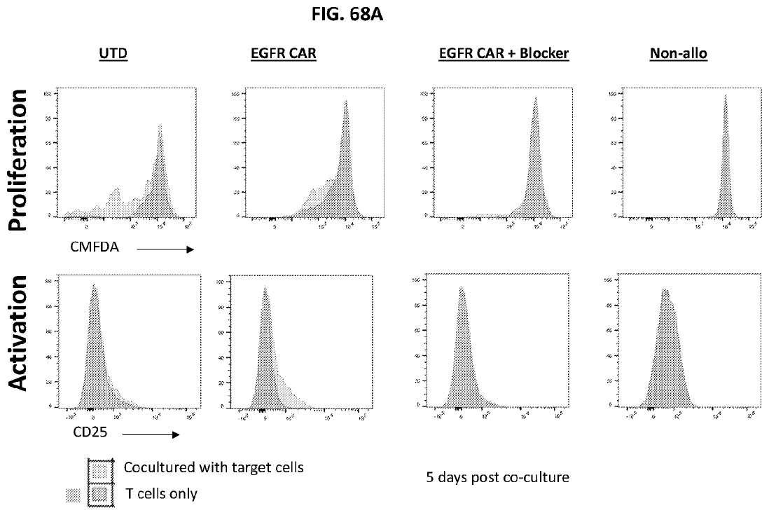

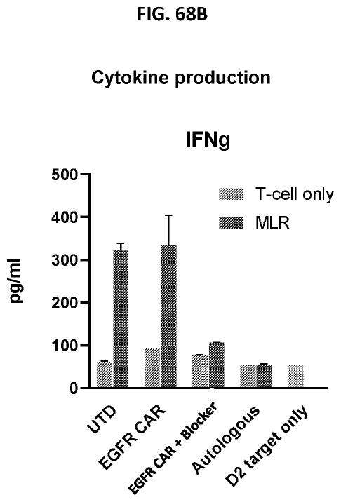

Similar results were seen when the primary T cells were also transduced with an EGFR chimeric antigen receptor (FIGS. 68A-68B). T cells were isolated from PMBC from two donors. Untransduced T cells (UTD), T cells expressing EGFR CAR alone, or EGFR CAR in combination with a blocker receptor, each from donor 1, were loaded with CMFDA dye and co-cultured with T-cell depleted PBMCs from donor 2 (UTD, EGFR CAR, EGFR+Blocker, respectively in FIG. 68A). Un transduced T cells (UTD) from donor 1 was also co-cultured with T-cell depleted PBMCs from donor 1 (Non-Allo in FIG. 68A). Proliferation of T cells from donor 1 was measured by monitoring decreasing of intensity of CMFDA (FIG. 68A, top row), and T cell activation was monitored by CD25 expression (FIG. 68A, bottom row). Cytokine (IFN-g) secretion was also measured in T cell only wells and co-cultured wells. Untransduced T cells, and EGFR CAR expressing T cells from donor 1 co-cultured with PBMC from donor 2 triggered T cell proliferation, activation and cytokine secretion. These activities were reduced in T cells expressing both the EGFR CAR and the blocker receptor, and the reduction was similar to the level similar to the “non-allo”, i.e. autologous, co-culture.

### Example 38: Mouse Model of Effect of Blocker Receptor on Allogeneic Transplanted Cells

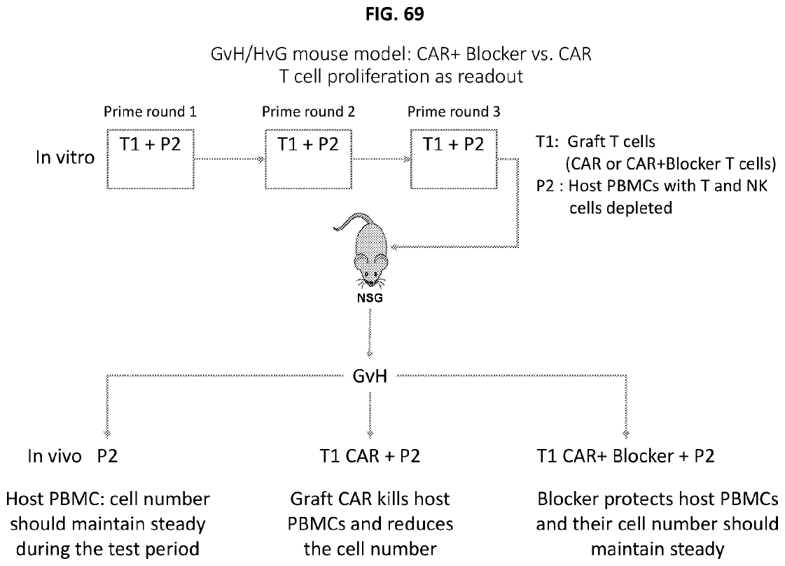

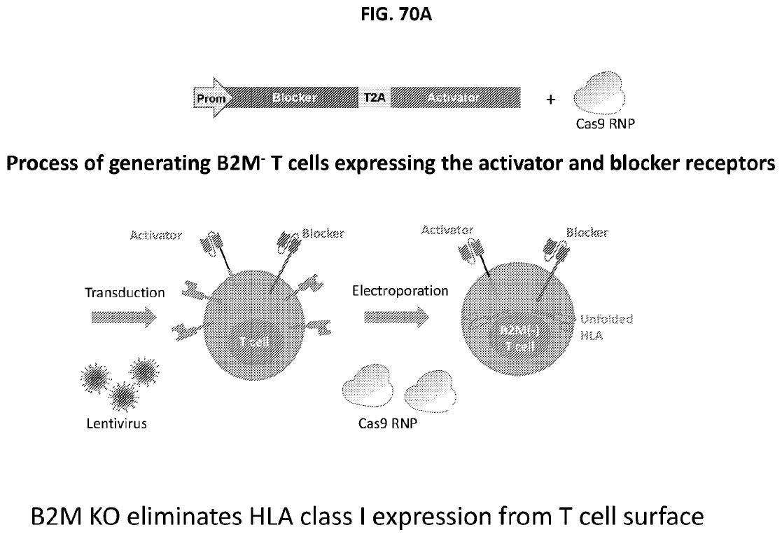

FIG. 69 is an experimental scheme for testing graft versus host and host versus graft allogeneic effects of T cells expressing both an activator and a blocker receptor in a mouse model. Allogeneic mouse T cells to be administered to a mouse (i.e., graft T cells) expressing the activator receptor, for example an EGFR CAR, or an activator receptor in combination with a blocker receptor, will undergo three rounds of co-culture in vitro with host PBMCs from a different mouse which have been depleted of T and NK cells. The effect of co-culture with host PBMCs on T cell proliferation, and optionally activation and cytokine secretion will be assayed as described in Example 37. In addition, NSG immunodeficient mice will be injected with (1) host (i.e., their own) PBMCs that have been T and NK cell depleted; (2) a combination of graft T cells (from a different mouse) that have been transduced with the CAR and the host PBMCs that have been T and NK cell depleted; or (3) a combination of graft T cells that have been transduced with the CAR and the blocker receptor, and the host PBMCs that have been T and NK cell depleted. It is expected that in the mice injected with (1) host PBMCs, cell numbers of host PBMCs should remain steady during the testing period. It is expected that in mice injected with (2) graft T cells expressing the CAR and host PBMCs, that the graft T cells expressing the CAR will kill host PBMCs and reduce their number. It is also expected that mice injected with (3) graft T cells expressing the CAR and the blocker, and host PBMCs, that the blocker receptor will protect the host PBMCs and their number should remain steady.

### Example 39: Knock-Out of B2M

Multiple methods can be used to mitigate host versus graft effects, including knock out of B2M using CRISPR/Cas9, or by incorporation of an interfering RNA such as an shRNA targeting B2M into a lentiviral vector, such as a lentiviral vector encoding the blocker and/or activator receptors. CRISPR/Cas9 knock out of B2M can be accomplished by delivery of the CRISPR/Cas9 ribonucleoprotein (RNP) targeting the B2M locus through transfection into T cells, or by encoding the CRISPR/Cas9 and gRNA components of the RNP using a lentiviral vector. Combinations of delivery methods can also be used. For example, the gRNA can be incorporated into a lentiviral vector, while the Cas9 protein (or a sequence encoding the Cas9 protein) is delivered by transfection.

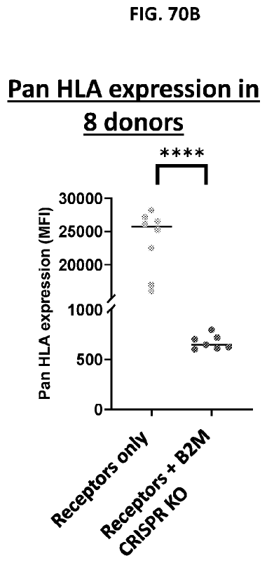

FIG. 70A shows one method for generating B2M(−) T cells, i.e. T cells in which the B2M locus has been knocked out by creation of a deletion caused by using CRISPR/Cas9 and one or more gRNAs specific to B2M that induces a double strand break in the locus. In this method, T cells are transduced with a lentiviral vector encoding the blocker and activator receptor under control of single promoter. The coding sequences of the two receptors are separated by a T2A self cleaving polypeptide. After transduction, the T cells are electroporated with Cas9 RNP with gRNAs that target B2M, resulting in B2M knockout. B2M knockout eliminates the expression of HLA class I MHC complexes from the T cell surface.

FIG. 70B shows pan-HLA expression in T cells from 8 donors in which the T cells had been transduced with the lentiviral vector shown in FIG. 70B, with and without B2M knockout. As can be seen in FIG. 70B, B2M knockout reduced HLA expression in all 8 donors.

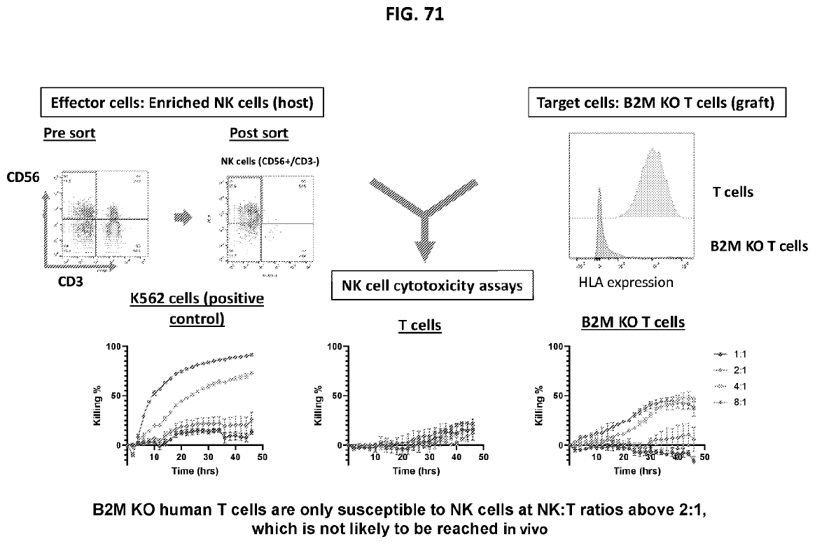

B2M knockout in T cells expressing the activator and blocker receptor pair produces T cells that are protected from T cell killing, and limited susceptibility to NK cell mediated killing. NK effector cells, which are CD3(−) CD56(+), from the donor 1 (host) were sorted using the CD3ξ and CD56 markers and FACS as shown in FIG. 71, upper left. These NK effector cells were co-cultured with T cells with or without B2M knockout from donor 2 (graft) (FIG. 71, bottom, middle and right panels). K562 cells were also co-cultured with the NK cells as positive control (FIG. 71, bottom row, left panel). T cells without B2M knockout showed minimal killing by NK cells at up to an 8:1 NK:T ratio. T cells with B2M knockout were killed by NK cells at >4:1 NK:T cell ratio. Even though this ratio may not be physiologically relevant, NK cell killing can be minimized through optimization. Co-culture was carried out in LymphoOne medium, for 48 hours, with no IL-2.

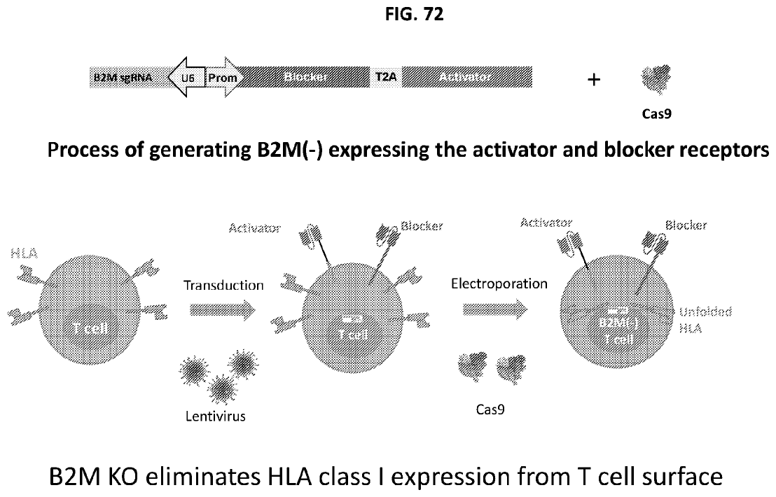

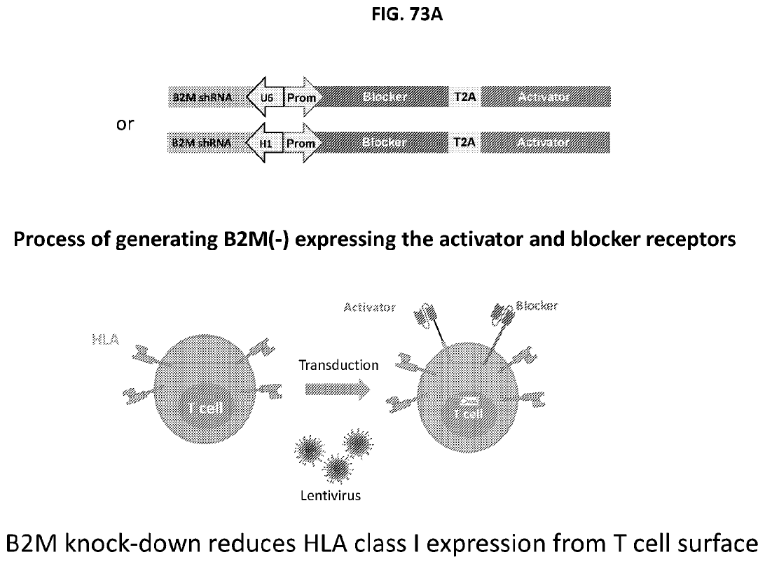

FIG. 72 shows another method for generating B2M(−) T cells expressing the activator and blocker receptor pair. In this method, B2M is again knocked out by CRISPR/Cas9. However, the gRNA and two receptors are encoded by a lentiviral vector such as the one shown, and the Cas9 protein is delivered by electroporation. One advantage of this method is that because the gRNA and the two receptors are delivered by the same lentiviral vector, HLA expression is linked to expression of both receptors. Purification/enrichment for cells expressing the two receptors can be done by HLA(+) cell depletion.

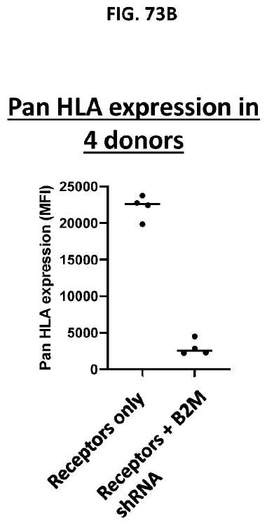

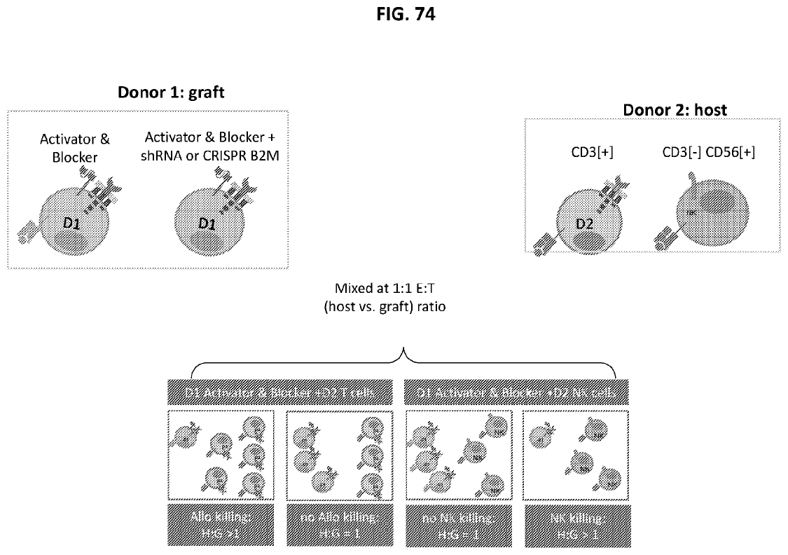

FIG. 73A shows method for generating B2M(−) T cells expressing the activator and blocker receptor pair using an interfering RNA, such as a short hairpin RNA (shRNA) specific to a B2M sequence to knock down B2M expression. In this method, the B2M shRNA and two receptors are encoded in a lentiviral vector. One advantage is that the reduction of HLA expression is again linked to expression of the activator and blocker receptor pair. Purification/enrichment for cells expressing the two receptors can be done by HLA(+) cell depletion.

FIG. 73B shows pan-HLA expression in T cells from 4 donors that have been transduced with a lentiviral vector encoding an activator and blocker receptor pair, or a lentiviral vector encoding an activator and blocker receptor pair and a B2M shRNA. As can be seen from FIG. 73B, transduction with a vector including the B2M shRNA reduced HLA expression in T cells from all four donors.

### Example 40: Effect of B2M Knock-out or Knock Down on NK and T Cell Killing by Host Cells

The effect that knocking out or knocking down B2M in graft T cells has on killing mediated by host NK and T cells was assayed used the experiment depicted in FIG. 74. B2M was knocked out, or knocked down, in graft T cells from a first donor (donor 1), as described in Example 39. The donor 1 T cells also expressed the activator and blocker receptors. CD3-CD56+NK cells from a second donor (host, or donor 2) were purified from PBMCs using FACS. The T cells from donor 1 were then mixed with the NK cells from donor 2 at a ratio of 1:1 host to graft cells and co-cultured under appropriate culture conditions, in LymphoOne medium, for 5 days, with no IL-2. The graft T cells from donor 1 were also mixed with the NK cells from donor 2 and co-cultured under similar conditions. On day 5, if the graft cells were killed, the E:T ratio on day 5 should increase to be greater than 1. If no killing of graft cells occurred, then the E:T ratio should be less than or equal to 1.

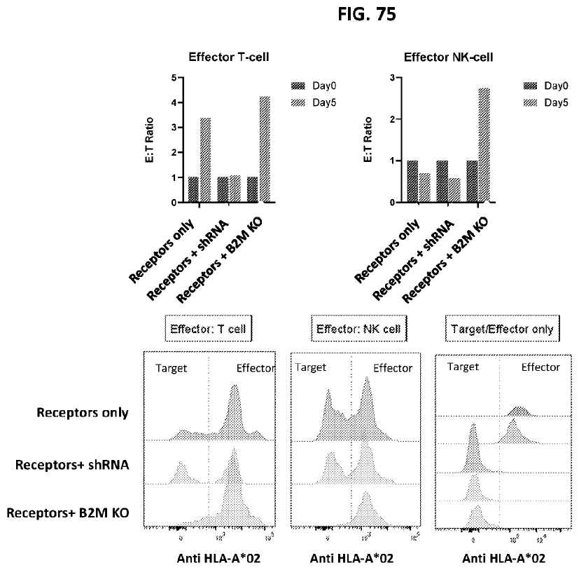

The results are shown in FIG. 75. T cells expressing the activator and blocker receptors, the activator and blocker receptors with a B2M shRNA, or the activator and blocker receptors with CRISPR/Cas9 mediated B2M knockdown from one donor (D1, graft) were co-cultured with T cells or NK cells from another donor (D2, host) at a host to graft ratio of 1:1 for 5 days, without IL-2. As seen in FIG. 75, donor T cells expressing both receptors, which were HLA(+), were killed by host T cells due to HLA mismatch. These donor T cells were not killed by NK cells. Donor T cells expressing the B2M shRNA, which had very low HLA, were not killed by host T cells as there was very low HLA mismatch. Nor were these cells killed by NK cells, as there was still enough HLA to prevent this from happening without cytokine. However, T cells in which B2M had been knocked out were also killed by host T cells and NK cells.

### Example 41: HLA-A Knock-Out Using CRISPR/Cas9 in Primary T Cells

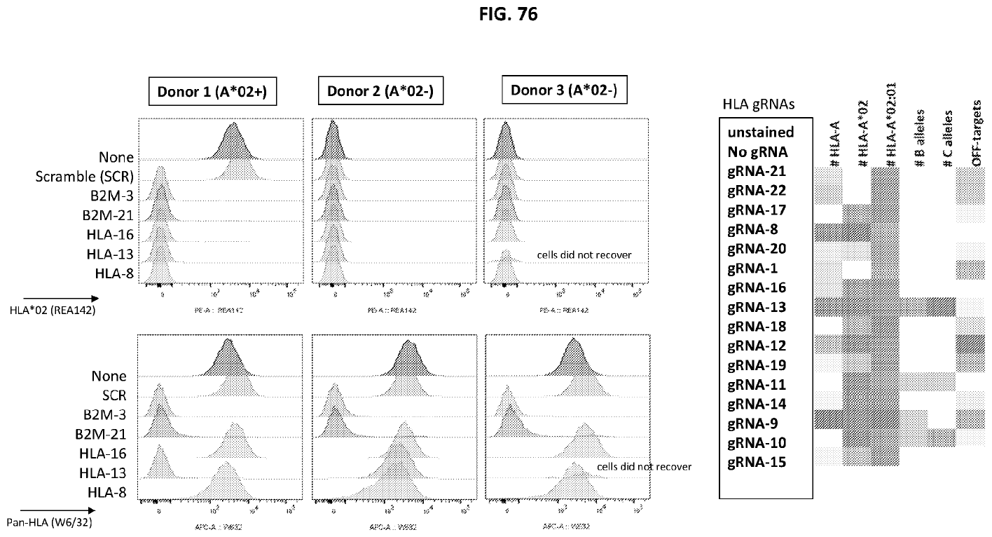

Primary T cells were isolated from PBMCs from three donors, one who was HLA-A*02+ and two who were HLA-A*02−, and transduced with an activator CAR, or an activator CAR in combination with an HLA-A*02 LIR1 inhibitory receptor, as described above. Transduced T cells were than transfected with gRNAs to HLA-A*02 or B2M, a scrambled gRNA as a control, and Cas9 protein. Untransduced control cells were also transfected with the gRNAs to HLA-A*02, B2M, or the scrambled gRNA and the Cas9. Transfected T cells then stained for HLA expression using an HLA-A*02 specific primary antibody (REA142, from Miltenyi Biotec), or a pan-HLA primary antibody (W6/32), and the expression levels were assayed using fluorescence activated cell sorting. The results are shown in FIG. 76. As shown in FIG. 76, when HLA-A*02 cell surface expression was assayed (top row, left 3 panels) HLA-A*02 levels were reduced by all HLA-A*02 gRNAs to a level comparable to that seen with two B2M gRNAs. When HLA expression was assayed with a pan-HLA antibody (FIG. 76, bottom row, left 3 panels), HLA gRNA-16 was able to reduce HLA surface expression in the HLA-A*02+ donor to a level comparable to that seen with the two B2M gRNAs. HLA gRNAs-13 and -008 did not achieve a similar reduction in total HLA, presumably due to the expression of the non-HLA-A*02 alleles by the HLA-A*02− donors. This result is an accordance with in silico specificity predicted for HLA gRNAs shown in the heat map in FIG. 76 at right (level of gray shading indicates predicted cutting at the indicated locus).

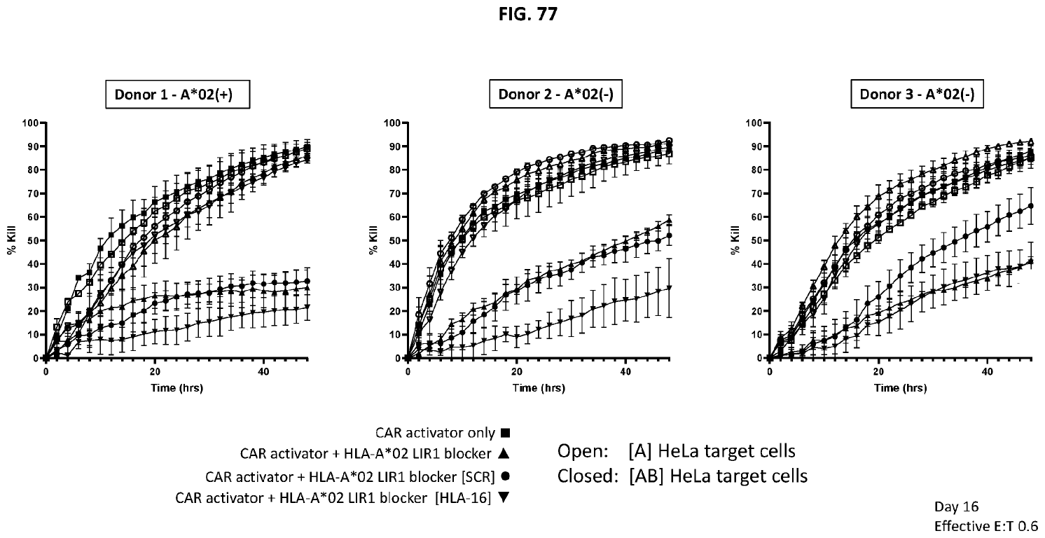

Primary T cells transduced with an activator CAR and/or HLA-A*02 LIR1 blocker receptor, and with HLA-A*02 knocked out using HLA gRNA-16, were assayed for their ability to selectively kill HeLa target cells. Transduced and transfected T cells were co-cultured for 16 days with HeLa cells expressing the CAR activator target (FIG. 77, referred to as [A]), or with HeLa cells expressing both the CAR activator target and HLA-A*02, the blocker receptor target ([AB] HeLa cells in FIG. 77). As shown in FIG. 77, primary T cells from an HLA-A*02+ donor which expressed the activator and blocker receptors, and which had HLA-A*02 knocked out using HLA gRNA-16, were able to selectively kill HeLa cells expressing the activator target, but not HeLa cells expressing both the activator and blocker targets. In equivalent primary T cells from HLA-A*02 negative donors, transfection with HLA gRNA-16 did not negatively affect selective killing of HeLa cells expressing the activator, but not the activator and blocker, targets.

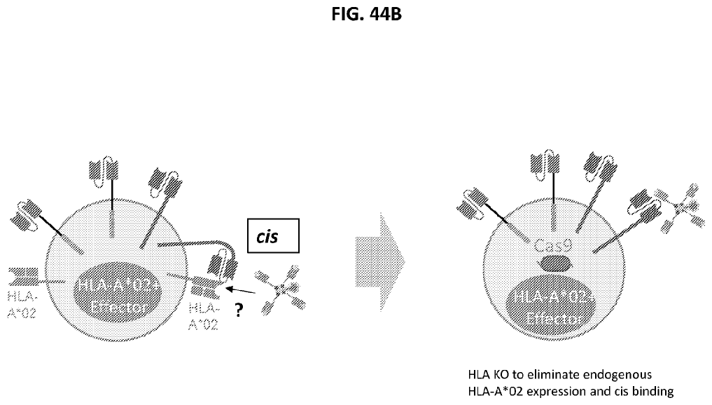

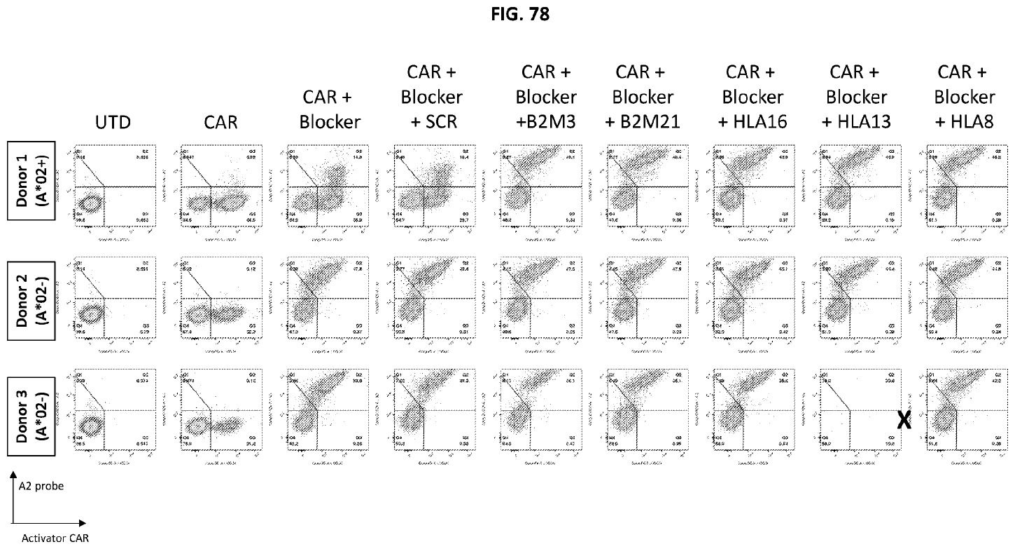

FIG. 78 shows that knocking out B2M or HLA-A*02 in T cells from an HLA-A*02+ donor restores HLA/peptide tetramer binding to the T cells (see FIG. 44B for a schematic). Cis-binding of cell surface HLA-A*02 to the HLA-A*02 blocker receptor. As can be seen in FIG. 78, when primary T cells from an HLA-A*02+ donor were probed with a labeled HLA-A*02 incorporated into HLA tetramer, labeling of the T cells through binding of the tetramer to the blocker receptor was inhibited, presumably by endogenous HLA-A*02 interacting with the blocker receptor. Upon knockdown of HLA or B2M with CRISPR/Cas9 and the indicated gRNA, labeling of T cells by labeled tetramer was restored, and equivalent to that seen with T cells from HLA-A*02− donors (FIG. 77).

### REPRESENTATIVE EMBODIMENTS

### Representative Embodiments, Set 1

1. An allogeneic immune cell comprising:
a. a first engineered receptor, the first engineered receptor comprising a transmembrane region and an extracellular region, the extracellular region comprising a first ligand binding domain capable of specifically binding a first ligand; and
b. a second engineered receptor, the second engineered receptor comprising a transmembrane region and an extracellular region, the extracellular region comprising a second ligand binding domain capable of specifically binding a second ligand, wherein binding of the first ligand binding domain to the first ligand activates or promotes activation of the immune cell by the first receptor, and wherein binding of the second ligand binding domain to the second ligand inhibits activation of the immune cell by the first receptor.
2. The allogeneic immune cell of embodiment 1, comprising a first modification that reduces or eliminates expression or function of a component of the major histocompatibility class I complex (MHC I).
3. The allogeneic immune cell of embodiment 2, wherein the component of MHC I is HLA-A, HLA-B, HLA-C or B2M.
4. The allogeneic immune cell of embodiment 2 or 3, wherein the first modification comprises a genetic modification of a HLA-A, HLA-B, HLA-C or B2M locus of the allogeneic immune cell genome.
5. The allogeneic immune cell of embodiment 3 or 4, wherein the first modification comprises a deletion, insertion, substitution or frameshift mutation in the HLA-A, HLA-B, HLA-C or B2M locus.
6. The allogeneic immune cell of embodiment 3-5, wherein the first modification reduces expression of a protein encoded by the HLA-A, HLA-B, HLA-C or B2M locus.
7. The allogeneic immune cell of embodiment 2-5, wherein the first modification eliminates expression of a functional protein encoded by the HLA-A, HLA-B, HLA-C or B2M locus.
8. The allogeneic immune cell of embodiment of any one of embodiments 2-7, wherein the first modification comprises using a nucleic acid guided endonuclease, a zinc finger nuclease or a TALEN.
9. The allogeneic immune cell of embodiment 8, wherein the nucleic acid guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasY, Csx3, Csx1, Csx15, Csf1, Csf2, Csf3, and Csf4.
10. The allogeneic immune cell of embodiment 8, wherein the nucleic acid guided endonuclease is Cas9.
11. The allogeneic immune cell of any one of embodiments 8-10, wherein the allogeneic immune cell is modified with a nucleic acid guided endonuclease in a complex with a guide nucleic acid (gNA) that specifically targets a sequence of the HLA-A, HLA-B, HLA-C or B2M locus.
12. The allogeneic immune cell of embodiment 2 or 3, wherein the first modification comprises expression of an interfering RNA.
13. The allogeneic immune cell of embodiment 12, wherein the interfering RNA is a small interfering RNA (siRNA), a short hairpin RNA (shRNA) or a microRNA.
14. The allogeneic immune cell of embodiment 12 or 13, wherein the interfering RNA comprises a sequence complementary to a target sequence of HLA-A, HLA-B, HLA-C or B2M.
15. The allogeneic immune cell of embodiment 14, wherein the target sequence of HLA-A, HLA-B, HLA-C or B2M is between 18 and 27 bp in length.
16. The allogeneic immune cell of any one of embodiments 12-15, wherein first modification reduces host versus graft disease when a plurality of the allogeneic immune cells are administered to a subject.
17. The allogeneic immune cell of any one of embodiments 1-16, comprising a second modification that reduces or eliminates expression or function of CD52.
18. The allogeneic immune cell of embodiment 17, wherein the second modification comprises a deletion, insertion, substitution or frameshift mutation in the CD52 locus of the allogeneic immune cell genome.
19. The allogeneic immune cell of embodiment of embodiment 17 or 18, wherein the second modification comprises using a nucleic acid guided endonuclease, a zinc finger nuclease or a TALEN.
20. The allogeneic immune cell of embodiment 19, wherein the nucleic acid guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasY, Csx3, Csx1, Csx15, Csf1, Csf2, Csf3, and Csf4.
21. The allogeneic immune cell of embodiment 19, wherein the nucleic acid endonuclease is Cas9.
22. The allogeneic immune cell of any one of embodiments 19-21, wherein the allogeneic immune cell is modified with a nucleic acid guided endonuclease in a complex with a guide nucleic acid (gNA) that specifically targets a sequence of the CD52 locus.
23. The allogeneic immune cell of embodiment 17, wherein the second modification comprises expression of an interfering RNA.
24. The allogeneic immune cell of embodiment 23, wherein the interfering RNA is a small interfering RNA (siRNA), a short hairpin RNA (shRNA) or a microRNA.
25. The allogeneic immune cell of embodiment 23 or 24, wherein the interfering RNA comprises a sequence complementary to a target sequence of CD52.
26. The allogeneic immune cell of embodiment 25, wherein the target sequence of CD52 is between 18 and 27 bp in length.
27. The allogeneic immune cell of any one of embodiments 1-26, comprising a third modification that reduces targeting of the allogeneic immune cell by NK cells of a subject.
28. The allogeneic immune cell of embodiment 27, wherein the third modification comprises overexpression of HLA-E, HLA-G or NKG2A.
29. The allogeneic immune cell of any one of embodiments 1-28, wherein the allogeneic immune cell comprises an endogenous TCR.
30. The allogeneic immune cell of embodiment 29, wherein expression of the second engineered receptor reduces function of the endogenous TCR.
31. The allogeneic immune cell of embodiment 29 or 30, wherein expression of the second engineered receptor reduces graft versus host disease when a plurality of the allogeneic immune cells are administered to a subject.
32. The allogeneic immune cell of any one of embodiments 1-28, wherein the allogeneic immune cell comprises a fourth modification that reduces or eliminates expression or function of an endogenous TCR.
33. The allogeneic immune cell of embodiment 32, wherein the fourth modification comprises a modification of a TRCA, TRB, CD3D, CD3E, CD3G or CD3Z locus of the allogeneic immune cell.
34. The allogeneic immune cell of embodiment 32 or 33, wherein the fourth modification comprises a deletion, insertion, substitution or frameshift mutation in TRCA, TRB, CD3D, CD3E, CD3G or CD3Z.
35. The allogeneic immune cell of embodiment 34, wherein the fourth modification reduces expression of a protein encoded by the TRCA, TRB, CD3D, CD3ε, CD3G or CD3Z locus.
36. The allogeneic immune cell of embodiment 34, wherein the fourth modification eliminates expression of a functional protein encoded by the TRCA, TRB, CD3D, CD3ε, CD3G or CD3Z locus.
37. The allogeneic immune cell of any one of embodiments 32-36, wherein the fourth modification comprises using a nucleic acid guided endonuclease, a zinc finger nuclease or a TALEN.
38. The allogeneic immune cell of embodiment 37, wherein the nucleic acid guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasY, Csx3, Csx1, Csx15, Csf1, Csf2, Csf3, and Csf4.
39. The allogeneic immune cell of embodiment 37, wherein the nucleic acid guided endonuclease is Cas9.
40. The allogeneic immune cell of any one of embodiments 37-39, wherein the allogeneic immune cell is modified with a nucleic acid guided endonuclease in a complex with a guide nucleic acid (gNA) that specifically targets a sequence of the TRCA, TRB, CD3D, CD3E, CD3G or CD3Z locus.
41. The allogeneic immune cell of embodiment 40, wherein the fourth modification comprises expression of an interfering RNA.
42. The allogeneic immune cell of embodiment 41, wherein the interfering RNA is a small interfering RNA (siRNA), a short hairpin RNA (shRNA) or a microRNA.
43. The allogeneic immune cell of embodiment 41 or 42, wherein the interfering RNA comprises a sequence complementary to a target sequence of TRCA, TRB, CD3D, CD3E, CD3G or CD3Z.
44. The allogeneic immune cell of embodiment 43, wherein the target sequence of TRCA, TRB, CD3D, CD3E, CD3G or CD3Z is between 15 and 30 bp in length.
45. The allogeneic immune cell of any one of embodiments 32-44, wherein the fourth modification reduces graft versus host disease when a plurality of the allogeneic immune cells are administered to a subject.
46. The allogeneic immune cell of any one of embodiments 1-45, wherein the second ligand not expressed in a target cell due to loss of heterozygosity of a gene encoding the second ligand.
47. The allogeneic immune cell of any one of embodiments 1-46, wherein the second ligand is an HLA class I allele or a minor histocompatibility antigen (MiHA).
48. The allogeneic immune cell of any one of embodiments 1-46, wherein the second ligand is not expressed in the target cell due to loss of Y chromosome.
49. The allogeneic immune cell of embodiment 47, wherein the MiHA is selected from the group of MiHAs in Tables 2 and 3.
50. The allogeneic immune cell of embodiment 47, wherein the MiHA is HA-1.
51. The allogeneic immune cell of embodiment 47, wherein the HLA class I allele comprises HLA-A, HLA-B or HLA-C.
52. The allogeneic immune cell of embodiment 51, wherein the HLA class I allele is an HLA-A*02 allele.
53. The allogeneic immune cell of embodiment 48, wherein the second ligand is encoded by a Y chromosome gene.
54. The allogeneic immune cell of any one of embodiments 1-50, wherein the first ligand and second ligand are not the same.
55. The allogeneic immune cell of any one of embodiments 1-54, wherein the first ligand is expressed by target cells.
56. The allogeneic immune cell of any one of embodiments 1-55, wherein the first ligand is expressed by target cells and a plurality of non-target cells.
57. The allogeneic immune cell of embodiment 56, wherein the plurality of non-target cells express both the first and second ligands.
58. The allogeneic immune cell of any one of embodiments 1-57, wherein the second ligand is not expressed by the target cells, and is expressed by the plurality of non-target cells.
59. The allogeneic immune cell of any one of embodiments 56-58, wherein the target cells are cancer cells and the non-target cells are non-cancerous cells.
60. The allogeneic immune cell of any one of embodiments 1-59, wherein the first ligand is selected from the group consisting of a cell adhesion molecule, a cell-cell signaling molecule, an extracellular domain, a molecule involved in chemotaxis, a glycoprotein, a G protein-coupled receptor, a transmembrane protein, a receptor for a neurotransmitter and a voltage gated ion channel, or a peptide antigen thereof.
61. The allogeneic immune cell of any one of embodiments 1-59, wherein the first ligand is a cancer antigen.
62. The allogeneic immune cell of any one of embodiments 1-59, wherein the first ligand is selected from the group of antigens in Table.
63. The allogeneic immune cell of embodiment 62, wherein the first ligand binding domain is isolated or derived from the antigen binding domain of an antibody in Table.
64. The allogeneic immune cell of any one of embodiments 1-59, wherein the first ligand is selected from the group consisting of transferrin receptor (TFRC), epidermal growth factor receptor (EGFR), CEA cell adhesion molecule 5 (CEA), CD19 molecule (CD19), erb-b2 receptor tyrosine kinase 2 (HER2), and mesothelin (MSLN), or a peptide antigen thereof.
65. The allogeneic immune cell of any one of embodiments 1-59, wherein the first ligand is a pan-HLA ligand.
66. The allogeneic immune cell of any one of embodiments 1-59, wherein the first ligand comprises HLA-A, HLA-B, HLA-C, HLA-E, HLA-F, of HLA-G.
67. The allogeneic immune cell of any one of embodiments 1-69, wherein the first engineered receptor is a T cell receptor (TCR) or a chimeric antigen receptor (CAR).
68. The allogeneic immune cell of any one of embodiments 1-67, wherein the second engineered receptor is a T cell receptor (TCR) or a chimeric antigen receptor (CAR).
69. The allogeneic immune cell of any one of embodiments 1-68, wherein the first ligand binding domain comprises a single chain FAT antibody fragment (scFv) or a β chain variable domain (Vβ).
70. The allogeneic immune cell of any one of embodiments 1-68, wherein the first ligand binding domain comprises a TCR α chain variable domain and a TCR β chain variable domain.
71. The allogeneic immune cell of any one of embodiments 1-68, wherein the first ligand binding domain comprises a variable heavy chain (VH) domain and a variable light chain (VL) domain.
72. The allogeneic immune cell of embodiment 69, wherein the first ligand is EGFR or a peptide antigen thereof, and the first ligand binding domain comprises a sequence of SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115 or SEQ ID NO: 381, or a sequence having at least 90%, at least 95% or at least 99% identity thereto.
73. The allogeneic immune cell of embodiment 69, wherein the first ligand is MSLN or a peptide antigen thereof, and the first ligand binding domain comprises a sequence of SEQ ID NO: 83, SEQ ID NO: 85, SEQ ID NO: 87 or SEQ ID NO: 89, or a sequence having at least 90%, at least 95% or at least 99% identity thereto.
74. The allogeneic immune cell of embodiment 69, wherein the first ligand is CEA or a peptide antigen thereof, and the first ligand binding domain comprises SEQ ID NO: 91, SEQ ID NO: 93, SEQ ID NO: 95, SEQ ID NO: 97, SEQ ID NO: 273, SEQ ID NO: 275, or SEQ ID NO: 277, or a sequence having at least 90%, at least 95% or at least 99% identity thereto.
75. The allogeneic immune cell of embodiment 69, wherein the first ligand is CD19 or a peptide antigen thereof, and the first ligand binding domain comprises SEQ ID NO: 266 or SEQ ID NO: 268, or a sequence having at least 90%, at least 95% or at least 99% identity thereto.
76. The allogeneic immune cell of embodiment 69, wherein the first ligand comprises a pan-HLA ligand, and the first ligand binding domain comprises a sequence of SEQ ID NO: 163, SEQ ID NO: 165, SEQ ID NO: 167, SEQ ID NO: 169, SEQ ID NO: 171, or SEQ ID NO: 173, or a sequence having at least 90%, at least 95% or at least 99% identity thereto.
77. The allogeneic immune cell of any one of embodiments 69-71, wherein the first ligand comprises EGFR or a peptide antigen thereof, and the first ligand binding domain comprises CDRs selected from SEQ ID NOs: 129-162.
78. The allogeneic immune cell of any one of embodiments 69-71, wherein the first ligand comprises a CEA ligand, or a peptide antigen thereof, and the first ligand binding domain comprises CDRs selected from SEQ ID NOs: 285-293.
79. The allogeneic immune cell of any one of embodiments 1-78, wherein the second ligand binding domain comprises an ScFv or a Vβ domain.
80. The allogeneic immune cell of any one of embodiments 1-78, wherein the second ligand binding domain comprises a TCR α chain variable domain and a TCR β chain variable domain.
81. The allogeneic immune cell of any one of embodiments 1-78, wherein the first ligand binding domain comprises a variable heavy chain (VH) domain and a variable light chain (VL) domain.
82. The allogeneic immune cell of embodiment 81, wherein the second ligand comprises HA-1, and wherein the second ligand binding domain comprises a TCR alpha variable domain comprising SEQ ID NO: 195 or a sequence having at least 90%, at least 95%, or at least 99% identity thereto, and a TCR beta variable domain comprising SEQ ID NO: 196 or a sequence having at least 90%, at least 95%, or at least 99% identity thereto.
83. The allogeneic immune cell of embodiment 79, wherein the second ligand comprises an HLA-A*02 allele, and wherein the second ligand binding domain comprises any one of SEQ ID NOs: 50-61 or a sequence having at least 90%, at least 95%, or at least 99% identity thereto.
84. The allogeneic immune cell of any one of embodiments 79-81, wherein the second ligand comprises an HLA-A*02 allele, and the second ligand binding domain comprises CDRs selected from SEQ ID NOs: 39-49.
85. The allogeneic immune cell of any one of embodiment 1-84, wherein the second engineered receptor comprises at least one immunoreceptor tyrosine-based inhibitory motif (ITIM).
86. The allogeneic immune cell of any one of embodiments 1-85, wherein the second engineered receptor comprises a LILRB1 intracellular domain or a functional variant thereof.
87. The allogeneic immune cell of embodiment 86, wherein the LILRB1 intracellular domain comprises a sequence at least 95% identical to SEQ ID NO: 73.
88. The allogeneic immune cell of any one of embodiments 1-87, wherein the second engineered receptor comprises a LILRB1 transmembrane domain or a functional variant thereof.
89. The allogeneic immune cell of embodiment 88, wherein the LILRB1 transmembrane domain or a functional variant thereof comprises a sequence at least 95% identical to SEQ ID NO: 82.
90. The allogeneic immune cell of any one of embodiments 1-89, wherein the second engineered receptor comprises a LILRB1 hinge domain or functional fragment or variant thereof.
91. The allogeneic immune cell of embodiment 90, wherein the LILRB1 hinge domain comprises a sequence at least 95% identical to SEQ ID NO: 81, SEQ ID NO: 74 or SEQ ID NO: 75.
92. The allogeneic immune cell of any one of embodiments 1-89, wherein the second engineered receptor comprises a LILRB1 intracellular domain and a LILRB1 transmembrane domain, or a functional variant thereof.
93. The allogeneic immune cell of embodiment 92, wherein the LILRB1 intracellular domain and LILRB1 transmembrane domain comprises SEQ ID NO: 77 or a sequence at least 95% identical to SEQ ID NO: 77.
94. The allogeneic immune cell of any one of embodiments 1-89, wherein the second engineered receptor comprises a first polypeptide comprising SEQ ID NO: 77 or a sequence at least 95% identity thereto fused to a TCR alpha variable domain, and a second polypeptide comprising SEQ ID NO: 77 or a sequence at least 95% identity thereto fused to a TCR beta variable domain.
95. The allogeneic immune cell of any one of embodiments 1-94, wherein the first and second receptors are expressed on the surface of the immune cell at a ratio between about 1:10 to 10:1 first receptor to second receptor.
96. The allogeneic immune cell of any one of embodiments 1-94, wherein the first and second receptors are expressed on the surface of the immune cell at a ratio between about 1:3 to 3:1 first receptor to second receptor.
97. The allogeneic immune cell of any one of embodiments 1-96, wherein the immune cell is selected form the group consisting of T cells, B cells and Natural Killer (NK) cells.
98. The allogeneic immune cell of any one of the preceding embodiments, wherein the immune cell is non-natural.
99. The allogeneic immune cells of any one of the preceding embodiments, wherein the immune cell is isolated.
100. The allogeneic immune cell of any one of the preceding embodiments, for use as a medicament.
101. The allogeneic immune cell of embodiment 100, wherein the medicament is for the treatment of cancer in a subject.
102. A pharmaceutical composition, comprising a plurality of the allogeneic immune cells of any one of embodiments 1-101.
103. The pharmaceutical composition of embodiment 102, comprising a pharmaceutically acceptable carrier, diluent or excipient.
104. The pharmaceutical composition of embodiment 101 or 102, comprising a therapeutically effective amount of the allogeneic immune cells.
105. A method of increasing the specificity of an adoptive cell therapy in a subject, comprising administering to the subject a plurality of the allogeneic immune cell of any one of embodiments 1-57 or the pharmaceutical composition of any one of embodiments 102-104.
106. A method of treating cancer with an adoptive cell therapy, comprising administering to the subject a plurality of the allogeneic immune cell of any one of embodiments 1-101 or the pharmaceutical composition of any one of embodiments 102-104.
107. The method of embodiment 106, wherein cells of the cancer express the first ligand.
108. The method of any one of embodiments 106 or 107, wherein cells of the cancer do not express the second ligand due to loss of heterozygosity or loss of Y chromosome.
109. The method of any one of embodiments 106-108, wherein non-target cells express both the first ligand and the second ligand.
110. The method of any one of embodiments 105-109, comprising administering a lymphodepletion agent to the subject.
111. The method of embodiment 110, wherein the lymphodepletion agent specifically targets CD52.
112. A method of making the allogeneic immune cell of any one of embodiments 1-101, comprising
a. providing a plurality of allogeneic immune cells; and
b. transforming the immune cells with a vector encoding a first engineered receptor comprising a transmembrane region and an extracellular region, the extracellular region comprising a first ligand binding domain capable of specifically binding a first ligand, and a vector encoding a second engineered receptor comprising a transmembrane region and an extracellular region, the extracellular region comprising a second ligand binding domain capable of specifically binding a second ligand;
wherein binding of the first ligand binding domain to the first ligand activates or promotes activation of the immune cell, and
wherein binding of the second ligand binding domain to a second ligand inhibits activation of the immune cell by the first ligand.
113. A kit comprising the immune cell of any one of embodiments 1-98 or the pharmaceutical composition of any one of embodiments 102-104.
114. The kit of embodiment 113, further comprising instructions for use.
115. A method for producing an allogeneic immune cell, the method comprising obtaining an immune cell from or derived from a donor and causing the cell to express activating and blocking receptors, wherein:
the activating receptors induce a cytotoxic response when the cell encounters a target cell; the blocking receptors block the cytotoxic response when the cell encounters a non-target cell and constitutively block immune responses endogenous to the donor.
116. The method of embodiment 115, wherein the immune cell expresses one or more T cell receptor (TCR) endogenous to the donor.
117. The method of embodiment 116, wherein the blocking receptors constitutively block an immune response caused by activation of the endogenous TCR when the immune cell encounters a non-target cell.
118. The method of embodiment 117, wherein the blocking receptors reduce activation of the endogenous TCR.
119. The method of embodiment 117, wherein the blocking receptors reduce expression of the endogenous TCR.
120. The method of embodiment 115, further comprising modifying the immune cell to reduce or eliminate expression of one or more endogenous TCR.
121. The method of embodiment 120, wherein modifying the obtained immune cell includes inducing a genetic modification of a TRCA, TRB, CD3D, CD3E CD3G, or CD3Z locus of the cell.
122. The method of embodiment 115, further comprising modifying the immune cell to reduce or eliminate expression or function of a component of the major histocompatibility class I complex (MHC I) of the cell.
123. The method of embodiment 122, wherein modifying the component of the MHC I reduces targeting of the immune cell by cytolytic T cells.
124. The method of embodiment 123, wherein the component of the MCH I is a B2M locus.
125. The method of embodiment 115, further comprising tuning an endogenous immune response by altering the amount of the blocking receptor expressed on the immune cell.
126. The method of embodiment 125, wherein tuning comprises increasing the amount of blocking receptor expressed to reduce or eliminate the endogenous immune response.
127. The method of embodiment 126, further comprising obtaining a sample comprising non-target cells from a patient, measuring the endogenous immune response of the immune cell in the presence of cells from the sample, and tuning the expression of the blocking receptor such that the level of the endogenous immune response falls below a threshold.
128. A method for treating cancer, the method comprising:
providing to a patient an allogeneic immune cell that expresses activating receptors and blocking receptors, wherein:
binding of the activating receptor to an activating ligand on a cancer cell promotes a cytotoxic response, and
expression of the blocking receptor eliminates or reduces an endogenous immune response in the immune cell.
129. The method of embodiment 128, wherein binding of the blocking receptor to a blocking ligand on a non-target cell inhibits the cytotoxic response promoted by the activating receptor.
130. The method of embodiment 129, wherein the reduced or eliminated endogenous immune response eliminates or reduces graft versus host disease (GvHD) caused by the immune cell in the patient.
131. The method of embodiment 130, wherein expression of the blocking receptor reduces or eliminates the expression of a TCR endogenous to the immune cell.
132. The method of embodiment 130, wherein expression of the blocking receptor reduces or eliminates activation of a TCR endogenous to the immune cell.
133. The method of embodiment 129, wherein the immune cell includes a modification of a component of the MHC I that reduces targeting of the immune cell by cytolytic T cells of the patient.
134. The method of embodiment 133, wherein the modification reduces or eliminates expression of B2M in the immune cell.
135. An engineered allogeneic immune cell, wherein the cell is from or derived from a donor and caused to express activating and blocking receptors, wherein:
the activating receptors induce a cytotoxic response when the cell encounters a target cell;
   the blocking receptors block the cytotoxic response when the cell encounters a non-target cell and constitutively block immune responses endogenous to the donor.
   136. The cell of embodiment 135, wherein the cell expresses one or more T cell receptors (TCRs) endogenous to the donor.
   137. The cell of embodiment 136, wherein the blocking receptors constitutively block an immune response caused by activation of the endogenous TCR when the immune cell encounters a non-target cell.
   138. The cell of embodiment 137, wherein the blocking receptors reduce activation of the endogenous TCR.
   139. The cell of embodiment 137, wherein the blocking receptors reduce expression of the endogenous TCR.
   140. The cell of embodiment 135, wherein the cell further includes a modification that reduces or eliminates expression of one or more endogenous TCR.
   141. The cell of embodiment 140, wherein the modification is a genetic modification of a TRCA, TRB, CD3D, CD3E, CD3G, or CD3Z locus of the cell.
   142. The cell of embodiment 135, wherein the cell further comprises a modification that reduces or eliminates expression of a component of the MHC I.
   143. The cell of embodiment 142, wherein the modification to the component of the MHC I reduces targeting of the immune cell by cytolytic T cells.

### Representative Embodiments—Set 2

1. An immune cell comprising

an inhibitory receptor comprising a ligand binding domain specific to a class I major histocompatibility complex (MHC-I) molecule, or a peptide-MHC complex thereof;

wherein the immune cell comprises one or more modifications that reduce autocrine binding/signaling by the receptor.
2. The immune cell of embodiment 1, wherein the one or more modifications comprise an inactivating mutation in an endogenous gene encoding an allele of an endogenous MHC class I polypeptide specifically bound by the inhibitory receptor.
3. The immune cell of embodiment 2, wherein the gene encoding the MHC class I polypeptide is HLA-A, HLA-B, and/or HLA-C.
4. The immune cell of embodiment 2, wherein the gene encoding the MHC class I polypeptide is HLA-A.
5. The immune cell of embodiment 2, wherein the gene encoding the MHC class I polypeptide is HLA-A*02.
6. The immune cell of embodiment 2, wherein the gene encoding the MHC class I polypeptide is HLA-A*02:01.
7. The immune cell of any one of embodiments 2-6, wherein modifying the gene encoding the MHC class I polypeptide comprises deleting all or a portion of the gene.
8. The immune cell of any one of embodiments 2-6, wherein modifying the gene encoding the MHC class I polypeptide comprises introducing a mutation in the gene.
9. The immune cell of embodiment 8, wherein the mutation comprises a deletion, insertion, substitution, or frameshift mutation.
10. The immune cell of any one of embodiments 7-9, wherein modifying the gene comprises using a nucleic acid guided endonuclease.
11. The immune cell of embodiment 10, wherein the nucleic acid guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof
12. The immune cell of embodiment 11, wherein the nucleic acid endonuclease is Cas9.
13. The immune cell of any one of embodiments 7-12, wherein the immune cell is modified with a nucleic acid guided endonuclease in a complex with at least one guide nucleic acids (gNAs) that specifically targets the sequence of an HLA-A locus.
14. The immune cell of embodiment 13, wherein the gNAs comprise a sequence that shares about 90%, about 95%, about 96%, about 97%, about 98%, or about 99% identity to a sequence selected from SEQ ID NOs: 390-3276.
15. The immune cell of embodiment 13, wherein the gNAs comprise a sequence selected from SEQ ID NOs: 390-3276.
16. The immune cell of any one of embodiments 7-12, wherein the immune cell is modified with a nucleic acid guided endonuclease in a complex with at least one guide nucleic acids (gNAs) that specifically targets a sequence of HLA-A*02 alleles.
17. The immune cell of embodiment 16, wherein the gNAs comprise a sequence that shares about 90%, about 95%, about 96%, about 97%, about 98%, or about 99% identity to a sequence selected from SEQ ID NOs: 390-1585.
18. The immune cell of embodiment 17, wherein the gNAs comprise a sequence selected from SEQ ID NOs: 390-1585.
19. The immune cell of any one of embodiments 7-12, wherein the immune cell is modified with a nucleic acid guided endonuclease in a complex with at least one guide nucleic acids (gNAs) that specifically targets a sequence of HLA-A*02:01 alleles.
20. The immune cell of embodiment 19, wherein the gNAs comprise a sequence that shares about 90%, about 95%, about 96%, about 97%, about 98%, or about 99% identity to a sequence selected from SEQ ID NOs: 390-1174.
21. The immune cell of embodiment 20, wherein the gNAs comprise a sequence selected from SEQ ID NOs: 390-1174.
22. The immune cell of any one of embodiments 7-12, wherein the immune cell is modified with a nucleic acid guided endonuclease in a complex with at least one guide nucleic acids (gNAs) that specifically targets a sequence of HLA-A*02:01:01 alleles.
23. The immune cell of embodiment 22, wherein the gNAs comprise a sequence that shares about 90%, about 95%, about 96%, about 97%, about 98%, or about 99% identity to a sequence selected from SEQ ID NOs: 390-1166.
24. The immune cell of embodiment 23, wherein the gNAs comprise a sequence selected from SEQ ID NOs: 390-1166.
25. The immune cell of any one of embodiments 7-12, wherein the immune cell is modified with a nucleic acid guided endonuclease in a complex with at least one guide nucleic acids (gNAs) that specifically targets a sequence of HLA-A*02:01:01:01 alleles.
26. The immune cell of embodiment 25, wherein the gNAs comprise a sequence that shares about 90%, about 95%, about 96%, about 97%, about 98%, or about 99% identity to a sequence selected from SEQ ID NOs: 390-1126.
27. The immune cell of embodiment 26, wherein the gNAs comprise a sequence selected from SEQ ID NOs: 390-1126.
28. The immune cell of any one of embodiments 7-12, wherein the immune cell is modified with a nucleic acid guided endonuclease in a complex with at least one guide nucleic acids (gNAs) that specifically targets a coding DNA sequence of HLA-A*02.
29. The immune cell of embodiment 28, wherein the gNAs comprise a sequence that shares about 90%, about 95%, about 96%, about 97%, about 98%, or about 99% identity to a sequence selected from SEQ ID NOs: 390-509.
30. The immune cell of embodiment 29, wherein the gNAs comprise SEQ ID NOs: 390-509.
31. The immune cell of any one of embodiments 7-12, wherein the immune cell is modified with a nucleic acid guided endonuclease in a complex with at least one guide nucleic acids (gNAs) that specifically targets a coding DNA sequence that is shared by more than 1000 HLA-A*02 alleles.
32. The immune cell of embodiment 31, wherein the gNAs comprise a sequence that shares about 90%, about 95%, about 96%, about 97%, about 98%, or about 99% identity to a sequence selected from SEQ ID NOs: 390-455.
33. The immune cell of embodiment 32, wherein the gNAs comprise a sequence selected from SEQ ID NOs: 390-455.
34. The immune cell of embodiment 33, wherein the gNAs comprise a sequence that shares about 90%, about 95%, about 96%, about 97%, about 98%, or about 99% identity to a sequence selected from SEQ ID NOs: 394, 407, 408, 414, 421, 423, 426, 429, 433, 435, 438, 440, 448, 451, 454.
35. The immune cell of embodiment 34, wherein the gNAs comprise SEQ ID NOs: 394, 407, 408, 414, 421, 423, 426, 429, 433, 435, 438, 440, 448, 451, 454.
36. The immune cell of embodiment 34, wherein the gNAs comprise a sequence that shares about 90%, about 95%, about 96%, about 97%, about 98%, or about 99% identity to SEQ ID NO: 394.
37. The immune cell of embodiment 36, wherein the gNAs comprise SEQ ID NO: 394.
38. The immune cell of embodiment 34, wherein the gNAs comprise a sequence that shares about 90%, about 95%, about 96%, about 97%, about 98%, or about 99% identity to SEQ ID NO: 423.
39. The immune cell of embodiment 38, wherein the gNAs comprise SEQ ID NO: 423.
40. The immune cell of any one of the preceding embodiments, wherein the immune cell is a T cell, B cell, or Natural Killer (NK) cell.
41. The immune cell of any one of the preceding embodiments, wherein the immune cell is non-natural.
42. The immune cell of any one of the preceding embodiments, wherein the immune cell is isolated.
43. The immune cell of any one of the preceding embodiments, for use as a medicament.
44. The immune cell of embodiment 43, wherein the medicament is for the treatment of cancer in a subject.
45. A pharmaceutical composition, comprising a plurality of the immune cells of any one of embodiments 1-44.
46. The pharmaceutical composition of embodiment 45, comprising a pharmaceutically acceptable carrier, diluent, or excipient.
47. The pharmaceutical composition of embodiment 45 or 46, comprising a therapeutically effective amount of the immune cells.
48. A method of treating cancer with an adoptive cell therapy, comprising administering to the subject a plurality of the immune cell of any one of embodiments 1-44 or the pharmaceutical composition of any one of embodiments 45-47.
49. The immune cell of any one of embodiments 1-44, wherein the immune cell is autologous to a subject.
50. The immune cell of any one of embodiments 1-44, wherein the immune cell is allogeneic to a subject.
51. A method of producing an immune cell with reduced autocrine binding/signaling comprising:
   a. transducing the immune cell with a vector comprising a sequence encoding a nucleic acid-guided endonuclease, thereby producing an immune cell expressing the nuclease; and
   b. transfecting the immune cell with at least one guide nucleic acid (gNA) complementary to a target sequence of a target gene selected from the group consisting of HLA-A, HLA-B, HLA-C, or an allele thereof,

wherein the gNA binds to the target sequence and the nuclease cleaves the target sequence, thereby producing a modified target gene.
52. The method of embodiment 51, wherein the target gene comprises HLA-A*02.
53. The method of embodiment 51 or 52, wherein the nuclease cleaves the target sequence to generate a single stranded or double stranded break.
54. The method of embodiment 53, wherein the double strand break is repaired by non-homologous end joining (NHEJ), thereby producing a deletion in the target gene.
55. The method of embodiment 54, wherein the deletion produces a truncated target gene or a frameshift mutation in the target gene.
56. The method of any one of embodiments 51-55, wherein the nuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.
57. The method of embodiment 56, wherein the nuclease is Cas9.
58. The method of any one of embodiments 51-57, wherein the immune cell is modified with a nucleic acid guided endonuclease in a complex with at least one guide nucleic acids (gNAs) that specifically targets the sequence of an HLA-A locus.
59. The method of embodiment 58, wherein the gNAs comprise a sequence that shares about 90%, about 95%, about 96%, about 97%, about 98%, or about 99% identity to a sequence selected from SEQ ID NOs: 4390-3276.
60. The method of embodiment 59, wherein the gNAs comprise a sequence selected from SEQ ID NOs: 390-3276.
61. The method of any one of embodiments 51-57, wherein the immune cell is modified with a nucleic acid guided endonuclease in a complex with at least one guide nucleic acids (gNAs) that specifically targets a sequence of HLA-A*02 alleles.
62. The method of embodiment 61, wherein the gNAs comprise a sequence that shares about 90%, about 95%, about 96%, about 97%, about 98%, or about 99% identity to a sequence selected from SEQ ID NOs: 390-1585.
63. The method of embodiment 62, wherein the gNAs comprise a sequence selected from SEQ ID NOs: 390-1585.
64. The method of any one of embodiments 51-57, wherein the immune cell is modified with a nucleic acid guided endonuclease in a complex with at least one guide nucleic acids (gNAs) that specifically targets a sequence of HLA-A*02:01 alleles.
65. The method of embodiment 64, wherein the gNAs comprise a sequence that shares about 90%, about 95%, about 96%, about 97%, about 98%, or about 99% identity to a sequence selected from SEQ ID NOs: 390-1174.
66. The method of embodiment 65, wherein the gNAs comprise a sequence selected from SEQ ID NOs: 390-1174.
67. The method of any one of embodiments 51-57, wherein the immune cell is modified with a nucleic acid guided endonuclease in a complex with at least one guide nucleic acids (gNAs) that specifically targets a sequence of HLA-A*02:01:01 alleles.
68. The method of embodiment 67, wherein the gNAs comprise a sequence that shares about 90%, about 95%, about 96%, about 97%, about 98%, or about 99% identity to a sequence selected from SEQ ID NOs: 390-1166.
69. The method of embodiment 68, wherein the gNAs comprise a sequence selected from SEQ ID NOs: 390-1166.
70. The method of any one of embodiments 51-57, wherein the immune cell is modified with a nucleic acid guided endonuclease in a complex with at least one guide nucleic acids (gNAs) that specifically targets a sequence of HLA-A*02:01:01:01 alleles.
71. The method of embodiment 70, wherein the gNAs comprise a sequence that shares about 90%, about 95%, about 96%, about 97%, about 98%, or about 99% identity to a sequence selected from SEQ ID NOs: 390-1126.
72. The method of embodiment 71, wherein the gNAs comprise a sequence selected from SEQ ID NOs: 390-1126.
73. The method of any one of embodiments 51-57, wherein the immune cell is modified with a nucleic acid guided endonuclease in a complex with at least one guide nucleic acids (gNAs) that specifically targets a coding DNA sequences of HLA-A*02.
74. The method of embodiment 73, wherein the gNAs comprise a sequence that shares about 90%, about 95%, about 96%, about 97%, about 98%, or about 99% identity to a sequence selected from SEQ ID NOs: 390-509.
75. The method of embodiment 74, wherein the gNAs comprise SEQ ID NOs: 390-509.
76. The method of any one of embodiments 51-57, wherein the immune cell is modified with a nucleic acid guided endonuclease in a complex with at least one guide nucleic acids (gNAs) that specifically targets a coding DNA sequence that is shared by more than 1000 HLA-A*02 alleles.
77. The method of embodiment 76, wherein the gNAs comprise a sequence that shares about 90%, about 95%, about 96%, about 97%, about 98%, or about 99% identity to a sequence selected from SEQ ID NOs: 390-455.
78. The method of embodiment 77, wherein the gNAs comprise a sequence selected from SEQ ID NOs: 390-455.
79. The method of embodiment 78, wherein the gNAs comprise a sequence that shares about 90%, about 95%, about 96%, about 97%, about 98%, or about 99% identity to a sequence selected from SEQ ID NOs: 394, 407, 408, 414, 421, 423, 426, 429, 433, 435, 438, 440, 448, 451, 454.
80. The method of embodiment 79, wherein the gNAs comprise SEQ ID NOs: 394, 407, 408, 414, 421, 423, 426, 429, 433, 435, 438, 440, 448, 451, 454.
81. The method of embodiment 80, wherein the gNAs comprise a sequence that shares about 90%, about 95%, about 96%, about 97%, about 98%, or about 99% identity to SEQ ID NO: 394.
82. The method of embodiment 81, wherein the gNAs comprise SEQ ID NO: 394.
83. The method of embodiment 80, wherein the gNAs comprise a sequence that shares about 90%, about 95%, about 96%, about 97%, about 98%, or about 99% identity to SEQ ID NO: 423.
84. The method of embodiment 83, wherein the gNAs comprise SEQ ID NO: 423.
85. The method of any one of embodiments 51-84, comprising transducing the immune cell with a first vector comprising a sequence encoding the activator receptor and a second vector comprising a sequence encoding the inhibitory receptor, thereby producing an immune cell expressing the activator and inhibitory receptors.
86. The method of any one of embodiments 51-84, wherein, prior to the transducing and/or transfecting steps, the immune cell comprises a polynucleotide encoding activator and inhibitory receptors.
87. The method of embodiments 85 or 86, wherein the inhibitory receptor specifically binds to an HLA-A*02 pMHC antigen and the target gene comprises HLA-A*02.
88. A method of manufacturing a composition comprising immune cells with reduced autocrine binding/signaling comprising:
   a. providing immune cells from a subject suffering from or at risk for cancer or a hematological malignancy
   b. transducing the immune cell with a vector comprising a sequence encoding a nucleic acid-guided endonuclease, thereby producing an immune cell expressing the nuclease; and
   c. transfecting the immune cell with at least one guide nucleic acid (gNA) complementary to a target sequence of a target gene selected from the group consisting of HLA-A, HLA-B, HLA-C, or an allele thereof,

wherein the gNA binds to the target sequence and the nuclease cleaves the target sequence, thereby producing a modified target gene.
89. The method of embodiment 88, wherein, prior to the transducing and/or transfecting steps, the immune cell comprises a polynucleotide encoding activator or inhibitory receptors.
90. The method of embodiments 88 or 89, further comprising transducing the immune cell with a first vector comprising a sequence encoding the activator receptor and a second vector comprising a sequence encoding the inhibitory receptor, thereby producing an immune cell expressing the activator and inhibitory receptors.
91. A method of treating a subject in need thereof comprising:
   a. providing immune cells from a subject suffering from or at risk for cancer or a hematological malignancy
   b. transducing the immune cell with a vector comprising a sequence encoding a nucleic acid-guided endonuclease, thereby producing an immune cell expressing the nuclease;
   c. transfecting the immune cell with at least one guide nucleic acid (gNA) complementary to a target sequence of a target gene selected from the group consisting of HLA-A, HLA-B, HLA-C, or an allele thereof, wherein the gNA binds to the target sequence and the nuclease cleaves the target sequence, thereby producing a modified target gene; and
   d. administering the immune cell to the subject.
92. A guide RNA comprising a targeting sequence, wherein the targeting sequence is complementary to the sequence of a target gene selected from the group consisting of HLA-A, HLA-B, HLA-C, or an allele thereof
93. The guide RNA of embodiment 92, wherein the target gene is a HLA-A locus.
94. The guide RNA of embodiment 93, wherein the targeting sequence shares about 90%, about 95%, about 96%, about 97%, about 98%, or about 99% identity to a sequence selected from SEQ ID NOs: 390-3276.
95. The guide RNA of embodiment 94, wherein the targeting sequence is selected from SEQ ID NOs: 390-3276.
96. The guide RNA of embodiment 92, wherein the target gene is a HLA-B locus.
97. The guide RNA of embodiment 92, wherein the target gene is a HLA-C locus.
98. The guide RNA of any one of embodiments 93-95, wherein the target gene is a HLA-A allele.
99. The guide RNA of embodiment 98, wherein the HLA-A allele comprises HLA-A*02,
100. HLA-A*02:01, HLA-A*02:01:01, and/or HLA-A*02:01:01:01.
100. The guide RNA of embodiment 99, wherein the HLA-A allele is HLA-A*02.
101. The guide RNA of embodiment 100, wherein the targeting sequence shares about 90%, about 95%, about 96%, about 97%, about 98%, or about 99% identity to a sequence selected from SEQ ID NOs: 390-1585.
102. The guide RNA of embodiment 101, wherein the targeting sequence is selected from SEQ ID NOs: 390-1585.
103. The guide RNA of embodiment 99, wherein the HLA-A allele is HLA-A*02:01.
104. The guide RNA of embodiment 103, wherein the targeting sequence shares about 90%, about 95%, about 96%, about 97%, about 98%, or about 99% identity to a sequence selected from SEQ ID NOs: 390-1174.
105. The guide RNA of embodiment 104, wherein the targeting sequence is selected from SEQ ID NOs: 390-1174.
106. The guide RNA of embodiment 99, wherein the HLA-A allele is HLA-A*02:01:01.
107. The guide RNA of embodiment 106, wherein the targeting sequence shares about 90%, about 95%, about 96%, about 97%, about 98%, or about 99% identity to a sequence selected from SEQ ID NOs: 390-1166.
108. The guide RNA of embodiment 107, wherein the targeting sequence is selected from SEQ ID NOs: 390-1166.
109. The guide RNA of embodiment 99, wherein the HLA-A allele is HLA-A*02:01:01:01.
110. The guide RNA of embodiment 109, wherein the targeting sequence shares about 90%, about 95%, about 96%, about 97%, about 98%, or about 99% identity to a sequence selected from SEQ ID NOs: 390-1126.
111. The guide RNA of embodiment 110, wherein the targeting sequence is selected from SEQ ID NOs: 390-1126.
112. The guide RNA of embodiment 99, wherein the targeting sequence targets a coding DNA sequences of the HLA-A*02 allele.
113. The guide RNA of embodiment 112, wherein the targeting sequence shares about 90%, about 95%, about 96%, about 97%, about 98%, or about 99% identity to a sequence selected from SEQ ID NOs: 390-509.
114. The guide RNA of embodiment 114, wherein the targeting sequence is selected from SEQ ID NOs: 390-509.
115. The guide RNA of embodiment 112, wherein the targeting sequence targets a coding DNA sequence that is shared by more than 1000 HLA-A*02 alleles.
116. The guide RNA of embodiment 115, wherein the targeting sequence shares about 90%, about 95%, about 96%, about 97%, about 98%, or about 99% identity to a sequence selected from SEQ ID NOs: 390-455.
117. The guide RNA of embodiment 116, wherein the targeting sequence is selected from SEQ ID NOs: 390-455.
118. The guide RNA of embodiment 115, wherein the targeting sequence shares about 90%, about 95%, about 96%, about 97%, about 98%, or about 99% identity to a sequence selected from SEQ ID NOs: 394, 407, 408, 14, 421, 423, 426, 429, 433, 435, 438, 440, 448, 451, 454.
119. The guide RNA of embodiment 118, wherein the targeting sequence is selected from SEQ ID NOs: 394, 407, 408, 414, 421, 423, 426, 429, 433, 435, 438, 440, 448, 451, 454.
120. The guide RNA of embodiment 115, wherein the targeting sequence shares about 90%, about 95%, about 96%, about 97%, about 98%, or about 99% identity to a sequence selected from SEQ ID NOs: 394.
121. The guide RNA of embodiment 120, wherein the targeting sequence is selected from SEQ ID NOs: 394.
122. The guide RNA of embodiment 115, wherein the targeting sequence shares about 90%, about 95%, about 96%, about 97%, about 98%, or about 99% identity to a sequence selected from SEQ ID NOs: 423.
123. The guide RNA of embodiment 122, wherein the targeting sequence is selected from SEQ ID NOs: 423.
124. The guide RNA of any one of embodiments 92 to 123, wherein the guide RNA scaffold sequence binds a nucleic acid guided endonuclease selected from Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof
125. The guide RNA of embodiment 124, wherein the nucleic acid guided endonuclease is

Cas9.
126. The guide RNA of embodiment 124 or 125, wherein the nucleic acid guided endonuclease binds a PAM sequence.
127. The guide RNA of embodiment 126, wherein the PAM sequence is NGG, NGCG, NGAG, NGAN, NGNG, NG, GAA, GAT, NNGRRT, NNGRRN, TTTV, TYCV, TATV, NNNNRYAC, NNNNGATT, NNAGAAW, NAAAAC, wherein N represents any nucleotide; V represents A, G, or C; R represents A or G; Y represents C or T; and W represents A or T.
128. The guide RNA of any one of embodiments 71-93, further comprising a scaffold sequence.
129. The guide RNA of embodiment 57, wherein the scaffold sequence is

(SEQ ID NO: 8345)
GTTTTAGAGCTAGAAATAGCAAGTTAAAATAAGGCTAGTCCGTTATCAAC
TTGAAAAAGTGGCACCGAGTCGGTGCTTTTTTT.

130. A polynucleotide encoding the guide RNA of embodiments 92-129.
131. The immune cell of any one of embodiments 7-12, wherein the immune cell is modified with a nucleic acid guided endonuclease in a complex with at least one guide nucleic acid (gNA) that specifically targets the sequence of an HLA-A, HLA-B, HLA-C, and HLA-G locus, or a combination thereof
132. The immune cell of embodiment 131, wherein the gNA comprise a sequence that shares about 90%, about 95%, about 96%, about 97%, about 98%, or about 99% identity to a sequence selected from SEQ ID NO: 408.
133. The immune cell of embodiment 132, wherein the gNA comprises SEQ ID NO: 408.
134. The method of any one of embodiments 51-57, wherein the immune cell is modified with a nucleic acid guided endonuclease in a complex with at least one guide nucleic acids (gNAs) that specifically targets the sequence of an HLA-A, HLA-B, HLA-C, and HLA-G locus, or a combination thereof
135. The method of embodiment 134, wherein the gNAs comprise a sequence that shares about 90%, about 95%, about 96%, about 97%, about 98%, or about 99% identity to a sequence selected from SEQ ID NO: 408.
136. The immune cell of embodiment 135, wherein the gNA comprises SEQ ID NO: 408.
137. The guide RNA of embodiment 92, wherein the target gene is a HLA-A, HLA-B, HLA-C, and HLA-G locus, or a combination thereof
138. The guide RNA of embodiment 137, wherein the targeting sequence shares about 90%, about 95%, about 96%, about 97%, about 98%, or about 99% identity to a sequence selected from SEQ ID NOs: 408.
139. The guide RNA of embodiment 138, wherein the targeting sequences comprises SEQ ID NO: 408.

### Representative Embodiments—Set 3

1. An immune cell comprising:

at least one engineered receptor comprising a ligand binding domain specific to a class I major histocompatibility complex (MHC-I) molecule, or a peptide-MHC complex thereof;

wherein the immune cell comprises one or more modifications that reduce autocrine binding/signaling by the engineered receptor.
2. The immune cell of embodiment 1, wherein the one or more modifications comprise an inactivating mutation to an endogenous gene encoding a beta 2 microglobulin (B2M) polypeptide.
3. The immune cell of embodiment 1, wherein the inactivating mutation to the gene encoding B2M is a deletion of all or a portion of the gene.
4. The immune cell of any one of embodiments 1-3, wherein the inactivating mutation to the gene encoding B2M is a deletion of all or a portion of the gene.
5. The immune cell of any one of embodiments 1-3, wherein the inactivating mutation to the gene encoding B2M is a mutation in the gene or a promoter of the gene.
6. The immune cell of embodiment 5, wherein the mutation comprises a deletion, insertion, substitution, or frameshift mutation.
7. The immune cell of any one of embodiments 4-6, wherein the inactivating mutation to the gene encoding B2M is introduced by a nucleic acid guided endonuclease.
8. The immune cell of embodiment 7, wherein the nucleic acid guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and homologs thereof, or modified versions thereof
9. The immune cell of embodiment 8, wherein the nucleic acid endonuclease is Cas9.
10. The immune cell of any one of embodiments 7-9, wherein the inactivating mutation is introduced with a nucleic acid guided endonuclease in a complex with at least one guide nucleic acid (gNA) that specifically targets a sequence within the B2M gene and/or a promoter of the B2M gene.
11. The immune cell of embodiment 10, wherein the gNA comprises a sequence that shares about 80%, about 85%, about 90%, about 95%, about 96%, about 97%, about 98%, or about 99% identity to a sequence selected from SEQ ID NOs: 8357-8470.
12. The immune cell of embodiment 10, wherein the gNA comprises a sequence selected from SEQ ID NOs: 8357-8470.
13. The immune cell of embodiment 10, wherein the inactivating mutation is introduced with a nucleic acid guided endonuclease in a complex with at least one guide nucleic acid (gNA) that specifically targets a coding sequence (CDS) of the B2M gene.
14. The immune cell of embodiment 13, wherein the gNA comprises a sequence that shares about 80%, about 85%, about 90%, about 95%, about 96%, about 97%, about 98%, or about 99% identity to a sequence selected from SEQ ID NOs: 8357-8397.
15. The immune cell of embodiment 13, wherein the gNA comprises a sequence selected from SEQ ID NOs: 8357-8397.
16. The immune cell of embodiment 10, wherein the inactivating mutation is introduced with a nucleic acid guided endonuclease in a complex with at least one guide nucleic acid (gNA) that specifically targets a promoter of the B2M gene.
17. The immune cell of embodiment 16, wherein the gNA comprises a sequence that shares about 80%, about 85%, about 90%, about 95%, about 96%, about 97%, about 98%, or about 99% identity to a sequence selected from SEQ ID NOs: 8398-8470.
18. The immune cell of embodiment 16, wherein the gNA comprises a sequence selected from SEQ ID NOs: 8398-8470.
19. The immune cell of embodiment 10, wherein the gNA comprises a sequence that shares about 80%, about 85%, about 90%, about 95%, about 96%, about 97%, about 98%, or about 99% identity to SEQ ID NO: 8357-8365.
20. The immune cell of embodiment 10, wherein the gNA comprises SEQ ID NO: 8357-8365.
21. The immune cell of any one of embodiments 1-20, wherein the at least one engineered receptor comprises an activator receptor.
22. The immune cell of embodiment 21, wherein the activator receptor is a chimeric antigen receptor (CAR) or T Cell Receptor (TCR).
23. The immune cell of embodiment 21 or 22, wherein the activator receptor binds to a ligand expressed by a cancer cell of a subject.
24. The immune cell of any one of embodiments 1-23, wherein the at least one engineered receptor comprises an inhibitory receptor.
25. The immune cell of embodiment 24, wherein the inhibitory receptor inhibits activation of the immune cell by the activator receptor.
26. The immune cell of embodiment 24 or 25, wherein the inhibitory receptor binds to a ligand that is lost in the cancer cell of the subject through loss of heterozygosity.
27. The immune cell of any one of the preceding embodiments, wherein the immune cell is a T cell, B cell, or Natural Killer (NK) cell.
28. The immune cell of any one of the preceding embodiments, wherein the immune cell is non-natural.
29. The immune cell of any one of embodiments 1-26, wherein the immune cell is autologous to the subject.
30. The immune cell of any one of embodiments 1-26, wherein the immune cell is allogeneic to the subject.
31. The immune cell of any one of the preceding embodiments, wherein the immune cell is isolated.
32. The immune cell of any one of the preceding embodiments, for use as a medicament.
33. The immune cell of embodiment 31, wherein the medicament is for the treatment of cancer in a subject.
34. A pharmaceutical composition, comprising a plurality of the immune cells of any one of embodiments 1-26.
35. The pharmaceutical composition of embodiment 34, comprising a pharmaceutically acceptable carrier, diluent, or excipient.
36. The pharmaceutical composition of embodiment 34 or 35, comprising a therapeutically effective amount of the immune cells.
37. A method of treating a subject with cancer with an adoptive cell therapy, comprising administering to the subject a plurality of the immune cell of any one of embodiments 1-33 or the pharmaceutical composition of any one of embodiments 34-36.
38. A method of producing an immune cell with reduced autocrine binding/signaling comprising:
   a. transducing the immune cell with a vector comprising a sequence encoding a nucleic acid-guided endonuclease, thereby producing an immune cell expressing the nuclease; and
   b. transfecting the immune cell with at least one guide nucleic acid (gNA) complementary to a target sequence within the B2M gene,

wherein the gNA binds to the target sequence and the nuclease cleaves the target sequence, thereby producing a modified B2M gene or B2M gene promoter.
39. The method of embodiment 38, wherein the nuclease cleaves the target sequence to generate a single stranded or double stranded break.
40. The method of embodiment 39, wherein the double strand break is repaired by non-homologous end joining (NHEJ), thereby producing a deletion in B2M
41. The method of embodiment 40, wherein the deletion produces a truncated B2M gene or a frameshift mutation in the B2M gene.
42. The method of any one of embodiments 38-41, wherein the nuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and homologs thereof, or modified versions thereof
43. The method of embodiment 42, wherein the nuclease is Cas9.
44. The method of any one of embodiments 38-43, wherein the gNA specifically targets a sequence within the B2M coding sequence, a B2M intron, a B2M regulatory element or a combination thereof.
45. The method of embodiment 44, wherein the gNA comprises a sequence that shares about 80%, about 85%, about 90%, about 95%, about 96%, about 97%, about 98%, or about 99% identity to a sequence selected from SEQ ID NOs: 8357-8470.
46. The method of embodiment 44, wherein the gNA comprises a sequence selected from SEQ ID NOs: 8357-8470.
47. The method of embodiment 44, wherein the gNA specifically targets the coding sequence (CDS) of the B2M gene.
48. The method of embodiment 47, wherein the gNA comprise a sequence that shares about 80%, about 85%, about 90%, about 95%, about 96%, about 97%, about 98%, or about 99% identity to a sequence selected from SEQ ID NOs: 8357-8397.
49. The method of embodiment 47, wherein the gNA comprises a sequence selected from SEQ ID NOs: 8357-8397.
50. The method of any one of embodiments 44, wherein the gNA specifically targets the promoter of the B2M gene.
51. The method of embodiment 50, wherein the gNA comprises a sequence that shares about 80%, about 85%, about 90%, about 95%, about 96%, about 97%, about 98%, or about 99% identity to a sequence selected from SEQ ID NOs: 8398-8470.
52. The method of embodiment 50, wherein the gNA comprises a sequence selected from SEQ ID NOs: 8398-8470.
53. The method of embodiment 44, wherein the gNA comprises a sequence that shares about 80%, about 85%, about 90%, about 95%, about 96%, about 97%, about 98%, or about 99% identity to SEQ ID NO: 8357-8365.
54. The method of embodiment 44, wherein the gNA comprises SEQ ID NO: 8357-8365.
55. The method of any one of embodiments 38-54, comprising transducing the immune cell with a first vector comprising a sequence encoding the activator receptor and a second vector comprising a sequence encoding the inhibitory receptor, thereby producing an immune cell expressing the activator and inhibitory receptors.
56. The method of any one of embodiments 38-55, wherein, prior to the transducing and/or transfecting steps, the immune cell comprises one or more polynucleotides encoding activator and/or inhibitory receptors.
57. The method of embodiments 55 or 56, wherein the inhibitory receptor specifically binds to an HLA-A*02 pMHC antigen.
58. A method of manufacturing a composition comprising plurality of immune cells with reduced autocrine binding/signaling comprising:
   a. providing immune cells from a subject suffering from or at risk of developing a cancer or a hematological malignancy
   b. transducing the plurality of immune cell with a vector comprising a sequence encoding a nucleic acid-guided endonuclease, thereby producing an immune cell expressing the nucleic acid-guided endonuclease; and
   c. transfecting the immune cell with at least one guide nucleic acid (gNA) complementary to a target sequence of a B2M gene,

wherein the gNA binds to the target sequence and the nuclease cleaves the target sequence, thereby producing a modified B2M gene.
59. The method of embodiment 58, wherein, prior to the transducing and/or transfecting steps, the immune cell comprises at least one polynucleotide encoding activator or inhibitory receptors.
60. The method of embodiments 58 or 59, further comprising transducing the immune cell with a first vector comprising a sequence encoding the activator receptor and a second vector comprising a sequence encoding the inhibitory receptor, thereby producing an immune cell expressing the activator and inhibitory receptors.
61. A method of treating a subject in need thereof comprising:
   a. providing a plurality of immune cells from a subject suffering from or at risk of developing a cancer or a hematological malignancy;
   b. transducing the plurality of immune cells with a vector comprising a sequence encoding a nucleic acid-guided endonuclease, thereby producing an immune cell expressing the nucleic acid-guided endonuclease;
   c. transfecting the plurality of immune cells with at least one guide nucleic acid (gNA) complementary to a target sequence of a B2M gene, wherein the gNA binds to the target sequence and the nuclease cleaves the target sequence, thereby producing a modified B2M gene; and
   d. administering the plurality of immune cells to the subject.
62. A guide nucleic acid (gNA) comprising a targeting sequence, wherein the targeting sequence is complementary to a sequence within the B2M gene or B2M gene promoter.
63. The gNA of embodiment 62, wherein the targeting sequence shares about 80%, about 85%, about 90%, about 95%, about 96%, about 97%, about 98%, or about 99% identity to a sequence selected from SEQ ID NOs: 8357-8470.
64. The gNA of embodiment 62, wherein the targeting sequence is selected from SEQ ID NOs: 8357-8470.
65. The gNA of embodiment 62, wherein the gNA specifically targets the coding sequence (CDS) of the B2M gene.
66. The gNA embodiment 65, wherein the targeting sequence shares about 80%, about 85%, about 90%, about 95%, about 96%, about 97%, about 98%, or about 99% identity to a sequence selected from SEQ ID NOs: 8357-8397.
67. The gNA of embodiment 65, wherein the target sequence comprises a sequence selected from SEQ ID NOs: 8357-8397.
68. The gNA of embodiment 62, wherein the gNA specifically targets a promoter of the B2M gene.
69. The gNA of embodiment 68, wherein the targeting sequence shares about 80%, about 85%, about 90%, about 95%, about 96%, about 97%, about 98%, or about 99% identity to a sequence selected from SEQ ID NOs: 8398-8470.
70. The gNA of embodiment 68, wherein the targeting sequence is selected from SEQ ID NOs: 8398-8470.
71. The gNA of embodiment 62, wherein the targeting sequence shares about 80%, about 85%, about 90%, about 95%, about 96%, about 97%, about 98%, or about 99% identity to SEQ ID NO: 8357-8365.
72. The gNA of embodiment 62, wherein the targeting sequence is selected from SEQ ID NO: 8357-8365.
73. The gNA of any one of embodiments 62 to 72, wherein the gNA scaffold sequence binds a nucleic acid guided endonuclease selected from the group consisting of Cast, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and homologs thereof, or modified versions thereof
74. The gNA of embodiment 73, wherein the nucleic acid guided endonuclease is Cas9.
75. The gNA of embodiment 73 or 74, wherein the target sequence is proximal to a PAM sequence.
76. The gNA of embodiment 75, wherein the PAM sequence is NGG, NGCG, NGAG, NGAN, NGNG, NG, GAA, GAT, NNGRRT, NNGRRN, TTTV, TYCV, TATV, NNNNRYAC, NNNNGATT, NNAGAAW, or NAAAAC, wherein N represents any nucleotide; V represents A, G, or C; R represents A or G; Y represents C or T; and W represents A or T.
77. The gNA of any one of embodiments 62-76, further comprising a scaffold sequence.
78. The gNA of embodiment 77, wherein the scaffold sequence is encoded by a sequence of

(SEQ ID NO: 8345)
GTTTTAGAGCTAGAAATAGCAAGTTAAAATAAGGCTAGTCCGTTATCAAC
TTGAAAAAGTGGCACCGAGTCGGTGCTTTTTTT.

79. A polynucleotide encoding the gNA of embodiments 62-78.
80. The method of any one of embodiments 37-71, comprising transfecting the immune cell with 2 or more guide nucleic acids (gNAs) complementary to different target sequences within the B2M gene.
81. A method of producing an immune cell with reduced autocrine binding/signaling comprising:

transfecting the immune cell with at least one guide nucleic acid (gNA) complementary to a target sequence within the B2M gene, wherein the gNA is in complex with a nucleic acid-guided endonuclease,

wherein the gNA binds to the target sequence and the nuclease cleaves the target

sequence, thereby producing a modified B2M gene.

### Representative Embodiments—Set 4

1. An immune cell comprising

an inhibitory receptor comprising a ligand binding domain specific to a class I major histocompatibility complex (MHC-I) molecule, or a peptide-MHC complex thereof;

wherein expression and/or function of a human leukocyte antigen (HLA) polypeptide, or an allele thereof, in said immune cell has been reduced or eliminated.
2. The immune cell of embodiment 1, wherein the HLA allele is an HLA-A, HLA-B, HLA-C, and/or HLA-E allele.
3. The immune cell of embodiment 2, wherein the HLA-A allele is selected from HLA-A*02, HLA-A*02:01, HLA-A*02:01:01, and HLA-A*02:01:01:01.
4. The immune cell of embodiment 3, wherein the HLA-A allele is HLA-A*02:01:01:01.
5. The immune cell of any one of embodiments 1-4, further comprising an interfering RNA, comprising a sequence complementary to a sequence of a HLA-A*02:01:01:01 mRNA.
6. The immune cell of embodiment 5, wherein the interfering RNA is capable of inducing RNAi-mediated degradation of the HLA-A*02:01:01:01 mRNA.
7. The immune cell of embodiment 6, wherein the interfering RNA is a short hairpin RNA (shRNA).
8. The immune cell of embodiment 7, wherein the shRNA comprises
   a. a first sequence, having from 5′ end to 3′ end a sequence complementary to the HLA-A*02:01:01:01 mRNA; and
   b. a second sequence, having from 5′ end to 3′ end a sequence complementary to the first sequence, wherein the first sequence and the second sequence form the shRNA.
9. The immune cell of embodiment 8, wherein the HLA-A*02:01:01:01 mRNA sequence comprises a coding sequence.
10. The immune cell of embodiment 8, wherein the HLA-A*02:01:01:01 mRNA sequence comprises an untranslated region.
11. The immune cell of any one of embodiments 1 to 10, wherein the first sequence is 18, 19, 20, 21, or 22 nucleotides.
12. The immune cell of embodiment 11, wherein the first sequence is complementary to a sequence selected from SEQ ID NOs: 8476-16870.
13. The immune cell of embodiment 11, wherein the first sequence has GC content greater than or equal to 25% and less than 60%.
14. The immune cell of embodiment 13, wherein the first sequence is complementary to a sequence selected from SEQ ID NOs: 8476-12066.
15. The immune cell of embodiment 13, wherein the first sequence does not comprise 4 nucleotides of the same base or a run of seven C or G bases.
16. The immune cell of embodiment 15, wherein the first sequence is complementary to a sequence selected from SEQ ID NOs: 8476-11584.
17. The immune cell of embodiment 15, wherein the first sequence is selected from SEQ ID NOs: 8476-8754 or SEQ ID NOs: 8476-8561.
18. The immune cell of any one of embodiments 8 to 17, wherein the first sequence and second sequence are present on a single stranded polynucleotide, wherein the first sequence and second sequence are separated by 4, 5, 6, 7, 8, 9, 10, 11, 12, 13, 14, or nucleotides, wherein the 4, 5, 6, 7, 8, 9, 10, 11, 12, 13, 14, or 15 nucleotides form a loop region in the shRNA.
19. The immune cell of embodiment 18, wherein the loop region comprises a sequence selected from SEQ ID NOs: 16872-16884 and 16895.
20. The immune cell of any one of embodiments 1 to 19, wherein the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence.
21. The immune cell of embodiment 20, wherein the 5′ flank sequence is selected from SEQ ID NO: 16885-16887.
22. The immune cell of embodiment 20 or 21, wherein the 3′ flank sequence is selected from SEQ ID NO: 16888, 16889, and 16896.
23. The immune cell of any one of embodiments 1 to 22, wherein the shRNA is operably linked to a promoter.
24. The immune cell of embodiment 23, wherein the promoter is selected from a U6 promoter, a wild-type H1 promoter, a wild-type 7SK promoter, an Ef1a promoter.
25. The immune cell of embodiment 24, wherein the promoter sequence is selected from SEQ ID NOs: 16890-16893.
26. The immune cell of any one of the preceding embodiments, wherein the immune cell is a T cell, B cell, or Natural Killer (NK) cell.
27. The immune cell of any one of embodiments 1 to 26, wherein the immune cell is autologous to a subject.
28. The immune cell of any one of embodiments 1 to 26, wherein the immune cell is allogeneic to a subject.
29. The immune cell of any one of embodiments 1 to 26, wherein the immune cell is non-natural.
30. The immune cell of any one of the preceding embodiments, wherein the immune cell is isolated.
31. The immune cell of any one of the preceding embodiments, for use as a medicament.
32. The immune cell of embodiment 31, wherein the medicament is for the treatment of cancer in a subject in need thereof
33. A pharmaceutical composition, comprising a plurality of the immune cells of any one of embodiments 1-30.
34. The pharmaceutical composition of embodiment 31, comprising a pharmaceutically acceptable carrier, diluent, or excipient.
35. The pharmaceutical composition of embodiment 33 or 34, comprising a therapeutically effective amount of the immune cells.
36. A method of treating cancer with an adoptive cell therapy, comprising administering to the subject a plurality of the immune cell of any one of embodiments 1-30 or the pharmaceutical composition of any one of embodiments 33-35.
37. A vector comprising a promoter operably linked to the interfering RNA of any one of embodiments 1 to 25.
38. A vector comprising an interfering RNA, comprising an shRNA that targets a HLA-A*02:01:01:01 mRNA sequence, wherein the shRNA comprises
   a. a first sequence, having from 5′ to 3′ a sequence complementary to the HLA-A*02:01:01:01 mRNA; and
   b. a second sequence, having from 5′ to 3′ end a sequence complementary to the first sequence, wherein the first sequence and the second sequence form the shRNA.
39. The vector embodiment 38, wherein the HLA-A*02:01:01:01 mRNA sequence comprises a coding sequence.
40. The vector of embodiment 38, wherein the HLA-A*02:01:01:01 mRNA sequence comprises an untranslated region.
41. The vector of any one of embodiments 38 to 40, wherein the first sequence is 18, 19, 21, or 22 nucleotides.
42. The vector of embodiment 41, wherein the first sequence is complementary to a sequence selected from SEQ ID NOs: 8476-16870.
43. The vector of embodiment 41, wherein the first sequence has GC content greater than or equal to 25% and less than 60%.
44. The vector of embodiment 41, wherein the first sequence is complementary to a sequence selected from SEQ ID NOs: 8476-12066.
45. The vector of embodiment 43, wherein the first sequence does not comprise 4 nucleotides of the same base or a run of seven C or G bases.
46. The vector of embodiment 43, wherein the first sequence is complementary to a sequence selected from SEQ ID NOs: 8476-11584.
47. The vector of embodiment 45, wherein the first sequence is complementary to a sequence selected from SEQ ID NOs: 8476-8754 or 8476-8561.
48. The vector of any one of embodiments 38 to 47, wherein the first sequence and second sequence are present on a single stranded polynucleotide, wherein the first sequence and second sequence are separated by 4, 5, 6, 7, 8, 9, 10, 11, 12, 13, 14, or nucleotides, wherein the 4, 5, 6, 7, 8, 9, 10, 11, 12, 13, 14, or 15 nucleotides form a loop region in the shRNA.
49. The vector cell of embodiment 48, wherein the loop region comprises a sequence selected from SEQ ID NOs: 16872-16884 and 16895.
50. The vector of any one of embodiments 38 to 49, wherein the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence.
51. The vector of embodiment 50, wherein the 5′ flank sequence is selected from SEQ ID NO: 16885-16887.
52. The vector of embodiment 50 or 51, wherein the 3′ flank sequence is selected from SEQ ID NO: 16888, 16889, and 16896.
53. The vector of any one of embodiments 38 to 52, wherein the shRNA is operably linked to a promoter.
54. The vector of embodiment 53, wherein the promoter is selected from a U6 promoter, a wild-type H1 promoter, a wild-type 7SK promoter, an Ef1a promoter.
55. The vector of embodiment 54, wherein the promoter sequence is selected from SEQ ID NOs: 16890-16893.
56. The vector of any one of embodiments 38 to 55, wherein the vector is a viral vector.
57. The vector of embodiment 56, wherein the viral vector is a lentiviral vector.
58. The vector of any one of embodiments 38 to 57, further comprising a polynucleotide encoding an inhibitory receptor comprising a ligand binding domain specific to a class I major histocompatibility complex (MHC-I) molecule, or a peptide-MHC complex thereof
59. A method of producing an immune cell with reduced autocrine binding/signaling comprising transducing and/or transfecting the immune cell with the vector of any one of embodiments 38 to 58.
60. The method of embodiment 59, comprising transducing the immune cell with a first vector comprising a sequence encoding an activator receptor and a second vector comprising a sequence encoding an inhibitory receptor, thereby producing an immune cell expressing the activator and inhibitory receptors.
61. The method of embodiment 59, wherein, prior to the transducing and/or transfecting steps, the immune cell comprises a polynucleotide or vector encoding an interfering RNA targeting a HLA-A mRNA.
62. The method of embodiments 60 or 61, wherein the inhibitory receptor specifically binds to an HLA-A*02 pMHC antigen and the target gene comprises HLA-A*02.
63. A method of manufacturing a composition comprising immune cells with reduced autocrine binding/signaling comprising:
   a. providing immune cells from a subject suffering from or at risk for cancer or a hematological malignancy; and
   b. transducing and/or transfecting the immune cell with the vector of embodiments 38 to 58.
64. The method of embodiment 63, wherein, prior to the transducing and/or transfecting steps, the immune cell comprises a polynucleotide encoding activator and/or inhibitory receptors.
65. The method of 63, further comprising transducing the immune cell with a first vector comprising a sequence encoding the activator receptor and a second vector comprising a sequence encoding the inhibitory receptor, thereby producing an immune cell expressing the activator and inhibitory receptors.
66. A method of treating a subject in need thereof comprising:
   a. providing immune cells from a subject suffering from or at risk for cancer or a hematological malignancy
   b. transducing the immune cell with the vector of embodiments 38 to 58; and
   c. administering the immune cell to the subject.

### Representative Embodiments—Set 5

1. An immune cell comprising

an inhibitory receptor comprising a ligand binding domain specific to a class I major histocompatibility complex (MHC-I) molecule, or a peptide-MHC complex thereof;

wherein expression and/or function of beta-2-microglobulin (B2M) in said immune cell has been reduced or eliminated.
2. The immune cell of embodiment 1, further comprising an interfering RNA, comprising a sequence complementary to a sequence of a B2M mRNA.
3. The immune cell of embodiment 2, wherein the interfering RNA is capable of inducing RNAi-mediated degradation of the B2M mRNA.
4. The immune cell of embodiment 3, wherein the interfering RNA is a short hairpin RNA (shRNA).
5. The immune cell of embodiment 4, wherein the shRNA comprises
   a. a first sequence, having from 5′ end to 3′ end a sequence complementary to the B2M mRNA; and
   b. a second sequence, having from 5′ end to 3′ end a sequence complementary to the first sequence,

wherein the first sequence and the second sequence form the shRNA.
6. The immune cell of embodiment 5, wherein the B2M mRNA sequence comprises a part of a coding sequence.
7. The immune cell of embodiment 5, wherein the B2M mRNA sequence comprises a part of an untranslated region.
8. The immune cell of any one of embodiments 1 to 7, wherein the first sequence is 18, 19, 20, 21, or 22 nucleotides.
9. The immune cell of embodiment 8, wherein the first sequence is complementary to a sequence selected from SEQ ID NOs: 16897-21508, 847-8474, and 8368-8370.
10. The immune cell of embodiment 8, wherein the first sequence has GC content greater than or equal to 25% and less than 60%.
11. The immune cell of embodiment 10, wherein the first sequence is complementary to a sequence selected from SEQ ID NOs: 16897-20484.
12. The immune cell of embodiment 10, wherein the first sequence does not comprise 4 nucleotides of the same base or a run of seven C or G bases.
13. The immune cell of embodiment 12, wherein the first sequence is complementary to a sequence selected from SEQ ID NOs: 16897-19888.
14. The immune cell of embodiment 12, wherein the first sequence is 21 nucleotides.
15. The immune cell of embodiment 14, wherein the first sequence is complementary to a sequence selected from SEQ ID NOs: 16897-17478.
16. The immune cell of embodiment 14, wherein the first sequence is selected from SEQ ID NOs: 16897-17178 or SEQ ID NOs: 16897-17034.
17. The immune cell of any one of embodiments 5 to 16, wherein the first sequence and second sequence are present on a single stranded polynucleotide, wherein the first sequence and second sequence are separated by 4, 5, 6, 7, 8, 9, 10, 11, 12, 13, 14, or nucleotides, wherein the 4, 5, 6, 7, 8, 9, 10, 11, 12, 13, 14, or 15 nucleotides form a loop region in the shRNA.
18. The immune cell of embodiment 17, wherein the loop region comprises a sequence selected from SEQ ID NOs: 16872-16884, and 16895.
19. The immune cell of any one of embodiments 1 to 18, wherein the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence.
20. The immune cell of embodiment 19, wherein the 5′ flank sequence is selected from SEQ ID NO: 16885-16887, and 16894.
21. The immune cell of embodiment 19 or 20, wherein the 3′ flank sequence is selected from SEQ ID NO: 16888, 16889, and 16896.
22. The immune cell of any one of embodiments 1 to 21, wherein the shRNA is operably linked to a promoter.
23. The immune cell of embodiment 22, wherein the promoter is a mammalian promoter, viral promoter or synthetic promoter.
24. The immune cell of embodiment 22, wherein the promoter is selected from a U6 promoter, a wild-type H1 promoter, a wild-type 7SK promoter, an Ef1a promoter.
25. The immune cell of embodiment 24, wherein the promoter sequence is selected from SEQ ID NOs: 16890-16893.
26. The immune cell of any one of the preceding embodiments, wherein the immune cell is a T cell, B cell, or Natural Killer (NK) cell.
27. The immune cell of any one of embodiments 1 to 26, wherein the immune cell is autologous to a subject.
28. The immune cell of any one of embodiments 1 to 26, wherein the immune cell is allogeneic to a subject.
29. The immune cell of any one of embodiments 1 to 26, wherein the immune cell is non-natural.
30. The immune cell of any one of the preceding embodiments, wherein the immune cell is isolated.
31. The immune cell of any one of the preceding embodiments, for use as a medicament.
32. The immune cell of embodiment 30, wherein the medicament is for the treatment of cancer in a subject in need thereof
33. A pharmaceutical composition, comprising a plurality of the immune cells of any one of embodiments 1-32.
34. The pharmaceutical composition of embodiment 33, comprising a pharmaceutically acceptable carrier, diluent, or excipient.
35. The pharmaceutical composition of embodiment 33 or 34, comprising a therapeutically effective amount of the immune cells.
36. A method of treating cancer with an adoptive cell therapy, comprising administering to the subject a plurality of the immune cell of any one of embodiments 1-32 or the pharmaceutical composition of any one of embodiments 33-35.
37. A vector comprising the interfering RNA of any one of embodiments 1 to 25.
38. A vector comprising an interfering RNA, wherein the interfering RNA comprises an shRNA that targets a B2M mRNA sequence, wherein the shRNA comprises
   a. a first sequence, having from 5′ to 3′ a sequence complementary to the B2M mRNA; and
   b. a second sequence, having from 5′ to 3′ end a sequence complementary to the first sequence,

wherein the first sequence and the second sequence form the shRNA.
39. The vector of embodiment 38, wherein the B2M mRNA sequence comprises a coding sequence.
40. The vector of embodiment 38, wherein the B2M mRNA sequence comprises an untranslated region.
41. The vector of any one of embodiments 38-40, wherein the first sequence is 18, 19, 20, 21, or 22 nucleotides.
42. The vector of embodiment 41, wherein the first sequence is complementary to a sequence selected from SEQ ID NOs: 16897-21508, 847-8474, and 8368-8370.
43. The vector of embodiment 41, wherein the first sequence has GC content greater than or equal to 25% and less than 60%.
44. The vector of embodiment 43, wherein the first sequence is complementary to a sequence selected from SEQ ID NOs: 16897-20484.
45. The vector of embodiment 43, wherein the first sequence does not comprise 4 nucleotides of the same base or a run of seven C or G bases.
46. The vector of embodiment 45, wherein the first sequence is complementary to a sequence selected from SEQ ID NOs: 16897-19888.
47. The vector of embodiment 45, wherein the first sequence is 21 nucleotides.
48. The vector of embodiment 47, wherein the first sequence is complementary to a sequence selected from SEQ ID NOs: 16897-17478.
49. The vector of embodiment 47, wherein the first sequence is complementary to a sequence selected from SEQ ID NOs: 16897-17178 or 16897-17034.
50. The vector of any one of embodiments 38-49, wherein the first sequence and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA.
51. The vector of embodiment 50, wherein the loop region comprises a sequence selected from SEQ ID NOs: 16872-16884, and 16895.
52. The vector of any one of embodiments 38-51, wherein the shRNA further comprises a flank sequence and a 3′ flank sequence, wherein the 5′ flank sequence is joined to the 5′ end of the first sequence, and wherein the 3′ flank sequence is joined to the 3′ end of the second sequence.
53. The vector of embodiment 52, wherein the 5′ flank sequence is selected from SEQ ID NO: 16885-16887, and 16894.
54. The vector of embodiment 52 or 53, wherein the 3′ flank sequence is selected from SEQ ID NO: 16888, 16889, and 16896.
55. The vector of any one of embodiments 38-54, wherein the shRNA is operably linked to a promoter.
56. The vector of embodiment 55, wherein the promoter is a mammalian promoter, viral promoter or synthetic promoter.
57. The vector of embodiment 55, wherein the promoter is selected from a U6 promoter, a wild-type H1 promoter, a wild-type 7SK promoter, an Ef1a promoter.
58. The vector of embodiment 55, wherein the promoter sequence is selected from SEQ ID NOs: 16890-16893.
59. The vector of any one of embodiments 38-58, wherein the vector is a viral vector.
60. The vector of embodiment 59, wherein the viral vector is a lentiviral vector.
61. The vector of any one of embodiments 38-60, further comprising a polynucleotide encoding an inhibitory receptor comprising a ligand binding domain specific to a class I major histocompatibility complex (MHC-I) molecule, or a peptide-MHC complex thereof
62. A method of producing an immune cell with reduced autocrine binding/signaling comprising transducing and/or transfecting the immune cell with the vector of any one of embodiments 38-61.
63. The method of embodiment 62, comprising transducing the immune cell with a first vector comprising a sequence encoding an activator receptor and a second vector comprising a sequence encoding an inhibitory receptor, thereby producing an immune cell expressing the activator and inhibitory receptors.
64. The method of embodiment 62, wherein, prior to the transducing and/or transfecting steps, the immune cell comprises a polynucleotide or vector encoding an interfering RNA targeting a B2M mRNA.
65. The method of embodiments 63 or 64, wherein the inhibitory receptor specifically binds to an HLA-A*02 pMHC antigen and the target gene comprises HLA-A*02.
66. A method of manufacturing a composition comprising immune cells with reduced autocrine binding/signaling comprising:
   a. providing immune cells from a subject suffering from or at risk for cancer or a hematological malignancy; and
   b. transducing and/or transfecting the immune cell with the vector of embodiments 38-61.
67. The method of embodiment 66, wherein, prior to the transducing and/or transfecting steps, the immune cell comprises a polynucleotide encoding activator and/or inhibitory receptors.
68. The method of 66, further comprising transducing the immune cell with a first vector comprising a sequence encoding the activator receptor and a second vector comprising a sequence encoding the inhibitory receptor, thereby producing an immune cell expressing the activator and inhibitory receptors.
69. A method of treating a subject in need thereof comprising:
   a. providing immune cells from a subject suffering from or at risk for cancer or a hematological malignancy;
   b. transducing the immune cell with the vector of embodiments 38-61; and
   c. administering the immune cell to the subject.

What is claimed is:

1. An allogeneic immune cell comprising:
a. a first engineered receptor, the first engineered receptor comprising a transmembrane region and an extracellular region, the extracellular region comprising a first ligand binding domain capable of specifically binding a first ligand; and
b. a second engineered receptor, the second engineered receptor comprising a transmembrane region and an extracellular region, the extracellular region comprising a second ligand binding domain capable of specifically binding a second ligand,
wherein binding of the first ligand binding domain to the first ligand activates or promotes activation of the immune cell by the first receptor,
wherein binding of the second ligand binding domain to the second ligand inhibits activation of the immune cell by the first receptor, and
wherein the second ligand is expressed by a host immune cell.

2. The allogeneic immune cell of claim 1, wherein the allogeneic immune cell expresses one or more endogenous T cell receptors (TCRs).

3. The allogeneic immune cell of claim 2, wherein the allogeneic immune cell has not been modified to reduce or eliminate the expression of an endogenous TRCA, TRB, CD3D, CD3E, CD3G and/or CD3Z gene product.

4. The allogeneic immune cell of claim 2 or 3, wherein binding of the second ligand binding domain to the second ligand inhibits activation of the immune cell by the endogenous TCR.

5. The allogeneic immune cell of claim 4, wherein expression of the second engineered receptor reduces graft versus host disease when a plurality of the allogeneic immune cells are administered to a subject.

6. The allogeneic immune cell of any one of claims 1-5, comprising a first modification that reduces or eliminates expression or function of a component of the major histocompatibility complex class I (MHC I).

7. The allogeneic immune cell of claim 6, wherein the component of MHC I is human leukocyte antigen A (HLA-A), human leukocyte antigen B (HLA-B), human leukocyte antigen C (HLA-C) or beta-2-microglobulin (B2M).

8. The allogeneic immune cell of claim 6 or 7, wherein the first modification comprises a genetic modification of a HLA-A, HLA-B, HLA-C or B2M locus of the allogeneic immune cell genome.

9. The allogeneic immune cell of claim 8, wherein the genetic modification comprises a deletion, insertion, substitution or frameshift mutation in the HLA-A, HLA-B, HLA-C or B2M locus.

10. The allogeneic immune cell of claim 6-9, wherein the first modification reduces expression of a functional protein encoded by the HLA-A, HLA-B, HLA-C or B2M locus.

11. The allogeneic immune cell of claim of any one of claims 6-10, wherein the first modification comprises using a nucleic acid guided endonuclease, a zinc finger nuclease or a TALEN.

12. The allogeneic immune cell of claim 11, wherein the nucleic acid guided endonuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, CasY, Csx3, Csx1, Csx15, Csf1, Csf2, Csf3, and Csf4.

13. The allogeneic immune cell of claim 11, wherein the nucleic acid guided endonuclease is Cas9.

14. The allogeneic immune cell of any one of claims 11-13, wherein the allogeneic immune cell is modified with a nucleic acid guided endonuclease in a complex with a guide nucleic acid (gNA) that specifically targets a sequence of the HLA-A, HLA-B, HLA-C or B2M locus.

15. The allogeneic immune cell of any one of claims 11-13, wherein the allogeneic immune cell is modified with a nucleic acid guided endonuclease in a complex with at least one guide gNA that specifically targets a sequence within the B2M locus and/or a promoter of the B2M gene.

16. The allogeneic immune cell of claim 15, wherein the at least one gNA comprises a sequence that shares about 80%, about 85%, about 90%, about 95%, about 96%, about 97%, about 98%, or about 99% identity to a sequence selected from the group consisting of SEQ ID NOs: 8357-8470.

17. The allogeneic immune cell of claim 15, wherein the at least one gNA comprises a sequence selected from the group consisting of SEQ ID NOs: 8357-8470.

18. The allogeneic immune cell of claim 15, wherein the at least one gNA specifically targets a coding sequence (CDS) of the B2M gene.

19. The allogeneic immune cell of claim 18, wherein the at least one gNA comprises a sequence that shares about 80%, about 85%, about 90%, about 95%, about 96%, about 97%, about 98%, or about 99% identity to a sequence selected from the group consisting of SEQ ID NOs: 8357-8397.

20. The allogeneic immune cell of claim 18, wherein the gNA comprises a sequence selected from the group consisting of SEQ ID NOs: 8357-8397.

21. The allogeneic immune cell of any one of claims 11-13, wherein the allogeneic immune cell is modified with a nucleic acid guided endonuclease in a complex with at least one guide gNA that specifically targets a sequence within the HLA-A locus and/or a promoter of the HLA-A gene.

22. The allogeneic immune cell of claim 21, wherein the at least one gNA is specific to a target sequence that shares about 90%, about 95%, about 96%, about 97%, about 98%, or about 99% identity to a sequence selected from the group consisting of SEQ ID NOs: 390-3276.

23. The allogeneic immune cell of claim 21, wherein the at least one gNA is specific to a target sequence selected from the group consisting of SEQ ID NOs: 390-3276.

24. The allogeneic immune cell of claim 21, wherein the allogeneic immune cell is modified with a nucleic acid guided endonuclease in a complex with at least one gNA that specifically targets a sequence of HLA-A*02 alleles.

25. The allogeneic immune cell of claim 24, wherein the at least one gNA is specific to a target sequence that shares about 90%, about 95%, about 96%, about 97%, about 98%, or about 99% identity to a sequence selected from the group consisting of SEQ ID NOs: 390-1585.

26. The allogeneic immune cell of claim 24, wherein the at least one gNA is specific to a target sequence selected from the group consisting of SEQ ID NOs: 390-1585.

27. The allogeneic immune cell claim 21, wherein the allogeneic immune cell is modified with a nucleic acid guided endonuclease in a complex with at least one gNA that specifically targets a sequence of HLA-A*02:01 alleles.

28. The allogeneic immune cell of claim 27, wherein the at least one gNA is specific to a sequence that shares about 90%, about 95%, about 96%, about 97%, about 98%, or about 99% identity to target a sequence selected from the group consisting of SEQ ID NOs: 390-1174.

29. The allogeneic immune cell of claim 27, wherein the at least one gNA is specific to a target sequence selected from the group consisting of SEQ ID NOs: 390-1174.

30. The allogeneic immune cell of claim 21, wherein the allogeneic immune cell is modified with a nucleic acid guided endonuclease in a complex with at least one gNA that specifically targets a sequence of HLA-A*02:01:01 alleles.

31. The allogeneic immune cell of claim 30, wherein the at least one gNA is specific to a target sequence that shares about 90%, about 95%, about 96%, about 97%, about 98%, or about 99% identity to a sequence selected from the group consisting of SEQ ID NOs: 390-1166.

32. The allogeneic immune cell of claim 30, wherein the at least one gNA is specific to a target sequence selected from the group consisting of SEQ ID NOs: 390-1166.

33. The allogeneic immune cell of claim 21, wherein the allogeneic immune cell is modified with a nucleic acid guided endonuclease in a complex with at least one gNA that specifically targets a sequence of HLA-A*02:01:01:01 alleles.

34. The allogeneic immune cell of claim 33, wherein the at least one gNA is specific to a target sequence that shares about 90%, about 95%, about 96%, about 97%, about 98%, or about 99% identity to a sequence selected from the group consisting of SEQ ID NOs: 390-1126.

35. The allogeneic immune cell of claim 33, wherein the at least one gNA is specific to a target sequence selected from the group consisting of SEQ ID NOs: 390-1126.

36. The allogeneic immune cell of claim 21, wherein the allogeneic immune cell is modified with a nucleic acid guided endonuclease in a complex with at least one guide nucleic acid (gNA) that specifically targets a coding DNA sequence of HLA-A*02.

37. The allogeneic immune cell of claim 36, wherein the at least gNA is specific to a target sequence that shares about 90%, about 95%, about 96%, about 97%, about 98%, or about 99% identity to a sequence selected from the group consisting of SEQ ID NOs: 390-509.

38. The allogeneic immune cell of claim 36, wherein the at least one gNA is specific to a target sequence selected from the group consisting of SEQ ID NOs: 390-509.

39. The allogeneic immune cell of claim 21, wherein the immune cell is modified with a nucleic acid guided endonuclease in a complex with at least one gNA that specifically targets a coding DNA sequence that is shared by more than 1000 HLA-A*02 alleles.

40. The allogeneic immune cell of claim 39, wherein the at least one gNA is specific to a target sequence that shares about 90%, about 95%, about 96%, about 97%, about 98%, or about 99% identity to a sequence selected from the group consisting of SEQ ID NOs: 390-455.

41. The allogeneic immune cell of claim 39, wherein the at least one gNA is specific to a target sequence selected from the group consisting of SEQ ID NOs: 390-455.

42. The allogeneic immune cell of claim 6, wherein the first modification comprises expression of an interfering RNA.

43. The allogeneic immune cell of claim 42, wherein the interfering RNA is a small interfering RNA (siRNA), a short hairpin RNA (shRNA) or a microRNA.

44. The allogeneic immune cell of claim 42 or 43, wherein the interfering RNA comprises a sequence complementary to a target sequence of HLA-A, HLA-B, HLA-C or B2M.

45. The allogeneic immune cell of claim 44, wherein the target sequence of HLA-A, HLA-B, HLA-C or B2M is between 18 and 27 bp in length.

46. The allogeneic of any one of claims 42-45, wherein the interfering RNA comprises an shRNA capable of inducing RNAi-mediated degradation of an HLA-A*02:01:01 mRNA.

47. The allogeneic immune cell of claim 46, wherein the shRNA comprises
a. a first sequence, having from 5′ end to 3′ end a sequence complementary to the HLA-A*02:01:01:01 mRNA; and
b. a second sequence, having from 5′ end to 3′ end a sequence complementary to the first sequence,
wherein the first sequence and the second sequence form the shRNA.

48. The allogeneic immune cell of claim 46 or 47, wherein the first sequence is 18, 19, 21, or 22 nucleotides.

49. The allogeneic immune cell of claim 48, wherein the first sequence is complementary to a sequence selected from SEQ ID NOs: 8476-16870.

50. The allogeneic immune cell of any one of claims 47-49, wherein the first sequence and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA.

51. The immune allogeneic cell of claim 50, wherein the loop region comprises a sequence selected from SEQ ID NOs: 16872-16884 and 16895.

52. The allogeneic immune cell of any one of claims 47-51, wherein the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence.

53. The allogeneic immune cell of claim 52, wherein the 5′ flank sequence is selected from the group consisting of SEQ ID NO: 16885-16887.

54. The allogeneic immune cell of claim 52 or 53, wherein the 3′ flank sequence is selected from the group consisting of SEQ ID NO: 16888, 16889, and 16896.

55. The allogeneic of any one of claims 42-45, wherein the interfering RNA comprises an shRNA capable of inducing RNAi-mediated degradation of a B2M mRNA.

56. The allogeneic immune cell of claim 55, wherein the shRNA comprises
a. a first sequence, having from 5′ end to 3′ end a sequence complementary to the B2M mRNA; and
b. a second sequence, having from 5′ end to 3′ end a sequence complementary to the first sequence,
wherein the first sequence and the second sequence form the shRNA.

57. The allogeneic immune cell of claim 56, wherein the first sequence is complementary to a sequence selected from SEQ ID NOs: 16897-21508, 847-8474, and 8368-8370.

58. The allogeneic immune cell of claim 56, wherein the first sequence is complementary to a sequence selected from the group consisting of SEQ ID NOs: 16897-20484.

59. The allogeneic immune cell of claim 56, wherein the first sequence is complementary to a sequence selected from the group consisting of SEQ ID NOs: 16897-19888.

60. The allogeneic immune cell of claim 56, wherein the first sequence is complementary to a sequence selected from the group consisting of SEQ ID NOs: 16897-17478.

61. The allogeneic immune cell of claim 56, wherein the first sequence is selected from the group consisting of SEQ ID NOs: 16897-17178 or SEQ ID NOs: 16897-17034.

62. The allogeneic immune cell of any one of claims 56-61, wherein the first sequence and second sequence are present on a single stranded polynucleotide, wherein the first sequence and second sequence are separated by 4, 5, 6, 7, 8, 9, 10, 11, 12, 13, 14, or 15 nucleotides, wherein the 4, 5, 6, 7, 8, 9, 10, 11, 12, 13, 14, or 15 nucleotides form a loop region in the shRNA.

63. The allogeneic immune cell of claim 62, wherein the loop region comprises a sequence selected from SEQ ID NOs: 16872-16884, and 16895.

64. The allogeneic immune cell of any one of claims 56-63, wherein the shRNA further comprises a 5′ flank sequence and a 3′ flank sequence, wherein the 5′ flank sequence is joined to the 5′ end of the first sequence, and wherein the 3′ flank sequence is joined to the 3′ end of the second sequence.

65. The allogeneic immune cell of claim 64, wherein the 5′ flank sequence is selected from SEQ ID NO: 16885-16887 and 16894.

66. The allogeneic immune cell of claim 65 or 66, wherein the 3′ flank sequence is selected from SEQ ID NO: 16888, 16889, and 16896.

67. The allogeneic immune cell of claim 56, wherein the shRNA comprises SEQ ID NOs: 21899-21901.

68. The allogeneic immune cell of any one of claims 42-67, wherein the interfering RNA is operably linked to a promoter.

69. The allogeneic immune cell of any one of claims 1-68, comprising a second modification that reduces or eliminates expression or function of CD52.

70. The allogeneic immune cell of claim 69, wherein the second modification comprises a deletion, insertion, substitution or frameshift mutation in the CD52 locus of the allogeneic immune cell genome.

71. The allogeneic immune cell of claim of claim 69 or 70, wherein the second modification comprises using a nucleic acid guided endonuclease, a zinc finger nuclease or a TALEN.

72. The allogeneic immune cell of claim 69, wherein the second modification comprises expression of an interfering RNA.

73. The allogeneic immune cell of claim 72, wherein the interfering RNA is a small interfering RNA (siRNA), a short hairpin RNA (shRNA) or a microRNA.

74. The allogeneic immune cell of claim 72 or 73, wherein the interfering RNA comprises a sequence complementary to a target sequence of CD52.

75. The allogeneic immune cell of any one of claims 1-74, comprising a third modification that reduces targeting of the allogeneic immune cell by NK cells of a subject.

76. The allogeneic immune cell of claim 75, wherein the third modification comprises overexpression of HLA-E, HLA-G or NKG2A.

77. The allogeneic immune cell of any one of claims 1-76, wherein the second ligand is not expressed in a target cell due to loss of heterozygosity of a gene encoding the second ligand.

78. The allogeneic immune cell of any one of claims 1-76, wherein the first ligand and second ligand are not the same.

79. The allogeneic immune cell of any one of claims 1-78, wherein the first ligand is expressed by target cells.

80. The allogeneic immune cell of any one of claims 1-79, wherein the first ligand is expressed by target cells and a plurality of non-target cells.

81. The allogeneic immune cell of claim 80, wherein the plurality of non-target cells express both the first and second ligands.

82. The allogeneic immune cell of any one of claims 1-81, wherein the second ligand is not expressed by the target cells, and is expressed by the plurality of non-target cells.

83. The allogeneic immune cell of any one of claims 80-82, wherein the target cells are cancer cells and the non-target cells are non-cancerous cells.

84. The allogeneic immune cell of any one of claims 1-83, wherein the first ligand is selected from the group consisting of a cell adhesion molecule, a cell-cell signaling molecule, an extracellular domain, a molecule involved in chemotaxis, a glycoprotein, a G protein-coupled receptor, a transmembrane protein, a receptor for a neurotransmitter and a voltage gated ion channel, or a peptide antigen thereof.

85. The allogeneic immune cell of any one of claims 1-83, wherein the first ligand is a cancer antigen.

86. The allogeneic immune cell of any one of claims 1-83, wherein the first ligand is selected from the group of antigens in Table 1.

87. The allogeneic immune cell of claim 86, wherein the first ligand binding domain is isolated or derived from the antigen binding domain of an antibody in Table 1.

88. The allogeneic immune cell of any one of claims 1-83, wherein the first ligand is selected from the group consisting of transferrin receptor (TFRC), epidermal growth factor receptor (EGFR), CEA cell adhesion molecule 5 (CEA), CD19 molecule (CD19), erb-b2 receptor tyrosine kinase 2 (HER2), and mesothelin (MSLN), or a peptide antigen thereof.

89. The allogeneic immune cell of any one of claims 1-83, wherein the first ligand is a pan-HLA ligand.

90. The allogeneic immune cell of any one of claims 1-83, wherein the first ligand comprises HLA-A, HLA-B, HLA-C, HLA-E, HLA-F, of HLA-G.

91. The allogeneic immune cell of any one of claims 1-90, wherein the first engineered receptor is a T cell receptor (TCR) or a chimeric antigen receptor (CAR).

92. The allogeneic immune cell of any one of claims 1-91, wherein the second engineered receptor is a T cell receptor (TCR) or a chimeric antigen receptor (CAR).

93. The allogeneic immune cell of any one of claims 1-92, wherein the first ligand binding domain comprises a single chain FAT antibody fragment (ScFv) or a β chain variable domain (Vβ).

94. The allogeneic immune cell of any one of claims 1-92, wherein the first ligand binding domain comprises a TCR α chain variable domain and a TCR β chain variable domain.

95. The allogeneic immune cell of any one of claims 1-92, wherein the first ligand binding domain comprises a variable heavy chain (VH) domain and a variable light chain (VL) domain.

96. The allogeneic immune cell of claim 93, wherein the first ligand is EGFR or a peptide antigen thereof, and the first ligand binding domain comprises a sequence of SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115 or SEQ ID NO: 381, or a sequence having at least 90%, at least 95% or at least 99% identity thereto.

97. The allogeneic immune cell of claim 93, wherein the first ligand is MSLN or a peptide antigen thereof, and the first ligand binding domain comprises a sequence of SEQ ID NO: 83, SEQ ID NO: 85, SEQ ID NO: 87 or SEQ ID NO: 89, or a sequence having at least 90%, at least 95% or at least 99% identity thereto.

98. The allogeneic immune cell of claim 93, wherein the first ligand is CEA or a peptide antigen thereof, and the first ligand binding domain comprises SEQ ID NO: 91, SEQ ID NO: 93, SEQ ID NO: 95, SEQ ID NO: 97, SEQ ID NO: 273, SEQ ID NO: 275, or SEQ ID NO: 277, or a sequence having at least 90%, at least 95% or at least 99% identity thereto.

99. The allogeneic immune cell of claim 93, wherein the first ligand is CD19 or a peptide antigen thereof, and the first ligand binding domain comprises SEQ ID NO: 266 or SEQ ID NO: 268, or a sequence having at least 90%, at least 95% or at least 99% identity thereto.

100. The allogeneic immune cell of claim 93, wherein the first ligand comprises a pan-HLA ligand, and the first ligand binding domain comprises a sequence of SEQ ID NO: 163, SEQ ID NO: 165, SEQ ID NO: 167, SEQ ID NO: 169, SEQ ID NO: 171, or SEQ ID NO: 173, or a sequence having at least 90%, at least 95% or at least 99% identity thereto.

101. The allogeneic immune cell of claim 93, wherein the first ligand comprises EGFR or a peptide antigen thereof, and the first ligand binding domain comprises CDRs selected from SEQ ID NOs: 129-162.

102. The allogeneic immune cell of claim 93, wherein the first ligand comprises a CEA ligand, or a peptide antigen thereof, and the first ligand binding domain comprises CDRs selected from SEQ ID NOs: 285-293.

103. The allogeneic immune cell of any one of claims 1-102, wherein the second ligand binding domain comprises an ScFv, a Vβ domain, or a TCR α chain variable domain and a TCR β chain variable domain.

104. The allogeneic immune cell of any one of claims 1-102, wherein the second ligand binding domain comprises a variable heavy chain (VH) domain and a variable light chain (VL) domain.

105. The allogeneic immune cell of claim 103, wherein the second ligand comprises an HLA-A*02 allele, and wherein the second ligand binding domain comprises any one of SEQ ID NOs: 50-61 or a sequence having at least 90%, at least 95%, or at least 99% identity thereto.

106. The allogeneic immune cell of claim 103, wherein the second ligand comprises an HLA-A*02 allele, and the second ligand binding domain comprises CDRs selected from SEQ ID NOs: 39-49.

107. The allogeneic immune cell of any one of claim 1-106, wherein the second engineered receptor comprises at least one immunoreceptor tyrosine-based inhibitory motif (ITIM).

108. The allogeneic immune cell of any one of claims 1-107, wherein the second engineered receptor comprises a LILRB1 intracellular domain or a functional variant thereof.

109. The allogeneic immune cell of claim 108, wherein the LILRB1 intracellular domain comprises a sequence at least 95% identical to SEQ ID NO: 73.

110. The allogeneic immune cell of any one of claims 1-109, wherein the second engineered receptor comprises a LILRB1 transmembrane domain or a functional variant thereof.

111. The allogeneic immune cell of claim 110, wherein the LILRB1 transmembrane domain or a functional variant thereof comprises a sequence at least 95% identical to SEQ ID NO: 82.

112. The allogeneic immune cell of any one of claims 1-111, wherein the second engineered receptor comprises a LILRB1 hinge domain or functional fragment or variant thereof.

113. The allogeneic immune cell of claim 112, wherein the LILRB1 hinge domain comprises a sequence at least 95% identical to SEQ ID NO: 81, SEQ ID NO: 74 or SEQ ID NO: 75.

114. The allogeneic immune cell of any one of claims 1-113, wherein the second engineered receptor comprises a LILRB1 intracellular domain and a LILRB1 transmembrane domain, or a functional variant thereof.

115. The allogeneic immune cell of claim 114, wherein the LILRB1 intracellular domain and LILRB1 transmembrane domain comprises SEQ ID NO: 77 or a sequence at least 95% identical to SEQ ID NO: 77.

116. The allogeneic immune cell of any one of claims 105-115, wherein the second inhibitory receptor comprises a sequence of SEQ ID NO: 21902 or a sequence having at least 90%, at least 95% or at least 99% identity thereto.

117. The allogeneic immune cell of any one of claims 1-116, wherein the immune cell is selected form the group consisting of T cells, B cells and Natural Killer (NK) cells.

118. The allogeneic immune cell of any one of the preceding claims, wherein the immune cell is non-natural.

119. The allogeneic immune cells of any one of the preceding claims, wherein the immune cell is isolated.

120. The allogeneic immune cell of any one of the preceding claims, for use as a medicament.

121. The allogeneic immune cell of claim 120, wherein the medicament is for the treatment of cancer in a subject.

122. A pharmaceutical composition, comprising a plurality of the allogeneic immune cells of any one of claims 1-121.

123. The pharmaceutical composition of claim 122, comprising a pharmaceutically acceptable carrier, diluent or excipient.

124. The pharmaceutical composition of claim 122 or 123, comprising a therapeutically effective amount of the allogeneic immune cells.

125. A method of increasing the specificity of an adoptive cell therapy in a subject, comprising administering to the subject a plurality of the allogeneic immune cell of any one of claims 1-121 or the pharmaceutical composition of any one of claims 122-124.

126. A method of treating a subject with cancer with an adoptive cell therapy, comprising administering to the subject a plurality of the allogeneic immune cells of any one of claims 1-121 or the pharmaceutical composition of any one of claims 122-124.

127. The method of claim 126, wherein cells of the cancer express the first ligand.

128. The method of any one of claim 126 or 127, wherein cells of the cancer do not express the second ligand due to loss of heterozygosity.

129. The method of any one of claims 126-128, wherein non-target cells express both the first ligand and the second ligand.

130. The method of any one of claims 126-129, wherein immune cells of the subject express the second ligand.

131. The method of any one of claims 125-130, comprising administering a lymphodepletion agent to the subject.

132. The method of claim 131, wherein the lymphodepletion agent specifically targets CD52.

133. A method of making the allogeneic immune cell of any one of claims 1-121, comprising
a. providing a plurality of allogeneic immune cells; and
b. contacting the immune cells with a vector comprising sequences encoding:
i. a first engineered receptor comprising a transmembrane region and an extracellular region, the extracellular region comprising a first ligand binding domain capable of specifically binding a first ligand, and
ii. a second engineered receptor comprising a transmembrane region and an extracellular region, the extracellular region comprising a second ligand binding domain capable of specifically binding a second ligand;
wherein binding of the first ligand binding domain to the first ligand activates or promotes activation of the immune cell, and
wherein binding of the second ligand binding domain to a second ligand inhibits activation of the immune cell by the first ligand.

134. The method of claim 133, wherein the sequences of the first and second engineered receptors are operably linked to a first promoter.

135. The method of claim 133 or 134, wherein the vector further comprises a sequence encoding a self-cleaving peptide between the sequence encoding the first engineered receptor and the sequence encoding the second engineered receptor.

136. The method of any one of claims 133-135, wherein the vector further comprises a sequence encoding a B2M or HLA-A shRNA operably linked to a sequence promoter.

137. The method of any one of claims 133-136, wherein the vector further comprises a sequence encoding a guide nucleic acid (gNA) comprising a targeting sequence specific to a B2M or HLA-A*02 target sequence, wherein the sequence encoding the gNA is operably linked to a second promoter.

138. The method of claim 137, wherein the vector is a lentiviral vector, and contacting the immune cells with the vector comprises transducing the immune cells.

139. The method of claim 138, further comprising transfecting the immune cells with a Cas9 protein or a nucleic acid comprising a sequence encoding a Cas9 protein.

140. A kit comprising the allogeneic immune cell of any one of claims 1-121 or the pharmaceutical composition of any one of claims 122-124.

141. The kit of claim 140, further comprising instructions for use.

142. A vector comprising:
a. a sequence encoding a first engineered receptor, the first engineered receptor comprising a transmembrane region and an extracellular region, the extracellular region comprising a first ligand binding domain capable of specifically binding a first ligand;
b. a self-cleaving polypeptide sequence; and
c. a sequence encoding second engineered receptor, the second engineered receptor comprising a transmembrane region and an extracellular region, the extracellular region comprising a second ligand binding domain capable of specifically binding a second ligand,
wherein binding of the first ligand binding domain to the first ligand activates or promotes activation of the immune cell by the first receptor, and
wherein binding of the second ligand binding domain to the second ligand inhibits activation of the immune cell by the first receptor.

143. A vector comprising
a. a first promoter operably linked to:
i. a sequence encoding a first engineered receptor, the first engineered receptor comprising a transmembrane region and an extracellular region, the extracellular region comprising a first ligand binding domain capable of specifically binding a first ligand,
ii. a self-cleaving polypeptide sequence, and
iii. a sequence encoding second engineered receptor, the second engineered receptor comprising a transmembrane region and an extracellular region, the extracellular region comprising a second ligand binding domain capable of specifically binding a second ligand; and
b. a second promoter operably linked to a sequence encoding a guide nucleic acid or an short interfering RNA (shRNA) capable of reducing expression of HLA-A or B2M by an immune cell;
wherein binding of the first ligand binding domain to the first ligand activates or promotes activation of the immune cell by the first receptor,
wherein binding of the second ligand binding domain to the second ligand inhibits activation of the immune cell by the first receptor.
